(19) United States
(12) Patent Application Publication
(10) Pub. No.: US 2007/0014810 A1
(43) Pub. Date: Jan. 18, 2007

(54) Inducing cellular immune responses to human papillomavirus using peptide and nucleic acid compositions

(76) Inventors: Denise Baker, Poway, CA (US); Scott D. Power, San Bruno, CA (US); Mark J. Newman, Carlsbad, CA (US); Robert Chesnut, Cardiff-by-the Sea, CA (US); Scott Southwood, Santee, CA (US); Bianca Mothe, Oceanside, CA (US); Manley T.F. Huang, Palo Alto, CA (US); Lilia Maria Babe, Emerald Hills, CA (US); Lawrence M. DeYoung, Montara, CA (US); Yiyou Chen, San Jose, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/027,670

(22) Filed: Jan. 3, 2005

Related U.S. Application Data
(60) Provisional application No. 60/584,652, filed on Jul. 2, 2004.
(60) Provisional application No. 60/533,211, filed on Dec. 31, 2003.

(51) Int. Cl. C12Q 1/70; C07H 21/04; A61K 39/12; C12N 7/00; C07K 14/025

(52) U.S. Cl. 424/186.1; 435/5; 435/235.1; 530/350; 536/23.72

(57) ABSTRACT

This invention uses our knowledge of the mechanisms by which antigen is recognized by T cells to identify and prepare human papillomavirus (HPV) epitopes, and to develop epitope-based vaccines directed towards HPV. More specifically, this application communicates our discovery of pharmaceutical compositions and methods of use in the prevention and treatment of HPV infection.

### CROSS-REFERENCE TO RELATED APPLICATIONS

This application claims the benefit of U.S. Provisional Application No. 60/533,211, filed Dec. 31, 2003, and U.S. Provisional Application No. 60/584,652, filed Jul. 2, 2004, both of which are incoporated herein by reference.

This application may be relevant to U.S. Ser. No. 09/189,702 filed Nov. 10, 1998, which is a CIP of U.S. Ser. No. 08/205,713 filed Mar. 4, 1994, which is a CIP of Ser. No. 08/159,184 filed Nov. 29, 1993 and now abandoned, which is a CIP of Ser. No. 08/073,205 filed Jun. 4, 1993 and now abandoned, which is a CIP of Ser. No. 08/027,146 filed Mar. 5, 1993 and now abandoned. The present application is also related to U.S. Ser. No. 09/226,775, which is a CIP of U.S. Ser. No. 08/815,396, which claims the benefit of U.S. Ser. No. 60/013,113, now abandoned. Furthermore, the present application is related to U.S. Ser. No. 09/017,735, which is a CIP of abandoned U.S. Ser. No. 08/589,108; U.S. Ser. No. 08/753,622, U.S. Ser. No. 08/822,382, abandoned U.S. Ser. No. 60/013,980, U.S. Ser. No. 08/454,033, U.S. Ser. No. 09/116,424, and U.S. Ser. No. 08/349,177. The present application is also related to U.S. Ser. No. 09/017,524, U.S. Ser. No. 08/821,739, abandoned U.S. Ser. No. 60/013,833, U.S. Ser. No. 08/758,409, U.S. Ser. No. 08/589,107, U.S. Ser. No. 08/451,913, U.S. Ser. No. 08/186,266, U.S. Ser. No. 09/116,061, and U.S. Ser. No. 08/347,610, which is a CIP of U.S. Ser. No. 08/159,339, which is a CIP of abandoned U.S. Ser. No. 08/103,396, which is a CIP of abandoned U.S. Ser. No. 08/027,746, which is a CIP of abandoned U.S. Ser. No. 07/926,666. The present application may also be relevant to U.S. Ser. No. 09/017,743, U.S. Ser. No. 08/753,615; U.S. Ser. No. 08/590,298, U.S. Ser. No. 09/115,400, and U.S. Ser. No. 08/452,843, which is a CIP of U.S. Ser. No. 08/344,824, which is a CIP of abandoned U.S. Ser. No. 08/278,634. The present application may also be related to provisional U.S. Ser. No. 60/087,192 and U.S. Ser. No. 09/009,953, which is a CIP of abandoned U.S. Ser. No. 60/036,713 and abandoned U.S. Ser. No. 60/037,432. In addition, the present application may be relevant to U.S. Ser. No. 09/098,584, and U.S. Ser. No. 09/239,043. The present application may also be relevant to co-pending U.S. Ser. No. 09/583,200 filed May 30, 2000, U.S. Ser. No. 09/260,714 filed Mar. 1, 1999, and U.S. Provisional Application No. 60/239,008, filed Oct. 6, 2000, and U.S. Provisional Application No. 60/166,529, filed Nov. 18, 1999. In addition, the present application may also be relevant to U.S. Provisional Application No. 60/239,008, filed Oct. 6, 2000, now abandoned; co-pending U.S. application Ser. No. 10/130,548, which is the U.S. Natl. Phase Application of PCT/US00/31856, filed Nov. 20, 2000 and published as WO 01/36452 on May 25, 2001; and co-pending U.S. application Ser. No. 10/116,118, filed Apr. 5, 2002. Each of the above applications is incorporated herein by reference.

### BACKGROUND OF THE INVENTION

Human papillomavirus (HPV) is a member of the papillomaviridae, a group of small DNA viruses that infect a variety of higher vertebrates. More than 80 types of HPVs have been identified. Of these, more than 30 can infect the genital tract. Some types, generally types 6 and 11, may cause genital warts, which are typically benign and rarely develop into cancer. Other strains of HPV, “cancer-associated”, or “high-risk” types, can more frequently lead to the development of cancer. The primary mode of transmission of these strains of HPV is through sexual contact.

The main manifestations of the genital warts are cauliflower-like condylomata acuminata that usually involve moist surfaces; keratotic and smooth papular warts, usually on dry surfaces; and subclinical “flat” warts, which are found on any mucosal or cutaneous surface (Handsfield, H., Am. J. Med. 102(5A):16-20 (1997)). These warts are typically benign but are a source of inter-individual spread of the virus (Ponten, J. and Guo, Z., Cancer Surv. 32:201-229 (1998)). At least three HPV strains associated with genital warts have been identified: type 6a (see, e.g., Hofmann, K. J., et al., Virology 209(2):506-518 (1995)), type 6b (see, e.g., Hofmann, K. J., et al., Virology 209(2):506-518 (1995)) and type 11 (see, e.g., Dartmann, K., et al., Virology 151(1):124-130 (1986)).

Cancer-associated HPVs have been linked with cancer in both men and women; they include, but are not limited to, HPV-16, HPV-18, HPV-31, HPV-33, HPV-45 and HPV-56. Other HPV strains, including types 6 and 11 as well as others, e.g., HPV-5 and HPV-8, are less frequently associated with cancer. The high risk types are typically associated with the development of cervical carcinoma and premalignant lesions of the cervix in women, but are also associated with similar malignant and premalignant lesions at other anatomic sites within the lower genital or anogenital tract. These lesions include neoplasia of the vagina, vulva, perineum, the penis, and the anus. HPV infection has also been associated with respiratory tract papillomas, and rarely, cancer, as well as abnormal growth or neoplasia in other epithelial tissues. See, e.g., Virology, 2nd Ed., Fields, et al., Eds. Raven Press, New York (1990), Chapters 58 and 59, for a review of HPV association with cancer.

The HPV genome consists of three functional regions, the early region, the late region, and the “long control region”. The early region gene products control viral replication, transcription and cellular transformation. They include the HPV E1 and E2 proteins, which play a role in HPV DNA replication, and the E6 and E7 oncoproteins, which are involved in the control of cellular proliferation. The late region include the genes that encode the structural proteins L1 and L2, which are the major and minor capsid proteins, respectively. The “long control region” contains such sequences as enhancer and promoter regulatory regions.

HPV expresses different proteins at different stages of the infection, for example early, as well as late, proteins. Even in latent infections, however, early proteins are often expressed and are therefore useful targets for vaccine-based therapies. For example, high-grade dysplasia and cervical squamous cell carcinoma continue to express E6 and E7, which therefore can be targeted to treat disease at both early and late stages of infection.

Treatment for HPV infection is often unsatisfactory because of persistence of virus after treatment and recurrence of clinically apparent disease is common. The treatment may require frequent visits to clinics and is not directed at elimination of the virus but at clearing warts. Because of persistence of virus after treatment, recurrence of clinically apparent disease is common.

Thus, a need exists for an efficacious vaccine to prevent and/or treat HPV infection and to prevent and/or treat cancer that is associated with HPV infection. Effective HPV vaccines would be a significant advance in the control of sexually transmissable infections and could also protect against clinical disease, particularly cancers such as cervical cancer. (see, e.g., Rowen, P. and Lacey, C., Dermatologic Clinics 16(4):835-838, 1998).

Virus-specific, human leukocyte antigen (HLA) class I-restricted cytotoxic T lymphocytes (CTL) are known to play a major role in the prevention and clearance of virus infections in vivo (Oldstone, et al., Nature 321:239, 1989; Jamieson, et al., J. Virol. 61:3930, 1987; Yap, et al., Nature 273:238, 1978; Lukacher, et al., J. Exp. Med. 160:814, 1994; McMichael, et al., N. Engl. J. Med. 309:13, 1983; Sethi, et al., J. Gen. Virol. 64:443, 1983; Watari, et al., J. Exp. Med. 165:459, 1987; Yasukawa, et al., J. Immunol. 143:2051, 1989; Tigges, et al., J. Virol. 66:1622, 1993; Reddenhase, et al., J. Virol. 55:263, 1985; Quinnan, et al., N. Engl. J. Med. 307:6, 1982). HLA class I molecules are expressed on the surface of almost all nucleated cells. Following intracellular processing of antigens, epitopes from the antigens are presented as a complex with the HLA class I molecules on the surface of such cells. CTL recognize the peptide-HLA class I complex, which then results in the destruction of the cell bearing the HLA-peptide complex directly by the CTL and/or via the activation of non-destructive mechanisms e.g., the production of interferon, that inhibit viral replication.

Virus-specific T helper lymphocytes are also known to be critical for maintaining effective immunity in chronic viral infections. Historically, HTL responses were viewed as primarily supporting the expansion of specific CTL and B cell populations; however, more recent data indicate that HTL may directly contribute to the control of virus replication. For example, a decline in CD4⁺ T cells and a corresponding loss in HTL function characterize infection with HIV (Lane, et al., N. Engl. J. Med. 313:79, 1985). Furthermore, studies in HIV infected patients have also shown that there is an inverse relationship between virus-specific HTL responses and viral load, suggesting that HTL plays a role in viremia (see, e.g., Rosenberg, et al., Science 278:1447, 1997).

The development of vaccines with prophylactic and/or therapeutic efficacy against HPV is ongoing. Early vaccine development was hampered by the inability to culture HPV. With the introduction of cloning techniques and protein expression, however, some attempts have been made to stimulate humoral and CTL response to HPV (See, e.g., Rowen, P. and Lacey, C., Dermatologic Clinics 16(4):835-838 (1998)). Studies to date, however, have been inconclusive.

Activation of T helper cells and cytotoxic lymphocytes (CTLs) in the development of vaccines has also been analyzed. Lehtinen, M., et al., for instance, has shown that some peptides from the E2 protein of HPV type 16 activate T helper cells and CTLs (Biochem. Biophys. Res. Comm. 209(2):541-6 (1995)). Similarly, Tarpey, et al., has shown that some peptides from HPV type 11 E7 protein can stimulate human HPV-specific CTLs in vitro (Immunology 81:222-227 (1994)) and Borysiewicz, et al. have reported a recombinant vaccinia virus expressing HPV 16 and HPV 17 E6 and E7 that stimulated CTL responses in at least one patient (Lancet 347:1347-57, 1996).

The epitope approach, as we describe herein, allows the incorporation of various antibody, CTL and HTL epitopes, from various proteins, in a single vaccine composition. Such a composition may simultaneously target multiple dominant and subdominant epitopes and thereby be used to achieve effective immunization in a diverse population.

The technology relevant to multi-epitope (“minigene”) vaccines is developing. Several independent studies have established that induction of simultaneous immune responses against multiple epitopes can be achieved. For example, responses against a large number of T cell specificities can be induced and detected. In natural situations, Doolan, et al. (Immunity, Vol. 7(1):97-112 (1997)) simultaneously detected recall T cell responses, against as many as 17 different P. falciparum epitopes using PBMC from a single donor. Similarly, Bertoni and colleagues (J. Clin. Invest., 100(3):503-13 (1997)) detected simultaneous CTL responses against 12 different HBV-derived epitopes in a single donor. In terms of immunization with multi-epitope nucleic acid vaccines, several examples have been reported where multiple T cell responses were induced. For example, minigene vaccines composed of approximately ten MRC Class I epitopes in which all epitopes were immunogenic and/or antigenic have been reported. Specifically, minigene vaccines composed of 9 EBV (Thomson, et al., Proc. Natl. Acad. Sci. USA, 92(13):5845-49 (1995)), 7 HIV (Woodberry, et al., J. Virol., 73(7):5320-25 (1999)), 10 murine (Thomson, et al., J. Immunol., 160(4):1717-23 (1998)) and 10 tumor-derived (Mateo, et al., J. Immunol., 163(7):4058-63 (1999)) epitopes have been shown to be active. It has also been shown that a multi-epitope DNA plasmid encoding nine different HLA-A2.1- and A11-restricted epitopes derived from HBV and HIV induced CTL against all epitopes (Ishioka, et al., J. Immunol., 162(7):3915-25 (1999)).

Recently, several multi-epitope DNA plasmid vaccines specific for HIV have entered clinical trials (Nanke, et al., Nature Med., 6:951-55 (2000); Wilson, C. C., et al., J. Immunol. 171(10):5611-23 (2003).

Thus, minigene vaccines containing multiple MHC Class I and Class II (i.e., CTL) epitopes can be designed, and presentation and recognition can be obtained for all epitopes. However, the immunogenicity of multi-epitope constructs appears to be strongly influenced by a number of variables, a number of which have heretofore been unknown. For example, the immunogenicity (or antigenicity) of the same epitope expressed in the context of different vaccine constructs can vary over several orders of magnitude. Thus, there exists a need to identify strategies to optimize multi-epitope vaccine constructs. Such optimization is important in terms of induction of potent immune responses and ultimately, for clinical efficacy. Accordingly, the present invention provides strategies to optimize antigenicity and immunogenicity of multi-epitope vaccines encompassing a large number of epitopes. The present invention also provides optimized multi-epitope vaccines, particularly minigene vaccines, generated in accordance with these strategies.

The following paragraphs provide a brief review of some of the main variables potentially influencing the immunogenicity, epitope processing, and presentation on antigen presenting cells (APCs) in association with Class I and Class II MHC molecules of one or more epitopes provided in a minigene construct.

Of the many thousand possible peptides that are encoded by a complex foreign pathogen, only a small fraction ends up in a peptide form capable of binding to MHC Class I antigens and thus of being recognized by T cells. This phenomenon, of obvious potential impact on the development of a multi-epitope vaccine, is known as immunodominance (Yewdell, et al., Ann. Rev. Immunol., 17:51-88 (1999)). Several major variables contribute to immunodominance. Herein, we describe variables affecting the generation of the appropriate peptides, both in qualitative and quantitative terms, as a result of intracellular processing.

A junctional epitope is defined as an epitope created due to the juxtaposition of two other epitopes. The junctional epitope is composed of a C-terminal section derived from a first epitope, and an N-terminal section derived from a second epitope. Creation of junctional epitopes is a potential problem in the design of multi-epitope minigene vaccines, for both Class I and Class II restricted epitopes for the following reasons. Firstly, when developing a minigene composed of, or containing, human epitopes, which are, typically tested for immunogenicity in HLA transgenic laboratory animals, the creation of murine epitopes could create undesired immunodominance effects. Secondly, the creation of new, unintended epitopes for human HLA Class I or Class II molecules could elicit in vaccine recipients, new T cell specificities that are not expressed by infected cells or tumors. These responses are by definition irrelevant and ineffective and could even be counterproductive to the intended vaccine response, by creating undesired immunodominance effects.

The existence of junctional epitopes has been documented in a variety of different experimental situations. Gefter and collaborators first demonstrated the effect in a system in which two different Class II restricted epitopes were juxtaposed and colinearly synthesized (Perkins, et al., J. Immunol., 146(7):2137-44 (1991)). The effect was so marked that the immune system recognition of the epitopes could be completely “silenced” by expression, processing, and immune response to these new junctional epitopes (Wang, et al., Cell Immunol., 143(2):284-97 (1992)). Helper T cells directed against junctional epitopes were also observed in humans as a result of immunization with a synthetic lipopeptide, which was composed of an HLA-A2-restricted HBV-derived immunodominant CTL epitope, and a universal Tetanus Toxoid-derived HTL epitope (Livingston, et al., J. Immunol., 159(3):1383-92 (1997)). Thus, the creation of junctional epitopes is a major consideration in the design of multi-epitope constructs.

In certain embodiments, the present invention provides methods of addressing this problem and avoiding or minimizing the occurrence of junctional epitopes.

Class I restricted epitopes are generated by a complex process (Yewdell, et al., Ann. Rev. Immunol., 17:51-88 (1999)). Limited proteolysis involving endoproteases and potential trimming by exoproteases is followed by translocation across the endoplasmic reticulum (ER) membrane by transporters associated with antigen processing (TAP) molecules. The major cytosolic protease complex involved in generation of antigenic peptides, and their precursors, is the proteosome (Niedermann, et al., Immunity, 2(3):289-99 (1995)), although ER trimming of CTL precursors has also been demonstrated (Paz, et al., Immunity, 11(2):241-51 (1999)). It has long been debated whether the residues immediately flanking the C- and N-termini of the epitope have an influence on the efficiency of epitope processing.

The yield and availability of processed epitope has been implicated as a major variable in determining immunogenicity and could thus clearly have a major impact on overall minigene potency in that the magnitude of immune response can be directly proportional to the amount of epitope bound by MHC and displayed for T cell recognition. Several studies have provided evidence that this is indeed the case. For example, induction of virus-specific CTL that is essentially proportional to epitope density (Wherry, et al., J. Immunol., 163(7):3735-45 (1999); Livingston, et. al., Vaccine, 19(32) 4652-60 (2001)) has been observed. Further, recombinant minigenes, which encode a preprocessed optimal epitope, have been used to induce higher levels of epitope expression than naturally observed with full-length protein (Anton, et al., J. Immunol., 158(6):2535-42 (1997)). In general, minigene priming has been shown to be more effective than priming with the whole antigen (Restifo, et al., J. Immunol., 154(9):4414-22 (1995); Ishioka, et al., J. Immunol., 162(7):3915-25 (1999)), even though some exceptions have been noted (Iwasaki, et al., Vaccine, 17(15-16):2081-88 (1999)).

Early studies concluded that residues within the epitope (Hahn, et al., J. Exp. Med., 176(5):1335-41 (1992)) primarily regulate immunogenicity. Similar conclusions were reached by other studies, mostly based on grafting an epitope into an unrelated gene, or in the same gene, but in a different location (Chimini, et al., J. Exp. Med., 169(1):297-302 (1989); Hahn, et al., J. Exp. Med., 174(3):733-36 (1991)). Other experiments however (Del Val, et al., Cell, 66(6):1145-53 (1991); Hahn, et al., J. Exp. Med., 176(5):1335-41 (1992)), suggested that residues localized directly adjacent to the CTL epitope can directly influence recognition (Couillin, et al., J. Exp. Med., 180(3):1129-34 (1994); Livingston, et al., Vaccine, 19(32) 4652-60 (2001)); Bergmann, et al., J. Virol., 68(8):5306-10 (1994)). In the context of minigene vaccines, the controversy has been renewed. Shastri and coworkers (J. Immunol., 155(9):4339-46 (1995)) found that T cell responses were not significantly affected by varying the N-terminal flanking residue but were inhibited by the addition of a single C-terminal flanking residue. The most dramatic inhibition was observed with isoleucine, leucine, cysteine, and proline as the C-terminal flanking residues. In contrast, Gileadi (Eur. J. Immunol., 29(7):2213-22 (1999)) reported profound effects as a function of the residues located at the N-terminus of mouse influenza virus epitopes. Bergmann and coworkers found that aromatic, basic and alanine residues supported efficient epitope recognition, while glycine and proline residues were strongly inhibitory (Bergmann, et al., J. Immunol., 157(8):3242-49 (1996)). In contrast, Lippolis (J. Virol., 69(5):3134-46 (1995)) concluded that substituting flanking residues did not effect recognition. However, Lippolis' observations may be tempered by the fact that only rather conservative substitutions were tested and such substituted residues are unlikely to affect proteosome specificity.

It appears that the specificity of these effects, and in general of natural epitopes, roughly correlates with proteosome specificity. For example, proteosome specificity is partly trypsin-like (Niedermann, et al., Immunity, 2(3):289-99 (1995)), with cleavage following basic amino acids. Nevertheless, efficient cleavage of the carboxyl side of hydrophobic and acidic residues is also possible. Consistent with these specificities are the studies of Sherman and collaborators, which found that an arginine to histidine mutation at the position following the C-terminus of a p53 epitope affects proteosome-mediated processing of the protein (Theobald, et al., J. Exp. Med., 188(6):1017-28 (1998)). Several other studies (Hanke, et al., J. Gen. Virol., 79 (Pt 1):83-90 (1998); Thomson, et al., Proc. Natl. Acad. Sci. USA, 92(13):5845-49 (1995)) indicated that minigenes can be constructed utilizing minimal epitopes, and that flanking sequences appear not to be required, although the potential for further optimization by the use of flanking regions was also acknowledged.

In sum, for HLA Class I epitopes, the effects of flanking regions on processing and presentation of CTL epitopes has yet to be fully defined. A systematic analysis of the effect of modulation of flanking regions has not been performed for minigene vaccines. Thus, analysis utilizing minigene vaccines encoding epitopes restricted by human Class I in general is needed. The present invention provides in part such an analysis of the effects of flanking regions on processing and presentation of CTL epitopes. Thus, in certain embodiments, the present invention provides multi-epitope vaccine constructs optimized from immunogenicity and antigenicity, and methods of designing such constructs.

HLA Class II peptide complexes are also generated as a result of a complex series of events distinct from HLA Class I processing. The processing pathway involves association with Invariant chain (Ii), its transport to specialized compartments, the degradation of Ii to CLIP, and HLA-DM catalyzed removal of CLIP (Blum, et al., Crit. Rev. Immunol., 17(5-6):411-17 (1997); and Arndt, et al., Immunol. Res., 16(3):261-72 (1997) for review. Moreover, there is a potentially crucial role of various cathepsins in general, and cathepsin S and L in particular, in Ii degradation (Nakagawa, et al., Immunity, 10(2):207-17 (1999)). In terms of generation of functional epitopes however, the process appears to be somewhat less selective (Chapman, H. A., Curr. Opin. Immunol., 10(1):93-102 (1998)), and peptides of many sizes can bind to MHC Class II (Hunt, et al., Science, 256(5065):1817-20 (1992)). Most or all of the possible peptides appear to be generated (Moudgil, et al., J. Immunol., 159(6):2574-49 (1997); and Thomson, et al., J. Virol., 72(3):2246-52 (1998)). Thus, as compared to the issue of flanking regions, the creation of junctional epitopes can be a more serious concern in particular embodiments.

One of the most formidable obstacles to the development of broadly efficacious epitope-based immunotherapeutics, however, has been the extreme polymorphism of HLA molecules. To date, effective non-genetically biased coverage of a population has been a task of considerable complexity; such coverage has required that epitopes be used that are specific for HLA molecules corresponding to each individual HLA allele. Impractically large numbers of epitopes would therefore have to be used in order to cover ethnically diverse populations. Thus, there has existed a need for peptide epitopes that are bound by multiple HLA antigen molecules for use in epitope-based vaccines. The greater the number of HLA antigen molecules bound, the greater the breadth of population coverage by the vaccine.

Furthermore, as described herein in greater detail, a need has existed to modulate peptide binding properties, e.g., so that peptides that are able to bind to multiple HLA antigens do so with an affinity that will stimulate an immune response. Identification of epitopes restricted by more than one HLA allele at an affinity that correlates with immunogenicity is important to provide thorough population coverage, and to allow the elicitation of responses of sufficient vigor to prevent or clear an infection in a diverse segment of the population. Such a response can also target a broad array of epitopes. In certain embodiments, the technology disclosed herein provides for such favored immune responses. The information provided in this section is intended to disclose the presently understood state of the art as of the filing date of the present application. Certain information is included in this section which was generated subsequent to the priority date of this application. Accordingly, information in this section is not intended, in any way, to delineate the priority date for the invention.

### SUMMARY OF THE INVENTION

This invention applies our knowledge of the mechanisms by which antigen is recognized by T cells, for example, to develop epitope-based vaccines directed towards HPV. More specifically, this application communicates our discovery of specific epitope compositions, specific epitope pharmaceutical compositions, and methods of use in the prevention and treatment of HPV infection, and/or HPV-associated cancers and other maladies.

The use of epitope-based vaccines has several advantages over current vaccines, particularly when compared to the use of whole antigens in vaccine compositions. There is evidence that the immune response to whole antigens is directed largely toward variable regions of the antigen, allowing for immune escape due to variability and/or mutations. The epitopes for inclusion in an epitope-based vaccine, such as those of the present invention, may be selected from conserved regions of viral or tumor-associated antigens, thereby reducing the likelihood of escape mutants. Furthermore, immunosuppressive epitopes that may be present in whole antigens can be avoided with the use of epitope-based vaccines, such as those of the present invention.

An additional advantage of the epitope-based vaccines and methods of the present invention, is the ability to combine selected epitopes (CTL and HTL), and further, to modify the composition of the epitopes, achieving, for example, enhanced immunogenicity. Accordingly, the vaccines and methods of the present invention are useful to modulate the immune response can be modulated, as appropriate, for the target disease. Similar engineering of the response is not possible with traditional approaches outside the scope of the present invention.

Another major benefit of epitope-based immune-stimulating vaccines of the present invention is their safety. The possible pathological side effects caused by infectious agents or whole protein antigens, which might have their own intrinsic biological activity, are eliminated.

Epitope-based vaccines of the present invention also provide the ability to direct and focus an immune response to multiple selected antigens from the same pathogen. Thus, in certain embodiments, patient-by-patient variability in the immune response to a particular pathogen may be alleviated by inclusion of epitopes from multiple antigens from the pathogen in a vaccine composition. In preferred embodiments of the present invention, epitopes derived from multiple strains of HPV may also be included. In a highly preferred embodiment of the present invention, epitopes derived from one or more of HPV strains 6a, 6b, 11a, 16, 18, 31, 33, 45, 52, 56, and 58 are included.

In a preferred embodiment, epitopes for inclusion in epitope compositions and/or vaccine compositions of the invention are selected by a process whereby protein sequences of known antigens are evaluated for the presence of motif or supermotif-bearing epitopes. Peptides corresponding to a motif- or supermotif-bearing epitope are then synthesized and tested for the ability to bind to the HLA molecule that recognizes the selected motif. Those peptides that bind at an intermediate or high affinity i.e., an IC₅₀ (or a KD value) of 500 nM or less for HLA class I molecules or an IC₅₀ of 1000 nM or less for HLA class II molecules, are further evaluated for their ability to induce a CTL or HTL response. Immunogenic peptide epitopes are selected for inclusion in epitope compositions and/or vaccine compositions.

In certain embodiments, supermotif-bearing peptides are tested for the ability to bind to multiple alleles within the HLA supertype family. In other related embodiments, peptide epitopes may be analoged to modify binding affinity and/or the ability to bind to multiple alleles within an HLA supertype.

The invention also includes embodiments comprising methods for monitoring or evaluating an immune response to HPV in a patient having a known HLA-type. Such methods comprise incubating a T lymphocyte sample from the patient with a peptide composition comprising an HPV epitope that has an amino acid sequence described in Tables 7-18 which binds the product of at least one HLA allele present in the patient, and detecting and/or measuring for the presence of a T lymphocyte that binds to the peptide. In certain embodiments, a CTL peptide epitope may, for example, be used as a component of a tetrameric complex for this type of analysis.

An alternative modality for defining the peptide epitopes in accordance with certain embodiments of the invention is to recite the physical properties, such as length; primary structure; or charge, which are correlated with binding to a particular allele-specific HLA molecule or group of allele-specific HLA molecules. A further modality of the invention for defining peptide epitopes is to recite the physical properties of an HLA binding pocket, or properties shared by several allele-specific HLA binding pockets (e.g. pocket configuration and charge distribution) and reciting that the peptide epitope fits and binds to the pocket or pockets.

Certain embodiments of the present invention are also directed to methods for selecting a variant of a peptide epitope which induces a CTL response against not only itself, but also against the peptide epitope itself and/or one or more other variants of the peptide epitope, by determining whether the variant comprises only conserved residues, as defined herein, at non-anchor positions in comparison to the other variant(s). Variants are referred to herein as “CTL epitopes” and “HTL epitopes” as well as “variants.”

In some embodiments, antigen sequences from a population of HPV (said antigens comprising variants of a peptide epitope) are optimally aligned (manually or by computer) along their length, preferably their full length. Variant(s) of a peptide epitope (preferably naturally occurring variants), each 8-11 amino acids in length and comprising the same MHC class I supermotif or motif, are identified manually or with the aid of a computer. In some embodiments, a variant is optimally chosen which comprises preferred anchor residues of said motif and/or which occurs with high frequency within the population of variants. In other embodiments, a variant is randomly chosen. The randomly or otherwise chosen variant is compared to from one to all the remaining variant(s) to determine whether it comprises only conserved residues in the non-anchor positions relative to from one to all the remaining variant(s).

The present invention is also directed to variants identified by the methods above; peptides comprising such variants; nucleic acids encoding such variants and peptides; cells comprising such variants, and/or peptides, and/or nucleic acids; compositions comprising such variants, and/or peptides, and/or nucleic acids, and/or cells; as well as prophylactic, therapeutic, and/or diagnostic methods for using such variants, peptides, nucleic acids, cells, and compositions.

The invention also provides multi-epitope nucleic acid constructs encoding a plurality of CTL and/or HTL epitopes (including variants in certain embodiments) and polypeptide constructs comprising a plurality of CTL and/or HTL epitopes (preferably encoded by the nucleic acid constructs), as well as cells comprising such nucleic acid constructs and/or polypeptide constructs, compositions comprising such nucleic acid constructs and/or polypeptide constructs and/or such cells, and methods for stimulating an immune response (e.g., therapeutic and/or prophylactic methods) utilizing such nucleic acid constructs and/or polypeptide constructs and/or compositions and/or cells.

In other embodiments, the invention provides cells comprising the nucleic acids and/or polypeptides above; compositions comprising the nucleic acids and/or polypeptides and/or cells; methods for making these nucleic acids, polypeptides, cells and compositions; and methods for stimulating an immune response (e.g. therapeutic and/or prophylactic methods) utilizing these nucleic acids and/or polypeptides and/or cells and/or compositions.

In other embodiments, the invention provides a polynucleotide selected from the following polynucleotides (a)-(m), each encoding the human papillomavirus (HPV) cytotoxic T lymphocyte (CTL) epitopes of Core Group HPV 64.

(a) A multi-epitope polynucleotide construct comprising nucleic acids encoding the human papillomavirus (HPV) cytotoxic T lymphocyte (CTL) epitopes of Core Group HPV 64. These epitopes are: HPV.31.E7.44.T2, HPV16.E6.106HPV16.E6.131, HPV16.E6.29.L2, HPV16.E6.68.R10, HPV16.E6.75.F9, HPV16.E6.80.D3, HPV16.E7.11.V10, HPV16.E7.2.T2, HPV16.E7.56.F10, HPV18.E6.126.F9, HPV18.E6.24, HPV18.E6.25.T2, HPV18.E6.33.F9, HPV18.E6.47, HPV18.E6.72.D3, HPV18.E6.83.R10, HPV18.E6.84.V10, HPV18.E6.89, HPV18.E7.59.R9, HPV18/45.E6.13, HPV18/45.E6.98.F9, HPV31.E6.15, HPV31.E6.46.T2, HPV31.E6.47, HPV31.E6.69, HPV31.E6.72, HPV31.E6.80, HPV31.E6.82.R9, HPV31.E6.83, HPV31.E6.90, HPV33.E6.42, HPV33.E6.53, HPV33.E6.61.V10, HPV33.E6.64, HPV33.E7.11.V10, HPV33.E7.6, HPV33.E7.81, HPV33/52.E6.68.V2, HPV33/58.E6.124.F9, HPV33/58.E6.72.R10, HPV33/58.E6.73.D3, HPV45.E6.24, HPV45.E6.25. T2, HPV45.E6.28, HPV45.E6.37, HPV45.E6.41.R10, HPV45.E6.44, HPV45.E6.71.F10, HPV45.E6.84.R9, HPV45.E7.20, HPV56.E6.25, HPV56.E6.45, HPV56.E6.55.K9, HPV56.E6.62.F10, HPV56.E6.70, HPV56.E6.72.T2, HPV56.E6.86, HPV56.E6.89, HPV56.E6.99.T2, HPV56.E7.84.V10, and HPV56.E7.92.L2, wherein the nucleic acids are directly or indirectly joined to one another in the same reading frame. Note that the nucleic acids encoding the epitopes listed above may be arranged in any order.

(b) A multi-epitope polynucleotide construct comprising nucleic acids encoding the human papillomavirus (HPV) cytotoxic T lymphocyte (CTL) epitopes of Core Group HPV 64 (hereinafter “the HPV 64 core construct”), and also encoding one or more additional CTL and/or HTL epitopes.

(c) The HPV 64 core construct as in (a) or (b), where the nucleic acids encoding the epitopes listed above are arranged in a specified order, but may have additional nucleic acids encoding additional epitopes and/or spacer amino acids dispersed therein.

(d) The HPV 64 core construct as in (a)-(c), where one or more epitope-encoding nucleic acids are flanked by spacer nucleotides, and/or other polynucleotide sequences as described herein or otherwise known in the art. Such spacer nucleotides encode one or more spacer amino acids so as to keep the multi-epitope construct in frame.

(e) The HPV 64 core construct as in (a)-(d), where the multi-epitopeconstruct is distinguished from other multi-epitopeconstructs according to whether the spacer nucleotides in one construct encode spacer amino acids which optimize epitope processing and/or minimize junctional epitopes with respect to other constructs as described herein or elsewhere.

(f) The HPV 64 core construct as in (a)-(e), where the multi-epitope construct encodes a polypeptide which is concomitantly optimized for epitope processing and junctional epitopes with respect to one or more other constructs as described herein.

(g) The HPV 64 core construct as in (a)-(f), where the multi-epitope-construct further comprises a PADRE HTL epitope, as described herein.

(h) The HPV 64 core construct as in (a)-(g), further comprising nucleic acids encoding HPV CTL epitopes HPV16.E6.30.T2 and HPV16.E6.59.

(i) The HPV 64 core construct as in (a)-(h), further comprising nucleic acids encoding HPV CTL epitopes HPV16.E6.75.L2 and HPV16.E6.77.

(j) The HPV 64 core construct as in (h), comprising or alternatively consisting of the multi-epitope construct HPV 64 gene 1 (See Tables 38A, 39A and 40A).

(k) The HPV 64 core construct as in (h), comprising or alternatively consisting of the multi-epitope construct HPV 64 gene 2 (See Tables 38B, 39B and 40B).

(l) The HPV 64 core construct as in (i), comprising or alternatively consisting of the multi-epitope construct HPV 64 gene 1R (See Tables 41A, 42A and 43A).

(m) The HPV 64 core construct as in (i), comprising or alternatively consisting of the multi-epitope construct HPV 64 gene 2R (See Tables 41B, 42B and 43B).

In other embodiments, the invention provides a polypeptide comprising HPV 64 CTL epitopes encoded by any of polynucleotides (a)-(m) listed above.

In other embodiments, the invention provides a polynucleotide selected from the following polynucleotides (a)-(m), each encoding the human papillomavirus (HPV) cytotoxic T lymphocyte (CTL) epitopes of Core Group HPV 43.

(a) A multi-epitope polynucleotide construct comprising nucleic acids encoding the human papillomavirus (HPV) cytotoxic T lymphocyte (CTL) epitopes of Core Group HPV 43. These epitopes are: HPV.31.E7.44.T2, HPV16.E6.106, HPV16.E6.131, HPV16.E6.29.L2, HPV16.E6.30.T2, HPV16.E6.75.F9, HPV16.E6.80.D3, HPV16.E7.11.V10, HPV16.E7.2.T2, HPV16.E7.56.F10, HPV18.E6.126.F9, HPV18.E6.24, HPV18.E6.25.T2, HPV18.E6.33.F9, HPV18.E6.47, HPV18.E6.72.D3, HPV18.E6.83.R10, HPV18.E6.84.V10, HPV18.E6.89, HPV18.E7.59.R9, HPV18/45.E6.13, HPV18/45.E6.98.F9, HPV31.E6.15, HPV31.E6.46.T2, HPV31.E6.47, HPV31.E6.69, HPV31.E6.80, HPV31.E6.82.R9, HPV31.E6.83, HPV31.E6.90, HPV33.E7.11.V10, HPV45.E6.24, HPV45.E6.25.T2, HPV45.E6.28, HPV45.E6.37, HPV45.E6.41.R10, HPV45.E6.44, HPV45.E6.71.F10, HPV45.E6.84.R9, and HPV45.E7.20, where the nucleic acids are directly or indirectly joined to one another in the same reading frame. Note that the nucleic acids encoding the epitopes listed above may be arranged in any order.

(b) A multi-epitope polynucleotide construct comprising nucleic acids encoding the human papillomavirus (HPV) cytotoxic T lymphocyte (CTL) epitopes of Core Group HPV 43 (hereinafter “the HPV 43 core construct”), and also encoding one or more additional CTL and/or HTL epitopes.

(c) The HPV 43 core construct as in (a)-(b), where the nucleic acids encoding the epitopes listed above are arranged in a specified order, but may have additional nucleic acids encoding additional epitopes and/or spacer amino acids dispersed therein.

(d) The HPV 43 core construct as in (a)-(c), where one or more epitope-encoding nucleic acids are flanked by spacer nucleotides, and/or other polynucleotide sequences as described herein or otherwise known in the art. Such spacer nucleotides encode one or more spacer amino acids so as to keep the multi-epitope construct in frame.

(e) The HPV 43 core construct as in (a)-(d), where the multi-epitopeconstruct is distinguished from other multi-epitopeconstructs according to whether the spacer nucleotides in one construct encode spacer amino acids which optimize epitope processing and/or minimize junctional epitopes with respect to other constructs as described herein or elsewhere.

(f) The HPV 43 core construct as in (a)-(e), where the multi-epitope construct encodes a polypeptide which is concomitantly optimized for epitope processing and junctional epitopes with respect to one or more other constructs as described herein.

(g) The HPV 43 core construct as in (a)-(f), where the multi-epitope-construct further comprises a PADRE HTL epitope, as described herein.

(h) The HPV 43 core construct as in (a)-(g), further comprising nucleic acids encoding HPV CTL epitopes HPV31.E6.72, HPV16.E6.59, and HPV16.E6.68.R10.

(i) The HPV 43 core construct as in (a)-(g), further comprising nucleic acids encoding HPV CTL epitopes HPV16.E6.75.L2, HPV16.E6.77, and HPV31.E6.73.D3.

(j) The HPV 43 core construct as in (h), comprising or alternatively consisting of the multi-epitope construct HPV 43 gene 3 (See Tables 38C, 39C and 40C).

(k) The HPV 43 core construct as in (h), comprising or alternatively consisting of the multi-epitope construct HPV 43 gene 4 (See Tables 38D, 39D and 40D).

(l) The HPV 43 core construct as in (i), comprising or alternatively consisting of the multi-epitope construct HPV 43 gene 3R (See Tables 41C, 42C and 43C).

(m) The HPV 43 core construct as in (i), comprising or alternatively consisting of the multi-epitope construct HPV 43 gene 4R (See Tables 41D, 42D and 43D).

In other embodiments, the invention provides a polypeptide comprising HPV 43 CTL epitopes encoded by any of polynucleotides (a)-(m) listed above.

In other embodiments, the invention provides a polynucleotide selected from the following polynucleotides (a)-(m), each encoding the human papillomavirus (HPV) cytotoxic T lymphocyte (CTL) epitopes of Core Group HPV 46.

(a) A multi-epitope polynucleotide construct comprising nucleic acids encoding the human papillomavirus (HPV) cytotoxic T lymphocyte (CTL) epitopes of Core Group HPV 46. These epitopes are: HPV16.E6.106, HPV16.E6.29.L2, HPV16.E6.68.R10, HPV16.E6.75.F9, HPV16.E6.75.L2, HPV16.E6.77, HPV16.E6.80.D3, HPV16.E7.11.V10, HPV16.E7.2.T2, HPV16.E7.56.F10, HPV16.E7.86.V8, HPV18.E6.24, HPV18.E6.25.T2, HPV18.E6.33.F9, HPV18.E6.53.K10, HPV18.E6.72.D3, HPV18.E6.83.R10, HPV18.E6.84.V10, HPV18.E6.92.V10, HPV18.E7.59.R9, HPV18/45.E6.13, HPV18/45.E6.98.F9, HPV31.E6.132.K10, HPV31.E6.15, HPV31.E6.72, HPV31.E6.73.D3, HPV31.E6.80, HPV31.E6.82.R9, HPV31.E6.83.F9, HPV31.E6.90, HPV.31.E7.44.T2, HPV33.E7.11.V10, HPV45.E6.24, HPV45.E6.25.T2, HPV45.E6.37, HPV45.E6.41.R10, HPV45.E6.44, HPV45.E6.54, HPV45.E6.54.V10, HPV45.E6.71.F10, HPV45.E6.84.R9, and HPV45.E7.20, where the nucleic acids are directly or indirectly joined to one another in the same reading frame. Note that the nucleic acids encoding the epitopes listed above may be arranged in any order.

(b) A multi-epitope polynucleotide construct comprising nucleic acids encoding the human papillomavirus (HPV) cytotoxic T lymphocyte (CTL) epitopes of Core Group HPV 46 (hereinafter “the HPV 46 core construct”), and also encoding one or more additional CTL and/or HTL epitopes.

(c) The HPV 46 core construct as in (a)-(b), where the nucleic acids encoding the epitopes listed above are arranged in a specified order, but may have additional nucleic acids encoding additional epitopes and/or spacer amino acids dispersed therein.

(d) The HPV 46 core construct as in (a)-(c), where one or more epitope-encoding nucleic acids are flanked by spacer nucleotides, and/or other polynucleotide sequences as described herein or otherwise known in the art. Such spacer nucleotides encode one or more spacer amino acids so as to keep the multi-epitope construct in frame.

(e) The HPV 46 core construct as in (a)-(d), where the multi-epitopeconstruct is distinguished from other multi-epitopeconstructs according to whether the spacer nucleotides in one construct encode spacer amino acids which optimize epitope processing and/or minimize junctional epitopes with respect to other constructs as described herein or elsewhere.

(f) The HPV 46 core construct as in (a)-(e), where the multi-epitope construct encodes a polypeptide which is concomitantly optimized for epitope processing and junctional epitopes with respect to one or more other constructs as described herein.

(g) The HPV 46 core construct as in (a)-(f), where the multi-epitope-construct further comprises a PADRE HTL epitope, as described herein.

(h) The HPV 46 core construct as in (a)-(g), further comprising nucleic acids encoding HPV CTL epitopes HPV31.E6.69, HPV16.E6.131, HPV18.E6.126.F9, and HPV18.E6.89.

(i) The HPV 46 core construct as in (a)-(h), further comprising nucleic acids encoding HPV CTL epitopes HPV31.E6.69, HPV16.E6.131, HPV18.E6.126.F9 and HPV18.E6.89.I2.

(j) The HPV 46 core construct as in (a)-(i), further comprising nucleic acids encoding HPV CTL epitopes HPV18.E6.89, HPV16.E7.2.T2, HPV18.E6.44, and HPV31.E6.69+R@68.

(k) The HPV 46 core construct as in (h), comprising or alternatively consisting of the multi-epitope construct HPV 46-5 (See Tables 47A and 49A).

(l) The HPV 46 core construct as in (h), comprising or alternatively consisting of the multi-epitope construct HPV 46-5.2 (See Tables 47C, 49C).

(m) The HPV 46 core construct as in (i), comprising or alternatively consisting of the multi-epitope construct HPV 46-6 (See Tables 47B, 49B).

(n) The HPV 46 core construct as in (j), comprising or alternatively consisting of the multi-epitope construct HPV 46-5.3 (See Table 73).

In other embodiments, the invention provides a polypeptide comprising HPV 46 CTL epitopes encoded by any of polynucleotides (a)-(n) listed above.

In other embodiments, the invention provides a polynucleotide selected from the following polynucleotides (a)-(m), each encoding the human papillomavirus (HPV) cytotoxic T lymphocyte (CTL) epitopes of Core Group HPV 47.

(a) A multi-epitope polynucleotide construct comprising nucleic acids encoding the human papillomavirus (HPV) cytotoxic T lymphocyte (CTL) epitopes of Core Group HPV 47. These epitopes are: HPV16.E1.214, HPV16.E1.254, HPV16.E1.314, HPV16.E1.420, HPV16.E1.585, HPV16.E2.130, HPV16.E2.329, HPV16/52.E2.151, HPV18.E1.592, HPV18.E2.136, HPV18.E2.142, HPV18.E2.15, HPV18.E2.154, HPV18.E2.168, HPV18.E2.230, HPV18/45.E1.321, HPV18/45.E1.491, HPV31.E1.272, HPV31.E1.349, HPV31.E1.565, HPV31.E2.11, HPV31.E2.130, HPV31.E2.138, HPV31.E2.205, HPV31.E2.291, HPV31.E2.78, HPV45.E1.232, HPV45.E1.252, HPV45.E1.399, HPV45.E1.411, HPV45.E1.578, HPV45.E2.137, HPV45.E2.144, HPV45.E2.17, HPV45.E2.332, and HPV45.E2.338, wherein the nucleic acids are directly or indirectly joined to one another in the same reading frame. Note that the nucleic acids encoding the epitopes listed above may be arranged in any order.

(b) A multi-epitope polynucleotide construct comprising nucleic acids encoding the human papillomavirus (HPV) cytotoxic T lymphocyte (CTL) epitopes of Core Group HPV 47 (hereinafter “the HPV 47 core construct”), and also encoding one or more additional CTL and/or HTL epitopes.

(c) The HPV 47 core construct as in (a)-(b), where the nucleic acids encoding the epitopes listed above are arranged in a specified order, but may have additional nucleic acids encoding additional epitopes and/or spacer amino acids dispersed therein.

(d) The HPV 47 core construct as in (a)-(c), where one or more epitope-encoding nucleic acids are flanked by spacer nucleotides, and/or other polynucleotide sequences as described herein or otherwise known in the art. Such spacer nucleotides encode one or more spacer amino acids so as to keep the multi-epitope construct in frame.

(e) The HPV 47 core construct as in (a)-(d), where the multi-epitopeconstruct is distinguished from other multi-epitopeconstructs according to whether the spacer nucleotides in one construct encode spacer amino acids which optimize epitope processing and/or minimize junctional epitopes with respect to other constructs as described herein or elsewhere.

(f) The HPV 47 core construct as in (a)-(e), where the multi-epitope construct encodes a polypeptide which is concomitantly optimized for epitope processing and junctional epitopes with respect to one or more other constructs as described herein.

(g) The HPV 47 core construct as in (a)-(f), where the multi-epitope-construct further comprises a PADRE HTL epitope, as described herein.

(h) The HPV 47 core construct as in (a)-(g), further comprising nucleic acids encoding HPV CTL epitopes HPV16.E1.493, HPV31/52.E1.557, HPV31.E2.131, HPV31.E2.127, HPV16.E2.335, HPV16.E2.37, HPV16.E2.93, HPV18.E2.211, HPV18.E2.61, HPV18.E1.266 and HPV18.E1.500.

(i) The HPV 47 core construct as in (a)-(h), further comprising nucleic acids encoding HPV CTL epitopes HPV16.E1.191, HPV16.E1.292, HPV16.E1.489, HPV16.E1.489, HPV6/52.E1.406, HPV18.E1.210, HPV18.E1.266, HPV18.E1.463, HPV31.E1.464, HPV18/45.E1.284 and HPV31.E1.441.

(j) The HPV 47 core construct as in (h), comprising or alternatively consisting of the multi-epitope construct 47-1 (See Tables 52A, 53A and 54A).

(k) The HPV 47 core construct as in (h), comprising or alternatively consisting of the multi-epitope construct 47-2 (See Tables 52B, 53B and 54B).

(l) The HPV 47 core construct as in (i), comprising or alternatively consisting of the multi-epitope construct 47-3 (See Tables 74, 76A and 76B).

(m) The HPV 47 core construct as in (i), comprising or alternatively consisting of the multi-epitope construct 47-4 (See Tables 75, 76C and 76D).

In other embodiments, the invention provides a polypeptide comprising HPV 46 CTL epitopes encoded by any of polynucleotides (a)-(m) listed above.

In other embodiments, the invention provides a polynucleotide selected from the following polynucleotides (a)-(p), each encoding the human papillomavirus (HPV) helper T lymphocyte (HTL) epitopes of Core Group HTL780-20/30.

(a) A multi-epitope polynucleotide construct comprising nucleic acids encoding the human papillomavirus (HPV) helper T lymphocyte (HTL) epitopes of Core Group HTL780-20/30. These epitopes are: HPV16.E6.13, HPV16.E6.130, HPV16.E7.13, HPV16.E7.46, HPV16.E7.76, HPV18.E6.43, HPV31.E6.132, HPV31.E6.42, HPV31.E6.78, HPV45.E6.127, HPV45.E7.10 and HPV45.E7.82, wherein the nucleic acids are directly or indirectly joined to one another in the same reading frame. Note that the nucleic acids encoding the epitopes listed above may be arranged in any order.

(b) A multi-epitope polynucleotide construct comprising nucleic acids encoding the human papillomavirus (HPV) cytotoxic T lymphocyte (CTL) epitopes of Core Group HTL780-20/30 (hereinafter “the HTL780-20/30 core construct”), and also encoding one or more additional CTL and/or HTL epitopes.

(c) The HTL780-20/30 core construct as in (a)-(b), where the nucleic acids encoding the epitopes listed above are arranged in a specified order, but may have additional nucleic acids encoding additional epitopes and/or spacer amino acids dispersed therein.

(d) The HTL780-20/30 core construct as in (a)-(c), where one or more epitope-encoding nucleic acids are flanked by spacer nucleotides, and/or other polynucleotide sequences as described herein or otherwise known in the art. Such spacer nucleotides encode one or more spacer amino acids so as to keep the multi-epitope construct in frame.

(e) The HTL780-20/30 core construct as in (a)-(d), where the multi-epitopeconstruct is distinguished from other multi-epitopeconstructs according to whether the spacer nucleotides in one construct encode spacer amino acids which optimize epitope processing and/or minimize junctional epitopes with respect to other constructs as described herein or elsewhere.

(f) The HTL780-20/30 core construct as in (a)-(e), where the multi-epitope construct encodes a polypeptide which is concomitantly optimized for epitope processing and junctional epitopes with respect to one or more other constructs as described herein.

(g) The HTL780-20/30 core construct as in (a)-(f), where the multi-epitope-construct further comprises a PADRE HTL epitope, as described herein.

(h) The HTL780-20/30 core construct as in (a)-(g), further comprising nucleic acids encoding HPV HTL epitopes HPV18.E6.52 and 53, HPV18.E6.94+Q, HPV18.E7.86 and HPV31.E7.76.

(i) The HTL780-20/30 core construct as in (a)-(h), further comprising nucleic acids encoding HPV HTL epitopes HPV18.E6.94, HPV18.E7.78, HPV31.E6.1 and HPV31.E7.36.

(j) The HTL780-20/30 core construct as in (h), comprising or alternatively consisting of the multi-epitope construct HTL 780-30 (See Tables 80 and 81).

(k) The HTL780-20/30 core construct as in (i), comprising or alternatively consisting of the multi-epitope construct HTL 780-20.

(l) The HTL780-20/30 core construct as in (a)-(k), further comprising any of the HPV 46 core constructs (a)-(m) as described above.

(m) The HTL780-20/30 core construct as in (a)-(1), further comprising nucleic acids encoding HPV CTL epitopes CTL epitopes HPV31.E6.69, HPV16.E6.131, HPV18.E6.126.F9, and HPV18.E6.89.

(n) The HTL780-20/30 core construct as in (a)-(m), further comprising nucleic acids encoding HPV CTL epitopes HPV18.E6.89, HPV16.E7.2.T2, HPV18.E6.44, and HPV31.E6.69+R@68.

(o) The HTL780-20/30 core construct as in (n), comprising or alternatively consisting of the multi-epitope construct HPV46-5.3/HTL780-20 (See Tables 71, 72 A and 72B).

(p) The HTL780-20/30 core construct as in (n), comprising or alternatively consisting of the multi-epitope construct HPV46-5.2/HTL780-20 (See Tables 70, 72E and 72F).

Further, certain embodiments comprising novel synthetic peptides produced by any of the methods described herein are also part of the invention. As will be apparent from the discussion below, certain embodiments comprising other methods and compositions are also contemplated as part of the present invention.

In some embodiments, the invention provides a polynucleotide comprising or alternatively consisting of:

(a) a multi-epitope construct comprising nucleic acids encoding the human papillomavirus (HPV) cytotoxic T lymphocyte (CTL) epitopes HPV16.E1.214, HPV16.E1.254, HPV16.E1.314, HPV16.E1.420, HPV16.E1.585, HPV16.E2.130, HPV16.E2.329, HPV16/52.E2.151, HPV18.E1.592, HPV18.E2.136, HPV18.E2.142, HPV18.E2.15, HPV18.E2.154, HPV18.E2.168, HPV18.E2.230, HPV18/45.E1.321, HPV18/45.E1.491, HPV31.E1.272, HPV31.E1.349, HPV31.E1.565, HPV31.E2.11, HPV31.E2.130, HPV31.E2.138, HPV31.E2.205, HPV31.E2.291, HPV31.E2.78, HPV45.E1.232, HPV45.E1.252, HPV45.E1.399, HPV45.E1.411, HPV45.E1.578, HPV45.E2.137, HPV45.E2.144, HPV45.E2.17, HPV45.E2.332, and HPV45.E2.338, wherein the nucleic acids are directly or indirectly joined to one another in the same reading frame;

(b) the multi-epitope construct of (a), further comprising nucleic acids encoding the human papillomavirus (HPV) cytotoxic T lymphocyte (CTL) epitopes HPV16.E1.493, HPV31/52.E1.557, HPV31.E2.131, HPV31.E2.127, HPV16.E2.335, HPV16.E2.37, HPV16.E2.93, HPV18.E2.211, HPV18.E2.61, HPV18.E1.266, and HPV18.E1.500, directly or indirectly joined in the same reading frame to said CTL epitope nucleic acids of (a);

(c) the multi-epitope construct of (a), further comprising nucleic acids encoding the human papillomavirus (HPV) cytotoxic T lymphocyte (CTL) epitopes HPV16.E1.1191, HPV16.E1.292, HPV16.E1.489, HPV16.E1.489, HPV16/52.E1.406, HPV18.E1.210, HPV18.E1.266, HPV18.E1.463, HPV31.E1.464, HPV18/45.E1.284, and HPV31.E1.441 directly or indirectly joined in the same reading frame to said CTL epitope nucleic acids of (a);

(d) the multi-epitope construct of (a), further comprising nucleic acids encoding the human papillomavirus (HPV) cytotoxic T lymphocyte (CTL) epitopes HPV16.E1.191, HPV16.E1.292, HPV16.E1.489, HPV16.E1.489, HPV16/52.E1.406, HPV18.E1.210, HPV18.E1.266, HPV18.E1.463, HPV31.E1.464, HPV18/45.E1.284, and HPV31.E1.441 directly or indirectly joined in the same reading frame to said CTL epitope nucleic acids of (a);

(e) a multi-epitope construct comprising nucleic acids encoding the human papillomavirus (HPV) cytotoxic T lymphocyte (CTL) epitopes HPV16.E6.106, HPV16.E6.29.L2, HPV16.E6.68.R10, HPV16.E6.75.F9, HPV16.E6.75.L2, HPV16.E6.77, HPV16.E6.80.D3, HPV16.E7.11.V10, HPV16.E7.2.T2, HPV16.E7.56.F10, HPV16.E7.86.V8, HPV18.E6.24, HPV18.E6.25.T2, HPV18.E6.53.K10, HPV18.E6.72.D3, HPV18.E6.83. R10, HPV18.E6.84.V10, HPV18.E6.89, HPV18.E6.92.V10, HPV18.E7.59. R9, HPV18/45.E6.13, HPV18/45.E6.98.F9, HPV31.E6.132.K10, HPV31.E6.15, HPV31.E6.72, HPV31.E6.73 D3, HPV31.E6.80, HPV31.E6.82R9, HPV31.E6.83, HPV31.E6.90, HPV31.E7.44.T2, HPV33.E7.11 V10, HPV45.E6.24, HPV45.E6.25 T2, HPV45.E6.37, HPV45.E6.41.R10, HPV45.E6.44, HPV45.E6.54, HPV45.E6.54.V10, HPV45.E6.71.F10, HPV45.E6.84.R9 and HPV45.E7.20, wherein the nucleic acids are directly or indirectly joined to one another in the same reading frame;

(f) the multi-epitope construct of (e), further comprising nucleic acids encoding the human papillomavirus (HPV) cytotoxic T lymphocyte (CTL) epitopes HPV16.E6.131, HPV18.E6.126.F9, HPV31.E6.69, HPV18.E6.33.F9, directly or indirectly joined in the same reading frame to said CTL epitope nucleic acids of (d);

(g) the the multi-epitope construct of (e), further comprising nucleic acids encoding the human papillomavirus (HPV) cytotoxic T lymphocyte (CTL) epitopes HPV18.E6.33, HPV16.E6.87, HPV18.E6.44, HPV31.E6.69+R@68, directly or indirectly joined in the same reading frame to said CTL epitope nucleic acids of (d);

(h) the multi-epitope construct of (a) or (b) or (c) or (d) or (e) or (f) or (g), further comprising one or more spacer nucleic acids encoding one or more spacer amino acids, directly or indirectly joined in the same reading frame to said CTL epitope nucleic acids;

(i) the multi-epitope construct of (h), wherein said one or more spacer nucleic acids are positioned between the CTL epitope nucleic acids of (a), between the CTL epitope nucleic acids of (b), between the CTL epitope nucleic acids of (c), between the CTL epitope nucleic acids of (d), between the CTL epitope nucleic acids of (a) and (b), between the CTL epitope nucleic acids of (a) and (c), between the CTL epitope nucleic acids of (a) and (d), between the CTL epitope nucleic acids of (e), between the CTL epitope nucleic acids of (f), between the CTL epitope nucleic acids of (g), between the CTL epitope nucleic acids of (e) and (f), or between the CTL epitope nucleic acids of (e) and (g);

(j) the multi-epitope construct of (h) or (i), wherein said one or more spacer nucleic acids each encode 1 to 8 amino acids;

(k) the multi-epitope construct of any of (h) to (O), wherein two or more of said spacer nucleic acids encode different (i.e., non-identical) amino acid sequences;

(l) the multi-epitope construct of any of (h) to (k), wherein two or more of said spacer nucleic acids encode an amino acid sequence different from an amino acid sequence encoded by one or more other spacer nucleic acids;

(m) the multi-epitope construct of any of (h) to (l), wherein two or more of the spacer nucleic acids encodes the identical amino acid sequence;

(n) the multi-epitope construct of any of (h) to (m), wherein one or more of said spacer nucleic acids encode an amino acid sequence comprising or consisting of three consecutive alanine (Ala) residues;

(o) the multi-epitope construct of any of (a) to (n), further comprising one or more nucleic acids encoding one or more HTL epitopes, directly or indirectly joined in the same reading frame to said CTL epitope nucleic acids and/or said spacer nucleic acids;

(p) the multi-epitope construct of (o), wherein said one or more HTL epitopes comprises a PADRE epitope;

(q) the multi-epitope construct of (o) or (p), wherein said one or more HTL epitopes comprise one or more HPV HTL epitopes;

(r) the multi-epitope construct of (q), wherein said one or more HPV HTL epitopes comprise HPV16.E1.319,HPV16.E1.337, HPV18.E1.258, HPV18.E1.458, HPV18.E2.140, HPV31.E1.015, HPV31.E1.317, HPV31.E2.67, HPV45.E1.484, HPV45.E1.510, and HPV45.E2.352;

(s) the multi-epitope construct of (r), wherein said one or more HPV HTL epitopes further comprise HPV16.E2.156, HPV16.E2.7, HPV18.E2.277, HPV31.E2.354, and HPV45.E2.67;

(t) the multi-epitope construct of (r), wherein said one or more HPV HTL epitopes further comprise HPV16.E2.160, HPV16.E2.19, HPV18.E2.127, HPV18.E2.340, and HPV31.E2.202;

(u) the multi-epitope construct of (q), wherein said one or more HPV HTL epitopes comprise HPV16.E6.13, HPV16.E6.130, HPV16.E7.13, HPV16.E7.46, HPV16.E7.76, HPV18.E6.43, HPV31.E6.132, HPV31.E6.42, HPV31.E6.78, HPV45.E6.127, and HPV45.E7.10;

(v) the multi-epitope construct of (u), wherein said one or more HPV HTL epitopes further comprise HPV18.E6.94, HPV18.E7.78, HPV31.E6.1, HPV31.E7.36, and HPV45.E7.82;

(w) the multi-epitope construct of (u), wherein said one or more HPV HTL epitopes further comprise HPV18.E6.52 and 53, HPV18.E6.94+Q, HPV18.E7.86, HPV31.E7.76, and HPV45.E6.52;

(x) the multi-epitope construct of any of (o) to (w), further comprising one or more spacer nucleic acids encoding one or more spacer amino acids directly or indirectly joined in the same reading frame between a CTL epitope and an HTL epitope or between HTL epitopes;

(y) the multi-epitope construct of (x), wherein said spacer nucleic acid encodes an amino acid sequence selected from the group consisting of: an amino acid sequence comprising or consisting of GPGPG (SEQ ID NO:______), an amino acid sequence comprising or consisting of PGPGP (SEQ ID NO:______), an amino acid sequence comprising or consisting of (GP)n, an amino acid sequence comprising or consisting of (PG)n, an amino acid sequence comprising or consisting of (GP)nG, and an amino acid sequence comprising or consisting of (PG)nP, where n is an integer between zero and eleven;

(z) the multi-epitope construct of any of (a) to (y), further comprising one or more MHC Class I and/or MHC Class II targeting nucleic acids;

(aa) the multi-epitope construct of (z), wherein said one or more targeting nucleic acids encode one or more targeting sequences selected from the group consisting of: an Ig kappa signal sequence, a tissue plasminogen activator signal sequence, an insulin signal sequence, an endoplasmic reticulum signal sequence, a LAMP-1 lysosomal targeting sequence, a LAMP-2 Tysosomal targeting sequence, an HLA-DM lysosomal targeting sequence, an HLA-DM-association sequence of HLA-DO, an Ig-a cytoplasmic domain, Ig-ss cytoplasmic domain, a ii protein, an influenza matrix protein, an HCV antigen, and a yeast Ty protein;

(bb) the multi-epitope construct of any of (a) to (aa), which is optimized for CTL and/or HTL epitope processing;

(cc) the multi-epitope construct of any of (a) to (bb), wherein said CTL nucleic acids are sorted to minimize the number of CTL and/or HTL junctional epitopes encoded therein;

(dd) the multi-epitope construct of any of (q) to (cc), wherein said HTL nucleic acids are sorted to minimize the number of CTL and/or HTL junctional epitopes encoded therein;

(ee) the multi-epitope construct of any of (a) to (dd) further comprising one or more nucleic acids encoding one or more flanking amino acid residues;

(ff) the multi-epitope construct of (ee), wherein said one or more flanking amino acid residues are selected from the group consisting of: K, R, N, Q, G, A, S, C, and T at a C+1 position of one of said CTL epitopes;

(gg) the multi-epitope construct of any of (e), (f), (h)-(n), (z)-(cc), (ee) or (ff), wherein said HPV CTL epitopes are directly or indirectly joined in the order shown in Table 47C;

(hh) the multi-epitope construct of any of (e), (g), (h)-(n), (z)-(cc), (ee) or (ff), wherein the HPV CTL epitopes are directly or indirectly joined in the order shown in Table 85;

(ii) the multi-epitope construct of any of (a), (b), (h)-(n), (z)-(cc), (ee) or (ff), wherein the HPV CTL epitopes are directly or indirectly joined in the order shown in Table 52A;

(jj) the multi-epitope construct of any of (a), (b), (h)-(n), (z)-(cc), (ee) or (ff), wherein the HPV CTL epitopes are directly or indirectly joined in the order shown in Table 52B;

(kk) the multi-epitope construct of any of (a), (c), (h)-(n), (z)-(cc), (ee) or (ff), wherein the HPV CTL epitopes are directly or indirectly joined in the order shown in Table 74;

(ll) the multi-epitope construct of any of (a), (c), (h)-(n), (z)-(cc), (ee) or (ff), wherein the HPV CTL epitopes are directly or indirectly joined in the order shown in Table 75;

(mm) the multi-epitope construct of any of (a), (d), (h)-(n), (z)-(cc), (ee) or (ff), wherein the HPV CTL epitopes are directly or indirectly joined in the order shown in Table 83;

(nn) the multi-epitope construct of any of (r), (t), (x)-(bb), (dd) or (ff), wherein the HPV HTL epitopes are directly or indirectly joined in the order shown in Table 58A;

(oo) the multi-epitope construct of any of (r), (t), (x)-(bb), (dd) or (ff), wherein the HPV HTL epitopes are directly or indirectly joined in the order shown in Table 58B;

(pp) the multi-epitope construct of any of (u), (v), (x)-(bb), (dd) or (ff), wherein the HPV HTL epitopes are directly or indirectly joined in the order of the HTL epitopes shown in Table 70;

(qq) the multi-epitope construct of any of (u), (w), (x)-(bb), (dd) or (ff), wherein the HPV HTL epitopes are directly or indirectly joined in the order shown in Table 80;

(rr) the multi-epitope construct of any of (e), (f), (h)-(n), (r), (s), or (x)-(ff), wherein the HPV HTL epitopes are directly or indirectly joined in the order shown in Table 78;

(ss) the multi-epitope construct of (e), (f), (h)-(n), (u), (v), or (x)-(ff), wherein said HPV CTL epitopes and said HPV HTL epitopes are directly or indirectly joined in the order shown in Table 70;

(tt) the multi-epitope construct of (e), (g), (h)-(n), (u), (v), or (x)-(ff), wherein said HPV CTL epitopes and said HPV HTL epitopes are directly or indirectly joined in the order shown in Table 71;

(uu) the multi-epitope construct of (a), (b), (h)-(n), (r), (t), or (x)-(ff), wherein said HPV CTL epitopes and said HPV HTL epitopes are directly or indirectly joined in the order shown in Table 63A;

(vv) the multi-epitope construct of (a), (b), (h)-(n), (r), (t), or (x)-(ff), wherein said HPV CTL epitopes and said HPV HTL epitopes are directly or indirectly joined in the order shown in Table 63C;

(ww) the multi-epitope construct of (a), (b), (h)-(n), (r), (t), or (x)-(ff), wherein said HPV CTL epitopes and said HPV HTL epitopes are directly or indirectly joined in the order shown in Table 63B;

(xx) the multi-epitope construct of (a), (b), (h)-(n), (r), (t), or (x)-(ff), wherein said HPV CTL epitopes and said HPV HTL epitopes are directly or indirectly joined in the order shown in Table 63D;

(yy) the multi-epitope construct of (a), (c), (h)-(n), (r), (s), or (x)-(ff), wherein said HPV CTL epitopes and said HPV HTL epitopes are directly or indirectly joined in the order shown in Table 84;

(zz) the multi-epitope construct of any of (a) to (ff), wherein said construct encodes a polypeptide comprising or consisting of an amino acid sequence selected from the group consisting of: the amino acid sequence shown in Table 50C, the amino acid sequence shown in Table 54A, the amino acid sequence shown in Table 54B, the amino acid sequence shown in Table 59, the amino acid sequence shown in Table 61, the amino acid sequence shown in Table 65A, the amino acid sequence shown in Table 65B, the amino acid sequence shown in Table 65C, the amino acid sequence shown in Table 65D, the amino acid sequence shown in Table 69, the amino acid sequence shown in Table 72A, the amino acid sequence shown in Table 72E, the amino acid sequence shown in Table 73A, the amino acid sequence shown in Table 76A, the amino acid sequence shown in Table 76C, the amino acid sequence shown in Table 79A, the amino acid sequence shown in Table 79B, the amino acid sequence shown in Table 81, and a combination of two or more of said amino acid sequences; and

(aaa) the multi-epitope construct of any of (a) to (ff), wherein said construct comprises a nucleic acid sequence selected from the group consisting of: the nucleotide sequence in Table 49C, the nucleotide sequence in Table 53A, the nucleotide sequence in Table 53B, the nucleotide sequence in Table 59, the nucleotide sequence in Table 61, the nucleotide sequence in Table 64A, the nucleotide sequence in Table 64B, the nucleotide sequence in Table 64C, the nucleotide sequence in Table 64D, the nucleotide sequence in Table 72B, the nucleotide sequence in Table 72F, the nucleotide sequence in Table 73B, the nucleotide sequence in Table 76B, the nucleotide sequence in Table 76D, the nucleotide sequence in Table 79A, the nucleotide sequence in Table 79B, the nucleotide sequence in Table 81, and a combination of two or more of said nucleotide sequences.

In some embodiments, the invention provides a polynucleotide comprising two multi-epitope constructs, the first comprising the HBV multi-epitope construct in any of (a) to (aaa), above, and the second comprising HBV HTL epitopes such as those in (r-w), wherein the first and second multi-epitope constructs are not directly joined, and/or are not joined in the same frame.

Each first and second multi-epitope construct may be operably linked to a regulatoru sequence such as a promoter or an IRES. The polynucleotide comprising the first and second multi-epitope contructs may comprise, e.g., at least one promoter and at least one IRES, one promoter and one IRES, two promoters, or two or more promoters and/or IRESs. The promoter may be a CMV promoter or other promoter described herein or known in the art. In preferred embodiments, the two multi-epitope constructs have the structure shown in any one of Tables 47C, 52B, 58A, 63A-D, 70, 71, 74, 75, 78, 80, 82, 83, 84, 85. The second multi-epitope construct may encode a peptide comprising or consisting of an amino acid sequence selected from the group consisting the amino acid sequence shown in Table 50C, the amino acid sequence shown in Table 54A, the amino acid sequence shown in Table 54B, the amino acid sequence shown in Table 59, the amino acid sequence shown in Table 61, the amino acid sequence shown in Table 65A, the amino acid sequence shown in Table 65B, the amino acid sequence shown in Table 65C, the amino acid sequence shown in Table 65D, the amino acid sequence shown in Table 69, the amino acid sequence shown in Table 72A, the amino acid sequence shown in Table 72E, the amino acid sequence shown in Table 73A, the amino acid sequence shown in Table 76A, the amino acid sequence shown in Table 76C, the amino acid sequence shown in Table 79A, the amino acid sequence shown in Table 79B, the amino acid sequence shown in Table 81, and a combination of two or more of said amino acid sequences. The second multi-epitope construct may comprises a nucleic acid sequence selected from the nucleotide sequence the nucleotide sequence in Table 49C, the nucleotide sequence in Table 53A, the nucleotide sequence in Table 53B, the nucleotide sequence in Table 59, the nucleotide sequence in Table 61, the nucleotide sequence in Table 64A, the nucleotide sequence in Table 64B, the nucleotide sequence in Table 64C, the nucleotide sequence in Table 64D, the nucleotide sequence in Table 72B, the nucleotide sequence in Table 72F, the nucleotide sequence in Table 73B, the nucleotide sequence in Table 76B, the nucleotide sequence in Table 76D, the nucleotide sequence in Table 79A, the nucleotide sequence in Table 79B, the nucleotide sequence in Table 81, and a combination of two or more of said nucleotide sequences.

In other embodiments, the invention provides peptides encoded by the polynucleotides described above, for example, a peptide comprising or alternatively consisting of:

(a) a multi-epitope construct comprising nucleic acids encoding the human papillomavirus (HPV) cytotoxic T lymphocyte (CTL) epitopes HPV16.E1.214, HPV16.E1.254, HPV16.E1.314, HPV16.E1.420, HPV16.E1.585, HPV16.E2.130, HPV16.E2.329, HPV16/52.E2.151, HPV18.E1.592, HPV18.E2.136, HPV18.E2.142, HPV18.E2.15, HPV18.E2.154, HPV18.E2.168, HPV18.E2.230, HPV18/45.E1.321, HPV18/45.E1.491, HPV31.E1.272, HPV31.E1.349, HPV31.E1.565, HPV31.E2.11, HPV31.E2.130, HPV31.E2.138, HPV31.E2.205, HPV31.E2.291, HPV31.E2.78, HPV45.E1.232, HPV45.E1.252, HPV45.E1.399, HPV45.E1.411, HPV45.E1.578, HPV45.E2.137, HPV45.E2.144, HPV45.E2.17, HPV45.E2.332, and HPV45.E2.338, wherein the nucleic acids are directly or indirectly joined to one another in the same reading frame;

(b) the multi-epitope construct of (a), further comprising nucleic acids encoding the human papillomavirus (HPV) cytotoxic T lymphocyte (CTL) epitopes HPV16.E1.493, HPV31/52.E1.557, HPV31.E2.131, HPV31.E2.127, HPV16.E2.335, HPV16.E2.37, HPV16.E2.93, HPV18.E2.211, HPV18.E2.61, HPV18.E1.266, and HPV18.E1.500, directly or indirectly joined in the same reading frame to said CTL epitope nucleic acids of (a);

(c) the multi-epitope construct of (a), further comprising nucleic acids encoding the human papillomavirus (HPV) cytotoxic T lymphocyte (CTL) epitopes HPV16.E1.191, HPV16.E1.292, HPV16.E1.489, HPV16.E1.489, HPV16/52.E1.406, HPV18.E1.210, HPV18.E1.266, HPV18.E1.463, HPV31.E1.464, HPV18/45.E1.284, and HPV31.E1.441 directly or indirectly joined in the same reading frame to said CTL epitope nucleic acids of (a);

(d) the multi-epitope construct of (a), further comprising nucleic acids encoding the human papillomavirus (HPV) cytotoxic T lymphocyte (CTL) epitopes HPV16.E1.191, HPV16.E1.292, HPV16.E1.489, HPV16.E1.489, HPV16/52.E1.406, HPV18.E1.210, HPV18.E1.266, HPV18.E1.463, HPV31.E1.464, HPV18/45.E1.284, and HPV31.E1.441 directly or indirectly joined in the same reading frame to said CTL epitope nucleic acids of (a);

(e) a multi-epitope construct comprising nucleic acids encoding the human papillomavirus (HPV) cytotoxic T lymphocyte (CTL) epitopes HPV16.E6.106, HPV16.E6.29.L2, HPV16.E6.68.R10, HPV16.E6.75.F9, HPV16.E6.75.L2, HPV16.E6.77, HPV16.E6.80.D3, HPV16.E7.11.V10, HPV16.E7.2.T2, HPV16.E7.56.F10, HPV16.E7.86.V8, HPV18.E6.24, HPV18.E6.25.T2, HPV18.E6.53.K10, HPV18.E6.72.D3, HPV18.E6.83.R10, HPV18.E6.84.V10, HPV18.E6.89, HPV18.E6.92.V10, HPV18.E7.59. R9, HPV18/45.E6.13, HPV18/45.E6.98.F9, HPV31.E6.132.K10, HPV31.E6.15, HPV31.E6.72, HPV31.E6.73 D3, HPV31.E6.80, HPV31.E6.82R9, HPV31.E6.83, HPV31.E6.90, HPV31.E7.44.T2, HPV33.E7.11V10, HPV45.E6.24, HPV45.E6.25 T2, HPV45.E6.37, HPV45.E6.41.R10, HPV45.E6.44, HPV45.E6.54, HPV45.E6.54.V10, HPV45.E6.71.F10, HPV45.E6.84.R9 and HPV45.E7.20, wherein the nucleic acids are directly or indirectly joined to one another in the same reading frame;

(f) the multi-epitope construct of (e), further comprising nucleic acids encoding the human papillomavirus (HPV) cytotoxic T lymphocyte (CTL) epitopes HPV16.E6.131, HPV18.E6.126.F9, HPV31.E6.69, HPV18.E6.33.F9, directly or indirectly joined in the same reading frame to said CTL epitope nucleic acids of (d);

(g) the the multi-epitope construct of (e), further comprising nucleic acids encoding the human papillomavirus (HPV) cytotoxic T lymphocyte (CTL) epitopes HPV18.E6.33, HPV16.E6.87, HPV18.E6.44, HPV31.E6.69+R@68, directly or indirectly joined in the same reading frame to said CTL epitope nucleic acids of (d);

(h) the multi-epitope construct of (a) or (b) or (c) or (d) or (e) or (f) or (g), further comprising one or more spacer nucleic acids encoding one or more spacer amino acids, directly or indirectly joined in the same reading frame to said CTL epitope nucleic acids;

(i) the multi-epitope construct of (h), wherein said one or more spacer nucleic acids are positioned between the CTL epitope nucleic acids of (a), between the CTL epitope nucleic acids of (b), between the CTL epitope nucleic acids of (c), between the CTL epitope nucleic acids of (d), between the CTL epitope nucleic acids of (a) and (b), between the CTL epitope nucleic acids of (a) and (c), between the CTL epitope nucleic acids of (a) and (d), between the CTL epitope nucleic acids of (e), between the CTL epitope nucleic acids of (f), between the CTL epitope nucleic acids of (g), between the CTL epitope nucleic acids of (e) and (f), or between the CTL epitope nucleic acids of (e) and (g);

(j) the multi-epitope construct of (h) or (i), wherein said one or more spacer nucleic acids each encode 1 to 8 amino acids;

(k) the multi-epitope construct of any of (h) to (j), wherein two or more of said spacer nucleic acids encode different (i.e., non-identical) amino acid sequences;

(l) the multi-epitope construct of any of (h) to (k), wherein two or more of said spacer nucleic acids encode an amino acid sequence different from an amino acid sequence encoded by one or more other spacer nucleic acids;

(m) the multi-epitope construct of any of (h) to (l), wherein two or more of the spacer nucleic acids encodes the identical amino acid sequence;

(n) the multi-epitope construct of any of (h) to (m), wherein one or more of said spacer nucleic acids encode an amino acid sequence comprising or consisting of three consecutive alanine (Ala) residues;

(o) the multi-epitope construct of any of (a) to (n), further comprising one or more nucleic acids encoding one or more HTL epitopes, directly or indirectly joined in the same reading frame to said CTL epitope nucleic acids and/or said spacer nucleic acids;

(p) the multi-epitope construct of (o), wherein said one or more HTL epitopes comprises a PADRE epitope;

(q) the multi-epitope construct of (o) or (p), wherein said one or more HTL epitopes comprise one or more HPV HTL epitopes;

(r) the multi-epitope construct of (q), wherein said one or more HPV HTL epitopes comprise HPV16.E1.319,HPV16.E1.337, HPV18.E1.258, HPV18.E1.458, HPV18.E2.140, HPV31.E1.015, HPV31.E1.317, HPV31.E2.67, HPV45.E1.484, HPV45.E1.510, and HPV45.E2.352;

(s) the multi-epitope construct of (r), wherein said one or more HPV HTL epitopes further comprise HPV16.E2.156, HPV16.E2.7, HPV18.E2.277, HPV31.E2.354, and HPV45.E2.67;

(t) the multi-epitope construct of (r), wherein said one or more HPV HTL epitopes further comprise HPV16.E2.160, HPV16.E2.19, HPV18.E2.127, HPV18.E2.340, and HPV31.E2.202;

(u) the multi-epitope construct of (q), wherein said one or more HPV HTL epitopes comprise HPV16.E6.13, HPV16.E6.130, HPV16.E7.13, HPV16.E7.46, HPV16.E7.76, HPV18.E6.43, HPV31.E6.132, HPV31.E6.42, HPV31.E6.78, HPV45.E6.127, and HPV45.E7.10;

(v) the multi-epitope construct of (u), wherein said one or more HPV HTL epitopes further comprise HPV18.E6.94, HPV18.E7.78, HPV31.E6.1, HPV31.E7.36, and HPV45.E7.82;

(w) the multi-epitope construct of (u), wherein said one or more HPV HTL epitopes further comprise HPV18.E6.52 and 53, HPV18.E6.94+Q, HPV18.E7.86, HPV31.E7.76, and HPV45.E6.52;

(x) the multi-epitope construct of any of (o) to (w), further comprising one or more spacer nucleic acids encoding one or more spacer amino acids directly or indirectly joined in the same reading frame between a CTL epitope and an HTL epitope or between HTL epitopes;

(y) the multi-epitope construct of (x), wherein said spacer nucleic acid encodes an amino acid sequence selected from the group consisting of: an amino acid sequence comprising or consisting of GPGPG (SEQ ID NO:______), an amino acid sequence comprising or consisting of PGPGP (SEQ ID NO:______), an amino acid sequence comprising or consisting of (GP)n, an amino acid sequence comprising or consisting of (PG)n, an amino acid sequence comprising or consisting of (GP)nG, and an amino acid sequence comprising or consisting of (PG)ₙP, where n is an integer between zero and eleven;

(z) the multi-epitope construct of any of (a) to (y), further comprising one or more MHC Class I and/or MHC Class II targeting nucleic acids;

(aa) the multi-epitope construct of (z), wherein said one or more targeting nucleic acids encode one or more targeting sequences selected from the group consisting of: an Ig kappa signal sequence, a tissue plasminogen activator signal sequence, an insulin signal sequence, an endoplasmic reticulum signal sequence, a LAMP-1 lysosomal targeting sequence, a LAMP-2 lysosomal targeting sequence, an HLA-DM lysosomal targeting sequence, an HLA-DM-association sequence of HLA-DO, an Ig-a cytoplasmic domain, Ig-ss cytoplasmic domain, a li protein, an influenza matrix protein, an HCV antigen, and a yeast Ty protein;

(bb) the multi-epitope construct of any of (a) to (aa), which is optimized for CTL and/or HTL epitope processing;

(cc) the multi-epitope construct of any of (a) to (bb), wherein said CTL nucleic acids are sorted to minimize the number of CTL and/or HTL junctional epitopes encoded therein;

(dd) the multi-epitope construct of any of (q) to (cc), wherein said HTL nucleic acids are sorted to minimize the number of CTL and/or HTL junctional epitopes encoded therein; (ee) the multi-epitope construct of any of (a) to (dd) further comprising one or more nucleic acids encoding one or more flanking amino acid residues;

(ff) the multi-epitope construct of (ee), wherein said one or more flanking amino acid residues are selected from the group consisting of: K, R, N, Q, G, A, S, C, and T at a C+1 position of one of said CTL epitopes;

(gg) the multi-epitope construct of any of (e), (f), (h)-(n), (z)-(cc), (ee) or (ff), wherein said HPV CTL epitopes are directly or indirectly joined in the order shown in Table 47C;

(hh) the multi-epitope construct of any of (e), (g), (h)-(n), (z)-(cc), (ee) or (ff), wherein the HPV CTL epitopes are directly or indirectly joined in the order shown in Table 85;

(ii) the multi-epitope construct of any of (a), (b), (h)-(n), (z)-(cc), (ee) or (ff), wherein the HPV CTL epitopes are directly or indirectly joined in the order shown in Table 52A;

(jj) the multi-epitope construct of any of (a), (b), (h)-(n), (z)-(cc), (ee) or (ff), wherein the HPV CTL epitopes are directly or indirectly joined in the order shown in Table 52B;

(kk) the multi-epitope construct of any of (a), (c), (h)-(n), (z)-(cc), (ee) or (ff), wherein the HPV CTL epitopes are directly or indirectly joined in the order shown in Table 74;

(ll) the multi-epitope construct of any of (a), (c), (h)-(n), (z)-(cc), (ee) or (ff), wherein the HPV CTL epitopes are directly or indirectly joined in the order shown in Table 75;

(mm) the multi-epitope construct of any of (a), (d), (h)-(n), (z)-(cc), (ee) or (ff), wherein the HPV CTL epitopes are directly or indirectly joined in the order shown in Table 83;

(nn) the multi-epitope construct of any of (r), (t), (x)-(bb), (dd) or (ff), wherein the HPV HTL epitopes are directly or indirectly joined in the order shown in Table 58A;

(oo) the multi-epitope construct of any of (r), (t), (x)-(bb), (dd) or (ff), wherein the HPV HTL epitopes are directly or indirectly joined in the order shown in Table 58B;

(pp) the multi-epitope construct of any of (u), (v), (x)-(bb), (dd) or (ff), wherein the HPV HTL epitopes are directly or indirectly joined in the order of the HTL epitopes shown in Table 70;

(qq) the multi-epitope construct of any of (u), (w), (x)-(bb), (dd) or (ff), wherein the HPV HTL epitopes are directly or indirectly joined in the order shown in Table 80;

(rr) the multi-epitope construct of any of (e), (f), (h)-(n), (r), (s), or (x)-(ff), wherein the HPV HTL epitopes are directly or indirectly joined in the order shown in Table 78;

(ss) the multi-epitope construct of (e), (f), (h)-(n), (u), (v), or (x)-(ff), wherein said HPV CTL epitopes and said HPV HTL epitopes are directly or indirectly joined in the order shown in Table 70;

(tt) the multi-epitope construct of (e), (g), (h)-(n), (u), (v), or (x)-(ff), wherein said HPV CTL epitopes and said HPV HTL epitopes are directly or indirectly joined in the order shown in Table 71;

(uu) the multi-epitope construct of (a), (b), (h)-(n), (r), (t), or (x)-(ff), wherein said HPV CTL epitopes and said HPV HTL epitopes are directly or indirectly joined in the order shown in Table 63A;

(vv) the multi-epitope construct of (a), (b), (h)-(n), (r), (t), or (x)-(ff), wherein said HPV CTL epitopes and said HPV HTL epitopes are directly or indirectly joined in the order shown in Table 63C;

(ww) the multi-epitope construct of (a), (b), (h)-(n), (r), (t), or (x)-(ff), wherein said HPV CTL epitopes and said HPV HTL epitopes are directly or indirectly joined in the order shown in Table 63B;

(xx) the multi-epitope construct of (a), (b), (h)-(n), (r), (t), or (x)-(ff), wherein said HPV CTL epitopes and said HPV HTL epitopes are directly or indirectly joined in the order shown in Table 63D;

(yy) the multi-epitope construct of (a), (c), (h)-(n), (r), (s), or (x)-(ff), wherein said HPV CTL epitopes and said HPV HTL epitopes are directly or indirectly joined in the order shown in Table 84;

(zz) the multi-epitope construct of any of (a) to (ff), wherein said construct encodes a polypeptide comprising or consisting of an amino acid sequence selected from the group consisting of: the amino acid sequence shown in Table 50C, the amino acid sequence shown in Table 54A, the amino acid sequence shown in Table 54B, the amino acid sequence shown in Table 59, the amino acid sequence shown in Table 61, the amino acid sequence shown in Table 65A, the amino acid sequence shown in Table 65B, the amino acid sequence shown in Table 65C, the amino acid sequence shown in Table 65D, the amino acid sequence shown in Table 69, the amino acid sequence shown in Table 72A, the amino acid sequence shown in Table 72E, the amino acid sequence shown in Table 73A, the amino acid sequence shown in Table 76A, the amino acid sequence shown in Table 76C, the amino acid sequence shown in Table 79A, the amino acid sequence shown in Table 79B, the amino acid sequence shown in Table 81, and a combination of two or more of said amino acid sequences; and

(aaa) the multi-epitope construct of any of (a) to (ff), wherein said construct comprises a nucleic acid sequence selected from the group consisting of: the nucleotide sequence in Table 49C, the nucleotide sequence in Table 53A, the nucleotide sequence in Table 53B, the nucleotide sequence in Table 59, the nucleotide sequence in Table 61, the nucleotide sequence in Table 64A, the nucleotide sequence in Table 64B, the nucleotide sequence in Table 64C, the nucleotide sequence in Table 64D, the nucleotide sequence in Table 72B, the nucleotide sequence in Table 72F, the nucleotide sequence in Table 73B, the nucleotide sequence in Table 76B, the nucleotide sequence in Table 76D, the nucleotide sequence in Table 79A, the nucleotide sequence in Table 79B, the nucleotide sequence in Table 81, and a combination of two or more of said nucleotide sequences.

In other embodiments, the invention provides cells comprising the polynucleotides and/or polypeptides above; compositions comprising the polynucleotides and/or polypeptides and/or cells; methods for making these polynucleotides, polypeptides, cells and compositions; and methods for stimulating an immune response (e.g. therapeutic and/or prophylactic methods) utilizing these polynucleotides and/or polypeptides and/or cells and/or compositions. The invention is described in further detail below.

### BRIEF DESCRIPTION OF THE DRAWINGS

FIG. 1 illustrates a computer system for performing automatic optimization of multi-epitope constructs in accordance with certain embodiments of the invention.

FIGS. 2A and 2B illustrate an exemplary input text file containing user input parameters used for executing a Junctional Analyzer program, in accordance with certain embodiments of the invention.

FIG. 3 illustrates a flow chart diagram of a software program of the invention for identifying optimal multi-epitope constructs, in accordance with certain embodiments of the invention.

FIGS. 4A, 4B, 4C, and 4D illustrate an exemplary output text file containing output results of a Junctional Analyzer program, in accordance with certain embodiments of the invention.

FIG. 5 illustrates allele specific motifs of five A3 supertype alleles: A*0301, A*1101, A*3101, A*3301, and A*6801. Individual residues, or groups of residues, associated for each non-anchor position with either good (“preferred”) or poor (“deleterious”) binding capacities to each individual allele are shown.

FIG. 6 illustrates the A3 supermotif. Numbers in parenthesis indicate the number of molecules for which the residue or residue group was preferred or deleterious.

FIGS. 7A and 7B summarize the motifs for the B7 supertype alleles (FIG. 7A) and for the B7 supermotif (FIG. 7B, first panel). The second panel of FIG. 7B illustrates the B7 supermotif. Values in parenthesis indicate the frequency that a residue or residue group was preferred or deleterious.

FIG. 8 illustrates relative average binding capacity of the A*0101 motif 9-mer peptides as a function of the different amino acid residues occurring at each of the non-anchor positions. The first two panels of FIG. 8 depict data, while the second two panels depict graphics. Data sets from either 2-9, 3-9 peptide sets were analyzed and tabulated. The 2-9 and 3-9 sets contained 101 and 85 different peptides, respectively. Maps of secondary effects influencing the binding capacity of 9-mer peptides carrying the 2-9, 3-9, and A*0101 motifs are also shown.

FIG. 9 illustrates relative average binding capacity of the A*0101 10-mer peptides as a function of the different amino acid residues occurring at each of the non-anchor positions. Data sets from either 2-10 or 3-10 motif sets of peptides were analyzed and tabulated. The 2-10 and 3-10 sets contained 91 and 89 different peptides, respectively. Maps of secondary effects influencing the binding capacity of 10-mer peptides carrying the 2-10 and/or 3-10 A1 motifs are also presented.

FIG. 10 illustrates preferred and deleterious secondary anchor residues for the refined A24 9-mer and 10-mer motifs.

FIGS. 11A and 11B illustrate immunogenicity data for peptides contained within the minigene constructs HPV43-3, HPV43-3R, HPV43-4 and HPV43-4R. Immunogenicity was assessed in ELISA assays by detecting the amount of secreted IFN-γ using a monoclonal antibody specific for murine IFN-γ. The IFN-γ ELISA data was converted to secretory units (“SU”) for evaluation. The SU calculation was based on the number of cells that secrete 100 pg of IFN-γ in response to a particular peptide, corrected for the background amount of IFN-γ produced in the absence of peptide.

FIGS. 12A and 12B illustrate immunogenicity data for peptides contained within the minigene constructs HPV43-3R, HPV43-3RC and HPV43-3RN. Immunogenicity was assessed using ELISA assays as described above.

FIGS. 13A and 13B illustrate immunogenicity data for peptides contained within the minigene constructs HPV43-3R, HPV43-3RC and HPV43-3RN. Immunogenicity was assessed in ELISPOT assays used to measure MHC class II restricted responses. Purified splenic cells (4×10⁵/well), isolated using MACS columns (Milteny), and irradiated splenocytes (1×10⁵ cells/well) were added to membrane-backed 96 well ELISA plates (Millipore) pre-coated with monoclonal antibody specific for murine IFN-γ (Mabtech). Cells were cultured with 10 μg/ml peptide for 20 hours at 37 degrees C. The IFN-γ secreting cells were detected by incubation with biotinylated anti-mouse IFN-γ antibody (Mabtech), followed by incubation with Avidin-Peroxidase Complex (Vectastain). The plates were developed using AEC (3-amino-9-ethyl-carbazole; Sigma), washed and dried. Spots were counted using the Zeiss KS ELISPOT reader. The results are presented as the number of IFN-γ spot forming cells (“SFC”) per 10⁶ T cells.

FIGS. 14A and 14B illustrate immunogenicity data for peptides contained within the minigene constructs HPV43-4R, HPV43-4RC and HPV43-4RN. Immunogenicity was assessed using ELISA assays as described above.

FIGS. 15A and 15B illustrate immunogenicity data for peptides contained within the minigene constructs HPV43-4R, HPV43-4RC and HPV43-4RN. Immunogenicity was assessed in ELISPOT assays as described above.

FIGS. 16A and 16B illustrate immunogenicity data for peptides contained within the minigene constructs HPV46-5 and HPV46-6. Immunogenicity was assessed using ELISA assays as described above.

FIGS. 17A and 17B illustrate immunogenicity data for peptides contained within the minigene constructs HPV46-5 and HPV46-6. Immunogenicity was assessed in ELISPOT assays as described above.

FIGS. 18A and 18B illustrate immunogenicity data for peptides contained within the minigene constructs HPV47-1 and HPV47-2. Immunogenicity was assessed using ELISA assays as described above.

FIGS. 19A and 19B illustrate immunogenicity data for peptides contained within the minigene constructs HPV46-5 and HPV46-5/HTL5. Immunogenicity was assessed in ELISPOT assays as described above.

FIGS. 20A and 20B illustrate immunogenicity data for peptides contained within the minigene constructs HPV64, HPV64R and a peptide pool. Immunogenicity was assessed using ELISA assays as described above.

FIGS. 21A and 21B illustrate immunogenicity data for peptides contained within the minigene constructs HPV46-5 and HPV46-5.2/HTL-20. Immunogenicity was assessed ELISPOT assays as described above.

FIGS. 22A and 22B illustrate immunogenicity data for peptides contained within the minigene constructs HPV46-5 and HPV46-5.2/HTL-20. Immunogenicity was assessed in ELISPOT assays as described above.

FIGS. 23A and 23B illustrate immunogenicity data for peptides contained within the minigene constructs HPV46-5 and HPV46-5.2 as compared to HPV 46-5.3. Immunogenicity was assessed in ELISPOT assays as described above.

FIGS. 24A and 24B illustrate immunogenicity data for peptides contained within the minigene constructs HPV46-5 and HPV46-5.2 as compared to HPV 46-5.3. Immunogenicity was assessed in ELISPOT assays as described above.

FIGS. 25A and 25B illustrate immunogenicity data for peptides contained within the minigene constructs HPV46-5 and HPV46-5.2 as compared to HPV 46-5.3. Immunogenicity was assessed in ELISPOT assays as described above.

FIGS. 26A and 26B illustrate immunogenicity data for peptides contained within the minigene constructs HPV46-5 and HPV46-5.2 as compared to HPV 46-5.3. Immunogenicity was assessed in ELISPOT assays as described above.

FIGS. 27A and 27B illustrate immunogenicity data for peptides contained within the minigene constructs HPV47-1 and HPV47-2. Immunogenicity was assessed in ELISPOT assays as described above.

FIG. 28 illustrates immunogenicity data for peptides contained within the minigene constructs HPV47-1 and HPV47-2. Immunogenicity was assessed in ELISPOT assays as described above.

FIG. 29 illustrates immunogenicity data for peptides contained within the minigene constructs HPV47-1 and HPV47-2. Immunogenicity was assessed in ELISPOT assays as described above.

FIG. 30 illustrates immunogenicity data for peptides contained within the minigene constructs E1/E2 HTL 780.21 and 780.22. Immunogenicity was assessed in ELISPOT assays as described above.

FIG. 31 illustrates immunogenicity data for peptides contained within the minigene constructs E1/E2 HTL 780.21 fix and 780.22 fix. Immunogenicity was assessed in ELISPOT assays as described above.

FIGS. 32A and 32B illustrate immunogenicity data for peptides contained within the minigene constructs HPV47-1, HPV47-1/HTL-21 and HPV47-1/HTL-22. Immunogenicity was assessed in ELISPOT assays as described above.

FIGS. 33A and 33B illustrate immunogenicity data for peptides contained within the minigene constructs HPV47-2, HPV47-2/HTL-21 and HPV47-2/HTL-22. Immunogenicity was assessed in ELISPOT assays as described above.

FIGS. 34A and 34B illustrate immunogenicity data for peptides contained within the minigene constructs HPV47-3 and HPV47-4. Immunogenicity was assessed in ELISPOT assays as described above.

FIG. 35 illustrates immunogenicity data for peptides contained within the minigene constructs HPV47-3 and HPV47-4. Immunogenicity was assessed in ELISPOT assays as described above.

FIG. 36 illustrates immunogenicity data for peptides contained within the minigene constructs HPV47-3 and HPV47-4. Immunogenicity was assessed in ELISPOT assays as described above.

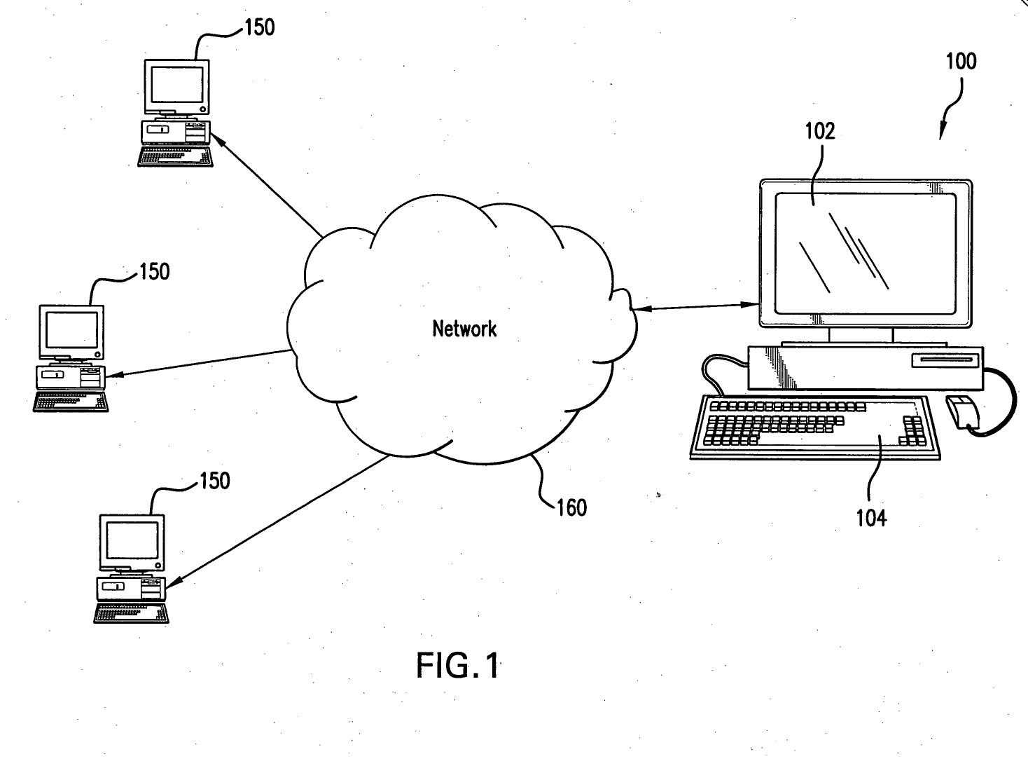

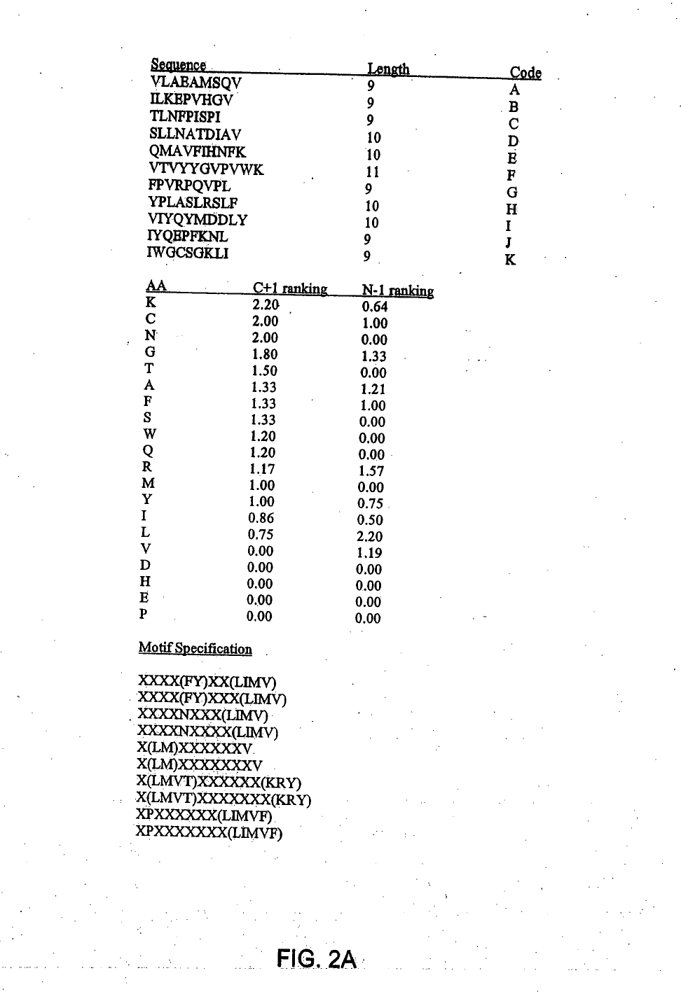

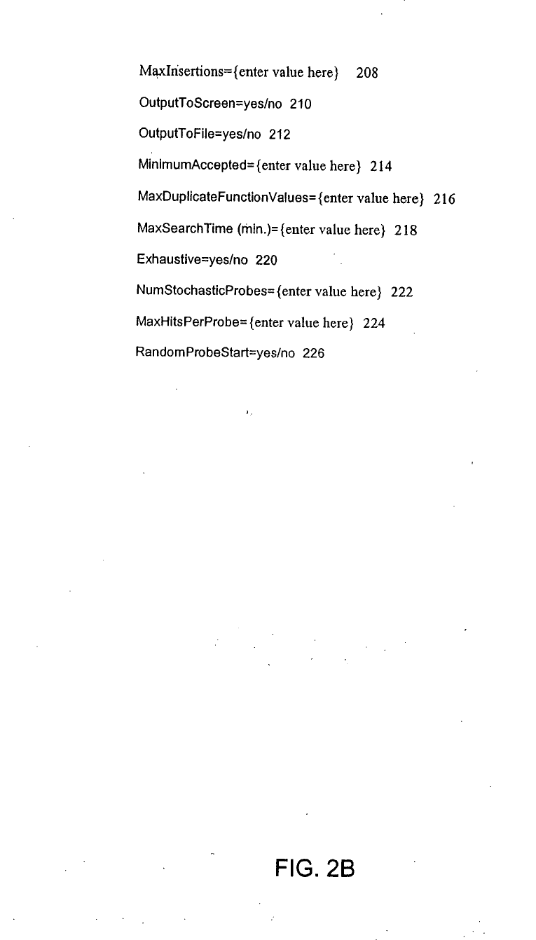

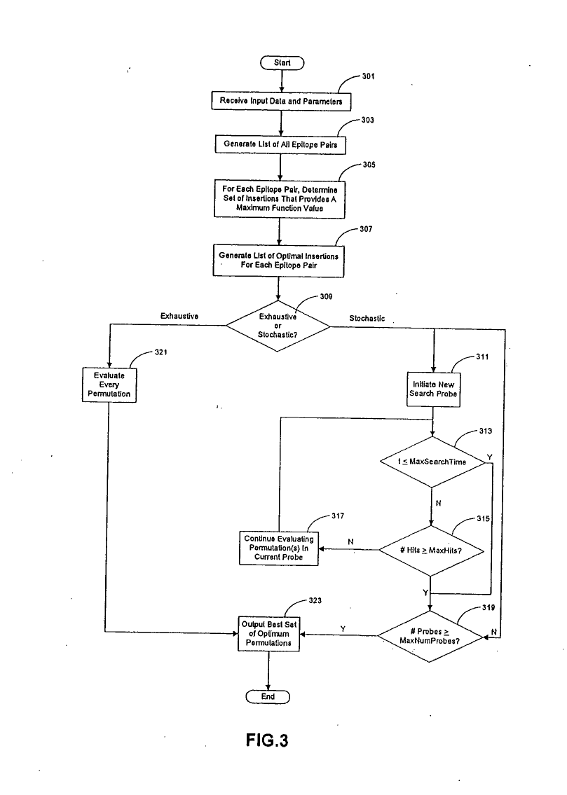

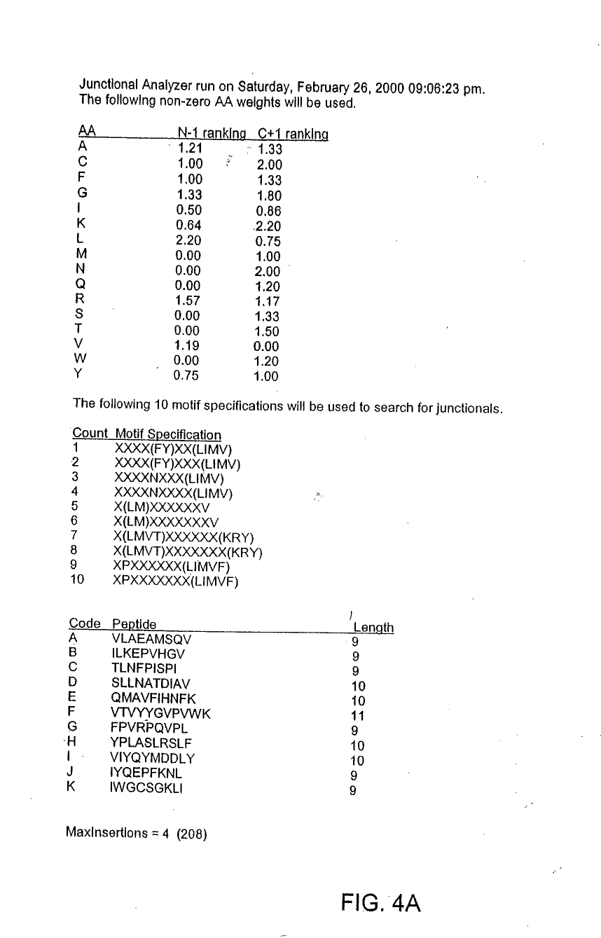

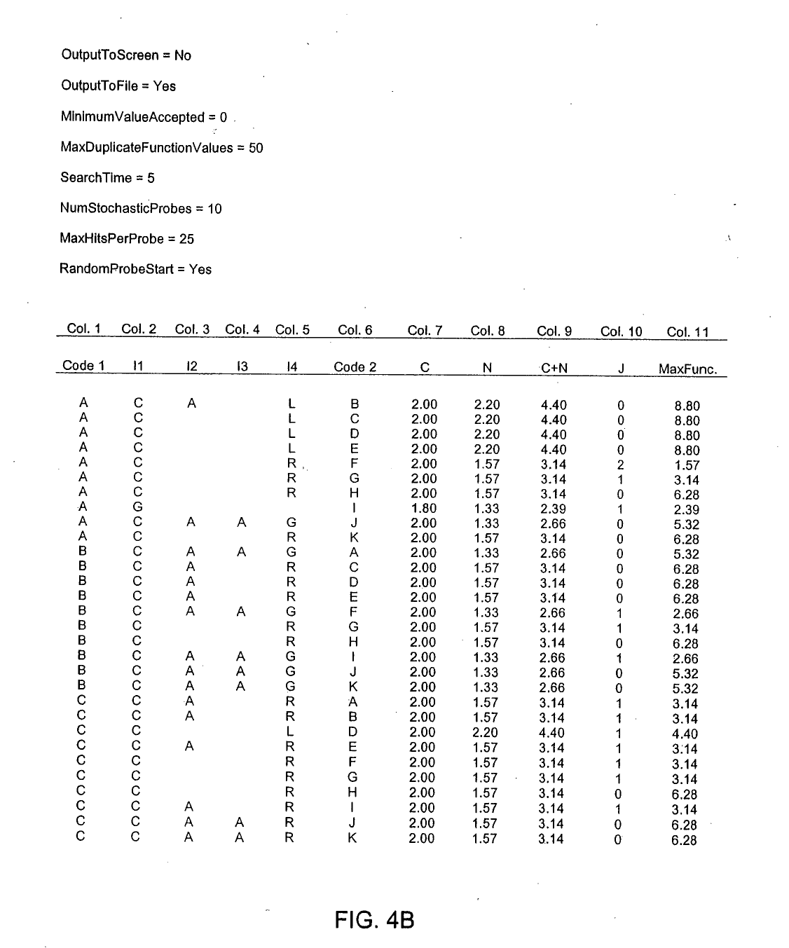

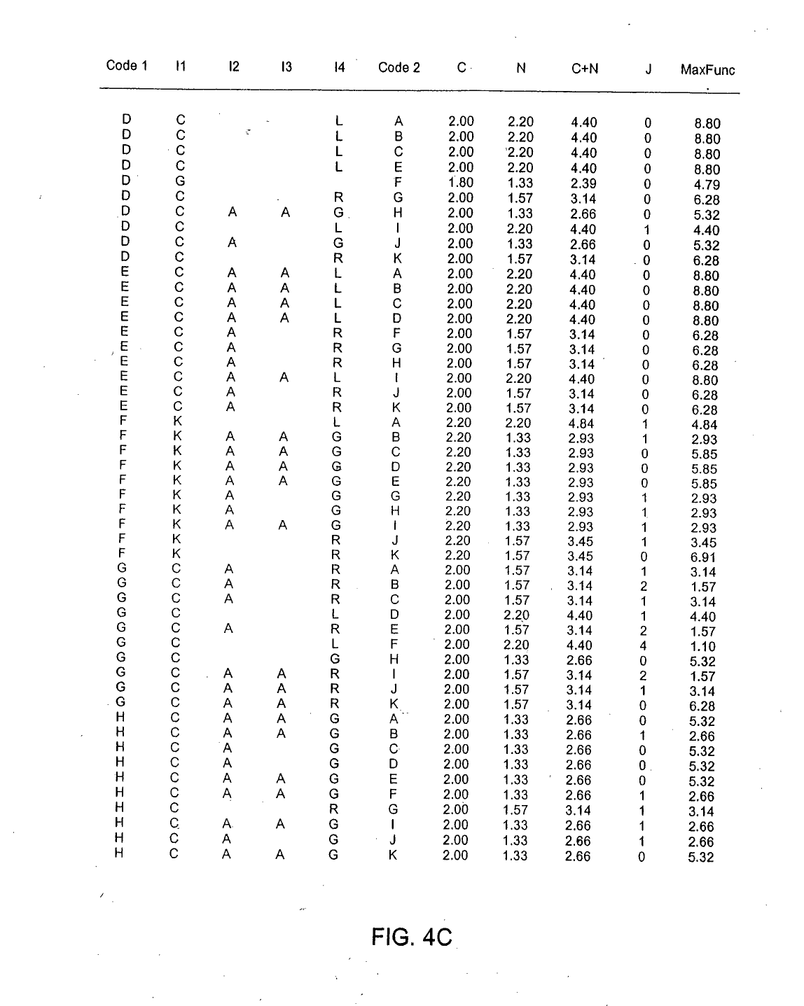

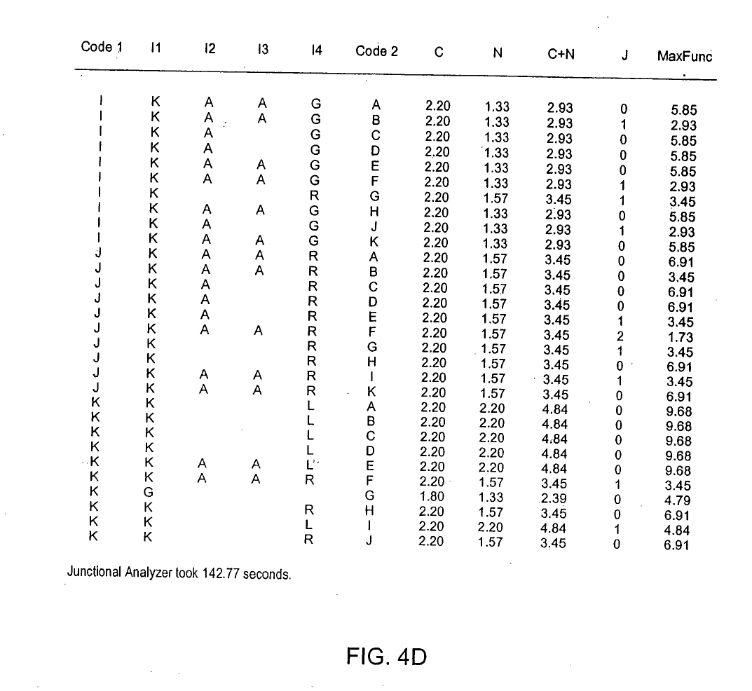

### DETAILED DESCRIPTION OF THE INVENTION

The peptides and corresponding nucleic acid compositions of the present invention are useful for stimulating an immune response to HPV by stimulating the production of CTL and/or HTL responses. The peptide epitopes, which are derived directly or indirectly from naturally occurring HPV protein amino acid sequences, are able to bind to HLA molecules and stimulate an immune response to HPV. The complete sequence of the HPV proteins to be analyzed can be obtained from Genbank. The complete sequences of HPV proteins analyzed with regard to certain embodiments of the invention as disclosed herein are provided herein in Table 1. Epitopes and analogs of HPV can also be identified from the HPV sequences provided in Table 1 according to the methods of the invention. In certain embodiments, epitopes and analogs can also be readily determined from sequence information that may subsequently be discovered for heretofore unknown variants of HPV, as will be clear from the disclosure provided below.

TABLE 1

HPV STRAINS AND AMINO ACID SEQUENCES OF
HPV PROTEINS

| Stain | | | |
|---|---|---|---|
| and | Acces- | SEQ | |
| Pro- | sion | ID | |
| tein | No. | NO | Sequence |
| HPV6A | | | MADDSGTENEGSGCTGWFMVEAIVQHPTGTQISD |
| E1 | | | DEDEEVEDSGYDMVDFIDDSNITHNSLEAQALFN |
| | | | RQEADTHYATVQDLKRKYLGSPYVSPINTIAEAV |
| | | | ESEISPRLDAIKLTRQPKKVKRRLFQTRELTDSG |
| | | | YGYSEVEAGTGTQVEKHGVPENGGDGQEKDTGRD |
| | | | IEGEEHTEAEAPTNSVREHAGTAGILELLKCKDL |
| | | | RAALLGKFKECFGLSFIDLIRPFKSDKTTCADWV |
| | | | VAGFGIHHSISEAFQKLIEPLSLYAHIQWLTNAW |
| | | | GMVLLVLVRFKVNKSRSTVARTLATLLNIPDNQM |
| | | | LIEPPKIQSGVAALYWFRTGISNASTVIGEAPEW |
| | | | ITRQTVIEHGLADSQFKLTEMVQWAYDNDICEES |
| | | | EIAFEYAQRGDFDSNARAFLNSNMQAKYVKDCAT |
| | | | MCRHYKHAEMRKMSIKQWIKHRGSKIEGTGNWKP |
| | | | IVQFLRHQNIEFIPFLSKFKLWLHGTPKKNCIAI |
| | | | VGPPDTGKSYFCMSLISFLGGTVISHVNSSSHFW |
| | | | LQPLVDAKVALLDDATQPCWIYMDTYMRNLLDGN |
| | | | PMSIDRKHKALTLIKCPPLLVTSNIDITKEEKYK |
| | | | YLHTRVTTFTFPNPFPFDRNGNAVYELSNANWKC |
| | | | FFERLSSSLDIQDSEDEEDGSNSQAFRCVPGTVV |
| | | | RTL |
| HPV6A | | | MEAIAKRLDACQEQLLELYEENSTDLNKHVLHWK |
| E2 | | | CMRHESVLLYKAKQMGLSHIGMQVVPPLKVSEAK |
| | | | GHNAIEMQMHLESLLKTEYSMEPWTLQETSYEM |
| | | | WQTPPKRCFKKRGKTVEVKFDGCANNTMDYVV |
| | | | WTDVYVQDTDSWVKVHSMVDAKGIYYTCGQFKT |
| | | | YYVNFVKEAEKYGSTKQWEVCYGSTVICSPASVS |
| | | | STTQEVSIPESTTYTPAQTSTPVSSSTQEDAVQT |
| | | | PPRKRARGVQQSPCNALCVAHIGPVDSGNHNLIT |
| | | | NNHDQHQRRNNSNSSATPIVQFQGESNCLKCFRY |
| | | | RLNDKHRHLFDLISSTWHWASPKAPHKHAIVTVT |
| | | | YHSEEQRQQFLNVVKIPPTIRHKLGFMSLHLL |
| HPV6A | | | MAAQLYVLLHLYLALHKKYPFLNLLHTPPHRPPP |
| E4 | | | LCPQAPRKTQCKRRLENEHEESNSHLATPCVWPT |
| | | | LDPWTVETTTSSLTITTSTKEGTTVTVQLRL |
| HPV6A | | | MEVVPVQIAAGTTSTLILPVIIAFVVCFVSIILI |
| E5 | | | VWISDFIVYTSVLVLTLLLYLLLWLLLTTPLQFF |
| | | | LLTLLVCYCPALYIHHYIVNTQQ |
| HPV6A | | | MESANASTSATTIDQLCKTFNLSMHTLQINCVFC |
| E6 | | | KNALTTAEIYSYAYKQLKVLFRGGYPYAACACCL |
| | | | EFHGKINQYRHFDYAGYATTVEEETKQDILDVLI |
| | | | RCYLCHKPLCEVEKVKHILTKARFIKLNCTWKGR |
| | | | CLHCWTTTCMEDMLP |
| HPV6A | | | MHGRHVTLKDIVLDLQPPDPVGLHCYEQLVDSSE |
| E7 | | | DEVDEVDGQDSQPLKQHFQIVTCCCGCDSNVRLV |
| | | | VQCTETDIREVQQLLLGTLDIVCPICAPKT |
| HPV6A | | | MWRPSDSTVYVPPPNPVSKVVATDAYVTRTNIFY |
| L1 | | | HASSSRLLAVGHPYFSIKRANKTVVPKVSGYQYR |
| | | | VFKVVLPDPNKFALPDSSLFDPTTQRLVWACTGL |
| | | | EVGRGQPLGVGVSGHPFLNKYDDVENSGSGGNPG |
| | | | QDNRVNVGMDYKQTQLCMVGCAPPLGEHWGKGKQ |
| | | | CTNTPVQAGDCPPLELITSVIQDGDMVDTGFGAM |
| | | | NFADLQTNKSDVPIDICGTTCKYPDYLQMAADPY |
| | | | GDRLFFFLRKEQMFARHFFNRAGEVGEPVPDTLI |
| | | | IKGSGNRTSVGSSIYVNTPSGSLVSSEAQLFNKP |
| | | | YWLQKAQGHNNGICWGNQLFVTVVDTTRSTNMTL |
| | | | CASVTTSSTYTNSDYKEYMRHVEEYDLQFIFQLC |
| | | | SITLSAEVMAYIHTMNPSVLEDWNFGLSPPPNGT |
| | | | LEDTYRYVQSQAITCQKPTPEKEKPDPYKNLSFW |
| | | | EVNLKEKFSSELDQYPLGRKFLLQSGYRGRSSIR |
| | | | TGVKRPAVSKASAAPKRKRAKTKR |
| HPV6A | | | MAHSRARRRKRASATQLYQTCKLTGTCPPDVIPK |
| L2 | | | VEHNTIADQILKWGSLGVFFGGLGIGTGSGTGGR |
| | | | TGYVPLGTSAKPSITSGPMARPPVVVEPVAPSDP |
| | | | SIVSLIEESAIINAGAPEIVPPAHGGFTITSSET |
| | | | TTPAILDVSVTSHTTTSIFRNPVFTEPSVTQPQP |
| | | | PVEANGHILISAPTITSHPIEEIPLDTFVISSSD |
| | | | SGPTSSTPVPGTAPRPRVGLYSRALHQVQVTDPA |
| | | | FLSTPQRLITYDNPVYEGEDVSVQFSHDSIHNAP |
| | | | DEAFMDIIRLHRPAIASRRGLVRYSRIGQRGSMH |
| | | | TRSGKHIGARIHYFYDISPIAQAAEEIEMHPLVA |
| | | | AQDDTFDIYAESFEPDINPTQHPVTNISDTYLTS |
| | | | TPNTVTQPWGNTTVPLSSIPNDLFLQSGPDITFP |
| | | | TAPMGTPFSPVTALPTGPVFITGSGFYLHPAWYF |
| | | | ARKRRKRIPLFFSDVAA |
| HPV6B | | | MADDSGTENEGSGCTGWFMVEAIVQHPTGTQISD |
| E1 | | | DEDEEVEDSGYDMVDFDDSNITHNSLEAQALFNR |
| | | | QEADTHYATVQDLKRKYLGSPYVSPINTIAEAVE |
| | | | SEISPRLDAIKLTRQPKKVKRRLFQTRELTDSGY |
| | | | GYSEVEAGTGTQVEKHGVPENGGDGQEKDTGRDI |
| | | | EGEEHTEAEAPTNSVREHAGTAGILELLKCKDLR |
| | | | AALLGKFKECFGLSFIDLIRPFKSDKTTCLDWVV |
| | | | AGFGIHHSISEAFQKLIEPLSLYAHIQWLTNAWG |
| | | | MVLLVLLRFKVNKSRSTVARTLATLLNIPENQML |
| | | | IEPPKIQSGVAALYWFRTGISNASTVIGEAPEWI |
| | | | TRQTVIEHGLADSQFKLTEMVQWAYDNDICEESE |
| | | | IAFEYAQRGDFDSNARAFLNSNMQAKYVKDCATM |
| | | | CRHYKHAEMRKMSIKQWIKHRGSKIEGTGNWKPI |
| | | | VQFLRHQNIEFIPFLTKFKLWLHGTPKKNCIAIV |
| | | | GPPDTGKSYFCMSLISFLGGTVISHVNSSSHFWL |
| | | | QPLVDAKVALLDDATQPCWIYMDTYMRNLLDGNP |
| | | | MSIDRKHKALTLIKCPPLLVTSNIDITKEDKYKY |
| | | | LHTRVTTFTFPNPFPFDRNGNAVYELSNTNWKCF |
| | | | FERLSSSLDIQDSEDEEDGSNSQAFRCVPGTVVR |
| | | | TL |
| HPV6B | | | MEAIAKRLDACQEQLLELYEENSTDLHKHVLHWK |
| E2 | | | CMRHESVLLYKAKQMGLSHIGMQVVPPLKVSEAK |
| | | | GHNAIEMQMHLESLLRTEYSMEPWTLQETSYEM |
| | | | WQTPPKRCFKKRGKTVEVKFDGCANNTMDYVV |
| | | | WTDVYVQDNDTWVKVHSMVDAKGIYYTCGQFK |
| | | | TYYVNFVKEAEKYGSTKHWEVCYGSTVICSPASV |
| | | | SSTTQEVSIPESTTYTPAQTSTLVSSSTKEDAVQ |
| | | | TPPRKRARGVQQSPCNALCVAHIGPVDSGNHNLI |
| | | | TNNHDQHQRRNNSNSSATPIVQFQGESNCLKCFR |
| | | | YRLNDRHRHLFDLISSTWHWASSKAPHKHAIVTV |
| | | | TYDSEEQRQQFLDVVKIPPTISHKLGFMSLHLL |
| HPV6B | | | MGAPNIGKYVMAAQLYVLLHLYLALHKKYPFLN |
| E4 | | | LLHTPPHRPPPLCPQAPRKTQCKRRLGNEHEESN |
| | | | SPLATPCVWPTLDPWTVETTTSSLTITTSTKDGT |
| | | | TVTVQLRL |
| HPV6B | | | MEVVPVQIAAGTTSTFILPVIIAFVVCFVSIILI |
| E5A | | | VWISEFIVYTSVLVLTLLLYLLLWLLLTTPLQFF |
| | | | LLTLLVCYCPALYIHYYIVTTQQ |
| HPV6B | | | MMLTCQFNDGDTWLGLWLLCAFIVGMLGLLLMH |
| E5B | | | YRAVQGDKHTKCKKCNKHNCNDDYVTMHYTTD |
| | | | GDYIYMN |
| HPV6B | | | MESANASTSATTIDQLCKTFNLSMHTLQINCVFC |
| E6 | | | KNALTTAEIYSYAYKHLKVLFRGGYPYAACACCL |
| | | | EFHGKINQYRHFDYAGYATTVEEETKQDILDVLI |
| | | | RCYLCHKPLCEVEKVKHILTKARFIKLNCTWKGR |
| | | | CLHCWTTCMEDMLP |
| HPV6B | | | MHGRHVTLKDIVLDLQPPDPVGLHCYEQLVDSSE |
| E7 | | | DEVDEVDGQDSQPLKQHFQIVTCCCGCDSNVRLV |
| | | | VQCTETDIREVQQLLLGTLNIVCPICAPKT |
| HPV6B | | | MWRPSDSTVYVPPPNPVSKVVATDAYVTRTNIFY |
| L1 | | | HASSSRLLAVGHPYFSIKRANKTVVPKVSGYQYR |
| | | | VFKVVLPDPNKFALPDSSLFDPTTQRLVWACTGL |
| | | | EVGRGQPLGVGVSGHPFLNKYDDVENSGSGGNPG |
| | | | QDNRVNVGMDYKQTQLCMVGCAPPLGEHWGKGKQ |
| | | | CTNTPVQAGDCPPLELITSVIQDGDMVDTGFGAM |
| | | | NFADLQTNKSDVPIDICGTTCKYPDYLQMAADPY |
| | | | GDRLFFFLRKEQMFARHFFNRAGEVGEPVPDTLI |
| | | | IKGSGNRTSVGSSIYVNTPSGSLVSSEAQLFNKP |
| | | | YWLQKAQGHNNGICWGNQLFVTVVDTTRSTNMTL |
| | | | CASVTTSSTYTNSDYKEYMRHVEEYDLQFIFQLC |
| | | | SITLSAEVMAYIHTMNPSVLEDWNFGLSPPPNGT |
| | | | LEDTYRYVQSQAITCQKPTPEKEKPDPYKNLSFW |
| | | | EVNLKEKFSSELDQYPLGRKFLLQSGYRGRSSIR |
| | | | TGVKRPAVSKASAAPKRKRAKTKR |
| HPV6B | | | MAHSRARRRKRASATQLYQTCKLTGTCPPDVIPK |
| L2 | | | VEHNTIADQILKWGSLGVFFGGLGIGTGSGTGGR |
| | | | TGYVPLQTSAKPSITSGPMARPPVVVEPVAPSDP |
| | | | SIVSLIEESAIINAGAPEIVPPAHGGFTITSSET |
| | | | TTPAILDVSVTSHTTTSIFRNPVFTEPSVTQPQP |
| | | | PVEANGHILISAPTVTSHPIEEIPLDTFVVSSSD |
| | | | SGPTSSTPVPGTAPRPRVGLYSRALHQVQVTDPA |
| | | | FLSTPQRLITYDNPVYEGEDVSVQFSHDSIHNAP |
| | | | DEAFMDIIRLHRPAIASRRGLVRYSRIGQRGSMH |
| | | | TRSGKHIGARIHYFYDISPIAQAAEEIEMHPLVA |
| | | | AQDDTFDIYAESFEPGINPTQHPVTNISDTYLTS |
| | | | TPNTVTQPWGNTTVPLSLPNDLFLQSGPDITFPT |
| | | | APMGTPFSPVTPALPTGPVFITGSGFYLHPAWYF |
| | | | ARKRRKRIPLFFSDVAA |
| HPV11 | | | MADDSGTENEGSGCTGWFMVEAIVEHTTGTQISE |
| E1 | | | DEEEEVEDSGYDMVDFIDDRHITQNSVEAQALFN |
| | | | RQEADAHYATVQDLKRKYLGSPYVSPISNVANAV |
| | | | ESEISPRLDAIKLTTQPKKVKRRLFETRELTDSG |
| | | | YGYSEVEAATQVEKHGDPENGGDGQERDTGRDIE |
| | | | GEGVEHREAEAVDDSTREHADTSGILELLKCKDI |
| | | | RSTLHGKFKDCFGLSFVDLIRPFKSDRTTCADWV |
| | | | VAGFGIHHSIADAFQKLIEPLSLYAHIQWLTNAW |
| | | | GMVLLVLIRFKVNKSRCTVARTLGTLLNIPENHM |
| | | | LIEPPKIQSGVRALYWFRTGISNASTVIGEAPEW |
| | | | ITRQTVIEHSLADSQFKLTEMVQWAYDNDICEES |
| | | | EIAFEYAQRGDFDSNARAFLNSNMQAKYVKDCAI |
| | | | MCRHYKHAEMKKMSIKQWIKYRGTKVDSVGNWKP |
| | | | IVQFLRHQNIEFIPFLSKLKLWLHGTPKKNCIAI |
| | | | VGPPDTGKSCFCMSLIKFLGGTVISYVNSCSHFW |
| | | | LQPLTDAKVALLDDATQPCWTYMDTYMRNLLDGN |
| | | | PMSIDRKHRALTLIKCPPLLVTSNIDISKEEKYK |
| | | | YLHSRVTTFTFPNPFPFDRNGNAVYELSDANWKC |
| | | | FFERLSSSLDIEDSEDEEDGSNSQAFRCVPGSVV |
| | | | RTL |
| HPV11 | | | MEAIAKRLDACQDQLLELYEENSIDIHKHIMHWK |
| E2 | | | CIRLESVLLHKAKQMGLSHIGLQVVPPLTVSETK |
| | | | GHNAIEMQMHLESLAKTQYGVEPWTLQDTSYEMW |
| | | | LTPPKRCFKKQGNTVEVKFDGCEDNVMEYVVWTH |
| | | | IYLQDNDSWVKVTSSVDAKGIYYTCGQFKTYYVN |
| | | | FNKEAQKYGSTNHWEVCYGSTVICSPASVSSTVR |
| | | | EVSIAEPTTYTPAQTTAPTVSACTTEDGVSAPPR |
| | | | KRARGPSTNNTLCVANIRSVDSTINNIVTDNYNK |
| | | | HQRRNNCHSAATPIVQLQGDSNCLKCFRYRLNDK |
| | | | YKHLFELASSTWHWASPEAPHKNAIVTLTYSSEE |
| | | | QRQQFLNSVKIPPTIRHKVGFMSLHLL |
| HPV11 | | | MVVPIIGKYVMAAQLYVLLHLYLALYEKYPLLNL |
| E4 | | | LHTPPHRPPPLQCPPAPRKTACRRRLGSEHVDRP |
| | | | LTTPCVWPTSDPWTVQSTTSSLTITTSTKEGTTV |
| | | | TVQLRL |
| HPV11 | | | MEVVPVQIAAATTTTLILPVVIAFAVCILSIVLI |
| E5A | | | ILISDFVVYTSVLVLTLLLYLLLWLLLTTPLQFF |
| | | | LLTLCVCYFPAFYIHIYIVQTQQ |
| HPV11 | | | MVMLTCHLNDGDTWLFLWLFTAFVVAVLGLLLL |
| E5B | | | HYRAVHGTEKTKCAKCKSNRNITVDYVYMSHGD |
| | | | NGDYVYMN |
| HPV11 | | | MESKDASTSATSIDQLCKTFNLSLHTLQIQCVFC |
| E6 | | | RNALTTAEIYAYAYKNLKVVWRDNFPFAACACCL |
| | | | ELQGKINQYRHFNYAAYAPTVEEETNEDILKVLI |
| | | | RCYLCHKPLCEIEKLKHILGKARFIKLNNQWKGR |
| | | | CLHCWTTCMEDLLP |
| HPV11 | | | MHGRLVTLKDIVLDLQPPDPVGLHCYEQLEDSSE |
| E7 | | | DEVDKVDKQDAQPLTQHYQILTCCCGCDSNVRLV |
| | | | VECTDGDIRQLQDLLLGTLNIVCPICAPKP |
| HPV11 | | | MWRPSDSTVYVPPPNPVSKVVATDAYVKRTNIFY |
| L1 | | | HASSSRLLAVGHPYYSIKKVNKTVVPKVSGYQYR |
| | | | VFKVVLPDPNKFALPDSSLFDPTTQRLVWACTGL |
| | | | EVGRGQPLGVGVSGHPLLNKYDDVENSGGYGGNP |
| | | | GQDNRVNVGMDYKQTQLCMVGCAPPLGEHWGK |
| | | | GTQCSNTSVQNGDCPPLELITSVIQDGDMVDTGF |
| | | | GAMNFADLQTNKSDVPLDICGTVCKYPDYLQMAA |
| | | | DPYGDRLFFYLRKEQMFARHFFNRAGTVGEPVPD |
| | | | DLLVKGGNNRSSVASSIYVHTPSGSLVSSEAQLF |
| | | | NKPYWLQKAQGHNNGICWGNHLFVTVVDTTRSTN |
| | | | MTLCASVSKSATYTNSDYKEYMRHVEEFDLQFIF |
| | | | QLCSITLSAEVMAYIHTMNPSVLEDWNFGLSPPP |
| | | | NGTLEDTYRYVQSQAITCQKPTPEKEKQDPYKDM |
| | | | SFWEVNLKEKFSSELDQFPLGRKFLLQSGYRGRT |
| | | | SARTGIKRPAVSKPSTAPKRKRTKTKK |
| HPV11 | | | MKPRARRRKRASATQLYQTCKATGTCPPDVIPKV |
| L2 | | | EHTTIADQILKWGSLGVFFGGLGIGTGAGSGGRA |
| | | | GYIPLGSSPKPAITGGPAARPPVLVEPVAPSDPS |
| | | | IVSLIEESAIINAGAPEVVPPTQGGFTITSSEST |
| | | | TPAILDVSVTNHTTTSVFQNPLFTEPSVIQPQPP |
| | | | VEASGHILISAPTITSQHVEDIPLDTFVVSSSDS |
| | | | GPTSSTPLPRAFPRPRVGLYSRALQQVQVTDPAF |
| | | | LSTPQRLVTYDNPVYEGEDVSLQFTHESIHNAPD |
| | | | EAFMDIIRLHRPAITSRRGLVRFSRIGQRGSMYT |
| | | | RSGQHIGARIHYFQDISPVTQAAEEIELHPLVAA |
| | | | ENDTFDIYAEPFDPIPDPVQHSVTQSYLTSTPNT |
| | | | LSQSWGNTTVPLSIPSDWFVQSGPDITFPTASMG |
| | | | TPFSPVTPALPTGPVFITGSDFYLHPTWYFARRR |
| | | | RKRIPLFFTDVAA |
| HPV16 | | | MADPAGTNGEEGTGCNGWFYVEAVVEKKTGDAI |
| E1 | | | SDDENENDSDTGEDLVDFIVNDNDYLTQAETETA |
| | | | HALFTAQEAKQHRDAVQVLKRKYLVSPLSDISGC |
| | | | VDNNISPRLKAICIEKQSRAAKRRLFESEDSGYG |
| | | | NTEVETQQMLQVEGRHETETPCSQYSGGSGGGCS |
| | | | QYSSGSGGEGVSERHTICQTPLTNILNVLKTSNA |
| | | | KAAMLAKFKELYGVSFSELVRPFKSNKSTCCDWC |
| | | | IAAFGLTPSIADSIKTLLQQYCLYLHIQSLACSW |
| | | | GMVVLLLVRYKCGKNRETIEKLLSKLLCVSPMCM |
| | | | MIEPPKLRSTAAALYWYKTGISNISEVYGDTPEW |
| | | | IQRQTVLQHSFNDCTFELSQMVQWAYDNDIVDDS |
| | | | EIAYKYAQLADTNSNASAFLKSNSQAKIVKDCAT |
| | | | MCRHYKRAEKKQMSMSQWIKYRCDRVDDGGDWKQ |
| | | | IVMFLRYQGVEFMSFLTALKRFLQGIPKKNCILL |
| | | | YGAANTGKSLFGMSLMKFLQGSVICFVNSKSHFW |
| | | | LQPLADAKIGMLDDATVPCWNYIDDNLRNALDGN |
| | | | LVSMDVKHRPLVQLKCPPLLITSNINAGTDSRWP |
| | | | YLHNRLVVFTFPNEFPFDENGNPVYELNDKNWKS |
| | | | FFSRTWSRLSLHEDEDKENDGDSLPTFKCVSGQN |
| | | | TNTL |
| HPV16 | W2WLHS | | METLCQRLNVCQDKILTHYENDSTDLRDHIDYWK |
| E2 | | | HMRLECAIYYKAREMGFKHINHQVVPTLAVSKNK |
| | | | ALQAIELQLTLETIYNSQYSNEKWTLQDVSLEVY |
| | | | LTAPTGCIKKHGYTVEVQFDGDICNTMHYTNWTH |
| | | | IYICEEASVTVVEGQVDYYGLYYVHEGIRTYFVQ |
| | | | FKDDAEKYSKNKVWEVHAGGQVILCPTSVFSSNE |
| | | | VSSPEIIRQHLANHPAATHTKAVALGTEETQTTI |
| | | | QRPRSEPDTGNPCHTTKLLHRDSVDSAPILTAFN |
| | | | SSHKGRINCNSNTTPIVHLKGDANTLKCLRYRFK |
| | | | KHCTLYTAVSSTWHWTGHNVKHKSAIVTLTYDSE |
| | | | WQRDQFLSQVKIPKTITVSTGFMSI |
| HPV16 | W5WLHS | | MTNLDTASTTLLACFLLCFCVLLCVCLLIRPLLL |
| E5 | | | SVSTYTSLIILVLLLWITAASAFRCFIVYIIFVY |
| | | | IPLFLIHTHARFLIT |
| HPV16 | | | MHQKRTAMFQDPQERPRKLPQLCTELQTTIHDII |
| E6 | | | LECVYCKQQLLRREVYDFAFRDLCIVYRDGNPYA |
| | | | VCDKCLKFYSKISEYRHYCYSLYGTTLEQQYNKP |
| | | | LCDLLIRCINCQKPLCPEEKQRHLDKKQRFHNIR |
| | | | GRWTGRCMSCCRSSRTRRETQL |
| HPV16 | | | MHGDTPTLHEYMLDLQPETTDLYCYEQLNDSSEE |
| E7 | | | EDEIDGPAGQAEPDRAHYNIVTFCCKCDSTLRLC |
| | | | VQSTHVDIRTLEDLLMGTLGIVCPICSQKP |
| HPV16 | AAD | | MQVTFIYILVITCYENDVNVYHIFFQMSLWLPSE |
| L1 | 33259 | | ATVYLPPVPVSKVVSTDEYVARTNIYYHAGTSRL |
| | | | LAVGHPYFPIKKPNNNKILVPKVSGLQYRVFRIH |
| | | | LPDPNKFGFPDTSFYNPDTQRLVWACVGVEVGRG |
| | | | QPLGVGISGHPLLNKLDDTENASAYAANAGVDNR |
| | | | ECISMDYKQTQLCLIGCKPPIGEHWGKGSPCTNV |
| | | | AVNPGDCPPLELINTVIQDGDMVDTGFGAMDFTT |
| | | | LQANKSEVPLDICTSICKYPDYIKMVSEPYGDSL |
| | | | FFYLRREQMFVRHLFNRAGAVGENVPDDLYIKGS |
| | | | GSTANLASSNYFPTPSGSMVTSDAQIFNKPYWLQ |
| | | | RAQGHNNGICWGNQLFVTVVDTTRSTNMSLCAAI |
| | | | STSETTYKNTNFKEYLRHGEEYDLQFIFQLCKIT |
| | | | LTADVMTYIHSMNSTILEDWNFGLQPPPGGTLED |
| | | | TYRFVTSQAIACQKHTPPAPKEDPLKKYTFWEVN |
| | | | LKEKFSADLDQFPLGRKFLLQAGLKAKPKFTLGK |
| | | | RKATPTTSSTSTTAKRKKRKL |
| HPV16 | AAD | | MRHKRSAKRTKRASATQLYKTCKQAGTCPPDIII |
| L2 | 33258 | | PKVEGKTIADQILQYGSMGVFFGGLGIGTGSGTG |
| | | | GRTGYIPLGTRPPTATDTLAPVRPPLTVDPVGPS |
| | | | DPSIVSLVEETSFIDAGAPTSVPSIPPDVSGFSI |
| | | | TTSTDTTPAILDINNTVTTVTTHNNPTFTDPSVL |
| | | | QPPTPAETGGHFTLSSSTISTHNYEEIPMDTFIV |
| | | | STNPNTVTSSTPIPGSRPVARLGLYSRTTQQVKV |
| | | | VDPAFITTPTKLITYDNPAYEGIDVDNTLYFSSN |
| | | | DNSINIAPDPDFLDIVALHRPALTSRRTGIRYSR |
| | | | IGNKQTLRTRSGKSIGAKVHYYYDFSTIDSAEEI |
| | | | ELQTITPSTYTTTSHAALPTSINNGLYDIYADDF |
| | | | ITDTSTTPVPSVPSTSLSGYIPANTTIPFGGAYN |
| | | | IPLVSGPDIPINITDQAPSLIPIVPGSPQYTIIA |
| | | | DAGDFYLHPSYYMLRKRRKRLPYFFSDVSLAA |
| HPV18 | | | MADPEGTDGEGTGCNGWFYVQAIVDKKTGDVISD |
| E1 | | | DEDENATDTGSDMVDFIDTQGTFCEQAELETAQA |
| | | | LFHAQEVHNDAQVLHVLKRKFAGGSTENSPLGER |
| | | | LEVDTELSPRLQEISLNSGQKKAKRRLFTISDSG |
| | | | YGCSEVEATQIQVTTNGEHGGNVCSGGSTEAIDN |
| | | | GGTEGNNSSVDGTSDNSNIENVNPQCTIAQLKDL |
| | | | LKVNNKQGAMLAVFKDTYGLSFTDLVRNFKSDKT |
| | | | TCTDWVTAIFGVNPTIAEGFKTLIQPFILYAHIQ |
| | | | CLDCKWGVLILALLRYKCGKSRLTVAKGLSTLLH |
| | | | VPETCMLIQPPKLRSSVAALYWYRTGISNISEVM |
| | | | GDTPEWIQRLTIIQHGIDDSNFDLSEMVQWAFDN |
| | | | ELTDESDMAFEYALLADSNSNAAAFLKSNCQAKY |
| | | | LKDCATMCKHYRRAQKRQMNMSQWIRFRCSKIDE |
| | | | GGDWRPIVQFLRYQQIEFITFLGALKSFLKGTPK |
| | | | KNCLVFCGPANTGKSYFGMSFIHFIQGAVISFVN |
| | | | STSHFWLEPLTDTKVAMLDDATTTCWTYFDTYMR |
| | | | NALDGNPISIDRKHKPLIQLKCPPILLTTNIHPA |
| | | | KDNRWPYLESRITVFEFPNAFPFDKNGNPVYEIN |
| | | | DKNWKCFFERTWSRLDLHEEEEDADTEGNPFGTF |
| | | | KLRAGQNHRPL |
| HPV18 | W2WL18 | | MQTPKETLSERLSCVQDKIIDHYENDSKDIDSQI |
| E2 | | | QYWQLIRWENAIFFAAREHGIQTLNHQVVPAYNI |
| | | | SKSKAHKAIELQMALQGLAQSRYKTEDWTLQDTC |
| | | | EELWNTEPTHCFKKGGQTVQVYFDGNKDNCMTYV |
| | | | AWDSVYYMTDAGTWDKTATCVSHRGLYYVKEGY |
| | | | NTFYIEFKSECEKYGNTGTWEVHFGNNVIDCNDS |
| | | | MCSTSDDTVSATQLVKQLQHTPSPYSSTVSVGTA |
| | | | KTYGQTSAATRPGHCGLAEKQHCGPVNPLLGAAT |
| | | | PTGNNKRRKLCSGNTTPIIHLKGDRNSLKCLRYR |
| | | | LRKHSDHYRDISSTWHWTGAGNEKTGILTVTYHS |
| | | | ETQRTKFLNTVAIPDSVQILVGYMTM |
| HPV18 | W5WL18 | | MLSLIFLFCFCVCMYVCCHVPLLPSVCMCAYAWV |
| E5 | | | LVFVYIVVITSPATAFTVYVFCFLLPMLLLHIHA |
| | | | ILSLQ |
| HPV18 | | | MARFEDPTRRPYKLPDLCTELNTSLQDIEITCVY |
| E6 | | | CKTVLELTEVFEFAFKDLFVVYRDSIPHAACHKC |
| | | | IDFYSRIRELRHYSDSVYGDTLEKLTNTGLYNLL |
| | | | IRCLRCQKPLNPAEKLRHLNEKRRFHNIAGHYRG |
| | | | QCHSCCNRARQERLQRRRETQV |
| HPV18 | | | MHGPKATLQDIVLHLEPQNEIPVDLLCHEQLSDS |
| E7 | | | EEENDEIDGVNHQHLPARRAEPQRHTMLCMCCKC |
| | | | EARIKLVVESSADDLRAFQQLFLNTLSFVCPWCA |
| | | | SQQ |
| HPV18 | CAA | | MCLYTRVLILHYHLLPLYGPLYHPRPLPLHSILV |
| L1 | 28671 | | YMVHIIICGHYIILFLRNVNVFPIFLQMALWRPS |
| | | | DNTVYLPPPSVARVVNTDDYVTPTSIFYHAGSSR |
| | | | LLTVGNPYFRVPAGGGNKQDIPKVSAYQYRVFRV |
| | | | QLPDPNKFGLPDTSIYNPETQRLVWACAGVEIGR |
| | | | GQPLGVGLSGHPFYNKLDDTESSHAATSNVSEDV |
| | | | RDNVSVDYKQTQLCILGCAPAIGEHWAKGTACKS |
| | | | RPLSQGDCPPLELKNTVLEDGDMVDTGYGAMDFS |
| | | | TLQDTKCEVPLDICQSICKYPDYLQMSADPYGDS |
| | | | MFFCLRREQLFARHFWNRAGTMGDTVPQSLYIKG |
| | | | TGMPASPGSCVYSPSPSGSIVTSDSQLFNKPYWL |
| | | | HKAQGHNNGVCWHNQLFVTVVDTTPSTNLTICAS |
| | | | TQSPVPGQYDATKFKQYSRHVEEYDLQFIFQLCT |
| | | | ITLTADVMSYIHSMNSSILEDWNFGVPPPPTTSL |
| | | | VDTYRFVQSVAITCQKDAAPAENKDPYDKLKFWN |
| | | | VDLKEKFSLDLDQYPLGRKFLVQAGLRRKPTIGP |
| | | | RKRSAPSATTSSKPAKRVRVRARK |
| HPV18 | P2WL18 | | MVSHRAARRKRASVTDLYKTCKQSGTCPPDVVPK |
| L2 | | | VEGTTLADKILQWSSLGIFLGGLGIGTGSGTGGR |
| | | | TGYIPLGGRSNTVVDVGPTRPPVVIEPVGPTDPS |
| | | | IVTLIEDSSVVTSGAPRPTFTGTSGFDITSAGTT |
| | | | TPAVLDITPSSTSVSISTTNFTNPAFSDPSIIEV |
| | | | PQTGEVAGNVFVGTPTSGTHGYEEIPLQTFASSG |
| | | | TGEEPISSTPLPTVRRVAGPRLYSRAYQQVSVAN |
| | | | PEFLTRPSSLITYDNPAFEPVDTTLTFDPRSDVP |
| | | | DSDFMDIIRLHRPALTSRRGTVRFSRLGQRATMF |
| | | | TRSGTQIGARVHFYHDISPIAPSPEYIELQPLVS |
| | | | ATEDNDLFDIYADDMDPAVPVPSRSTTSFAFFKY |
| | | | SPTISSASSYSNVTVPLTSSWDVPVYTGPDITLP |
| | | | STTSVWPIVSPTAPASTQYIGHGTHYYLWPLYYF |
| | | | IPKKRKRVPYFFADGFVAA |
| HPV31 | W1WL31 | | MADPAGTDGEGTGCNGWFYVEAVIDRQTGDNISE |
| E1 | | | DENEDSSDTGEDMVDFIDNCNVYNNQAEAETAQA |
| | | | LFHAQEAEEHAEAVQVLKRKYVGSPLSDISSCVD |
| | | | YNISPRLKAICIENNSKTAKRRLFELPDSGYGNT |
| | | | EVETQQMVQVEEQQTTLSCNGSDGTHSERENETP |
| | | | TRNILQVLKTSNGKAAMLGKFKELYGVSFMELIR |
| | | | PFQSNKSTCTDWCVAAFGVTGTVAEGFKTLLQPY |
| | | | CLYCHLQSLACSWGMVMLMLVRFKCAKNRITIEK |
| | | | LLEKLLCISTNCMLIQPPKLRSTAAALYWYRTGM |
| | | | SNISDVYGETPEWIERQTVLQHSFNDTTFDLSQM |
| | | | VQWAYDNDVMDDSEIAYKYAQLADSDSNACAFLK |
| | | | SNSQAKIVKDCGTMCRHYKRAEKRQMSMGQWIKS |
| | | | RCDKVSDEGDWRDIVKFLRYQQIEFVSFLSALKL |
| | | | FLKGVPKKNCILIHGAPNTGKSYFGMSLISFLQG |
| | | | CIISYANSKSHFWLQPLADAKIGMLDDATTPCWH |
| | | | YIDNYLRNALDGNPVSIDVKHKALMQLKCPPLLI |
| | | | TSNINAGKDDRWPYLHSRLVVFTFPNPFPFDKNG |
| | | | NPVYELSDKNWKSFFSRTWCRLNLHEEEDKENDG |
| | | | DSFSTFKCVSGQNIRTL |
| HPV31 | W2WL31 | | METLSQRLNVCQDKILEHYENDSKRLCDHIDYWK |
| E2 | | | HIRLECVLMYKAREMGIHSINHQVVPALSVSKAK |
| | | | ALQAIELQMMLETLNNTEYKNEDWTMQQTSLELY |
| | | | LTAPTGCLKKHGYTVEVQFDGDVHNTMHYTNWKF |
| | | | IYLCIDGQCTVVEGQVNCKGIYYVHEGHITYFVN |
| | | | FTEEAKKYGTGKKWEVHAGGQVIVFPESVFSSDE |
| | | | ISFAGIVTKLPTANNTTTSNSKTCALGTSEGVRR |
| | | | ATTSTKRPRTEPEHRNTHHPNKLLRGDSVDSVNC |
| | | | GVISAAACTNQTRAVSCPATTPIIHLKGDANILK |
| | | | CLRYRLSKYKQLYEQVSSTWHWTCTDGKHKNAIV |
| | | | TLTYISTSQRDDFLNTVKIPNTVSVSTGYMTI |
| HPV31 | W5WL31 | | MIELNISTVSIVLCFLLCFCVLLFVCLVIRPLVL |
| E5 | | | SVSVYATLLLLIVILWVIATSPLRCFCIYVVFIY |
| | | | LPLFVIHTHASFLSQQ |
| HPV31 | W6WL31 | | MFKNPAERPRKLHELSSALEIPYDELRLNCVYCK |
| E6 | | | GQLTETEVLDFAFTDLTIVYRDDTPHGVCTKCLR |
| | | | FYSKVSEFRWYRYSVYGTYLEKLTNKGICDLLIR |
| | | | CITCQRPLCPEEKQRHLDKKKRFHNIGGRWTGRC |
| | | | IACWRRPRTETQV |
| HPV31 | W7WL31 | | MRGETPTLQDYVLDLQPEATDLHCYEQLPDSSDE |
| E7 | | | EDVIDSPAGQAEPDTSNYNIVTFCCQCKSTLRLC |
| | | | VQSTQVDIRILQELLMGSFGIVCPNCSTRL |
| HPV31 | P1WL31 | | MSLWRPSEATVYLPPVPVSKVVSTDEYVTRTNIY |
| L1 | | | YHAGSARLLTVGHPYYSIPKSDNPKKIVVPKVSG |
| | | | LQYRVFRVRLPDPNKFGFPDTSFYNPETQRLVWA |
| | | | CVGLEVGRGQPLGVGISGHPLLNKFDDTENSNRY |
| | | | AGGPGTDNRECISMDYKQTQLCLLGCKPPIGEHW |
| | | | GKGSPCSNNAITPGDCPPLELKNSVIQDGDMVDT |
| | | | GFGAMDFTALQDTKSNVPLDICNSICKYPDYLKM |
| | | | VAEPYGDTLFFYLRREQMFVRHFFNRSGTVGESV |
| | | | PTDLYIKGSGSTATLANSTYFPTPSGSMVTSDAQ |
| | | | IFNKPYWMQRAQGHNNGICWGNQLFVTVVDTTRS |
| | | | TNMSVCAAIANSDTTFKSSNFKEYLRHGEEFDLQ |
| | | | FIFQLCKITLSADIMTYIHSMNPAILEDWNIFGL |
| | | | TTPPSGSLEDTYRFVTSQAITCQKTAPQKPKEDP |
| | | | FKDYVFWEVNLKEKFSADLDQFPLGRKFLLQAGY |
| | | | RARPKEKAGKRSAPSASTTTPAKRKKTKK |
| HPV31 | P2WL31 | | MRSKRSTKRTKRASATQLYQTCKAAGTCPSDVIP |
| L2 | | | KIEHTTIADQILRYGSMGVFFGGLGIGSGSGTGG |
| | | | RTGYVPLSTRPSTVSEASIPIRPPVSIDPVGPLD |
| | | | PSIVSLVEESGIVDVGAPAPIPHPPTTSGFDIAT |
| | | | TADTTPAILDVTSVSTHENPTFTDPSVLQPPTPA |
| | | | ETSGHLLLSSSSISTHNYEEIPNDTFIVSTNNEN |
| | | | ITSSTPIPGVRRPARLGLYSKATQQVKVIDPTFL |
| | | | SAPKQLITYENPAYETVNAEESLYFSNTSHNIAP |
| | | | DPDFLDIIALHRPALTSRRNTVRYSRLGNKQTLR |
| | | | TRSGATIGARVHYYYDISSINPAGESIEMQPLGA |
| | | | SATTTSTLNDGLYDIYADTDFTVDTPATHNVSPS |
| | | | TAVQSTSAVSAYVPTNTTVPLSTGFDIPIFSGPD |
| | | | VPIEHAPTQVFPFPLAPTTPQVSIFVDGGDFYLH |
| | | | PSYYMLKRRRKRVSYFFTDVSVAA |
| HPV33 | W1WL33 | | MADPEGTNGAGMGCTGWFEVEAVIERRTGDNISE |
| E1 | | | DEDETADDSGTDLLEFIDDSMENSIQADTEAARA |
| | | | LFNIQEGEDDLNAVCALKRKFAACSQSAAEDVVD |
| | | | RAANPCRTSINKNKECTYRKRKIDELEDSGYGNT |
| | | | EVETQQMVQQVESQNGDTNLNDLESSGVGDDSEV |
| | | | SCETNVDSCENVTLQEISNVLHSSNTKANILYKF |
| | | | KEAYGISFMELVRPFKSDKTSCTDWCITGYGISP |
| | | | SVAESLKVLIKQHSLYTHLQCLTCDRGIIILLLI |
| | | | RFRCSKNRLTVAKLMSNLLSIPETCMVIEPPKLR |
| | | | SQTCALYWFRTAMSNISDVQGTTPEWIDRLTVLQ |
| | | | HSFNPNIFDLSEMVQWAYDNELTDDSDIAYYYAQ |
| | | | LADSNSNAAAFLKSNSQAKIVKDCGIMCRHYKKA |
| | | | EKRKMSIGQWIQSRCEKTNDGGNWRPIVQLLRYQ |
| | | | NIEFTAFLGAFKKFLKGIPKKSCMLICGPANTGK |
| | | | SYFGMSLIQFLKGCVISCVNSKSHFWLQPLSDAK |
| | | | IGMIDDVTPISWTYIDDYMRNALDGNEISIDVKH |
| | | | RALVQLKCPPLLLTSNTNAGTDSRWPYLHSRLTV |
| | | | FEFKNPFPFDENGNPVYAINDENWKSFFSRTWCK |
| | | | LDLIEEEDKENHGGNISTFKCSAGENTRSLRS |
| HPV33 | W2WL33 | | MEEISARLNAVQEKILDLYEADKTDLPSQIEHWK |
| E2 | | | LIRMECALLYTAKQMGFSHLCHQVVPSLLASKTK |
| | | | AFQVIELQMALETLSKSQYSTSQWTLQQTSLEVW |
| | | | LCEPPKCFKKQGETVTVQYDNDKKNTMDYTNWGE |
| | | | IYIIEEDTCTMVTGKVDYIGMYYIHNCEKVYFKY |
| | | | FKEDAAKYSKTQMWEVHVGGQVIVCPTSISSNQI |
| | | | STTETADIQTDNDNRPPQAAAKRRRPADTTDTAQ |
| | | | PLTKLFCADPALDNRTARTATNCTNKQRTVCSSN |
| | | | VAPIVHLKGESNSLKCLRYRLKPYKELYSSMSST |
| | | | WHWTSDNKNSKNGIVTVTFVTEQQQQMFLGTVKI |
| | | | PPTVQISTGFMTL |
| HPV33 | W5WL33 | | MIFVFVLCFILFLCLSLLLRPLILSISTYAWLLV |
| E5 | | | LVLLLWVFVGSPLKIFFCYLLFLYLPMMCINFHA |
| | | | QHMTQQE |
| HPV33 | W6WL33 | | MFQDTEEKPRTLHDLCQALETTIHNIELQCVECK |
| E6 | | | KPLQRSEVYDFAFADLTVVYREGNPFGICKLCLR |
| | | | FLSKISEYRHYNYSVYGNTLEQTVKKPLNEILIR |
| | | | CIICQRPLCPQEKKRHVDLNKRFHNISGRWAGRC |
| | | | AACWRSRRRETAL |
| HPV33 | W7WL33 | | MRGHKPTLKEYVLDLYPEPTDLYCYEQLSDSSDE |
| E7 | | | DEGLDRPDGQAQPATADYYIVTCCHTCNTTVRLC |
| | | | VNSTASDLRTIQQLLMGTVNIVCPTCAQQ |
| HPV33 | P1WL33 | | MSVWRPSEATVYLPPVPVSKVVSTDEYVSRTSIY |
| L1 | | | YYAGSSRLLAVGHPYFSIKNPTNAKKLLVPKVSG |
| | | | LQYRVFRVRLPDPNKFGFPDTSFYNPDTQRLVWA |
| | | | CVGLEIGRGQPLGVGISGHPLLNKFDDTETGNKY |
| | | | PGQPGADNRECLSMDYKQTQLCLLGCKPPTGEHW |
| | | | GKGVACTNAAPANDCPPLELINTIIEDGDMVDTG |
| | | | FGCMDFKTLQANKSDVPIDICGSTCKYPDYLKMT |
| | | | SEPYGDSLFFFLRREQMFVRHFFNRAGTLGEAVP |
| | | | DDLYIKGSGTTASIQSSAFFPTPSGSMVTSESQL |
| | | | FNKPYWLQRAQGHNNGICWGNQVFVTVVDTTRST |
| | | | NMTLCTQVTSDSTYKNENFKEYIRHVEEYDLQFV |
| | | | FQLCKVTLTAEVMTYIHAMNPDILEDWQFGLTPP |
| | | | PSASLQDTYRFVTSQAITCQKTVPPKEKEDPLGK |
| | | | YTFWEVDLKEKFSADLDQFPLGRKFLLQAGLKAK |
| | | | PKLKRAAPTSTRTSSAKRKKVKK |
| HPV33 | P2WL33 | | MRHKRSTRRKRASATQLYQTCKATGTCPPDVIPK |
| L2 | | | VEGSTIADQILKYGSLGVFFGGLGIGTGSGSGGR |
| | | | TGYVPIGTDPPTAAIPLQPIRPPVTVDTVGPLDS |
| | | | SIVSLIEETSFIEAGAPAPSIPTPSGFDVTTSAD |
| | | | TTPAIINVSSVGESSIQTISTHLNPTFEPSVLHP |
| | | | PAPAEASGHFIFSSPTVSTQSYENIPMDTFVVST |
| | | | DSSNVTSSTPIPGSRPVARLGLYSRNTQQVKVVD |
| | | | PAFLTSPHKLITYDNPAFESFDPEDTLQFQHSDI |
| | | | SPAPDPDFLDIIALHRPAITSRRHTVRFSRVGQK |
| | | | ATLKTRSGKQIGARIHYYQDLSPIVPLDHTVPNE |
| | | | QYELQPLHDTSTSSYSINDGLYDVYADDVDNVHT |
| | | | PMQHSYSTFATTRTSNVSIPLNTGFDTPVMSGPD |
| | | | IPSPLFPTSSPFVPISPFFPFDTIVVDGADFVLH |
| | | | PSYFILRRRRKRFPYFFTDVRVAA |
| HPV45 | S36563 | | MADPEGTDGEGTGCNGWFFVETIVEKKTGDVISD |
| E1 | | | DEDETATDTGSDMVDFIDTQLSICEQAEQETAQA |
| | | | LFHAQEVQNDAQVLHLLKRKFAGGSKENSPLGEQ |
| | | | LSVDTDLSPRLQEISLNSGHKKAKRRLFTISDSG |
| | | | YGCSEVEAAETQVTVNTNAENGGSVHSTQSSGGD |
| | | | SSDNAENVDPHCSITELKELLQASNKKAAMLAVF |
| | | | KDIYGLSFTDLVRNFKSDKTTCTDWVMAIFGVNP |
| | | | TVAEGFKTLIKPATLYAHIQCLDCKWGVLILALL |
| | | | RYKCGKNRLTVAKGLSTLLHVPETCMLIEPPKLR |
| | | | SSVAALYWYRTGISNISEVSGDTPEWIQRLTIIQ |
| | | | HGIDDSNFDLSDMVQWAFDNDLTDESDMAFQYAQ |
| | | | LADCNSNAAAFLKSNCQAKYLKDCAVMCRHYKRA |
| | | | QKRQMNMSQWIKYRCSKIDEGGDWRPIVQFLRYQ |
| | | | GVEFISFLRALKEFLKGTPKKNCILLYGPANTGK |
| | | | SYFGMSFIHFLQGAIISFVNSNSHFWLEPLADTK |
| | | | VAMLDDATHTCWTYFDNYMRNALDGNPISIDRKH |
| | | | KPLLQLKCPPILLTSNIDPAKDNKWPYLESRVTV |
| | | | FTFPHAFPFDKNGNPVYEINDKNWKCFFERTWSR |
| | | | LDLHEDDEDADTEGIPFGTFKCVTGQNTRPL |
| HPV45 | S36564 | | MKMQTPKESLSERLSALQDKILDHYENDSKDINS |
| E2 | | | QISYWQLIRLENAILFTAREHGITKLNHQVVPPI |
| | | | NISKSKAHKAIELQMALKGLAQSKYNNEEWTLQD |
| | | | TCEELWNTEPSQCFKKGGKTVHVYFDGNKDNCMN |
| | | | YVVWDSIYYITETGIWDKTAACVSYWGVYYIKDG |
| | | | DTTYYVQFKSECEKYGNSNTWEVQYGGNVIDCND |
| | | | SMCSTSDDTVSATQIVRQLQHASTSTPKTASVGT |
| | | | PKPHIQTPATKRPRQCGLTEQHHGRVNTHVHNPL |
| | | | LCSSTSNNKRRKVCSGNTTPIIHLKGDKNSLKCL |
| | | | RYRLRKYADHYSEISSTWHWTGCNKNTGILTVTY |
| | | | NSEVQRNTFLDVVTIPNSVQISVGYMTI |
| HPV45 | CAB | | MARFDDPTQRPYKLPDLCTELNTSLQDVSIACVY |
| E6 | 44706 | | CKATLERTEVYQFAFKDLFIVYRDCIAYAACHKC |
| | | | IDFYSRIRELRYYSNSVYGETLEKITNTELYNLL |
| | | | IRCLRCQKPLNPAEKRRHLKDKRRFHSIAGQYRG |
| | | | QCNTCCDQARQERLRRRRETQV |
| HPV45 | CAB | | MHGPRATLQEIVLHLEPQNELDPVDLLCYEQLSE |
| E7 | 44707 | | SEEENDEADGVSHAQLPARRAEPQRHKILCVCCK |
| | | | CDGRIELTVESSADDLRTLQQLFLSTLSFVCPWC |
| | | | ATNQ |
| HPV45 | CAB | | MAHNIIYGHGIIIFLKNVNVFPIFLQMALWRPSD |
| L1 | 44705 | | STVYLPPPSVARVVNTDDYVSRTSIFYHAGSSRL |
| | | | LTVGNPYFRVVPSGAGNKQAVPKVSAYQYRVFRV |
| | | | ALPDPNKFGLPDSTIYNPETQRLVWACVGMEIGR |
| | | | GQPLGIGLSGHPFYNKLDDTESAHAATAVITQDV |
| | | | RDNVSVDYKQTQLCILGCVPAIGEHWAKGTLCKP |
| | | | AQLQPGDCPPLELKNTIIEDGDMVDTGYGAMDFS |
| | | | TLQDTKCEVPLDICQSICKYPDYLQMSADPYGDS |
| | | | MFFCLRREQLFARHFWNRAGVMGDTVPTDLYIKG |
| | | | TSANMRETPGSCVYSPSPSGSITTSDSQLFNKPY |
| | | | WLHKAQGHNNGICWHNQLFVTVVDTTRSTNLTLC |
| | | | ASTQNPVPNTYDPTKFKHYSRHVEEYDLQFIFQL |
| | | | CTITLTAEVSYIHSMNSSILENWNFGVPPPPTTS |
| | | | LVDTYRFVQSVAVTCQKDTTPPEKQDPYDKLKFW |
| | | | TVDLKEKFSSDLDQYPLGRKFLVQAGLRRRPTIG |
| | | | PRKRPAASTSTASRPAKRVRIRSKK |
| HPV45 | S36565 | | MVSHRAARRKRASATDLYRTCKQSGTCPPDVINK |
| L2 | | | VEGTTLADKILQWSSLGIFLGGLGIGTGSGSGGR |
| | | | TGYVPLGGRSNTVVDVGPTRPPVVIEPVGPTDPS |
| | | | IVTLVEDSSVVASGAPVPTFTGTSGFEITSSGTT |
| | | | TPAVLDITPTVDSVSISSTSFTNPAFSDPSIIEV |
| | | | PQTGEVSGNIFVGTPTSGSHGYEEIPLQTFASSG |
| | | | SGTEPISSTPLPTVRRVRGPRLYSRANQQVRVST |
| | | | SQFLTHPSSLVTFDNPAYEPLDTTLSFEPTSNVP |
| | | | DSDFMDIIRLHRPALSSRRGTVRFSRLGQRATMF |
| | | | TRSGKQIGGRVHFYHDISPIAATEEIELQPLISA |
| | | | TNDSDLFDVYADFPPPASTTPSTIHKSFTYPKYS |
| | | | LTMPSTAASSYSNVTVPLTSAWDVPIYTGPDIIL |
| | | | PSHTPMWPSTSPTNASTTTYIGIHGTQYYLWPWY |
| | | | YYFPKKRKRIPYFFADGFVAA |
| HPV52 | X74481 | | MEDPEGTEGEREGCTGWFEVEAIIEKQTGDNISE |
| E1 | | | DEDENAYDSGTDLIDFIDDSNINNEQAEHEAARA |
| | | | LFNAQEGEDDLHAVSAVKRKFTSSPESAGQDGVE |
| | | | KHGSPRAKHICVNTECVLPKRKPCHVEDSGYGNS |
| | | | EVEAQQMADQVDGQNGDWQSNSSQSSGVGASNSD |
| | | | VSCTSIEDNEENSNRTLKSIQNIMCENSIKTTVL |
| | | | FKFKETYGVSFMELVRPFKSNRSSCTDWCIIGMG |
| | | | VTPSVAEGLKVLIQPYSIYAHLQCLTCDRGVLIL |
| | | | LLIRFKCGKNRLTVSKLMSQLLNIPETHMVIEPP |
| | | | KLRSATCALYWYRTGLSNISEVYGTTPEWIEQQT |
| | | | VLQHSFDNSIFDFGEMVQWAYDHDITDDSDIAYK |
| | | | YAQLADVNSNAAAFLKSNSQAKIVKDCATMCRHY |
| | | | KRAERKHMNIGQWIQYRCDRIDDGGDWRPIVRFL |
| | | | RYQDIEFTAFLDAFKKFLKGIPKKNCLVLYGPAN |
| | | | TGKSYFGMSLIRFLSGCVISYVNSKSHFWLQPLT |
| | | | DAKVGMIDDVTPICWTYIDDYMRNALDGNDISVD |
| | | | VKHRALVQIKCPPLILTTNTNAGTDPRWPYLHSR |
| | | | LVVFHFKNPFPFDENGNPIYEINNENWKSFFSRT |
| | | | WCKLDLIQEEDKENDGVDTGTFKCSAGKNTRSIR |
| | | | S |
| HPV52 | | | MESIPARLNAVQEKILDLYEADSNDLNAQIEHWK |
| E2 | | | LTRMECVLFYKAKELGITHIGHQVVPPMAVSKAK |
| | | | ACQAIELQLALEALNKTQYSTDGWTLQQTSLEMW |
| | | | RAEPQKYFKKHGYTITVQYDNDKNNTMDYTNWKE |
| | | | IYLLGECECTIVEGQVDYYGLYYWCDGEKIYFVK |
| | | | ESNDAKQYCVTGVWEVHVGGQVIVCPASVSSNEV |
| | | | STTETAVHLCTETSKTSAVSVGAKDTHLQPPQKR |
| | | | RRPDVTDSRNTKYPNNLLRGQQSVDSTTRGLVTA |
| | | | TECTNKGRVAHTTCTAPIIHLKGDPNSLKCLRYR |
| | | | VKTHKSLYVQISSTWHWTSNECTNNKLGIVTITY |
| | | | SDETQRQQFLKTVKIPNTVQVIQGVMSL |
| HPV52 | | | MFEDPATRPRTLHELCEVLEESVHEIRLQCVQCK |
| E6 | | | KELQRREVYKFLFTDLRIVYRDNNPYGVCIMCLR |
| | | | FLSKISEYRHYQYSLYGKTLEERVKKPLSEITIR |
| | | | CIICQTPLCPEEKERHVNANKRFHNIMGRWTGRC |
| | | | SECWRPRPVTQV |
| HPV52 | | | MRGDKATIKDYILDLQPETTDLHCYEQLGDSSDE |
| E7 | | | EDTDGVDRPDGQAEQATSNYYIVTYCHSCDSTLR |
| | | | LCIHSTATDLRTLQQMLLGTLQVVCPGCARL |
| HPV52 | | | MVQILFYILVIFYYVAGVNVFHIFLQMSVWRPSE |
| L1 | | | ATVYLPPVPVSKVVSTDEYVSRTSIYYYAGSSRL |
| | | | LTVGHPYFSIKNTSSGNGKKVLVPKVSGLQYRVF |
| | | | RIKLPDPNKFGFPDTSFYNPETQRLVWACTGLEI |
| | | | GRGQPLGVGISGHPLLNKFDDTETSNKYAGKPGI |
| | | | DNRECLSMDYKQTQLCILGCKPPIGEHWGKGTPC |
| | | | NNNSGNPGDCPPLQLINSVIQDGDMVDTGFGCMD |
| | | | FNTLQASKSDVPDICSSVCKYPDYLQMASEPYGD |
| | | | SLFFFLRREQMFVRHFFNRAGTLGDPVPGDLYIQ |
| | | | GSNSGNTATVQSSAFFPTPSGSMVTSESQLFNKP |
| | | | YWLQRAQGHNNGICWGNQLFVTVVDTTRSTNMTL |
| | | | CAEVKKESTYKNENEKEYLRHGEEFDLQFIFQLC |
| | | | KITLTADVMTYIHKMDATILEDWQFGLTPPPSAS |
| | | | LEDTYRFVTSTAITCQKNTPPKGKEDPLKDYMFW |
| | | | EVDLKEKFSADLDQFPLGRKFLLQAGLQARPKLK |
| | | | RPASSAPRTSTKKKKVKR |
| HPV52 | | | MRYRRSTRHKRASATQLYQTCKASGTCPPDVIPK |
| L2 | | | VEGTTIADQLLKYGSLGVFFGGLGIGTGAGSGGR |
| | | | AGYVPLSTRPPTSSITTSTIRPPVTVEPIGPLEP |
| | | | SIVSMIEETTFIESGAPAPSIPSATGFDVTTSAN |
| | | | NTPAIINVTSIGESSVQSVSTHLNPTFTEPSIIQ |
| | | | PPAPAEASGHVLFSSPTISTHTYEEIPMDTFVTS |
| | | | TDSSSVTSSTPIPGSRPTTRLGLYSRATQQVKVV |
| | | | DPAFMSSPQKLVTYNNPVFEGVDTDETIIFDRSQ |
| | | | LLPAPDPDFLDIIALHRPALTSRRGTVRFSRLGN |
| | | | KATLRTRSGKQIGARVHYYHDISPIQPAEVQEDI |
| | | | ELQPLLPQSVSPYTINDGLYDVYADSLQQPTFHL |
| | | | PSTLSTHNNTFTVPINSGIDFVYQPTMSIESGPD |
| | | | IPLPSLPTHTPFVPIAPTAPSTSIIVDGTDFILH |
| | | | PSYFLLRRRRKRFPYFFTDVRVAA |
| HPV56 | | | |
| E1 | | | |
| HPV56 | S36581 | | MVPCLQVCKAKACSAIEVQIALESLSTTIYNNEE |
| E2 | | | WTLRDTCEELWLTEPKKCFKKEGQHIEVWFDGSK |
| | | | NNCMQYVAWKYIYYNGDCGWQKVCSGVDYRGIY |
| | | | YVHDGHKTYYTDFEQEAKKFGCKNIWEVHMENE |
| | | | SIYCPDSVSSTCRYNVSPVETVNEYNTHKTTTTT |
| | | | STSVGNQDAAVSHRPGKRPRLRESEFDSSRESHA |
| | | | KCVTTHTHISDTDNTDSRSRSINNNNHPGDKTTP |
| | | | VVHLKGEPNRLKCCRYRFQKYKTLFVDVTSTYHW |
| | | | TSTDNKNYSIITIIYKDETQRNSFLSHVKIPVVY |
| | | | RLVWDK |
| HPV56 | W6WL56 | | MEPQFNNPQERPRSLHHLSEVLEIPLIDLRLSCV |
| E6 | | | YCKKELTRAEVYNFACTELKLVYRDDFPYAVCRV |
| | | | CLLFYSKVRKYRYYDYSVYGATLESITKKQLCDL |
| | | | LIRCYRCQSPLTPEEKQLHCDRKRRFHLIAHGWT |
| | | | GSCLGCWRQTSREPRESTV |
| HPV56 | S36580 | | MHGKVPTLQDVVLELTPQTEIDLQCNEQLDSSED |
| E7 | | | EDEDEVDHLQERPQQARQAKQHTCYLIHVPCCEC |
| | | | KFVVQLDIQSTKEDLRVVQQLLMGALTVTCPLCA |
| | | | SSN |
| HPV56 | S38563 | | MMLPMMYIYRDPPLHYGLCIFLDVGAVNVFPIFL |
| L1 | | | QMATWRPSENKVYLPPTPVSKVVATDSYVKRTSI |
| | | | FYHAGSSRLLAVGHPYYSVTKDNTKTNIPKVSAY |
| | | | QYRVFRVRLPDPNKFGLPDTNIYNPDQERLVWAC |
| | | | VGLEVGRGQPLGAGLSGHPLFNRLDDTESSNLAN |
| | | | NNVIEDSRDNISVDGKQTQLCIVGCTPAMGEHWT |
| | | | KGAVCKSTQVTTGDCPPLALINTPIEDGDMIDTG |
| | | | FGAMDFKVLQESKAEVPLDIVQSTCKYPDYLKMS |
| | | | ADAYGDSMWFYLRREQLFARHYFNRAGKVGETIP |
| | | | AELYLKGSNGREPPPSSVYVATPSGSMITSEAQL |
| | | | FNKPYWLQRAQGHNNGICWGNQLFVTVVDTTRST |
| | | | NMTISTATEQLSKYDARKINQYLRHVEEYELQFV |
| | | | FQLCKITLSAEVMAYLHNMNANLLEDWNIGLSPP |
| | | | VATSLEDKYRYVRSTAITCQREQPPTEKQDPLAK |
| | | | YKFWDVNLQDSFSTDLDQFPLGRKFLMQLGTRSK |
| | | | PAVATSKKRSAPTSTSTPAKRKRR |
| HPV56 | S36582 | | MVAHRATRRKRASATQLYKTCKLSGTCPEDVVN |
| L2 | | | KIEQKTWADKILQWGSLFTYFGGLGIGTGTGSGG |
| | | | RAGYVPLGSRPSTIVDVTPARPPIVVESVGPTDP |
| | | | SIVTLVEESSVIESGAGIPNFTGSGGFEITSSST |
| | | | TTPAVLDITPTSSTVHVSSTHITNPLFIDPPVIE |
| | | | APQTGEVSGNILISTPTSGIHSYEEIPMQTFAVH |
| | | | GSGTEPISSTPIPGFRRIAAPRLYRKAFQQVKVT |
| | | | DPAFLDRPATLVSADNPLFEGTDTSLAFSPSGVA |
| | | | PDPDFMNIVALHRPAFTTRRGGVRFSRLGRKATI |
| | | | QTRRGTQIGARVHYYYDISPIAQAEEIEMQPLLS |
| | | | ANNSFDGLYDIYANIDDEAPGLSSQSVATPSAHL |
| | | | PIKPSTLSFASNTTNVTAPLGNVWETPFYSGPDI |
| | | | VLPTGPSTWPFVPQSPYDVTHDVYIQGSSFALWP |
| | | | VYFFRRRRRKRIPYFFADGDVAA |
| HPV58 | D90400 | | MDDPEGTNGVGAGCTGWFEVEAVIERRTGDNISD |
| E1 | | | DEDETADDSGTDLIEFIDDSVQSTTQAEAEAARA |
| | | | LFNVQEGVDDINAVCALKRKFAACSESAVEDCVD |
| | | | RAANVCVSWKYKNKECTHRKRKIIELEDSGYGNT |
| | | | EVETEQMAHQVESQNGDADLNDSESSGVGASSDV |
| | | | SSETDVDSCNTVPLQNISNILHNSNTKATLLYKF |
| | | | KEAYGVSFMELVRPFKSDKTSCTDWCITGYGISP |
| | | | SVAESLKVLIKQHSIYTHLQCLTCDRGIILLLIR |
| | | | FKCSKNRLTVAKLMSNLLSIPETCMIIEPPKLRS |
| | | | QACALYWFRTAMSNISDVQGTTPEWIDRLTVLQH |
| | | | SFNDDIFDLSEMIQWAYDNDITDDSDIAYKYAQL |
| | | | ADVNSNAAAFLRSNAQAKIVKDCGVMCRHYKRAE |
| | | | KRGMTMGQWIQSRCEKTNDGGNWRPIVQFLRYQN |
| | | | IEFTAFLVAFKQFLQGVPKKSCMLLCGPANTGKS |
| | | | YFGMSLIHFLKGCIISYVNSKSHFWLQPLSDAKL |
| | | | GMIDDVTAISWTYIDDYMRNALDGNDISIDVKHR |
| | | | ALVQLKCPPLIITSNTNAGKDSRWPYLHSRLTVF |
| | | | EFNNPFPFDANGNPVYKINDENWKSFFSRTWCKL |
| | | | GLIEEEDKENPGGNISTFKCSAGQNPRHIRS |
| HPV58 | | | MEEISARLSAVQDKILDIYEADKNDLTSQIEHWK |
| E2 | | | LIRMECAIMYTARQMGISHLCHQVVPSLVASKTK |
| | | | AFQVIELQMALETLNASPYKTDEWTLQQTSLEVW |
| | | | LSEPQKCFKKKGITVTVQYDNDKANTMDYTNWSE |
| | | | IYIIEETTCTLVAGEVDYVGLYYIHGNEKTYFKY |
| | | | FKEDAKKYSKTQLWEVHVGSRVIVCPTSIPSDQI |
| | | | STTETADPKTTEATNNESTQGTKRRRLDLPDSRD |
| | | | NTQYSTKYTDCAVDSRPRGGGLHSTTNCTYKGRN |
| | | | VCSSKVSPIVHLKGDPNSLKCLRYRLKPFKDLYC |
| | | | NMSSTWHWTSDDKGDKVGIVTVTYTTETQRQLFL |
| | | | NTVKIPPTVQISTGVMSL |
| HPV58 | | | MFQDAEEKPRTLHDLCQALETSVHEIELKCVECK |
| E6 | | | KTLQRSEVYDFVFADLRIVYRDGNPFAVCKVCLR |
| | | | LLSKISEYRHYNYSLYGDTLEQTLKKCLNEILIR |
| | | | CIICQRPLCPQEKKRHVDLNKREHNISGRWTGRC |
| | | | AVCWRPRRRQTQV |
| HPV58 | | | MRGNNPTLREYILDLHPEPTDLFCYEQLCDSSDE |
| E7 | | | DEIGLDGPDGQAQPATANYYIVTCCYTCGTFVRL |
| | | | CINSTTTDVRTLQQLLMGTCTIVCPSCAQQ |
| HPV58 | | | MVLILCCTLAILFCVADVNVFHIFLQMSVWRPSE |
| L1 | | | ATVYLPPVPVSKVVSTDEYVSRTSIYYYAGSSRL |
| | | | LAVGNPYFSIKSPNNNKKVLVPKVSGLQYRVFRV |
| | | | RLPDPNKFGFPDTSFYNPDTQRLVWACVGLEIGR |
| | | | GQPLGVGVSGHPYLNKFDDTETSNRYPAQPGSDN |
| | | | RECLSMDYKQTQLCLIGCKPPTGEHWGKGVACNN |
| | | | NAAATDCPPLELFNSIIEDGDMVDTGFGCMDFGT |
| | | | LQANKSDVPIDICNSTCKYPDYLKMASEPYGDSL |
| | | | FFFLRREQMFVRHFFNRAGKLGEAVPDDLYIKGS |
| | | | GNTAVIQSSAFFPTPSGSIVTSESQLFNKPYWLQ |
| | | | RAQGHNNGICWGNQLFVTVVDTTRSTNMTLCTEV |
| | | | TKEGTYKNDNFKEYVRHVEEYDLQFVFQLCKITL |
| | | | TAEIMTYIHTMDSNILEDWQFGLTPPPSASLQDT |
| | | | YRFVTSQAITCQKTAPPKEKEDPLNKYTFWEVNL |
| | | | KEKFSADLDQFPLGRKFLLQSGLKAKPRLKRSAP |
| | | | TTRAPSTKRKKVKK |
| HPV58 | | | MRHKRSTRRKRASATQLYQTCKASGTCPPDVIPK |
| L2 | | | VEGTTIADQILRYGSLGVFFGGLGIGTGSGTGGR |
| | | | TGYVPLGSTPPSEAIPLQPIRPPVTVDTVGPLDS |
| | | | SIVSLIEESSFIDAGAPAPSIPTPSGFDITTSAD |
| | | | TTPAILNVSSIGESSIQTVSTHLNPSFTEPSVLR |
| | | | PPAPAEASGHLIFSSPTVSTHSYENIPMDTFVIS |
| | | | TDSGNVTSSTPIPGSRPVARLGLYSRNTQQVKVV |
| | | | DPAFLTSPHRLVTYDNPAFEGFNPEDTLQFQHSD |
| | | | ISPAPDPDFLDIVALHRPALTSRRGTVRYSRVGQ |
| | | | KATLRTRSGKQIGAKVHYYQDLSPIQPVQEQVQQ |
| | | | QQQFELQSLNTSVSPYSINDGLYDIYADDADTIH |
| | | | DFQSPLHSHTSFATTRTSNVSIPLNTGFDTPLVS |
| | | | LEPGPDIASSVTSMSSFIPISPLTPFNTIIVDGA |
| | | | DFMLHPSYFILRRRRKRFPYFFADVRVAA |

The epitopes of the invention have been identified in a number of ways, as will be discussed below. Also discussed in greater detail is that peptide analogs derived from naturally occurring HPV sequences exhibit binding to HLA molecules and immunogenicity due to the modification of specific amino acid residues with respect to the naturally occurring HPV sequence. Further, the present invention provides compositions and combinations of compositions that enable epitope-based vaccines that are capable of interacting with HLA molecules encoded by various genetic alleles to provide broader population coverage than prior vaccines.

Definitions

The invention can be better understood with reference to the following definitions, which are listed alphabetically:

An “antigen” refers to a polypeptide encoded by the genome of an infectious agent, in this case, HPV. Examples of HPV antigens include E1, E2, E3, E4, E5, E6, E7, L1, and L2.

The designation of a residue position in an epitope as the “carboxyl terminus” or the “carboxyl terminal position” refers to the residue position at the carboxy terminus of the epitope, which is designated using conventional nomenclature as defined below. The “carboxyl terminal position” of the epitope occurring at the carboxyl end of the multi-epitope construct may or may not actually correspond to the carboxyl terminal end of a polypeptide. “C+1” refers to the residue or position immediately following the C-terminal residue of the epitope, i.e., refers to the residue flanking the C-terminus of the epitope. In preferred embodiments, the epitopes employed in the optimized multi-epitope constructs of the invention are motif-bearing epitopes and the carboxyl terminus of the epitope is defined with respect to primary anchor residues corresponding to a particular motif. In preferred embodiments, the carboxyl terminus of the epitope is defined as positions +8, +9, +10 or +11.

The designation of a residue position in an epitope as “amino terminus” or “amino-terminal position” refers to the residue position at the amino terminus of the epitope, which is designated using conventional nomenclature as defined below. The “amino terminal position” of the epitope occurring at the amino terminal end of the multi-epitope construct may or may not actually correspond to the amino terminal end of the polypeptide. “N−1” refers to the residue or position immediately adjacent to the epitope at the amino terminal end of an epitope. In preferred embodiments, the epitopes employed in the optimized multi-epitope constructs of the invention are motif-bearing epitopes and the amino terminus of the epitope is defined with respect to primary anchor residues corresponding to a particular motif. In preferred embodiments, the amino terminus of the epitope is defined as position +1.

A “computer” or “computer system” generally includes: a processor; at least one information storage and/or retrieval apparatus such as, for example, a hard drive, a disk drive or a tape drive; at least one input apparatus such as, for example, a keyboard, a mouse, a touch screen, or a microphone; and display structure. Additionally, the computer may include a communication channel in communication with a network such that remote users may communicate with the computer via the network to perform multi-epitope construct optimization functions disclosed herein. Such a computer may include more or less than what is listed above. The network may be a local area network (LAN), wide area network (WAN) or a global network such as the world wide web (e.g., the internet).

A “construct” as used herein generally denotes a composition that does not occur in nature. A construct may be a “polynucleotide construct” or a “polypeptide construct.” A construct can be produced by synthetic technologies, e.g., recombinant DNA preparation and expression or chemical synthetic techniques for nucleic or amino acids or peptides or polypeptides. A construct can also be produced by the addition or affiliation of one material with another such that the result is not found in nature in that form. Although a “construct” is not naturally occurring, it may comprise peptides that are naturally occurring.

The term “multi-epitope construct” when referring to nucleic acids and polynucleotides can be used interchangeably with the terms “minigene,” “minigene construct,” “multi-epitope nucleic acid vaccine,” “multi-epitope vaccine,” and other equivalent phrases (e.g., “epigene”), and comprises multiple epitope-encoding nucleic acids that encode peptide epitopes of any length that can bind to a molecule functioning in the immune system, preferably a class I HLA and a T-cell receptor or a class II HLA and a T-cell receptor. The nucleic acids encoding the epitopes in a multi-epitope construct can encode class I HLA epitopes and/or class II HLA epitopes. Class I HLA epitope-encoding nucleic acids are referred to as CTL epitope-encoding nucleic acids, and class II HLA epitope-encoding epitope nucleic acids are referred to as HTL epitope-encoding nucleic acids. Some multi-epitope constructs can have a subset of the multi-epitope-encoding nucleic acids encoding class I HLA epitopes and another subset of the multi-epitope-encoding nucleic acids encoding class II HLA epitopes. The CTL epitope-encoding nucleic acids preferably encode an epitope peptide of about 15 residues in length, less than about 15 residues in length, or less than about 13 amino acids in length, or less than about 11 amino acids in length, preferably about 8 to about 13 amino acids in length, more preferably about 8 to about 11 amino acids in length (e.g., 8, 9, 10, or 11), and most preferably about 9 or 10 amino acids in length. The HTL epitope nucleic acids can encode an epitope peptide of about 50 residues in length, less than about 50 residues in length, and usually consist of about 6 to about 30 residues, more usually between about 12 to 25, and often about 15 to 20, and preferably about 7 to about 23, preferably about 7 to about 17, more preferably about 11 to about 15 (e.g., 11, 12, 13, 14 or 15), and most preferably about 13 amino acids in length. The multi-epitope constructs described herein preferably include 5 or more, 10 or more, 15 or more, 20 or more, 25 or more, 30 or more, 35 or more, 40 or more, 45 or more, 50 or more, 55 or more, 60 or more, 65 or more, 70 or more, or 75 or more epitope-encoding nucleic acid sequences. All of the epitope-encoding nucleic acids in a multi-epitope construct may be from one organism (e.g., the nucleotide sequence of every epitope-encoding nucleic acid may be present in HPV strains), or the multi-epitope construct may include epitope-encoding nucleic acid sequences present in two or more different organisms (e.g., the nucleotide sequence of some epitope encoding nucleic acid sequences from HPV, and/or some from HPV, and/or some from HIV, and/or some from HCV). The epitope-encoding nucleic acid molecules in a multi-epitope construct may also be from multiple strains or types of an organism (e.g., HPV Types 16, 18, 31, 33, 45, 52, 58 and/or 56). The term “minigene” is used herein to refer to certain multi-epitope constructs. As described hereafter, one or more epitope-encoding nucleic acids in the multi-epitope construct may be flanked by spacer nucleotides, and/or other polynucleotide sequences also described herein or otherwise known in the art.

The term “multi-epitope construct,” when referring to polypeptides, can be used interchangeably with the terms “minigene construct,” multi-epitope vaccine,” and other equivalent phrases, and comprises multiple peptide epitopes of any length that can bind to a molecule functioning in the immune system, preferably a class I HLA and a T-cell receptor or a class II HLA and a T-cell receptor. The epitopes in a multi-epitope construct can be class I HLA epitopes and/or class II HLA epitopes. Class I HLA epitopes are referred to as CTL epitopes, and class II HLA epitopes are referred to as HTL epitopes. Some multi-epitope constructs can have a subset of class I HLA epitopes and another subset of class II HLA epitopes. The CTL Epitopes preferably are about 15 amino acid residues in length, less than about 15 amino acid residues in length, or less than about 13 amino acid residues in length, or less than about 11 amino acid residues in length, and preferably encode an epitope peptide of about 8 to about 13 amino acid residues in length, more preferably about 8 to about 11 amino acid residues in length (e.g., 8, 9, 10 or 11), and most preferably about 9 or 10 amino acid residues in length. The HTL epitopes are about 50 amino acid residues in length, less than about 50 amino acid residues in length, and usually consist of about 6 to about 30 amino acid residues in length, more usually between about 12 to about 25 amino acid residues in length, and preferably about 7 to about 23 amino acid residues in length, preferably about 7 to about 17 amino acid residues in length, more preferably about 11 to about 15 amino acid residues in length (e.g., 11, 12, 13, 14 or 15), and most preferably about 13 amino acid residues in length. The multi-epitope constructs described herein preferably include 5 or more, 10 or more, 15 or more, 20 or more, 25 or more, 30 or more, 35 or more, 40 or more, 45 or more, 50 or more, 55 or more, 60 or more, 65 or more, 70 or more, or 75 or more epitopes. All of the epitopes in a multi-epitope construct may be from one organism (e.g., every epitope may be present in one or more HPV strains), or the multi-epitope construct may include epitopes present in two or more different organisms (e.g., some epitopes from HPV and/or some from HIV, and/or some from HCV, and/or some from HBV). The epitopes in a multi-epitope construct may also be from multiple strains or types of an organism (e.g., HPV Types 6a, 6b, 11a, 16, 18, 31, 33, 45, 52, 56 and/or 58). The term “minigene” is used herein to refer to certain multi-epitope constructs. As described hereafter, one or more epitopes in the multi-epitope construct may be flanked by a spacer sequence, and or other sequences also described herein or otherwise known in the art.

“Cross-reactive binding” indicates that a peptide can bind more than one HLA molecule; a synonym is degenerate binding.

A “cryptic epitope” elicits a response by immunization with an isolated peptide, but the response is not cross-reactive in vitro when intact whole protein which comprises the epitope is used as an antigen.

A “dominant epitope” is an epitope that induces an immune response upon immunization with a whole native antigen (see, e.g., Sercarz, et al., Ann. Rev. Immunol. 11:729-66, 1993). Such a response is cross-reactive in vitro with an isolated peptide epitope.

An “epitope” is a set of amino acid residues linked together by amide bonds in a linear fashion. In the context of immunoglobulins, an “epitope” is involved in recognition and binding to a particular immunoglobulin. In the context of T cells, an “epitope” is those amino acid residues necessary for recognition by T cell receptor proteins and/or Major Histocompatibility Complex (MHC) receptors. In both contexts, in vivo or in vitro, an epitope is the collective features of a molecule, such as primary, secondary and tertiary peptide structure, and charge, that together form an entity recognized by an immunoglobulin, T cell receptor or HLA molecule. Throughout this disclosure “epitope,” “peptide epitope,” and “peptide” are often used interchangeably. It is to be appreciated, however, that isolated or purified protein or peptide molecules larger than and comprising an epitope of the invention are still within the bounds of the invention.

A “flanking residue” is an amino acid residue that is positioned next to an epitope. A flanking residue can be introduced or inserted at a position adjacent to the N-terminus or the C-terminus of an epitope, or that occurs naturally in the intact protein.

“Heteroclitic analogs” are defined herein as peptides with increased potency for a specific T cell, as measured by increased responses to a given dose, or by a requirement of lesser amounts to achieve the same response. Advantages of heteroclitic analogs include that the epitopes can be more potent, or more economical (since a lower amount is required to achieve the same effect). In addition, modified epitopes might overcome antigen-specific T cell unresponsiveness (T cell tolerance). (See, e.g., PCT Publication No. WO01/36452, which is hereby incorporated by reference in its entirety.)

The term “homology,” as used herein, refers to a degree of complementarity between two nucleotide sequences. The word “identity” may substitute for the word “homology” when a polynucleotide has the same nucleotide sequence as another polynucleotide. Sequence homology and sequence identity can also be determined by hybridization studies under high stringency and/or low stringency, are disclosed herein and encompassed by the invention, are polynucleotides that hybridize to the multi-epitope constructs under low stringency or under high stringency. Also, sequence homology and sequence identity can be determined by analyzing sequences using algorithms and computer programs known in the art (e.g., BLAST). Such methods be used to assess whether a polynucleotide sequence is identical or homologous to the multi-epitope constructs disclosed herein. The invention pertains in part to nucleotide sequences having 80% or more, 85% or more, 90% or more, 95% or more, 97% or more, 98% or more, or 99% or more identity to the nucleotide sequence of a multi-epitope construct disclosed herein. In a preferred embodiment, a nucleotide sequence of the invention will have 70%, 80%, 85%, 90%, 91%, 92%, 93%, 94%, 95%, 96%, 97%, 98% or 99% identity to a reference sequence. In a more preferred embodiment, a nucleotide sequence of the invention will have 90%, 91%, 92%, 93%, 94%, 95%, 96%, 97%, 98% or 99% identity to a reference sequence. In a more preferred embodiment, a nucleotide sequence of the invention will have 95%, 96%, 97%, 98% or 99% identity to a reference sequence.

As used herein, the term “stringent conditions” refers to conditions which permit hybridization between nucleotide sequences and the nucleotide sequences of the disclosed multi-epitope constructs. Suitable stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature. For example, hybridization under high stringency conditions could occur in about 50% formamide at about 37° C. to 42° C. In particular, hybridization could occur under high stringency conditions at 42° C. in 50% formamide, 5×SSPE, 0.3% SDS, and 200 μg/ml sheared and denatured salmon sperm DNA or at 42° C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. Hybridization could occur under reduced stringency conditions in about 35% to 25% formamide at about 30° C. to 35° C. For example, reduced stringency conditions could occur at 35° C. in 35% formamide, 5×SSPE, 0.3% SDS, and 200 μg/ml sheared and denatured salmon sperm DNA. The temperature range corresponding to a particular level of stringency can be further narrowed by calculating the purine to pyrimidine ratio of the nucleic acid of interest and adjusting the temperature accordingly. Variations on the above ranges and conditions are well known in the art.

In addition to utilizing hybridization studies to assess sequence identity or sequence homology, known computer programs may be used to determine whether a particular polynucleotide sequence is homologous to a multi-epitope construct disclosed herein. An example of such a program is the Bestfit program (Wisconsin Sequence Analysis Package, Version 8 for Unix, Genetics Computer Group, University Research Park, 575 Science Drive, Madison, Wis. 53711), and other sequence alignment programs are known in the art and may be utilized for determining whether two or more nucleotide sequences are homologous. Bestfit uses the local homology algorithm of Smith and Waterman (Adv. Appl. Mathematics 2: 482-89 (1981)), to find the best segment of homology between two sequences. When using Bestfit or any other sequence alignment program to determine whether a particular sequence is, for instance, 95% identical to a reference sequence, the parameters may be set such that the percentage of identity is calculated over the full length of the reference nucleotide sequence and that gaps in homology of up to 5% of the total number of nucleotides in the reference sequence are allowed.

“Human Leukocyte Antigen” or “HLA” is a human class I or class II Major Histocompatibility Complex (MHC) protein (see, e.g., Stites, et al., Immunology, 8th Ed., Lange Publishing, Los Altos, Calif. (1994)).

An “HLA supertype or family,” as used herein, describes sets of HLA molecules grouped on the basis of shared peptide-binding specificities. HLA class I molecules that share somewhat similar binding affinity for peptides bearing certain amino acid motifs are grouped into HLA supertypes. The terms “HLA superfamily,” “HLA supertype family,” “HLA family,” and “HLA xx-like molecules” (where xx denotes a particular HLA type), are synonyms.

Throughout this disclosure, binding data results are often expressed in terms of “IC₅₀.” IC₅₀ is the concentration of peptide in a binding assay at which 50% inhibition of binding of a reference peptide is observed. Given the conditions in which the assays are run (i.e., limiting HLA proteins and labeled peptide concentrations), these values approximate KD values. Assays for determining binding are described in detail, e.g., in PCT publications WO 94/20127 and WO 94/03205, which are hereby incorporated by reference in their entireties. It should be noted that IC₅₀ values can change, often dramatically, if the assay conditions are varied, and depending on the particular reagents used (e.g., HLA preparation, etc.). For example, excessive concentrations of HLA molecules will increase the apparent measured IC₅₀ of a given ligand.

Notwithstanding this fact, binding in the disclosure provided herein is expressed relative to a reference peptide. Although a particular assay may become more, or less, sensitive, and the IC₅₀'s of the peptides tested may change somewhat, the binding relative to the reference peptide will not significantly change. For example, in an assay run under conditions such that the IC₅₀ of the reference peptide increases 10-fold, the IC₅₀ values of the test peptides will also shift commensurately (i.e., approximately 10-fold in this example). Therefore, to avoid ambiguities, the assessment of whether a peptide is a “good,” “intermediate,” “weak,” or “negative” binder is generally based on its IC₅₀, relative to the IC₅₀ of a standard peptide.

Binding may also be determined using other assay systems including those using: live cells (e.g., Ceppellini, et al., Nature 339:392, 1989; Christnick, et al., Nature 352:67, 1991; Busch, et al., Int. Immunol. 2:443, 1990; Hill, et al., J. Immunol. 147:189, 1991; del Guercio, et al., J. Immunol. 154:685, 1995), cell free systems using detergent lysates (e.g., Cerundolo, et al., J. Immunol. 21:2069, 1991), immobilized purified MHC (e.g., Hill, et al., J. Immunol. 152, 2890, 1994; Marshall, et al., J. Immunol. 152:4946, 1994), ELISA systems (e.g., Reay, et al., EMBO J. 11:2829, 1992), surface plasmon resonance (e.g., Khilko, et al., J. Biol. Chem. 268:15425, 1993); high flux soluble phase assays (Hammer, et al., J. Exp. Med. 180:2353, 1994), and measurement of class I MHC stabilization or assembly (e.g., Ljunggren, et al., Nature 346:476, 1990; Schumacher, et al., Cell 62:563, 1990; Townsend, et al., Cell 62:285, 1990; Parker, et al., J. Immunol. 149:1896, 1992).

As used herein with respect to HLA class I molecules, “high affinity” is defined as binding with an IC₅₀, or KD value, of 50 nM or less; “intermediate affinity” is binding with an IC₅₀ or KD value of between about 50 and about 500 nM. With respect to binding to HLA class II molecules, “high affinity” is defined as binding with an IC₅₀ or KD value of 100 nM or less; “intermediate affinity” is binding with an IC₅₀ or KD value of between about 100 and about 1000 nM.

A peptide epitope occurring with “high frequency” is one that occurs in at least 30%, at least 40%, at least 50%, at least 60%, at least 70%, at least 80%, or at least 90% of the infectious agents in a population. A “high frequency” peptide epitope is one of the more common in a population, preferably the first most common, second most common, third most common, or fourth most common in a population of variant peptide epitopes.

The terms “identical” or percent “identity,” in the context of two or more peptide or nucleic acid sequences, refers to two or more sequences or subsequences that are the same or have a specified percentage of amino acid residues that are the same, when compared and aligned for maximum correspondence over a comparison window, as measured using a sequence comparison algorithm (e.g., BLAST) or by manual alignment and visual inspection.

An “immunogenic peptide” or “immunogenic peptide epitope” is a peptide that comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Introducing” an amino acid residue at a particular position in a multi-epitope construct, e.g., adjacent, at the C-terminal side, to the C-terminus of the epitope, encompasses configuring multiple epitopes such that a desired residue is at a particular position, e.g., adjacent to the epitope, or such that a deleterious residue is not adjacent to the C-terminus of the epitope. The term also includes inserting an amino acid residue, preferably a preferred or intermediate amino acid residue, at a particular position. An amino acid residue can also be introduced into a sequence by substituting one amino acid residue for another. Preferably, such a substitution is made in accordance with analoging principles set forth, e.g., in co-pending U.S. patent application Ser. No. 09/260,714, filed Mar. 1, 1999; PCT Application No. PCT/US00/19774; and/or PCT Application No. PCT/US00/31856; each of which is hereby incorporated in its entirety.

“Link” or “join” refers to any method known in the art for functionally connecting peptides, including, without limitation, recombinant fusion, covalent bonding, disulfide bonding, ionic bonding, hydrogen bonding, and electrostatic bonding.

“Major Histocompatibility Complex” or “MHC” is a cluster of genes that plays a role in control of the cellular interactions responsible for physiologic immune responses. In humans, the MHC complex is also known as the HLA complex. For a detailed description of the MHC and HLA complexes, see, Paul, Fundamental Immunology, 3rd Ed., Raven Press, New York, 1993.

As used herein, “middle of the peptide” is a position in a peptide that is neither an amino nor a carboxyl terminus.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “negative binding residue” or “deleterious residue” is an amino acid which, if present at certain positions (typically not primary anchor positions) in a peptide epitope, results in decreased binding affinity of the peptide for the peptide's corresponding HLA molecule.

A “non-native” sequence or “construct” refers to a sequence that is not found in nature, i.e., is “non-naturally occurring”. Such sequences include, e.g., peptides that are lipidated or otherwise modified, and polyepitopic compositions that contain epitopes that are not contiguous to the same epitopic and non-epitopic sequences found in a native protein sequence.

The phrase “operably linked” refers to a linkage in which a nucleotide sequence is connected to another nucleotide sequence (or sequences) in such a way as to be capable of altering the functioning of the sequence (or sequences). For example, a nucleic acid or multi-epitope nucleic acid construct which is operably linked to a regulatory sequence such as a promoter/operator places expression of the polynucleotide sequence of the construct under the influence or control of the regulatory sequence. Two nucleotide sequences (such as a protein encoding sequence and a promoter region sequence linked to the 5′ end of the coding sequence) are said to be operably linked if induction of promoter function results in the transcription of the protein coding sequence mRNA and if the nature of the linkage between the two nucleotide sequences does not (1) result in the introduction of a frame-shift mutation nor (2) prevent the expression regulatory sequences to direct the expression of the mRNA or protein. Thus, a promoter region would be operably linked to a nucleotide sequence if the promoter were capable of effecting transcription of that nucleotide sequence under appropriate conditions.

“Optimizing” refers to increasing the immunogenicity or antigenicity of a multi-epitope construct having at least one epitope pair by sorting epitopes to minimize the occurrence of junctional epitopes, inserting flanking residues that flank the C-terminus and/or N-terminus of an epitope, and inserting one or more spacer residues to further prevent the occurrence of junctional epitopes and/or to provide one or more flanking residues. An increase in immunogenicity or antigenicity of an optimized multi-epitope construct is measured relative to a multi-epitope construct that has not been constructed based on the optimization parameters using assays known to those of skill in the art, e.g., assessment of immunogenicity in HLA transgenic mice, ELISPOT, inteferon-gamma release assays, tetramer staining, chromium release assays, and/or presentation on dendritic cells.

The term “peptide” is used interchangeably with “oligopeptide” in the present specification to designate a series of residues, typically 1-amino acids, connected one to the other, typically by peptide bonds between the α-amino and carboxyl groups of adjacent amino acids. The preferred CTL-inducing peptides of the invention are about 15 residues in length, less than about 15 residues in length, and preferably 13 residues or less in length and preferably are about 8 to about 13 amino acids in length (e.g., 8, 9, 10, or 11), and usually consist of between about 8 and about 11 residues, preferably 9 or 10 residues. The preferred HTL-inducing oligopeptides are about 50 residues in length, less than about 50 residues in length, usually about 6 to about 30 residues, and usually consist of between about 6 and about 30 residues, more usually between about 12 and 25, and often between about 15 and 20 residues, or about 7 to about 23, preferably about 7 to about 17, more preferably about 11 to about 15 (e.g., 11, 12, 13, 14, or 15), and most preferably about 13 amino acids in length. The multi-epitope constructs described herein preferably include 5 or more, 10 or more, 15 or more, 20 or more, 25 or more, 30 or more, 35 or more, 40 or more, 45 or more, 50 or more, 55 or more, 60 or more, 65 or more, 70 or more, 75 or more epitope-encoding nucleic acids. In highly preferred embodiments, the multi-epitope constructs described herein include 30 or more (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75) epitope-encoding nucleic acids.

The nomenclature used to describe peptide, polypeptide, and protein compounds follows the conventional practice wherein the amino group is presented to the left (the N-terminus) and the carboxyl group to the right (the C-terminus) of each amino acid residue. When amino acid residue positions are referred to in a peptide epitope they are numbered in an amino to carboxyl direction with position one being the position at the amino terminal end of the epitope, or the peptide or protein of which it may be a part. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.) In addition to these symbols, “B”in the single letter abbreviations used herein designates α-amino butyric acid. Symbols for the amino acids are shown below in Table 2.

TABLE 2

| Single Letter Symbol | Three Letter Symbol | Amino Acids |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Amino acid “chemical characteristics” are defined as: Aromatic (F, W, Y); Aliphatic-hydrophobic (L, I, V, M); Small polar (S, T, C); Large polar (Q, N); Acidic (D, E); Basic (R, H, K); Proline; Alanine; and Glycine.

It is to be appreciated that protein or peptide molecules that comprise an epitope of the invention as well as additional amino acid residues are within the bounds of the invention. In certain embodiments, there is a limitation on the length of a peptide of the invention which is not otherwise a construct as defined herein. An embodiment that is length-limited occurs when the protein/peptide comprising an epitope of the invention comprises a region (i.e., a contiguous series of amino acid residues) having 100% identity with a native sequence. In order to avoid a recited definition of epitope from reading, e.g., on whole natural molecules, the length of any region that has 100% identity with a native peptide sequence is limited. Thus, for a peptide comprising an epitope of the invention and a region with 100% identity with a native peptide sequence (and which is not otherwise a construct), the region with 100% identity to a native sequence generally has a length of: less than or equal to 600 amino acid residues, often less than or equal to 500 amino acid residues, often less than or equal to 400 amino acid residues, often less than or equal to 250 amino acid residues, often less than or equal to 100 amino acid residues, often less than or equal to 85 amino acid residues, often less than or equal to 75 amino acid residues, often less than or equal to 65 amino acid residues, and often less than or equal to 50 amino acid residues, often less than 40, 30, 25, 20, 19, 18, 17, 16, 15, 14, 13, 12, 11, 10 or 9 amino acid residues. In certain embodiments, an “epitope” of the invention which is not a construct is comprised by a peptide having a region with less than 51 amino acid residues that has 100% identity to a native peptide sequence, in any increment down to 5 amino acid residues (e.g., 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6 or 5 amino acid residues).

Certain peptide or protein sequences longer than 600 amino acids are within the scope of the invention. Such longer sequences are within the scope of the invention provided that they do not comprise any contiguous sequence of more than 600 amino acids that have 100% identity with a native peptide sequence, or if longer than 600 amino acids, they are a construct. For any peptide that has five contiguous residues or less that correspond to a native sequence, there is no limitation on the maximal length of that peptide in order to fall within the scope of the invention. It is presently preferred that a CTL epitope of the invention be less than 600 residues long in any increment down to eight amino acid residues.

The terms “PanDR binding peptide,” “PanDR binding epitope,” “PADRE® peptide,” and “PADRE® epitope,” refer to a type of HTL peptide which is a member of a family of molecules that binds more than one HLA class II DR molecule. PADRE® peptides bind to most HLA-DR molecules and stimulate in vitro and in vivo human helper T lymphocyte (HTL) responses. The pattern that defines the PADRE® family of molecules can be thought of as an HLA Class II supermotif. For example, a PADRE® peptide may comprise the formula: aKXVAAWTLKAAa, where “X” is either cyclohexylalanine, phenylalanine or tyrosine and “a” is either D-alanine or L-alanine, has been found to bind to most HLA-DR alleles, and to stimulate the response of T helper lymphocytes from most individuals, regardless of their HLA type. An alternative of a PADRE® epitope comprises all “L” natural amino acids which can be provided in peptide/polypeptide form and in the form of nucleic acids that encode the epitope, e.g., in multi-epitope constructs. Specific examples of PADRE® peptides are also disclosed herein. Polynucleotides encoding PADRE® peptides are also contemplated as part of the present invention. PADRE® epitopes are described in detail in U.S. Pat. Nos. 5,679,640, 5,736,142, and 6,413,935; each of which is hereby incorporated by reference in its entirety.

“Pharmaceutically acceptable” refers to a non-toxic, inert, and/or physiologically compatible composition.

A “pharmaceutical excipient” comprises a material such as an adjuvant, a carrier, pH-adjusting and buffering agents, tonicity adjusting agents, wetting agents, preservatives, and the like.

“Presented to an HLA Class I processing pathway” means that the multi-epitope constructs are introduced into a cell such that they are largely processed by an HLA Class I processing pathway. Typically, multi-epitope constructs are introduced into the cells using expression vectors that encode the multi-epitope constructs. HLA Class II epitopes that are encoded by such a multi-epitope construct are also presented on Class II molecules, although the mechanism of entry of the epitopes into the Class II processing pathway is not defined.

A “primary anchor residue” or a “primary MHC anchor” is an amino acid at a specific position along a peptide sequence which is understood to provide a contact point between the immunogenic peptide and the HLA molecule. One, two or three, usually two, primary anchor residues within a peptide of defined length generally define a “motif” for an immunogenic peptide. These residues are understood to fit in close contact with peptide binding grooves of an HLA molecule, with their side chains buried in specific pockets of the binding grooves themselves. In one embodiment, for example, the primary anchor residues of an HLA class I epitope are located at position 2 (from the amino terminal position, wherein the N-terminal amino acid residue is at position +1) and at the carboxyl terminal position of a 9-residue peptide epitope in accordance with the invention. The primary anchor positions for each motif and supermotif disclosed herein are set forth in Table 3 herein or in Tables I and III of PCT/US00/27766, or PCT/US00/19774.

TABLE 3

| | | | POSITION |
|---|---|---|---|
| | POSITION | POSITION | C Terminus |
| | 2 (Primary | 3 (Primary | (Primary |
| | Anchor) | Anchor) | Anchor) |
| SUPERMOTIFS | | | |
| A1 | T, I, L, V, M, S | | F, W, Y |
| A2 | L, I, V, M, A, T, | | I, V, M, A, T, |
| | Q | | L |
| A3 | V, S, M, A, T, L, | | R, K |
| | I | | |
| A24 | Y, F, W, I, V, L, | | F, I, Y, W, L, |
| | M, T | | M |
| B7 | P | | V, I, L, F, M, W, |
| | | | Y, A |
| B27 | R, H, K | | F, Y, L, W, M, I, |
| | | | V, A |
| B44 | E, D | | F, W, L, I, M, V, |
| | | | A |
| B58 | A, T, S | | F, W, Y, L, I, V, |
| | | | M, A |
| B62 | Q, L, I, V, M, P | | F, W, Y, M, I, V, |
| | | | L, A |
| MOTIFS | | | |
| A1 | T, S, M | | Y |
| A1 | | D, E, A, S | Y |
| A2.1 | L, M, V, Q, I, A, | | V, L, I, M, A, T |
| | T | | |
| A3 | L, M, V, I, S, A, | | K, Y, R, H, F, A |
| | T, F, C, G, D | | |
| A11 | V, T, M, L, I, S, | | K, R, Y, H |
| | A, G, N, C, D, F | | |
| A24 | Y, F, W, M | | F, L, I, W |
| A*3101 | M, V, T, A, L, I, | | R, K |
| | S | | |
| A*3301 | M, V, A, L, F, I, | | R, K |
| | S, T | | |
| A*6801 | A, V, T, M, S, L, | | R, K |
| | I | | |
| B*0702 | P | | L, M, F, W, Y, A, |
| | | | I, V |
| B*3501 | P | | L, M, F, W, Y, I, |
| | | | V, A |
| B51 | P | | L, I, V, F, W, Y, |
| | | | A, M |
| B*5301 | P | | I, M, F, W, Y, A, |
| | | | L, V |
| B*5401 | P | | A, T, I, V, L, M, |
| | | | F, W, Y |

Bolded residues are preferred, italicized residues are tolerated: A peptide is considered motif-bearing if it has primary anchors at each primary anchor position for a motif or supermotif as specified in the above table.

Preferred amino acid residues that can serve as primary anchor residues for most Class II epitopes consist of methionine and phenylalanine in position one and V, M, S, T, A and C in position six. Tolerated amino acid residues that can occupy these positions for most Class II epitopes consist of L, I, V, W, and Y in position one and P, L and I in position six. The presence of these amino acid residues in positions one and six in Class 1 epitopes defines the HLA-DR1, 4, 7 supermotif. The HLA-DR3 binding motif is defined by preferred amino acid residues from the group consisting of L, I, V, M, F, Y and A in position one and D, E, N, Q, S and T in position four and K, R and H in position six. Other amino acid residues may be tolerated in these positions but they are not preferred. For example, analog peptides can be created by altering the presence or absence of particular residues in these primary anchor positions. Such analogs are used to modulate the binding affinity of a peptide comprising a particular motif or supermotif.

A “preferred primary anchor residue” is an anchor residue of a motif or supermotif that is associated with optimal binding. Preferred primary anchor residues are indicated in bold-face in Table 3. “Promiscuous recognition” is where a distinct peptide is recognized by the same T cell clone in the context of various HLA molecules. Promiscuous recognition or binding is synonymous with cross-reactive binding.

A “protective immune response” or “therapeutic immune response” refers to a CTL and/or an HTL response to an antigen derived from an infectious agent or a tumor antigen, which prevents or at least partially arrests or reverses disease symptoms, side effects, or progression either in part or in full. The immune response may also include an antibody response which has been facilitated by the stimulation of helper T cells.

By “ranking” the variants in a population of peptide epitopes is meant ordering each variant by its frequency of occurrence relative to the other variants.

By “regulatory sequence” is meant a polynucleotide sequence that contributes to or is necessary for the expression of an operably associated polynucleotide or polynucleotide construct in a particular host organism. The regulatory sequences that are suitable for prokaryotes, for example, include a promoter, optionally an operator sequence, and a ribosome binding site. Eukaryotic cells are known to utilize e.g., promoters, polyadenylation signals, and enhancers. In a preferred embodiment, a promoter is a CMV promoter. In less preferred embodiments, a promoter is another promoter described herein or known in the art. Regulatory sequences include IRESs. Other specific examples of regulatory sequences are described herein and otherwise known in the art.

The term “residue” refers to an amino acid or amino acid mimetic incorporated into an oligopeptide by an amide bond or amide bond mimetic.

A “secondary anchor residue” is an amino acid residue at a position other than a primary anchor position in a peptide which may influence peptide binding. A secondary anchor residue occurs at a significantly higher frequency among bound peptides than would be expected by random distribution of amino acid residues at one position.

The secondary anchor residues are said to occur at “secondary anchor positions.” A secondary anchor residue can be identified as a residue which is present at a higher frequency among high or intermediate affinity binding peptides, or a residue otherwise associated with high or intermediate affinity binding. For example, in certain embodiments of the present invention, analog peptides are created by altering the presence or absence of particular residues in one or more secondary anchor positions. Such analogs are used to finely modulate the binding affinity of a peptide comprising a particular motif or supermotif. The terminology “fixed peptide” is sometimes used to refer to an analog peptide.

“Sorting epitopes” refers to determining or designing an order of the epitopes in a multi-epitope construct according to methods of the present invention.

A “spacer” (or “spacer sequence”) refers to one or more amino acid residues (or nucleotides encoding such residues) inserted between two epitopes in a multi-epitope construct to prevent the occurrence of junctional epitopes and/or to increase the efficiency of processing. A multi-epitope construct may have one or more spacer regions. In some embodiments, a spacer region may flank each epitope-encoding nucleic acid sequence in a construct, or the ratio of spacer nucleotides to epitope-encoding nucleotides may be about 2 to 10, about 5 to 10, about 6 to 10, about 7 to 10, about 8 to 10, or about 9 to 10, where a ratio of about 8 to 10 has been determined to yield favorable results for some constructs.

The spacer nucleotides may encode one or more amino acids. A spacer nucleotide sequence flanking a class I HLA epitope in a multi-epitope construct is preferably of a length that encodes between one and about eight amino acids. A spacer nucleotide sequence flanking a class II HLA epitope in a multi-epitope construct is preferably of a length that encodes greater than five, six, seven, or more amino acids, and more preferably five or six amino acids.

The number of spacers in a construct, the number of amino acid residues in a spacer, and the amino acid composition of a spacer can be selected to optimize epitope processing and/or minimize junctional epitopes. It is preferred that spacers are selected by concomitantly optimizing epitope processing and junctional motifs. Suitable amino acids for optimizing epitope processing are described herein. Also, suitable amino acid spacing for minimizing the number of junctional epitopes in a construct are described herein for class I and class II HLAs. For example, spacers flanking class II HLA epitopes preferably include G, P, and/or N residues as these are not generally known to be primary anchor residues (see, e.g., PCT Application NO. PCT/US00/19774). A particularly preferred spacer for flanking a class II HLA epitope includes alternating G and P residues, for example, (GP)n, (PG)n, (GP)nG, (PG)nP, and so forth, where n is an integer between zero and eleven (e.g., 1, 2, 3, 4, 5, 6, 7, 8, 9, 10 or 11), preferably two or about two, and where a specific example of such a spacer is GPGPG (SEQ ID NO:______). A preferred spacer, particularly for class I HLA epitopes, comprises one, two, three or more consecutive alanine (A) residues.

In some multi-epitope constructs, it is sufficient that each spacer nucleic acid encodes the same amino acid sequence. In multi-epitope constructs having two spacer nucleic acids encoding the same amino acid sequence, the spacer nucleic acids encoding those spacers may have the same or different nucleotide sequences, where different nucleotide sequences may be preferred to decrease the likelihood of unintended recombination events when the multi-epitope construct is inserted into cells.

In other multi-epitope constructs, one or more of the spacer nucleotides may encode different amino acid sequences. While many of the spacer nucleotides may encode the same amino acid sequence in a multi-epitope construct, one, two, three, four, five or more spacer nucleotides may encode different amino acid sequences, and it is possible that all of the spacer nucleotides in a multi-epitope construct encode different amino acid sequences. Spacer nucleotides may be optimized with respect to the epitope nucleic acids they flank by determining whether a spacer sequence will maximize epitope processing and/or minimize junctional epitopes, as described herein.

In certain embodiments, multi-epitope constructs are distinguished from one another according to whether the spacers in one construct optimize epitope processing or minimize junctional epitopes with respect to another construct. In preferred embodiments, constructs are distinguished where one construct is concomitantly optimized for epitope processing and junctional epitopes with respect to one or more other constructs. Computer assisted methods and in vitro and in vivo laboratory methods for determining whether a construct is optimized for epitope processing and junctional motifs are described herein.

A “subdominant epitope” is an epitope which evokes little or no response upon immunization with whole antigens which comprise the epitope, but for which a response can be obtained by immunization with an isolated peptide, and this response (unlike the case of cryptic epitopes) is detected when whole protein is used to recall the response in vitro or in vivo.

A “supermotif” is a peptide binding specificity shared by HLA molecules encoded by two or more HLA alleles. Preferably, a supermotif-bearing peptide is recognized with high or intermediate affinity (as defined herein) by two or more HLA antigens.

“Synthetic peptide” refers to a peptide that is man-made using such methods as chemical synthesis or recombinant DNA technology.

A “tolerated primary anchor residue” is an anchor residue of a motif or supermotif that is associated with binding to a lesser extent than a preferred residue. Tolerated primary anchor residues are indicated in italicized text in Table 3.

As used herein, a “vaccine” is a composition that contains one or more peptides of the invention. There are numerous embodiments of vaccines in accordance with the invention, such as by a cocktail of one or more peptides; one or more epitopes of the invention comprised by a polyepitopic peptide; or nucleotides that encode such peptides or polypeptides, e.g., a minigene that encodes a polyepitopic peptide. The “one or more peptides” can include any whole unit integer from 1-150, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 80, 85, 90, 95, 100, 105, 110, 115, 120, 125, 130, 135, 140, 145, or 150 or more peptides of the invention. The peptides or polypeptides can optionally be modified, such as by lipidation, addition of targeting or other sequences. In other embodiments, polynucleotides or minigenes of the invention are modified to include signals for targeting, processing or other sequences. HLA class I-binding peptides of the invention can be admixed with, or linked to, HLA class II-binding peptides, to facilitate activation of both cytotoxic T lymphocytes and helper T lymphocytes. Vaccines can also comprise peptide-pulsed antigen presenting cells, e.g., dendritic cells.

A “variant of a peptide epitope” refers to a peptide that is identified from a different viral strain at the same position in an aligned sequence, and that varies by one or more amino acid residues from the parent peptide epitope. Examples of peptide epitope variants of HPV include those shown in Table 9 of International Patent Application No. PCT/US04/009510, filed Mar. 29, 2004, which claims benefit of priority to U.S. Application No. 60/458,026, filed Mar. 28, 2003.

A “variant of an antigen” refers to an antigen that comprises at least one variant of a peptide epitope. Examples of antigen variants of HPV include those listed herein.

A “variant of an infectious agent” refers to an infectious agent whose genome encodes at least one variant of an antigen. Variants of infectious agents are related viral strains or isolates that comprise sequence variations, but cause some or all of the same disease symptoms. Examples of HPV infectious agents or variants include HPV strains 1-92 (preferably HPV strains 16, 18, 31, 33, 45, 52, 56, and 58).

A “TCR contact residue” or “T cell receptor contact residue” is an amino acid residues in an epitope that is understood to be bound by a T cell receptor; these are defined herein as not being any primary MHC anchor residues. T cell receptor contact residues are defined as the position/positions in the peptide where all analogs tested induce or reduce T-cell recognition relative to that induced with a wildtype peptide.

Acronyms used herein are defined as follows:

| | |
|---|---|
| APC: | Antigen presenting cell |
| CD3: | Pan T cell marker |
| CD4: | Helper T lymphocyte marker |
| CD8: | Cytotoxic T lymphocyte marker |
| CEA: | Carcinoembryonic antigen |
| CFA: | Complete Freund's Adjuvant |
| CTL: | Cytotoxic T lymphocytes |
| DC: | Dendritic cells. DC functioned as potent antigen |
| | presenting cells by stimulating cytokine release from |
| | CTL lines that were specific for a model peptide |
| | derived from hepatitis B virus (HBV). In vitro |
| | experiments using DC pulsed ex vivo with an HBV |
| | peptide epitope have stimulated CTL immune responses |
| | in vitro following delivery to naive mice. |
| DMSO: | Dimethylsulfoxide |
| ELISA: | Enzyme-linked immunosorbant assay |
| E:T: | Effector:target ratio |
| FCS: | Fetal calf serum |
| G-CSF: | Granulocyte colony-stimulating factor |
| GM-CSF: | Granulocyte-macrophage (monocyte)-colony |
| | stimulating factor |
| HBV: | Hepatitis B virus |
| HER2/Neu: | c-erbB-2 |
| HLA: | Human leukocyte antigen |
| HLA-DR: | Human leukocyte antigen class II |
| HPLC: | High Performance Liquid Chromatography |
| HPV: | Human Papillomavirus |
| HTC: | Helper T cells |
| HTL: | Helper T Lymphocyte |
| ID: | Identity |
| IFA: | Incomplete Freund's Adjuvant |
| IFNγ: | Interferon gamma |
| IL-4: | Interleukin-4 cytokine |
| IV: | Intravenous |
| LU30%: | Cytotoxic activity required to achieve 30% lysis at a |
| | 100:1 |
| | (E:T) ratio |
| MAb: | Monoclonal antibody |
| MAGE: | Melanoma antigen |
| MLR: | Mixed lymphocyte reaction |
| MNC: | Mononuclear cells |
| PB: | Peripheral blood |
| PBMC: | Peripheral blood mononuclear cell |
| SC: | Subcutaneous |
| S.E.M.: | Standard error of the mean |
| QD: | Once a day dosing |
| TAA: | Tumor associated antigen |
| TCR: | T cell receptor |
| TNF: | Tumor necrosis factor |
| WBC: | White blood cells |

Stimulation of CTL and HTL Responses

The mechanism by which T cells recognize antigens has begun to be thoroughly delineated during the past fifteen years. Based on our understanding of the immune system we have developed efficacious peptide epitope vaccine compositions that can induce a therapeutic or prophylactic immune response to HPV in a broad population. For an understanding of the value and efficacy of the claimed compositions, a brief review of immunology-related technology is provided.

A complex of an HLA molecule and a peptide antigen acts as the ligand recognized by HLA-restricted T cells (Buus, S. et al., Cell 47:1071, 1986; Babbitt, B. P. et al., Nature 317:359, 1985; Townsend, A. and Bodmer, H., Ann. Rev. Immunol. 7:601, 1989; Germain, R. N., Ann. Rev. Immunol. 11:403, 1993). Through the study of single amino acid substituted antigen analogs and the sequencing of endogenously bound, naturally processed peptides, critical residues that correspond to motifs required for specific binding to HLA antigen molecules have been identified (see e.g., Southwood, et al., J. Immunol. 160:3363-3373 (1998); Rammensee, et al., Immunogenetics 41:178 (1995); Rammensee et al., Sette, A. and Sidney, J. Curr. Opin. Immunol. 10:478 (1998); Engelhard, V. H., Curr. Opin. Immunol. 6:13 (1994); Sette, A. and Grey, H. M., Curr. Opin. Immunol. 4:79 (1992); Sinigaglia, F. and Hammer, J. Curr. Biol. 6:52 (1994); Ruppert et al., Cell 74:929-937 (1993); Kondo et al., J. Immunol. 155:4307-4312 (1995); Sidney et al., J. Immunol. 157:3480-90 (1996); Sidney et al., Human Immunol. 45:79-93 (1996); Sette, A. and Sidney, J. Immunogenetics 50(3-4):201-212 (1999) Review).

Furthermore, x-ray crystallographic analysis of HLA-peptide complexes has revealed pockets within the peptide binding cleft of HLA molecules which accommodate, in an allele-specific mode, residues borne by peptide ligands; these residues in turn determine the HLA binding capacity of the peptides in which they are present. (See, e.g., Madden, D. R. Annu. Rev. Immunol. 13:587, 1995; Smith, et al., Immunity 4:203, 1996; Fremont et al., Immunity 8:305, 1998; Stern et al., Structure 2:245, 1994; Jones, E. Y. Curr. Opin. Immunol. 9:75, 1997; Brown, J. H. et al., Nature 364:33, 1993; Guo, H. C. et al., Proc. Natl. Acad. Sci. USA 90:8053, 1993; Guo, H. C. et al., Nature 360:364, 1992; Silver, M. L. et al., Nature 360:367, 1992; Matsumura, M. et al., Science 257:927, 1992; Madden et al., Cell 70:1035, 1992; Fremont, D. H. et al., Science 257:919, 1992; Saper, M. A., Bjorkman, P. J. and Wiley, D. C., J. Mol. Biol. 219:277, 1991.)

Accordingly, the definition of class I and class II allele-specific HLA binding motifs, or class I or class II supermotifs allows identification of regions within a protein that have the potential of binding particular HLA antigen(s).

The present inventors have found that the correlation of binding affinity with immunogenicity, which is disclosed herein, is an important factor to be considered when evaluating candidate peptides. Thus, by a combination of motif searches, HLA-peptide binding assays, and in vivo immunogenicity analyses, candidates for epitope-based vaccines have been identified. After determining their binding affinity, additional confirmatory work can be performed to select, among these vaccine candidates, epitopes with preferred characteristics in terms of population coverage, antigenicity, and immunogenicity.

Various strategies can be utilized to evaluate immunogenicity, including, by non-limiting example, the following:

(1) Evaluation of primary T cell cultures from normal individuals (see, e.g., Wentworth, P. A. et al., Mol. Immunol. 32:603, 1995; Celis, E. et al., Proc. Natl. Acad. Sci. USA 91:2105, 1994; Tsai, V. et al., J. Immunol. 158:1796, 1997; Kawashima, I. et al., Human Immunol. 59:1, 1998); This procedure involves the stimulation of peripheral blood lymphocytes (PBL) from normal subjects with a test peptide in the presence of antigen presenting cells in vitro over a period of several weeks. T cells specific for the peptide become activated during this time and are detected using, e.g., a lymphokine- or ⁵¹Cr-release assay involving peptide sensitized target cells.

(2) Immunization of HLA transgenic mice (see, e.g., Wentworth, P. A. et al., J. Immunol. 26:97, (1996); Wentworth, P. A. et al., Int. Immunol. 8:651, (1996); Alexander, J. et al., J. Immunol. 159:4753, (1997); McKinney, D., et al., J. Immunol. Methods 237:105-17 (2000)). In this method, peptides in incomplete Freund's adjuvant are administered subcutaneously to HLA transgenic mice. Several weeks following immunization, splenocytes are removed and cultured in vitro in the presence of test peptide for approximately one week. Peptide-specific T cells are detected using, e.g., a lymphokine or ⁵¹Cr-release assay involving peptide sensitized target cells and target cells expressing endogenously generated antigen.

(3) Demonstration of recall T cell responses from immune individuals who have effectively been vaccinated, recovered from infection, and/or from chronically infected patients (see, e.g., Rehermann, B. et al., J. Exp. Med. 181:1047, 1995; Doolan, D. L. et al., Immunity 7:97, 1997; Bertoni, R. et al., J. Clin. Invest. 100:503, 1997; Threlkeld, S. C. et al., J. Immunol. 159:1648, 1997; Diepolder, H. M. et al., J. Virol. 71:6011, 1997); In applying this strategy, recall responses are detected by culturing PBL from subjects that have been naturally exposed to the antigen, for instance through infection, and thus have generated an immune response “naturally”, or from patients who were vaccinated against the infection. PBL from subjects are cultured in vitro for 1 day to 2 weeks in the presence of test peptide plus antigen presenting cells (APC) to allow activation of “memory” T cells, as compared to “naive” T cells. At the end of the culture period, T cell activity is detected using assays for T cell activity including ⁵¹Cr release involving peptide-sensitized targets, T cell proliferation, or lymphokine release.

Binding Affinity of Peptide Epitopes for HLA Molecules

As indicated herein, the large degree of HLA polymorphism is an important factor to be taken into account with the epitope-based approach to vaccine development. To address this factor, epitope selection encompassing identification of peptides capable of binding at high or intermediate affinity to multiple HLA molecules is preferably utilized, most preferably these epitopes bind at high or intermediate affinity to two or more allele-specific HLA molecules.

CTL-inducing peptides of interest for vaccine compositions preferably include those that have an IC₅₀ or binding affinity value for class I HLA molecules of 500 nM or better (i.e., the value is ≦500 nM). HTL-inducing peptides preferably include those that have an IC₅₀ or binding affinity value for class II HLA molecules of 1000 nM or better, (i.e., the value is ≦1,000 nM). For example, peptide binding is assessed by testing the capacity of a candidate peptide to bind to a purified HLA molecule in vitro. Peptides exhibiting high or intermediate affinity are then considered for further analysis. Selected peptides are tested on other members of the supertype family. In preferred embodiments, peptides that exhibit cross-reactive binding are then used in cellular screening analyses or vaccines.

As disclosed herein, higher HLA binding affinity is correlated with greater immunogenicity. Greater immunogenicity can be manifested in several different ways. Immunogenicity corresponds to whether an immune response is elicited at all, and to the vigor of any particular response, as well as to the extent of a population in which a response is elicited. For example, a peptide might elicit an immune response in a diverse array of the population, yet in no instance produce a vigorous response. In accordance with these principles, close to 90% of high binding peptides have been found to be immunogenic, as contrasted with about 50% of the peptides which bind with intermediate affinity. Moreover, higher binding affinity peptides lead to more vigorous immunogenic responses. As a result, less peptide is required to elicit a similar biological effect if a high affinity binding peptide is used. Thus, in preferred embodiments of the invention, high affinity binding epitopes are particularly useful.

The relationship between binding affinity for HLA class I molecules and immunogenicity of discrete peptide epitopes on bound antigens has been determined for the first time in the art by the present inventors. The correlation between binding affinity and immunogenicity was analyzed in two different experimental approaches (see, e.g., Sette, et al., J. Immunol. 153:5586-92, 1994). In the first approach, the immunogenicity of potential epitopes ranging in HLA binding affinity over a 10,000-fold range was analyzed in HLA-A*0201 transgenic mice. In the second approach, the antigenicity of approximately 100 different hepatitis B virus (HBV)-derived potential epitopes, all carrying A*0201 binding motifs, was assessed by using PBL from acute hepatitis patients. Pursuant to these approaches, it was determined that an affinity threshold value of approximately 500 nM (preferably 50 nM or less) determines the capacity of a peptide epitope to elicit a CTL response. These data are true for class I binding affinity measurements for naturally processed peptides and for synthesized T cell epitopes. These data also indicate the important role of determinant selection in the shaping of T cell responses (see, e.g., Schaeffer, et al. Proc. Natl. Acad. Sci. USA 86:4649-53, 1989).

An affinity threshold associated with immunogenicity in the context of HLA class II DR molecules has also been delineated (see, e.g., Southwood, et al. J. Immunology 160:3363-3373 (1998), and U.S. Pat. No. 6,413,517; each of which is hereby incorporated by reference in its entirety). In order to define a biologically significant threshold of DR binding affinity, a database of the binding affinities of 32 DR-restricted epitopes for their restricting element (i.e., the HLA molecule that binds the motif) was compiled. In approximately half of the cases (15 of 32 epitopes), DR restriction was associated with high binding affinities, i.e. binding affinity values of 100 nM or less. In the other half of the cases (16 of 32), DR restriction was associated with intermediate affinity (binding affinity values in the 100-1,000 nM range). In only one of 32 cases was DR restriction associated with an IC₅₀ of 1,000 nM or greater. Thus, 1,000 nM can be defined as an affinity threshold associated with immunogenicity in the context of DR molecules.

In the case of tumor-associated antigens (TAAs), many CTL peptide epitopes that have been shown to induce CTL that lyse peptide-pulsed target cells and tumor cell targets endogenously expressing the epitope exhibit binding affinity or IC₅₀ values of 200 nM or less. In a study that evaluated the association of binding affinity and immunogenicity of a small set of such TAA epitopes, 100% (i.e., 10 out of 10) of the high binders, i.e., peptide epitopes binding at an affinity of 50 nM or less, were immunogenic and 80% (i.e., 8 out of 10) of them elicited CTLs that specifically recognized tumor cells. In the 51 to 200 nM range, very similar figures were obtained. With respect to analog peptides, CTL inductions positive for wildtype peptide and tumor cells were noted for 86% (i.e., 6 out of 7) and 71% (i.e., 5 out of 7) of the peptides, respectively. In the 201-500 nM range, most peptides (i.e., 4 out of 5 wildtype) were positive for induction of CTL recognizing wildtype peptide, but tumor recognition was not detected.

The binding affinity of peptides for HLA molecules can be determined as described in Example 1, below.

Peptide Epitope Binding Motifs and Supermotifs

Through the study of single amino acid substituted antigen analogs and the sequencing of endogenously bound, naturally processed peptides, critical residues required for allele-specific binding to HLA molecules have been identified. The presence of these residues correlates with binding affinity for HLA molecules. The identification of motifs and/or supermotifs that correlate with high and intermediate affinity binding is an important issue with respect to the identification of immunogenic peptide epitopes for the inclusion in a vaccine. Kast, et al. (J. Immunol. 152:3904-3912, 1994) have shown that motif-bearing peptides account for 90% of the epitopes that bind to allele-specific HLA class I molecules. In this study all possible peptides of 9 amino acids in length and overlapping by eight amino acids (240 peptides), which cover the entire sequence of the E6 and E7 proteins of human papillomavirus type 16, were evaluated for binding to five allele-specific HLA molecules that are expressed at high frequency among different ethnic groups. This unbiased set of peptides allowed an evaluation of the predictive value of HLA class I motifs. From the set of 240 peptides, 22 peptides were identified that bound to an allele-specific HLA molecule with high or intermediate affinity. Of these 22 peptides, 20 (i.e. 91%) were motif-bearing. Thus, this study demonstrates the value of motifs for the identification of peptide epitopes for inclusion in a vaccine: application of motif-based identification techniques will identify about 90% of the potential epitopes in a target antigen protein sequence. Such peptide epitopes are identified in Tables 13-24 described below.

Peptides of the present invention may also comprise epitopes that bind to MHC class II DR molecules. Such peptide epitopes are identified in Tables 13-24 described below. A greater degree of heterogeneity in both size and binding frame position of the motif, relative to the N- and C-termini of the peptide, exists for class II peptide ligands. This increased heterogeneity of HLA class II peptide ligands is due to the structure of the binding groove of the HLA class II molecule which, unlike its class I counterpart, is open at both ends. Crystallographic analysis of HLA class II DRB*0101-peptide complexes showed that the major energy of binding is contributed by peptide residues complexed with complementary pockets on the DRB*0101 molecules. An important anchor residue engages the deepest hydrophobic pocket (see, e.g., Madden, D. R. Ann. Rev. Immunol. 13:587, 1995) and is referred to as position 1 (P1). P1 may represent the N-terminal residue of a class II binding peptide epitope, but more typically is flanked towards the N-terminus by one or more residues. Other studies have also pointed to an important role for the peptide residue in the sixth position towards the C-terminus, relative to P1, for binding to various DR molecules.

In the past few years evidence has accumulated to demonstrate that a large fraction of HLA class I and class II molecules can be classified into a relatively few supertypes, each characterized by largely overlapping peptide binding repertoires, and consensus structures of the main peptide binding pockets. Thus, peptides of the present invention are identified by any one of several HLA-specific amino acid motifs (see, e.g., Tables 13-24), or if the presence of the motif corresponds to the ability to bind several allele-specific HLA antigens, a supermotif. The HLA molecules that bind to peptides that possess a particular amino acid supermotif are collectively referred to as an HLA “supertype.” A recitation of motifs that are encompassed by supermotifs of the invention is provided in Table 4.

TABLE 4
Allelle-specific HLA-supertype members

| HLA- | | |
|---|---|---|
| supertype | Verified^{a} | Predicted^{b} |
| A1 | A*0101, A*2501, A*2601, | A*0102, A*2604, A*3601, |
| | A*2602, A*3201 | A*4301, A*8001 |
| A2 | A*0201, A*0202, A*0203, | A*0208, A*0210, A*0211, |
| | A*0204, A*0205, A*0206, | A*0212, A*0213 |
| | A*0207, A*0209, A*0214, | |
| | A*6802, A*6901 | |
| A3 | A*0301, A*1101, A*3101, | A*0302, A*1102, A*2603, |
| | A*3301, A*6801 | A*3302, A*3303, A*3401, |
| | | A*3402, A*6601, A*6602, |
| | | A*7401 |
| A24 | A*2301, A*2402, A*3001 | A*2403, A*2404, A*3002, |
| | | A*3003 |
| B7 | B*0702, B*0703, B*0704, | B*1511, B*4201, B*5901 |
| | B*0705, B*1508, B*3501, | |
| | B*3502, B*3503, B*3503, | |
| | B*3504, B*3505, B*3506, | |
| | B*3507, B*3508, B*5101, | |
| | B*5102, B*5103, B*5104, | |
| | B*5105, B*5301, B*5401, | |
| | B*5501, B*5502, B*5601, | |
| | B*5602, B*6701, B*7801 | |
| B27 | B*1401, B*1402, B*1509, | B*2701, B*2707, B*2708, |
| | B*2702, B*2703, B*2704, | B*3802, B*3903, B*3904, |
| | B*2705, B*2706, B*3801, | B*3905, B*4801, B*4802, |
| | B*3901, B*3902, B*7301 | B*1510, B*1518, B*1503 |
| B44 | B*1801, B*1802, B*3701, | B*4101, B*4501, B*4701, |
| | B*4402, B*4403, B*4404, | B*4901, B*5001 |
| | B*4001, B*4002, B*4006 | |
| B58 | B*5701, B*5702, B*5801, | |
| | B*5802, B*1516, B*1517 | |
| B62 | B*1501, B*1502, B*1513, | B*1301, B*1302, B*1504, |
| | B*5201 | B*1505, B*1506, B*1507, |
| | | B*1515, B*1520, B*1521, |
| | | B*1512, B*1514, B*1510 |

^{a}Verified alleles include alleles whose specificity has been determined by pool sequencing analysis, peptide binding assays, or by analysis of the sequences of CTL epitopes.
^{b}Predicted alleles are alleles whose specificity is predicted on the basis of B and F pocket structure to overlap with the supertype specificity.

The peptide motifs and supermotifs described below, and summarized in Table 4, provide guidance for the identification and use of peptide epitopes, in accordance with the invention.

Examples of peptide epitopes bearing a respective supermotif or motif are included in Tables 13-24 as designated in the description of each motif or supermotif below. The Tables include a binding affinity ratio listing for some of the peptide epitopes. The ratio may be converted to IC₅₀ by using the following formula: IC₅₀ of the standard peptide/ratio=IC₅₀ of the test peptide (i.e., the peptide epitope). The IC₅₀ values of standard peptides used to determine binding affinities for Class I peptides are shown below in Table 5. Under each supertype, the prototype allele is shown in bold. The IC₅₀ values of standard peptides used to determine binding affinities for Class II peptides are shown below in Table 6.

TABLE 5

| | | | | |
|---|---|---|---|---|
| | | | | Standard |
| | | Peptide | | Peptide IC₅₀ |
| Supertype | Allele | Sequence | SEQ ID NO | (nM) |

| | | | |
|---|---|---|---|
| A01 | A*0101 | YTAVVPLVY | 5 |
| | A*2601 | ETFGFEIQSY | 1 |
| | A*2902 | YTAVVPLVY | 5 |
| | A*3002 | RISGVDRYY | 3 |
| A02 | A*0201 | FLPSDYFPSV | 5 |
| | A*0202 | FLPSDYFPSV | 4.3 |
| | A*0203 | FLPSDYFPSV | 10 |
| | A*0206 | FLPSDYFPSV | 3.7 |
| | A*6802 | YVIKVSARV | 8 |
| A03, A11 | A*0301 | KVFPYALINK | 11 |
| | A*1101 | AVDLYHFLK | 6 |
| | A*3101 | KVFPYALINK | 18 |
| | A*3301 | ILYKRETTR | 29 |
| | A*6801 | KVFPYALINK | 8 |
| A24 | A*2301 | AYIDNYNKF | 4.9 |
| | A*2402 | AYIDNYNKF | 6 |
| | A*2902 | YTAVVPLVY | 5 |
| | A*3002 | RISGVDRYY | 3 |
| B07 | B*0702 | APRTLVYLL | 5.5 |
| | B*3501 | FPFKYAAAF | 7.2 |
| | B*5101 | FPFKYAAAF | 5.5 |
| | B*5301 | FPFKYAAAF | 9.3 |
| | B*5401 | FPFKYAAAF | 10 |
| B44 | B*1801 | SEIDLILGY | 3.1 |
| | B*4001 | YEFLQPILL | 1.6 |
| | B*4002 | YEFLQPILL | 1.7 |
| | B*4402 | SEIDLILGY | 9.2 |
| | B*4403 | SEIDLILGY | 6.8 |
| | B*4501 | AEFKYIAAV | 4.9 |

TABLE 6

| | | | | |
|---|---|---|---|---|
| | | | SEQ | Standard |
| | | | ID | Peptide IC₅₀ |
| Antigen | Allele | Peptide Sequence | NO | (nM) |

| | | | |
|---|---|---|---|
| DR1 | DRB1*0101 | PKYVKQNTLKLAT | 5 |
| DR3 | DRB1*0301 | YKTIAFDEEARR | 90 |
| DR4 | DRB1*0401 | YARFQSQTTLKQKT | 8 |
| DR4 | DRB1*0404 | YARFQSQTTLKQKT | 20 |
| DR4 | DRB1*0405 | YARFQSQTTLKQKT | 38 |
| DR7 | DRB1*0701 | PKYVKQNTIKLAT | 25 |
| DR8 | DRB1*0802 | KSKYKLATSVLAGLL | 49 |
| DR9 | DRB1*0901 | AKFVAAWTLKAAA | 75 |
| DR11 | DRB1*1101 | PKFVKQNTLKGAT | 20 |
| DR12 | DRB1*1201 | EALIHQLKLNPYVLS | 45 |
| DR13 | DRB1*1302 | QYIKANAKFIGITE | 3.5 |
| DR15 | DRB1*1501 | GRTQDENPVVHFFKNI | 9.1 |
| | | VTPRTPPP | |
| DR52 | DRB3*0101 | NGQIGNDPNRDIL | 100 |
| DR53 | DRB4*0101 | YARFQSQTTLKQKT | 58 |
| DR51 | DRB5*0101 | AKFVAAWTLKAAA | 20 |
| DQ | DQB1*0201 | YPFIEQEGPEFFDQE | 25 |
| DQ | DQB1*0301 | YAHAAHAAHAAHAAH | 21 |
| | | AA | |
| DQ | DQB1*0302 | EEDIEIIPIQEEEY | 21 |

For example, where an HLA-A2.1 motif-bearing peptide shows a relative binding ratio of 0.01 for HLA-A*0201, the IC₅₀ value is 500 nM, and where an HLA-A2.1 motif-bearing peptide shows a relative binding ratio of 0.1 for HLA-A*0201, the IC₅₀ value is 50 nM. The peptides used as standards for the binding assays described herein are examples of standards; alternative standard peptides can also be used when performing binding studies.

To obtain the peptide epitope sequences listed in Tables 13-24, protein sequence data for HPV types 6a, 6b, 11a, 16, 18, 31, 33, 45, 52, 56, and 58 were evaluated for the presence of the designated supermotif or motif. Seven HPV structural and regulatory proteins, E1, E2, E5, E6, E7, L1 and L2 were included in the analysis. E4 was also included in the evaluation of some of the strains. Peptide epitopes can additionally be evaluated on the basis of their conservancy (i.e., the amount of variance) among the available protein sequences for each HPV antigen.

In the Tables, motif- and/or supermotif-bearing amino acid sequences identified in the indicated HPV strains are designated by position number and length of the epitope with reference to the HPV sequences and numbering provided below. For each sequence, the following information is provided: Column 1 (labeled “Peptide”) recites a Peptide No. (internal identification number); Column 2 (labeled “Sequence”) recites the peptide epitope amino acid sequence; Column 3 (labeled “Source”) recites the HPV Type, the protein in which the motif-bearing sequence is found, and the amino acid number of the first residue in the motif-bearing sequence, e.g., “HPV16.E1.163” indicates that the peptide epitope is obtained from HPV Type 16, protein E1, beginning at position 163 of this protein; Column 4 (labeled “xxx PIC” wherein xxx is the HLA allele recited in the title of the Table) recites the predictive IC₅₀ binding value (“PIC”) of the motif-bearing sequence; Column 5 (labeled “Len”) indicates the length of the peptide sequence, e.g., “9” indicates that the peptide comprises 9 amino acid residues; all remaining Columns, excluding the final column, indicate the IC₅₀ binding value of each peptide epitope; the final Column (labeled “Degeneracy”) indicates the number of HLA alleles analyzed to which the peptide epitope is characterized as a “strong binder.” Amino acid substitutions made within a peptide epitope can also be indicated, i.e. “HPV.E6.29 L2” indicates that a Leucine is at position 2 within the epitope.

For HPV strain 11, the number and position listed for protein E5 refers to either the HPV11 E5a or HPV11 E5b sequence set out below. Because the epitope must include the designated motif or supermotif, e.g., HLA-A2, it can readily be determined whether the sequence refers to HPV11 E5a or E5b by checking the amino acid sequences of both E5a and E5b and selecting the sequence that conforms to the motif listed in Table 3.

HLA-A1 Supermotif and HLA-A1 Motif

The HLA-A1 supermotif is characterized by the presence in peptide ligands of a small (T or S) or hydrophobic (L, I, V, or M) primary anchor residue in position 2, and an aromatic (Y, F, or W) primary anchor residue at the C-terminal position of the epitope. The corresponding family of HLA molecules that bind to the A1 supermotif (i.e., the HLA-A1 supertype) is comprised of at least A*0101, A*2601, A*2602, A*2501, and A*3201 (see, e.g., DiBrino, M. et al., J. Immunol. 151:5930, 1993; DiBrino, M. et al., J. Immunol. 152:620, 1994; Kondo, A. et al., Immunogenetics 45:249, 1997). Other allele-specific HLA molecules predicted to be members of the A1 superfamily are shown in Table 4. Peptides binding to each of the individual HLA proteins can be modulated by substitutions at primary and/or secondary anchor positions, preferably choosing respective residues specified for the supermotif.

The HLA-A1 motif is characterized by the presence in peptide ligands of T, S, or M as a primary anchor residue at position 2 and the presence of Y as a primary anchor residue at the C-terminal position of the epitope. An alternative allele-specific A1 motif is characterized by a primary anchor residue at position 3 rather than position 2. This motif is characterized by the presence of D, E, A, or S as a primary anchor residue in position 3, and a Y as a primary anchor residue at the C-terminal position of the epitope (see, e.g., DiBrino et al., J. Immunol., 152:620, 1994; Kondo et al., Immunogenetics 45:249, 1997; and Kubo et al., J. Immunol. 152:3913, 1994 for reviews of relevant data). Peptide binding to HLA A1 can be modulated by substitutions at primary and/or secondary anchor positions, preferably choosing respective residues specified for the motif.

Representative peptide epitopes from the HPV E1 and E2 proteins that comprise the A1 supermotif; a subset of which comprise either one or both of the two A1 motifs referenced above, are set forth in Table 13. Representative peptide epitopes from the HPV E6 and E7 proteins that comprise the A1 supermotif; a subset of which comprise either one or both of the two A1 motifs referenced above, are set forth in Table 14.

HLA-A2 Supermotif and HLA-A2*0201 Motif

Primary anchor specificities for allele-specific HLA-A2.1 molecules (see, e.g., Falk, et al., Nature 351:290-96, 1991; Hunt, et al., Science 255:1261-63, 1992; Parker, et al., J. Immunol. 149:3580-87, 1992; Ruppert, et al., Cell 74:929-37, 1993) and cross-reactive binding among HLA-A2 and -A28 molecules have been described. (See, e.g., Fruci, et al., Human Immunol. 38:187-92, 1993; Tanigaki, et al., Human Immunol. 39:155-62, 1994; Del Guercio, et al., J. Immunol. 154:685-93, 1995; Kast, et al., J. Immunol. 152:3904-12, 1994, for reviews of relevant data.) These primary anchor residues define the HLA-A2 supermotif; which presence in peptide ligands corresponds to the ability to bind several different HLA-A2 and -A28 molecules. The HLA-A2 supermotif comprises peptide ligands with L, I, V, M, A, T, or Q as a primary anchor residue at position 2 and L, I, V, M, A, or T as a primary anchor residue at the C-terminal position of the epitope.

The corresponding family of HLA molecules (i.e., the HLA-A2 supertype that binds these peptides) is comprised of at least: A*0201, A*0202, A*0203, A*0204, A*0205, A*0206, A*0207, A*0209, A*0214, A*6802, and A*6901. Other allele-specific HLA molecules predicted to be members of the A2 superfamily are shown in Table 4. As explained in detail below, binding to each of the individual allele-specific HLA molecules can be modulated by substitutions at the primary anchor and/or secondary anchor positions, preferably choosing respective residues specified for the supermotif.

An HLA-A2*0201 motif was determined to be characterized by the presence in peptide ligands of L or M as a primary anchor residue in position 2, and L or V as a primary anchor residue at the C-terminal position of a 9-residue peptide (see, e.g., Falk, et al., Nature 351:290-296, 1991) and was further found to comprise an I at position 2 and I or A at the C-terminal position of a nine amino acid peptide (see, e.g., Hunt, et al., Science 255:1261-63, 1992; Parker, et al., J. Immunol. 149:3580-3587, 1992). The A*0201 allele-specific motif has also been defined by the present inventors to additionally comprise V, A, T, or Q as a primary anchor residue at position 2, and M or T as a primary anchor residue at the C-terminal position of the epitope (see, e.g., Kast et al., J. Immunol. 152:3904-3912, 1994). Thus, the HLA-A*0201 motif comprises peptide ligands with L, I, V, M, A, T, or Q as primary anchor residues at position 2 and L, I, V, M, A, or T as a primary anchor residue at the C-terminal position of the epitope. The preferred and tolerated residues that characterize the primary anchor positions of the HLA-A*0201 motif are identical to the residues describing the A2 supermotif. (For reviews of relevant data, see, e.g., Del Guercio, et al., J. Immunol. 154:685-93, 1995; Ruppert, et al., Cell 74:929-37, 1993; Sidney, et al., Immunol. Today 17:261-66, 1996; Sette and Sidney, Curr. Opin. in Immunol. 10:478-82, 1998). Secondary anchor residues that characterize the A*0201 motif have additionally been defined (see, e.g., Ruppert, et al., Cell 74:929-937, 1993). Peptide binding to HLA-A*0201 molecules can be modulated by substitutions at primary and/or secondary anchor positions, preferably choosing respective residues specified for the motif.

Representative peptide epitopes from the HPV E1 and E2 proteins that comprise an A2 supermotif; a subset of which also comprise an A*0201 motif, are set forth in Table 15. Representative peptide epitopes from the HPV E6 and E7 proteins that comprise an A2 supermotif; a subset of which also comprise an A*0201 motif, are set forth in Table 16. The motifs comprising the primary anchor residues V, A, T, or Q at position 2 and L, I, V, A, or T at the C-terminal position are those most particularly relevant to the invention claimed herein.

HLA-A3 Supermotif, the HLA-A3 Motif, and the HLA-A11 Motif

The HLA-A3 supermotif is characterized by the presence in peptide ligands of A, L, I, V, M, S, or, T as a primary anchor at position 2, and a positively charged residue, R or K, at the C-terminal position of the epitope, e.g., in position 9 of 9-mers (see, e.g., Sidney, et al., Hum. Immunol. 45:79, 1996). Exemplary members of the corresponding family of HLA molecules (the HLA-A3 supertype) that bind the A3 supermotif include at least A*0301, A*1101, A*3101, A*3301, and A*6801. Other allele-specific HLA molecules predicted to be members of the A3 supertype are shown in Table 4. As explained in detail below, peptide binding to each of the individual allele-specific HLA proteins can be modulated by substitutions of amino acids at the primary and/or secondary anchor positions of the peptide, preferably choosing respective residues specified for the supermotif.

The HLA-A3 motif is characterized by the presence in peptide ligands of L, M, V, I, S, A, T, F, C, G, or D as a primary anchor residue at position 2, and the presence of K, Y, R, H, F, or A as a primary anchor residue at the C-terminal position of the epitope (see, e.g., DiBrino, et al., Proc. Natl. Acad. Sci USA 90:1508, 1993; and Kubo, et al., J. Immunol. 152:3913-24, 1994). Peptide binding to HLA-A3 can be modulated by substitutions at primary and/or secondary anchor positions, preferably choosing respective residues specified for the motif.

The HLA-A11 motif is characterized by the presence in peptide ligands of V, T, M, L, I, S, A, G, N, C, D, or F as a primary anchor residue in position 2, and K, R, Y, or H as a primary anchor residue at the C-terminal position of the epitope (see, e.g., Zhang, et al., Proc. Natl. Acad. Sci USA 90:2217-21, 1993; and Kubo, et al., J. Immunol. 152:3913-24, 1994). Peptide binding to HLA-A11 can be modulated by substitutions at primary and/or secondary anchor positions, preferably choosing respective residues specified for the motif.

Representative peptide epitopes from the HPV E1 and E2 proteins that comprise the A3 supermotif, a subset of which comprise the A3 motif and/or the A11 motif, are set forth in Table 17. Representative peptide epitopes from the HPV E6 and E7 proteins that comprise the A3 supermotif, a subset of which comprise the A3 motif and/or the A11 motif, are set forth in Table 18. The A3 supermotif primary anchor residues comprise a subset of the A3- and A11-allele specific motif primary anchor residues. Representative peptide epitopes that comprise the A3 and A11 motifs are set forth in Tables 17-18 because of the extensive overlap between the A3 and A11 motif primary anchor specificities.

HLA-A24 Supermotif and the HLA-A24 Motif

The HLA-A24 supermotif is characterized by the presence in peptide ligands of an aromatic (F, W, or Y) or hydrophobic aliphatic (L, I, V, M, or T) residue as a primary anchor in position 2, and Y, F, W, L, I, or M as primary anchor at the C-terminal position of the epitope (see, e.g., Sette and Sidney, Immunogenetics 1999 November; 50(3-4):201-12, Review). The corresponding family of HLA molecules that bind to the A24 supermotif (i.e., the A24 supertype) includes at least A*2402, A*3001, and A*2301. Other allele-specific HLA molecules predicted to be members of the A24 supertype are shown in Table 4. Peptide binding to each of the allele-specific HLA molecules can be modulated by substitutions at primary and/or secondary anchor positions, preferably choosing respective residues specified for the supermotif.

The HLA-A24 motif is characterized by the presence in peptide ligands of Y, F, W, or M as a primary anchor residue in position 2, and F, L, I, or W as a primary anchor residue at the C-terminal position of the epitope (see, e.g., Kondo, et al., J. Immunol. 155:4307-12, 1995; and Kubo, et al., J. Immunol. 152:3913-24, 1994). Peptide binding to HLA-A24 molecules can be modulated by substitutions at primary and/or secondary anchor positions; preferably choosing respective residues specified for the motif.

Representative peptide epitopes from the HPV E1 and E2 proteins that comprise the A24 Supermotif, a subset of which comprise the A24 motif, are set forth in Table 19. Representative peptide epitopes from the HPV E6 and E7 proteins that comprise the A24 Supermotif, a subset of which comprise the A24 motif, are set forth in Table 20.

HLA-B7 Supermotif

The HLA-B7 supermotif is characterized by peptides bearing proline in position 2 as a primary anchor, and a hydrophobic or aliphatic amino acid (L, I, V, M, A, F, W, or Y) as the primary anchor at the C-terminal position of the epitope. The corresponding family of HLA molecules that bind the B7 supermotif (i.e., the HLA-B7 supertype) is comprised of at least twenty six HLA-B proteins including: B*0702, B*0703, B*0704, B*0705, B*1508, B*3501, B*3502, B*3503, B*3504, B*3505, B*3506, B*3507, B*3508, B*5101, B*5102, B*5103, B*5104, B*5105, B*5301, B*5401, B*5501, B*5502, B*5601, B*5602, B*6701, and B*7801 (see, e.g., Sidney, et al., J. Immunol. 154:247, 1995; Barber, et al., Curr. Biol. 5:179, 1995; Hill, et al., Nature 360:434, 1992; Rammensee, et al., Immunogenetics 41:178, 1995, for reviews of relevant data). Other allele-specific HLA molecules predicted to be members of the B7 supertype are shown in Table 4. As explained in detail below, peptide binding to each of the individual allele-specific HLA proteins can be modulated by substitutions at the primary and/or secondary anchor positions of the peptide, preferably choosing respective residues specified for the supermotif.

Representative peptide epitopes from the HPV E6 and E7 proteins that comprise the B7 supermotif are set forth in Table 21.

HLA-B44 Supermotif

The HLA-B44 supermotif is characterized by the presence in peptide ligands of negatively charged (D or E) residues as a primary anchor in position 2, and hydrophobic residues (F, W, Y, L, I, M, V, or A) as a primary anchor at the C-terminal position of the epitope (see, e.g., Sidney, et al., Immunol. Today 17:261, 1996). Exemplary members of the corresponding family of HLA molecules that bind to the B44 supermotif (i.e., the B44 supertype) include at least: B*1801, B*1802, B*3701, B*4001, B*4002, B*4006, B*4402, B*4403, and B*4006. Other allele-specific HLA molecules predicted to be members of the B44 supertype are shown in Table 4. Peptide binding to each of the allele-specific HLA molecules can be modulated by substitutions at primary and/or secondary anchor positions; preferably choosing respective residues specified for the supermotif.

Representative peptide epitopes from the HPV E6 and E7 proteins that comprise the B44 supermotif are set forth in Table 22.

HLA DR-1-4-7 Supermotif and HLA DR-3 Motif

Motifs have also been identified for peptides that bind to three common HLA class II allele-specific HLA molecules: HLA DRB1*0401, DRB1*0101, and DRB1*0701 (see, e.g., Southwood, et al., J. Immunology 160:3363-3373 (1998)). Collectively, the common residues from these motifs delineate the HLA DR-1-4-7 supermotif. Peptides that bind to these DR molecules carry a supermotif characterized by a large aromatic or hydrophobic residue (Y, F, W, L, I, V, or M) as a primary anchor residue in position 1, and a small, non-charged residue (S, T, C, A, P, V, I, L, or M) as a primary anchor residue in position 6 of a 9-mer core region. Allele-specific secondary effects and secondary anchors for each of these HLA types have also been identified (Southwood, et al., J. Immunol. 160:3363-3373 (1998)). These are set forth in Tables 7, 8, and 9. Peptide binding to HLA-DRB1*0401, DRB1*0101, and/or DRB1*0701 can be modulated by substitutions at primary and/or secondary anchor positions, preferably choosing respective residues specified for the supermotif.

TABLE 7

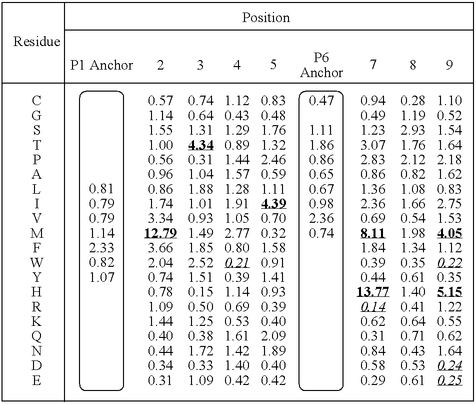

DRB1 *0401 algorithm: ARB values. ARB values of peptides bearing the P1-P6 primary anchors as a function of the different residues at nonanchor positions to DRB1 *0401. The panel was composed of 384 peptides based on naturally occurring and non-natural sequences derived from various viral, tumor or bacterial origins. Values ≧4.00 are indicated by bold type. Values ≦0.25 are indicated by italicized type and underlines.

TABLE 8

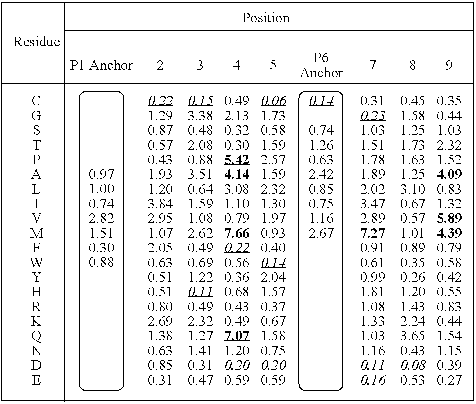

DRB1 *0101 algorithm: ARB values. ARB values of peptides bearing the P1-P6 primary anchors as a function of the different residues an nonanchor positions to DRB1 *0101. The panel was composed of 384 peptides based on naturally occurring and non-natural sequences derived from various derived from various viral, tumor or bacterial origins. Values ≧4.00 are indicated by bold type. Values ≦0.25 are indicated by italicized type and underlines.

TABLE 9

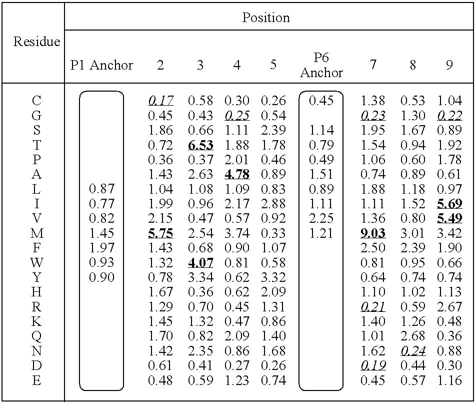

DRB1 *0701 algorithm: ARB values. ARB values of peptides bearing the P1-P6 primary anchors as a function of the different residues an nonanchor positions to DRB1 *0101. The panel was composed of 384 peptides based on naturally occurring and non-natural sequences derived from various derived from various viral, tumor or bacterial origins. Values ≧4.00 are indicated by bold type. Values ≦0.25 are indicated by italicized type and underlines.

Two alternative motifs (i.e., submotifs) characterize peptide epitopes that bind to HLA-DR3 molecules (see, e.g., Geluk et al., J. Immunol. 152:5742, 1994). In the first motif (submotif DR3A) a large, hydrophobic residue (L, I, V, M, F, or Y) is present in anchor position 1 of a 9-mer core, and D is present as an anchor at position 4, towards the carboxyl terminus of the epitope. As in other class II motifs, core position 1 may or may not occupy the peptide N-terminal position.

The alternative DR3 submotif provides for lack of the large, hydrophobic residue at anchor position 1, and/or lack of the negatively charged or amide-like anchor residue at position 4, by the presence of a positive charge at position 6 towards the carboxyl terminus of the epitope. Thus, for the alternative allele-specific DR3 motif (submotif DR3B): L, I, V, M, F, Y, A, or Y is present at anchor position 1; D, N, Q, E, S, or T is present at anchor position 4; and K, R, or H is present at anchor position 6. Peptide binding to HLA-DR3 can be modulated by substitutions at primary and/or secondary anchor positions, preferably choosing respective residues specified for the motif.

Representative epitopes from the HPV E1 and E2 proteins comprising the DR-1-4-7 supermotif, and representative epitopes from the HPV E1 and E2 proteins comprising the HLA-DR-3a and DR3b motifs, wherein position 1 of the supermotif is at position 1 of the nine-residue core, are set forth in Table 23. Representative epitopes from the HPV E6 and E7 proteins comprising the DR-1-4-7 supermotif, and representative epitopes from the HPV E6 and E7 proteins comprising the HLA-DR-3a and DR3b motifs, wherein position 1 of the supermotif is at position 1 of the nine-residue core, are set forth in Table 24. Exemplary epitopes of 15 amino acids in length that comprises the nine residue core include the three residues on either side that flank the nine residue core. HTL epitopes that comprise the core sequences can also be of lengths other than 15 amino acids, supra. Accordingly, epitopes of the invention include sequences that typically comprise the nine residue core plus 1, 2, 3 (as in the exemplary 15-mer), 4, or 5 flanking residues on either side of the nine residue core.

Each of the HLA class I or class II epitopes set out in the Tables herein are deemed singly to be an inventive embodiment of this application. Further, it is also an inventive embodiment of this application that each epitope may be used in combination with any other epitope.

Enhancing Population Coverage of the Vaccine

Vaccines that have broad population coverage are preferred because they are more commercially viable and generally applicable to the most people. Broad population coverage can be obtained using the peptides of the invention (and nucleic acid compositions that encode such peptides) through selecting peptide epitopes that bind to HLA alleles which, when considered in total, are present in most of the population. Table 10 lists the overall frequencies of the HLA class I supertypes in various ethnicities (Section A) and the combined population coverage achieved by the A2-, A3-, and B7-supertypes (Section B). The A2-, A3-, and B7 supertypes are each present on the average of over 40% in each of these five major ethnic groups. Coverage in excess of 80% is achieved with a combination of these supermotifs. These results suggest that effective and non-ethnically biased population coverage is achieved upon use of a limited number of cross-reactive peptides. Although the population coverage reached with these three main peptide specificities is high, coverage can be expanded to reach 95% population coverage and above, and more easily achieve truly multi-specific responses upon use of additional supermotif or allele-specific motif bearing peptides.

The B44-, A1-, and A24-supertypes are each present, on average, in a range from 25% to 40% in these major ethnic populations (Section A). While less prevalent overall, the B27-, B58-, and B62 supertypes are each present with a frequency >25% in at least one major ethnic group (section A). In Section B, Table 10 summarizes the estimated prevalence of combinations of HLA supertypes that have been identified in five major ethnic groups. The incremental coverage obtained by the inclusion of A1,- A24-, and B44-supertypes to the A2, A3, and B7 coverage and coverage obtained with all of the supertypes described herein, is shown.

The data presented herein, together with the previous definition of the A2-, A3-, and B7-supertypes, indicates that all antigens, with the possible exception of A29, B8, and B46, can be classified into a total of nine HLA supertypes. By including epitopes from the six most frequent supertypes, an average population coverage of 99% is obtained for five major ethnic groups.

TABLE 10
Population coverage with combined HLA Supertypes

| | PHENOTYPIC FREQUENCY | | | | | |
|---|---|---|---|---|---|---|
| | | North | | | | |
| | | American | | | | |
| HLA-SUPERTYPES | Caucasian | Black | Japanese | Chinese | Hispanic | Average |
| A. Individual | | | | | | |
| Supertypes | | | | | | |
| A2 | 45.8 | 39.0 | 42.4 | 45.9 | 43.0 | 43.2 |
| A3 | 37.5 | 42.1 | 45.8 | 52.7 | 43.1 | 44.2 |
| B7 | 43.2 | 55.1 | 57.1 | 43.0 | 49.3 | 49.5 |
| A1 | 47.1 | 16.1 | 21.8 | 14.7 | 26.3 | 25.2 |
| A24 | 23.9 | 38.9 | 58.6 | 40.1 | 38.3 | 40.0 |
| B44 | 43.0 | 21.2 | 42.9 | 39.1 | 39.0 | 37.0 |
| B27 | 28.4 | 26.1 | 13.3 | 13.9 | 35.3 | 23.4 |
| B62 | 12.6 | 4.8 | 36.5 | 25.4 | 11.1 | 18.1 |
| B58 | 10.0 | 25.1 | 1.6 | 9.0 | 5.9 | 10.3 |
| B. Combined | | | | | | |
| Supertypes | | | | | | |
| A2, A3, B7 | 84.3 | 86.8 | 89.5 | 89.8 | 86.8 | 87.4 |
| A2, A3, B7, | 99.5 | 98.1 | 100.0 | 99.5 | 99.4 | 99.3 |
| A24, B44, A1 | | | | | | |
| A2, A3, B7, | 99.9 | 99.6 | 100.0 | 99.8 | 99.9 | 99.8 |
| A24, B44, A1, | | | | | | |
| B27, B62, B58 | | | | | | |

Immune Response-Stimulating Peptide Analogs

In general, CTL and HTL responses to whole antigens are not directed against all possible epitopes. Rather, they are restricted to a few “immunodominant” determinants (Zinkernagel, et al., Adv. Immunol. 27:5159, 1979; Bennink, et al., J. Exp. Med. 168:1935-39, 1988; Rawle, et al., J. Immunol. 146:3977-84, 1991). It has been recognized that immunodominance (Benacerraf, et al., Science 175:273-79, 1972) could be explained by either the ability of a given epitope to selectively bind a particular HLA protein (determinant selection theory) (Vitiello, et al., J. Immunol. 131:1635, 1983); Rosenthal, et al., Nature 267:156-158, 1977), or to be selectively recognized by the existing TCR (T cell receptor) specificities (repertoire theory) (Klein, J., IMMUNOLOGY, THE SCIENCE OF SELF-NONSELF DISCRIMINATION, John Wiley & Sons, New York, pp. 270-310, 1982). It has been demonstrated that additional factors, mostly linked to processing events, can also play a key role in dictating, beyond strict immunogenicity, which of the many potential determinants will be presented as immunodominant (Sercarz, et al., Ann. Rev. Immunol. 11:729-766, 1993).

The concept of dominance and subdominance is relevant to immunotherapy of both infectious diseases and cancer. For example, in the course of chronic viral disease, recruitment of subdominant epitopes can be important for successful clearance of the infection, especially if dominant CTL or HTL specificities have been inactivated by functional tolerance, suppression, mutation of viruses and other mechanisms (Franco, et al., Curr. Opin. Immunol. 7:524-531, 1995). In the case of cancer and tumor antigens, CTLs recognizing at least some of the highest binding affinity peptides might be functionally inactivated. Lower binding affinity peptides are preferentially recognized at these times, and may therefore be preferred in therapeutic or prophylactic anti-cancer vaccines.

In particular, it has been noted that a significant number of epitopes derived from known non-viral tumor associated antigens (TAA) bind HLA class I with intermediate affinity (IC₅₀ in the 50-500 nM range). For example, it has been found that 8 of 15 known TAA peptides recognized by tumor infiltrating lymphocytes (TIL) or CTL bound in the 50-500 nM range. (These data are in contrast with estimates that 90% of known viral antigens were bound by HLA class I molecules with IC₅₀ of 50 nM or less, while only approximately 10% bound in the 50-500 nM range (Sette, et al., J. Immunol., 153:558-92, 1994). In the cancer setting this phenomenon is probably due to elimination or functional inhibition of the CTL recognizing several of the highest binding peptides, presumably because of T cell tolerization events.

Without intending to be bound by theory, it is believed that because T cells to dominant epitopes may have been clonally deleted, selecting subdominant epitopes may allow existing T cells to be recruited, which will then lead to a therapeutic or prophylactic response. However, the binding of HLA molecules to subdominant epitopes is often less vigorous than to dominant ones. Accordingly, there is a need to be able to modulate the binding affinity of particular immunogenic epitopes for one or more HLA molecules, and thereby to modulate the immune response elicited by the peptide, for example to prepare analog peptides which elicit a more vigorous response. This ability would greatly enhance the usefulness of peptide epitope-based vaccines and therapeutic agents.

Although peptides with suitable cross-reactivity among all alleles of a superfamily are identified by the screening procedures described above, cross-reactivity is not always as complete as possible, and in certain cases procedures to increase cross-reactivity of peptides can be useful; moreover, such procedures can also be used to modify other properties of the peptides such as binding affinity or peptide stability. Having established the general rules that govern cross-reactivity of peptides for HLA alleles within a given motif or supermotif, modification (i.e., analoging) of the structure of peptides of particular interest in order to achieve broader (or otherwise modified) HLA binding capacity can be performed. More specifically, peptides which exhibit the broadest cross-reactivity patterns, can be produced in accordance with the teachings herein. The present concepts related to analog generation are set forth in greater detail in co-pending U.S. patent application Ser. No. 09/226,775, filed Jan. 6, 1999, and PCT Application No. PCT/US00/31856, filed Nov. 20, 2000 (published as PCT Publication No. WO01/36452).

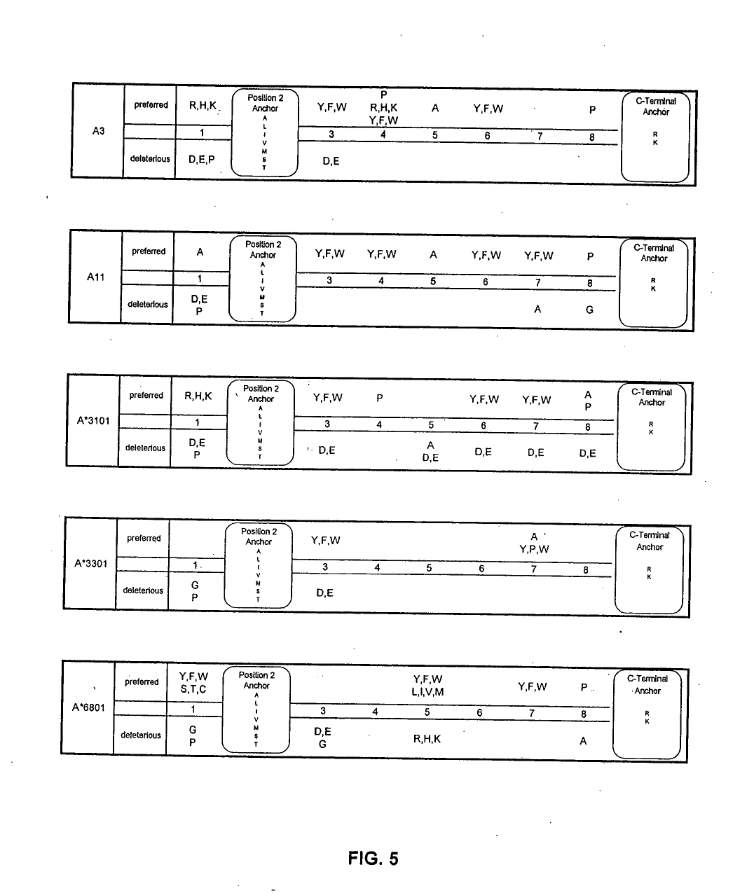

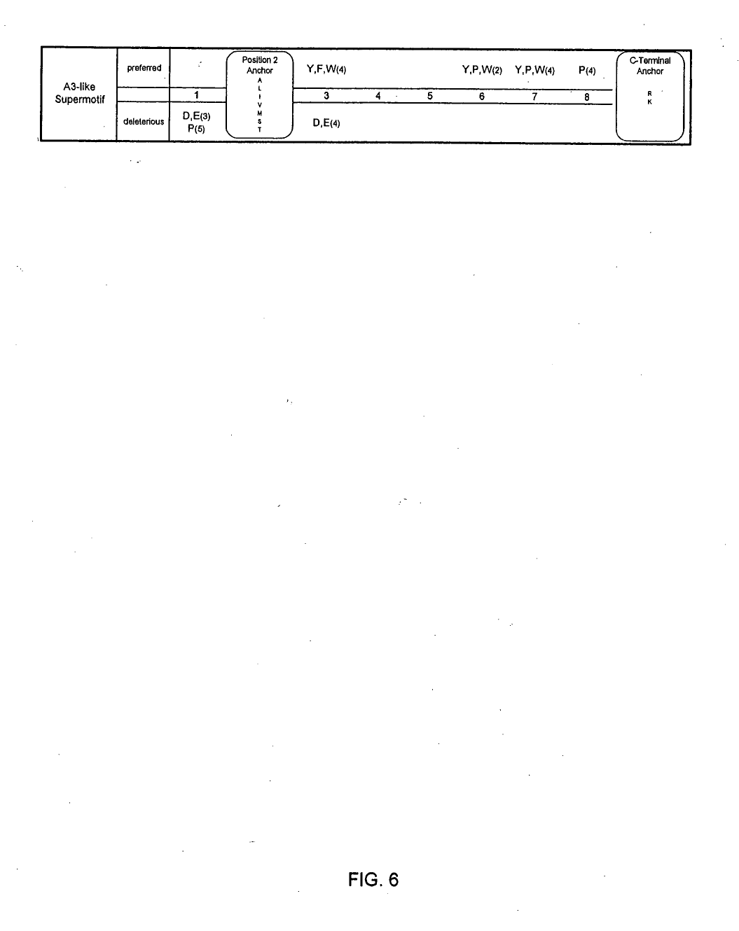

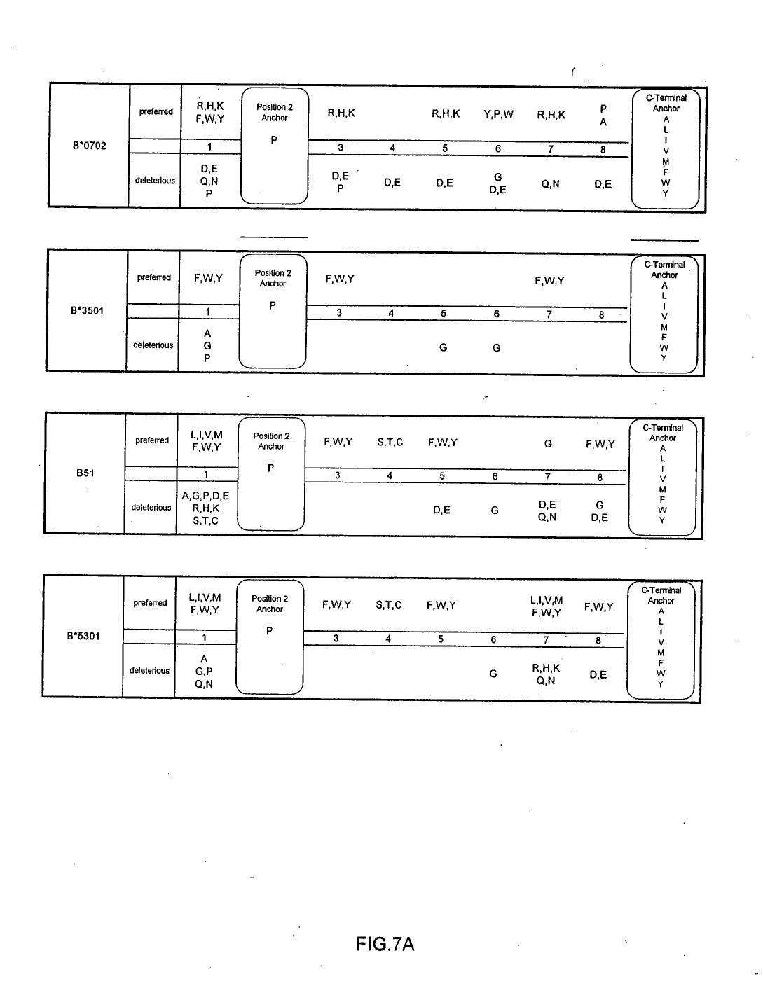

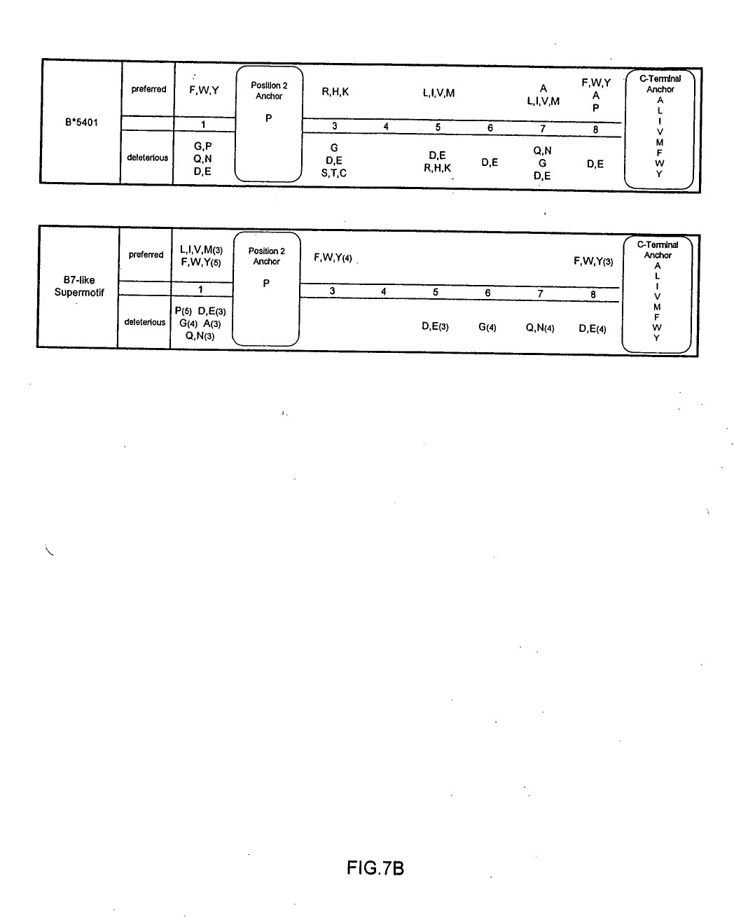

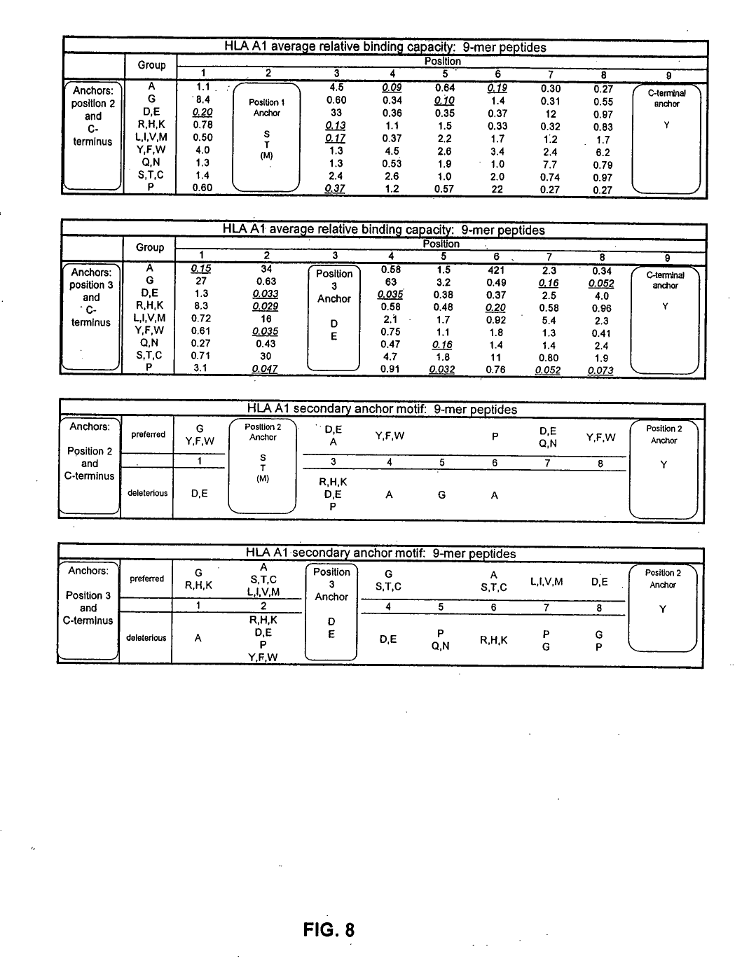

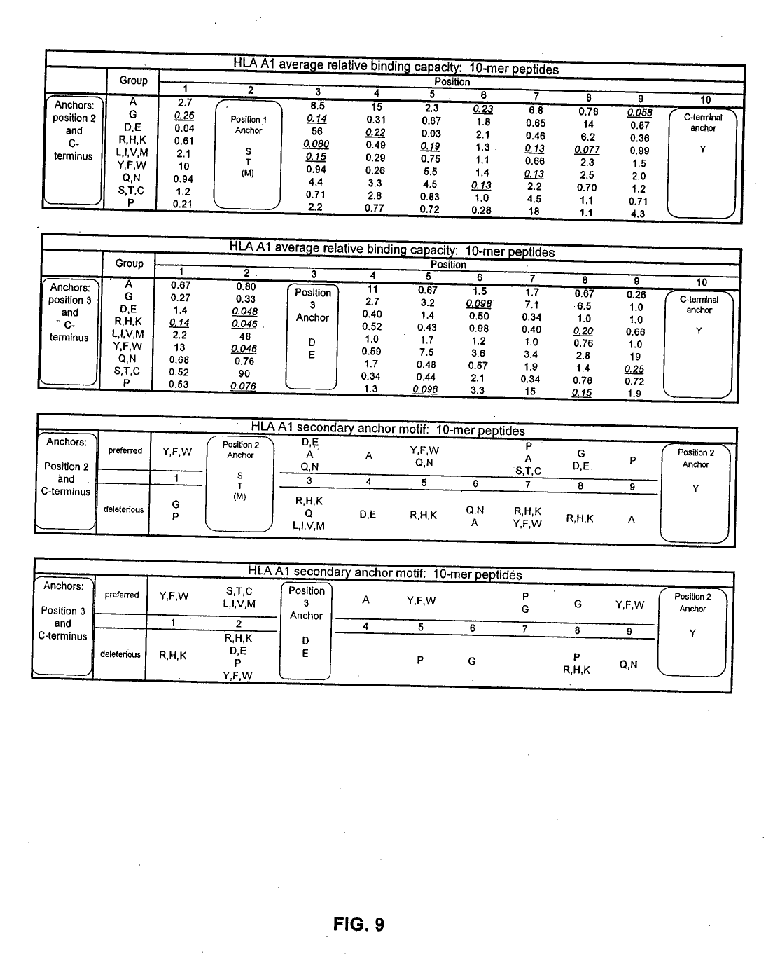

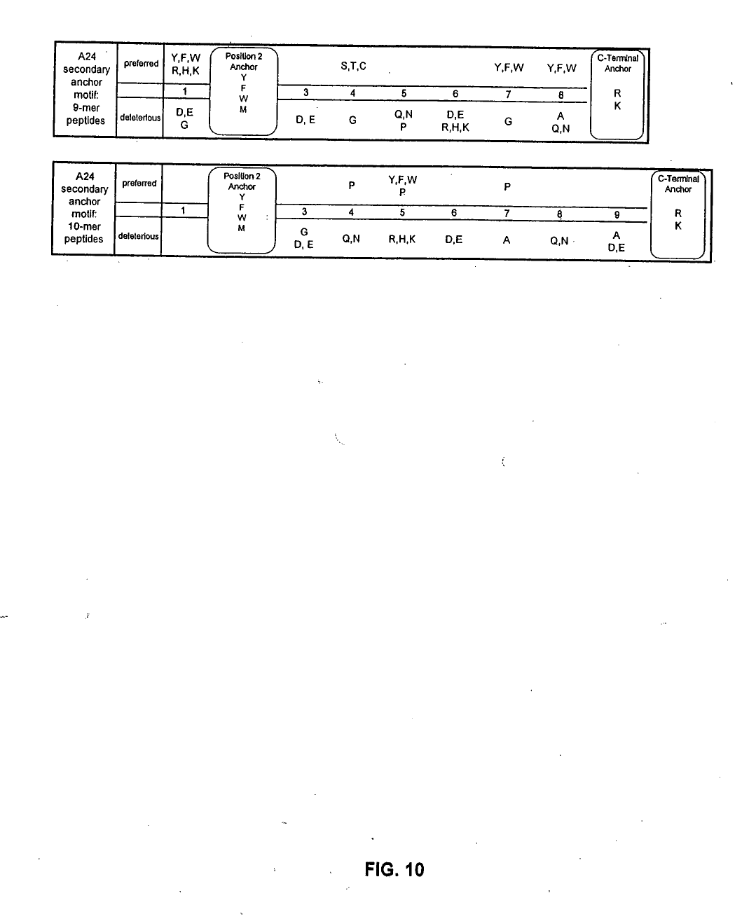

In brief, the strategy employed utilizes the motifs or supermotifs which correlate with binding to certain HLA molecules. The motifs or supermotifs are defined by having primary anchors, and in many cases secondary anchors. Analog peptides can be created by substituting amino acid residues at primary anchor, secondary anchor, or at primary and secondary anchor positions. Generally, analogs are made for peptides that already bear a motif or supermotif. Preferred secondary anchor residues of supermotifs and motifs that have been defined for HLA class I and class II binding peptides are shown in FIGS. 5, 6, 7A, 7B, 8, 9, and 10.

For a number of the motifs or supermotifs in accordance with the invention, residues are defined which are deleterious to binding to allele-specific HLA molecules or members of HLA supertypes that bind the respective motif or supermotif. Accordingly, removal of such residues that are detrimental to binding can be performed in accordance with the present invention. For example, in the case of the A3 supertype, when all peptides that have such deleterious residues are removed from the population of peptides used in the analysis, the incidence of cross-reactivity increased from 22% to 37% (see, e.g., Sidney, J. et al., Hu. Immunol. 45:79, 1996). Thus, one strategy to improve the cross-reactivity of peptides within a given supermotif is simply to delete one or more of the deleterious residues present within a peptide and substitute a small “neutral” residue such as Ala (that may not influence T cell recognition of the peptide). An enhanced likelihood of cross-reactivity is expected if, together with elimination of detrimental residues within a peptide, “preferred” residues associated with high affinity binding to an allele-specific HLA molecule or to multiple HLA molecules within a superfamily are inserted.

To ensure that an analog peptide, when used as a vaccine, actually elicits a CTL response to the native epitope in vivo (or, in the case of class II epitopes, elicits helper T cells that cross-react with the wild type peptides), the analog peptide may be used to immunize T cells in vitro from individuals of the appropriate HLA allele. Thereafter, the capacity of the immunized cells to induce lysis of wild type peptide sensitized target cells is evaluated. It will be desirable to use as antigen presenting cells, cells that have been either infected, or transfected with the appropriate genes, or, in the case of class II epitopes only, cells that have been pulsed with whole protein antigens, to establish whether endogenously produced antigen is also recognized by the relevant T cells.

Another embodiment of the invention is to create analogs of weak binding peptides, to thereby ensure adequate numbers of cross-reactive cellular binders. Class I binding peptides exhibiting binding affinities of 500-5000 nM, and carrying an acceptable, but suboptimal, primary anchor residue at one or both positions can be “fixed” by substituting preferred anchor residues in accordance with the respective supertype. The analog peptides can then be tested for cross-binding activity.

Another embodiment for generating effective peptide analogs involves the substitution of residues that have an adverse impact on peptide stability or solubility in, e.g., a liquid environment. This substitution may occur at any position of the peptide epitope. For example, a cysteine (C) can be substituted out in favor of α-amino butyric acid. Due to its chemical nature, cysteine has the propensity to form disulfide bridges and sufficiently alter the peptide structurally so as to reduce binding capacity. Substituting α-amino butyric acid for C not only alleviates this problem, but actually improves binding and cross-binding capability in certain instances (see, e.g., the review by Sette et al., In: Persistent Viral Infections, Eds. R. Ahmed and I. Chen, John Wiley & Sons, England, 1999). Substitution of cysteine with α-amino butyric acid may occur at any residue of a peptide epitope, i.e. at either anchor or non-anchor positions.

Computer Screening of Protein Sequences from Disease-Related Antigens for Supermotif- or Motif-Bearing Peptides

In order to identify supermotif- or motif-bearing epitopes in a target antigen, a native protein sequence, e.g., a tumor-associated antigen, or sequences from an infectious organism, or a donor tissue for transplantation, is screened using a means for computing, such as an intellectual calculation or a computer, to determine the presence of a supermotif or motif within the sequence. The information obtained from the analysis of native peptide can be used directly to evaluate the status of the native peptide or may be utilized subsequently to generate the peptide epitope.

Computer programs that allow the rapid screening of protein sequences for the occurrence of the subject super-motifs or motifs are encompassed by the present invention; as are programs that permit the generation of analog peptides. These programs are implemented to analyze any identified amino acid sequence or operate on an unknown sequence and simultaneously determine the sequence and identify motif-bearing epitopes thereof; analogs can be simultaneously determined as well. Generally, the identified sequences will be from a pathogenic organism or a tumor-associated peptide. For example, the target molecules considered herein include, without limitation, the E1, E2, E4, E5a, E5b, E6, E7, L1 and L2 proteins of HPV.

In cases where the sequences of multiple variants of the same target protein are available, potential peptide epitopes can also be selected on the basis of their conservancy. For example, a criterion for conservancy may define that the entire sequence of an HLA class I binding peptide or the entire 9-mer core of a class II binding peptide, be conserved in a designated percentage, of the sequences evaluated for a specific protein antigen.

To target a broad population that may be infected with a number of different strains, it is preferable to include in vaccine compositions epitopes that are representative of HPV antigen sequences from different HPV strains. As appreciated by those in the art, regions with greater or lesser degrees of conservancy among HPV strains can be employed as appropriate for a given antigenic target. In preferred embodiments of the present invention, one or more of HPV Types 6a, 6b, 11a, 16, 18, 31, 33, 45, 52, 56, and/or 58 are comprised by a given peptide epitope of the present invention.

It is important that the selection criteria utilized for prediction of peptide binding are as accurate as possible, to correlate most efficiently with actual binding. Prediction of peptides that bind, for example, to HLA-A*0201, on the basis of the presence of the appropriate primary anchors, is positive at about a 30% rate (see, e.g., Ruppert, J. et al. Cell 74:929, 1993). However, by extensively analyzing peptide-HLA binding data disclosed herein, data in related patent applications, and data in the art, the present inventors have developed a number of allele-specific polynomial algorithms that dramatically increase the predictive value over identification on the basis of the presence of primary anchor residues alone. These algorithms take into account not only the presence or absence of primary anchors, but also consider the positive or deleterious presence of secondary anchor residues (to account for the impact of different amino acids at different positions). The algorithms are essentially based on the premise that the overall affinity (or ΔG) of peptide-HLA interactions can be approximated as a linear polynomial function of the type:
ΔG=aₗᵢ×a₂ᵢ×a₃ᵢ . . . ×aₙᵢ

where aⱼᵢ is a coefficient that represents the effect of the presence of a given amino acid (j) at a given position (i) along the sequence of a peptide of n amino acids. An important assumption of this method is that the effects at each position are essentially independent of each other. This assumption is justified by studies that demonstrated that peptides are bound to HLA molecules and recognized by T cells in essentially an extended conformation. Derivation of specific algorithm coefficients has been described, for example, in Gulukota, K., et al., J. Mol. Biol. 267:1258-67, 1997.

Additional methods to identify preferred peptide sequences, which also make use of specific motifs, include the use of neural networks and molecular modeling programs (see, e.g., Milik, et al., Nature Biotechnology 16:753, 1998; Altuvia, et al., Hum. Immunol. 58:1, 1997; Altuvia, et al., J. Mol. Biol. 249:244, 1995; Buus, S. Curr. Opin. Immunol. 11:209-213, 1999; Brusic, V., et al., Bioinformatics 14:121-130, 1998; Parker, et al., J. Immunol. 152:163, 1993; Meister, et al., Vaccine 13:581, 1995; Hammer, et al., J. Exp. Med. 180:2353, 1994; Stumiolo, et al., Nature Biotechnol. 17:555 1999).

For example, it has been shown that in sets of A*0201 motif-bearing peptides containing at least one preferred secondary anchor residue while avoiding the presence of any deleterious secondary anchor residues, 69% of the peptides will bind A*0201 with an IC₅₀ less than 500 nM (Ruppert, J., et al. Cell 74:929, 1993). In certain embodiments, the algorithms of the invention are also flexible in that cut-off scores may be adjusted to select sets of peptides with greater or lower predicted binding properties, as desired.

In utilizing computer screening to identify peptide epitopes, a protein sequence or translated sequence may be analyzed using software developed to search for motifs, for example the “FINDPATTERNS’ program (Devereux, et al. Nucl. Acids Res. 12:387-395, 1984) or MotifSearch 1.4 software program (D. Brown, San Diego, Calif.) to identify potential peptide sequences containing appropriate HLA binding motifs. The identified peptides can be scored using customized polynomial algorithms to predict their capacity to bind specific HLA class I or class II alleles. As appreciated by one of ordinary skill in the art, a large array of computer programming software and hardware options are available in the relevant art which can be employed to implement the motifs of the invention in order to evaluate (e.g., without limitation, to identify epitopes, identify epitope concentration per peptide length, or to generate analogs) known or unknown peptide sequences.

In accordance with the procedures described above, HPV peptide epitopes that are able to bind HLA supertype groups or allele-specific HLA molecules have been identified (Tables 13-24).

Preparation of Peptide Epitopes

Peptides in accordance with the invention can be prepared synthetically, by recombinant DNA technology or chemical synthesis, or from natural sources such as native tumors or pathogenic organisms. Peptide epitopes may be synthesized individually or as polyepitopic peptides. Although the peptide will preferably be substantially free of other naturally occurring host cell proteins and fragments thereof, in some embodiments the peptides may be synthetically conjugated to native fragments or particles.

The peptides in accordance with the invention can be a variety of lengths, and either in their neutral (uncharged) forms or in forms which are salts. The peptides in accordance with the invention are either free of modifications such as glycosylation, side chain oxidation, or phosphorylation; or they contain these modifications, subject to the condition that modifications do not destroy the biological activity of the peptides as described herein.

When possible, it may be desirable to optimize HLA class I binding epitopes of the invention, such as can be used in a polyepitopic construct, to a length of about 8 to about 13 amino acid residues, often 8 to 11 amino acid residues, and, preferably, 9 to 10 amino acids. HLA class II binding peptide epitopes of the invention may be optimized to a length of about 6 to about 30 amino acid residues in length, preferably to between about 13 and about 20 amino acid residues. Preferably, the peptide epitopes are commensurate in size with endogenously processed pathogen-derived peptides or tumor cell peptides that are bound to the relevant HLA molecules, however, the identification and preparation of peptides that comprise epitopes of the invention can also be carried out using the techniques described herein.

In alternative embodiments, epitopes of the invention can be linked as a polyepitopic peptide, or as a minigene that encodes a polyepitopic peptide.

In another embodiment, it is preferred to identify native peptide regions that contain a high concentration of class I and/or class II epitopes. Such a sequence is generally selected on the basis that it contains the greatest number of epitopes per amino acid length. It is to be appreciated that epitopes can be present in a nested or overlapping manner, e.g. a 10 amino acid long peptide could contain two 9 amino acid long epitopes and one 10 amino acid long epitope; upon intracellular processing, each epitope can be exposed and bound by an HLA molecule upon administration of such a peptide. This larger, preferably multi-epitopic, peptide can be generated synthetically, recombinantly, or via cleavage from the native source.

The peptides of the invention can be prepared in a wide variety of ways. For the preferred relatively short size, the peptides can be synthesized in solution or on a solid support in accordance with conventional techniques. Various automatic synthesizers are commercially available and can be used in accordance with known protocols. (See, for example, Stewart & Young, SOLID PHASE PEPTIDE SYNTHESIS, 2D. ED., Pierce Chemical Co., 1984). Further, individual peptide epitopes can be joined using chemical ligation to produce larger peptides that are still within the bounds of the invention.

Alternatively, recombinant DNA technology can be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook, et al., MOLECULAR CLONING, A LABORATORY MANUAL, Cold Spring Harbor Press, Cold Spring Harbor, N.Y. (1989). Thus, recombinant polypeptides which comprise one or more peptide sequences of the invention can be used to present the appropriate T cell epitope.

The nucleotide coding sequence for peptide epitopes of the preferred lengths contemplated herein can be synthesized by chemical techniques, for example, the phosphotriester method of Matteucci, et al., J. Am. Chem. Soc. 103:3185 (1981). Peptide analogs can be made simply by substituting the appropriate and desired nucleic acid base(s) for those that encode the native peptide sequence; exemplary nucleic acid substitutions are those that encode an amino acid defined by the motifs/supermotifs herein. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast, insect or mammalian cell hosts may also be used, employing suitable vectors and control sequences.

Assays to Detect T-Cell Responses

Once HLA binding peptides are identified, they can be tested for the ability to elicit a T-cell response. The preparation and evaluation of motif-bearing peptides are described in PCT publications WO 94/20127 and WO 94/03205. Briefly, peptides comprising epitopes from a particular antigen are synthesized and tested for their ability to bind to the appropriate HLA proteins. These assays may involve evaluating the binding of a peptide of the invention to purified HLA class I molecules in relation to the binding of a radioiodinated reference peptide. Alternatively, cells expressing empty class I molecules (i.e. lacking peptide therein) may be evaluated for peptide binding by immunofluorescent staining and flow microfluorimetry. Other assays that may be used to evaluate peptide binding include peptide-dependent class I assembly assays and/or the inhibition of CTL recognition by peptide competition. Those peptides that bind to the class I molecule, typically with an affinity of 500 nM or less, are further evaluated for their ability to serve as targets for CTLs derived from infected or immunized individuals, as well as for their capacity to induce primary in vitro or in vivo CTL responses that can give rise to CTL populations capable of reacting with selected target cells associated with a disease.

Analogous assays are used for evaluation of HLA class II binding peptides. HLA class II motif-bearing peptides that are shown to bind, typically at an affinity of 1000 nM or less, are further evaluated for the ability to stimulate HTL responses.

Conventional assays utilized to detect T cell responses include proliferation assays, lymphokine secretion assays, direct cytotoxicity assays, and limiting dilution assays. For example, antigen-presenting cells that have been incubated with a peptide can be assayed for the ability to induce CTL responses in responder cell populations. Antigen-presenting cells can be normal cells such as peripheral blood mononuclear cells or dendritic cells. Alternatively, mutant non-human mammalian cell lines that are deficient in their ability to load class I molecules with internally processed peptides and that have been transfected with the appropriate human class I gene, may be used to test for the capacity of the peptide to induce in vitro primary CTL responses.

Peripheral blood mononuclear cells (PBMCs) may be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived.

Additionally, a method has been devised which allows direct quantification of antigen-specific T cells by staining with Fluorescein-labeled HLA tetrameric complexes (Altman, J. D., et al., Proc. Natl. Acad. Sci. USA 90:10330, 1993; Altman, J. D. et al., Science 274:94, 1996). Other relatively recent technical developments include staining for intracellular lymphokines, and interferon release assays or ELISPOT assays. Tetramer staining, intracellular lymphokine staining and ELISPOT assays all appear to be at least 10-fold more sensitive than more conventional assays (Lalvani, A., et al., J. Exp. Med. 186:859, 1997; Dunbar, P. R., et al., Curr. Biol. 8:413, 1998; Murali-Krishna, K., et al., Immunity 8:177, 1998).

HTL activation may also be assessed using such techniques known to those in the art such as T cell proliferation and secretion of lymphokines, e.g. IL-2 (see, e.g. Alexander, et al., Immunity 1:751-61, 1994).

Alternatively, immunization of HLA transgenic mice can be used to determine immunogenicity of peptide epitopes. Several transgenic mouse models including mice with human A2.1, A11 (which can additionally be used to analyze HLA-A3 epitopes), and B7 alleles have been characterized and others (e.g., transgenic mice for HLA-A1 and A24) are being developed. HLA-DR1 and HLA-DR3 mouse models have also been developed. Additional transgenic mouse models with other HLA alleles may be generated as necessary. Mice may be immunized with peptides emulsified in Incomplete Freund's Adjuvant and the resulting T cells tested for their capacity to recognize peptide-pulsed target cells and target cells transfected with appropriate genes. CTL responses may be analyzed using cytotoxicity assays described above. Similarly, HTL responses may be analyzed using such assays as T cell proliferation or secretion of lymphokines.

Use of Peptide Epitopes as Diagnostic Agents and for Evaluating Immune Responses

In certain embodiments of the invention, HLA class I and class II binding peptides as described herein can be used as reagents to evaluate an immune response. The immune response to be evaluated is induced by using as an immunogen any agent that may result in the production of antigen-specific CTLs or HTLs that recognize and bind to the peptide epitope(s) to be employed as the reagent. The peptide reagent need not be used as the immunogen. Assay systems that are used for such an analysis include relatively recent technical developments such as tetramers, staining for intracellular lymphokines and interferon release assays, or ELISPOT assays.

For example, a peptide of the invention is used in a tetramer staining assay to assess peripheral blood mononuclear cells for the presence of antigen-specific CTLs following exposure to a pathogen or immunogen. The HLA-tetrameric complex is used to directly visualize antigen-specific CTLs (see, e.g., Ogg, et al., Science 279:2103-06, 1998; and Altman, et al., Science 174:94-96, 1996) and determine the frequency of the antigen-specific CTL population in a sample of peripheral blood mononuclear cells.

A tetramer reagent using a peptide of the invention is generated as follows: A peptide that binds to an HLA molecule is refolded in the presence of the corresponding HLA heavy chain and β₂-Microglobulin to generate a trimolecular complex. The complex is biotinylated at the carboxyl terminal end of the heavy chain at a site that was previously engineered into the protein. Tetramer formation is then induced by the addition of streptavidin. By means of fluorescently labeled streptavidin, the tetramer can be used to stain antigen-specific cells. The cells can then be readily identified, for example, by flow cytometry. Such procedures are used for diagnostic or prognostic purposes. Cells identified by the procedure can also be used for therapeutic purposes.

Peptides of the invention are also used as reagents to evaluate immune recall responses. (see, e.g., Bertoni, et al., J. Clin. Invest. 100:503-13, 1997 and Penna, et al., J. Exp. Med. 174:1565-70, 1991.) For example, patient PBMC samples from individuals infected with HPV are analyzed for the presence of antigen-specific CTLs or HTLs using specific peptides. A blood sample containing mononuclear cells may be evaluated by cultivating the PBMCs and stimulating the cells with a peptide of the invention. After an appropriate cultivation period, the expanded cell population may be analyzed, for example, for CTL or for HTL activity.

The peptides are also used as reagents to evaluate the efficacy of a vaccine. PBMCs obtained from a patient vaccinated with an immunogen are analyzed using, for example, either of the methods described above. The patient is HLA typed, and peptide epitope reagents that recognize the allele-specific molecules present in that patient are selected for the analysis. The immunogenicity of the vaccine is indicated by the presence of HPV epitope-specific CTLs and/or HTLs in the PBMC sample.

The peptides of the invention are also used to make antibodies, using techniques well known in the art (see, e.g. CURRENT PROTOCOLS IN IMMUNOLOGY, Wiley/Greene, NY; and Antibodies A Laboratory Manual Harlow, Harlow and Lane, Cold Spring Harbor Laboratory Press, 1989), which may be useful as reagents to diagnose HPV infection. Such antibodies include those that recognize a peptide in the context of an HLA molecule, i.e., antibodies that bind to a peptide-MHC complex.

Selection of Peptide Epitopes from Multiple HPV Types Using Optimal Variant Technology

The present invention is directed to methods for selecting a variant of a peptide epitope which induces a CTL response against another variant(s) of the peptide epitope, by determining whether the variant comprises only conserved residues, as defined herein, at non-anchor positions in comparison to the other variant(s).

In some embodiments, antigen sequences from a population of HPV, said antigens comprising variants of a peptide epitope, are optimally aligned (manually or by computer) along their length, preferably their full length. Variant(s) of a peptide epitope (preferably naturally occurring variants), each 8-11 amino acids in length and comprising the same MHC class I supermotif or motif, are identified manually or with the aid of a computer. In some embodiments, a variant is optimally chosen which comprises preferred anchor residues of said motif and/or which occurs with high frequency within the population of variants. In other embodiments, a variant is randomly chosen. The randomly or otherwise chosen variant is compared to from one to all the remaining variant(s) to determine whether it comprises only conserved residues in the non-anchor positions relative to from one to all the remaining variant(s).

The present invention is also directed to variants identified by the methods above; peptides comprising such variants; nucleic acids encoding such variants and peptides; cells comprising such variants, and/or peptides, and/or nucleic acids; compositions comprising such variants, and/or peptides, and/or nucleic acids, and/or cells; as well as therapeutic and diagnostic methods for using such variants, peptides, nucleic acids, cells, and compositions.

In some embodiments, the invention is directed to a method for identifying a candidate peptide epitope which induces a HLA class I CTL response against variants of said peptide epitope, comprising:
(a) identifying, from a particular antigen of HPV, variants of a peptide epitope 8-11 amino acids in length, each variant comprising primary anchor residues of the same HLA class I binding motif; and
(b) determining whether one of said variants comprises only conserved non-anchor residues in comparison to at least one remaining variant, thereby identifying a candidate peptide epitope.
In some embodiments, (b) comprises identifying a variant which comprises only conserved non-anchor residues in comparison to at least 25%, at least 50%, at least 75%, at least 80%, at least 85%, at least 90%, at least 95%, at least 97%, or at least 99% of the remaining variants.

In some embodiments, the invention is directed to a method for identifying a candidate peptide epitope which induces a HLA class I CTL response against variants of said peptide epitope, comprising:
(a) identifying, from a particular antigen of HPV, variants of a peptide epitope 8-11 amino acids in length, each variant comprising primary anchor residues of the same HLA class I binding motif;
(b) determining whether each of said variants comprises conserved, semi-conserved or non-conserved non-anchor residues in comparison to each of the remaining variants; and
(c) identifying a variant which comprises only conserved non-anchor residues in comparison to at least one remaining variant.

In some embodiments, (c) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

In some embodiments, the invention is directed to a method for identifying a candidate peptide epitope which induces a HLA class I CTL response against variants of said peptide epitope, comprising:
(a) identifying, from a particular antigen of HPV, a population of variants of a peptide epitope 8-11 amino acids in length, each peptide epitope comprising primary anchor residues of the same HLA class I binding motif;
(b) choosing a variant selected from the group consisting of:
a variant which comprises preferred primary anchor residues of said motif;

(c) a variant which occurs with high frequency within the population of variants; and
(d) determining whether the variant of (b) comprises only conserved non-anchor residues in comparison to at least one remaining variant, thereby identifying a candidate peptide epitope.

In some embodiments, (c) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

In some embodiments, the invention is directed to method for identifying a candidate peptide epitope which induces a HLA class I CTL response against variants of said peptide epitope, comprising:
(a) identifying, from a particular antigen of HPV, a population of variants of a peptide epitope 8-11 amino acids in length, each peptide epitope comprising primary anchor residues of the same HLA class I binding motif;

(b) choosing a variant selected from the group consisting of:
(c) a variant which comprises preferred primary anchor residues of said motif;
(d) a variant which occurs with high frequency within the population of variants;
(e) determining whether the variant of (b) comprises conserved, semi-conserved or non-conserved non-anchor residues in comparison to each of the remaining variants; and
(f) identifying a variant which comprises only conserved non-anchor residues in comparison to at least one remaining variant.

In some embodiments, (d) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

In some embodiments, (a) comprises aligning the sequences of said antigens. In a preferred embodiment, (a) comprises aligning the sequences of HPV E1 proteins obtained from HPV Types 16, 18, 31, 33, 45, 52, 56, and 58 (see e.g., Table 25). In a further preferred embodiment, (a) comprises aligning the sequences of HPV E2 proteins obtained from HPV Types 16, 18, 31, 33, 45, 52, 56, and 58 (see e.g., Table 26). In a preferred embodiment, (a) comprises aligning the sequences of HPV E6 proteins obtained from HPV Types 16, 18, 31, 33, 45, 52, 56, and 58 (see e.g., Table 27). In a preferred embodiment, (a) comprises aligning the sequences of HPV E7 proteins obtained from HPV Types 16, 18, 31, 33, 45, 52, 56, and 58 (see e.g., Table 28).

In some embodiments, (b) comprises choosing a variant which comprises preferred primary anchor residues of said motif.

In some embodiments, (b) comprises choosing a variant which occurs with high frequency within said population.

In some embodiments, (b) comprises ranking said variants by frequency of occurrence within said population.

In some embodiments, (b) comprises choosing a variant which comprises preferred primary anchor residues of said motif and which occurs with high frequency within said population.

In some embodiments, (b) comprises ranking said variants by frequency of occurrence within said population.

In some embodiments, the identified variant comprises the fewest conserved anchor residues in comparison to each of the remaining variants.

In some embodiments, the remaining variants comprise 1, 2, 3, 4, 5, 6, 7, 8, 9, 10, 11, 12, 13, 14, 15, 16, 17, 18, 19, 20, 21, 22, 23, 24, 25, 26, 27, 27, 28, 30, 35, 40, 45, 50, 55, 60, 65, 70, 75, 80, 85, 90, 95, 100, 110, 120, 130, 140, 150, 160, 170, 180, 190, 200, 220, 240, 260, 280, or 300 variants.

In some embodiments, the HPV antigen is selected from the group consisting of: E1, E2, E3, E4, E5, E6, E7, L1, and L2.

In some embodiments, the selected variant and the at least one remaining variant comprise different primary anchor residues of the same motif or supermotif.

In some embodiments, the motif or supermotif is selected from the group consisting of those in Table 4.

In some embodiments, the conserved non-anchor residues are at any of positions 3-7 of said variant.

In some embodiments, the variant comprises only 1-3 conserved non-anchor residues compared to at least one remaining variant.

In some embodiments, the variant comprises only 1-2 conserved non-anchor residues compared to at least one remaining variant.

In some embodiments, the variant comprises only 1 conserved non-anchor residue compared to at least one remaining variant.

In some embodiments, the HPV infectious agent is selected from the group consisting of HPV strains 6a, 6b, 11a, 16, 18, 31, 33, 45, 52, 56, and 58.

In some embodiments, the variants are a population of naturally occurring variants.

Optionally, antigen sequences, either full-length or partial, may be aligned manually or by computer (“optimal alignment”). Convenient computer programs for aligning multiple sequences include Omiga, Oxford software, version 1.1.3, using ClustalW alignment, using an open gap penalty of 10.0, extend gap penalty of 0.05, and delay divergent sequences of 40.0 (see, e.g., Tables 19, 20, 21, and 22, herein); and BLASTP 2.2.5 (Nov. 16, 2002) (Altschul, S. F., et al., Nucl. Acid Res. 25:3389-3402 (1997)) using a cutoff=3e-88 (to select human sequences). Alternatively, alignments may be obtained through publicly available sources such as published journal articles and published patent documents.

Vaccine Compositions

Vaccines and methods of preparing vaccines that contain an immunogenically effective amount of one or more peptides as described herein are further embodiments of the invention. Once appropriately immunogenic epitopes have been defined, they can be sorted and delivered by various means, herein referred to as “vaccine” compositions. Such vaccine compositions can include, for example, lipopeptides (e.g., Vitiello, A. et al., J. Clin. Invest. 95:341, 1995), peptide compositions encapsulated in poly(DL-lactide-co-glycolide) (“PLG”) microspheres (see, e.g., Eldridge, et al., Molec. Immunol. 28:287-94, 1991: Alonso, et al., Vaccine 12:299-306, 1994; Jones, et al., Vaccine 13:675-681, 1995), peptide compositions contained in immune stimulating complexes (ISCOMS) (see, e.g., Takahashi, et al., Nature 344:873-75, 1990; Hu, et al., Clin Exp Immunol. 113:235-43, 1998), multiple antigen peptide systems (MAPs) (see e.g., Tam, J. P., Proc. Natl. Acad. Sci. U.S.A. 85:5409-13, 1988; Tam, J. P., J. Immunol. Methods 196:17-32, 1996), peptides formulated as multivalent peptides; peptides for use in ballistic delivery systems, typically crystallized peptides, viral delivery vectors (Perkus, M. E., et al., In: Concepts in vaccine development, Kaufmann, S. H. E., Ed., p. 379, 1996; Chakrabarti, S. et al., Nature 320:535, 1986; Hu, S. L., et al., Nature 320:537, 1986; Kieny, M.-P., et al., AIDS Bio/Technology 4:790, 1986; Top, F. H., et al., J. Infect. Dis. 124:148, 1971; Chanda, P. K., et al., Virology 175:535, 1990), particles of viral or synthetic origin (e.g., Kofler, N., et al., J. Immunol. Methods. 192:25, 1996; Eldridge, J. H., et al., Sem. Hematol. 30:16, 1993; Falo, L. D., Jr., et al., Nature Med. 7:649, 1995), adjuvants (Warren, H. S., Vogel, F. R., and Chedid, L., A. Annu. Rev. Immunol. 4:369, 1986; Gupta, R. K. et al., Vaccine 11:293, 1993), liposomes (Reddy, R., et al., J. Immunol. 148:1585, 1992; Rock, K. L., Immunol. Today 17:131, 1996), or, naked or particle absorbed cDNA (Ulmer, J. B. et al., Science 259:1745, 1993; Robinson, H. L., Hunt, L. A., and Webster, R. G., Vaccine 11:957, 1993; Shiver, J. W., et al., In: Concepts in vaccine development, Kaufmann, S. H. E., Ed., p. 423, 1996; Cease, K. B., and Berzofsky, J. A., Ann. Rev. Immunol. 12:923, 1994 and Eldridge, J. H., et al., Sem. Hematol. 30:16, 1993). Toxin-targeted delivery technologies, also known as receptor mediated targeting, such as those of Avant Immunotherapeutics, Inc. (Needham, Mass.) may also be used.

Vaccine compositions of the invention include nucleic acid-mediated modalities. DNA or RNA encoding one or more of the peptides of the invention can also be administered to a patient. This approach is described, for instance, in Wolff, et. al., Science 247:1465 (1990) as well as U.S. Pat. Nos. 5,580,859; 5,589,466; 5,804,566; 5,739,118; 5,736,524; and 5,679,647; and PCT Publication No. WO 98/04720 (each of which is hereby incorporated by reference in its entirety); and in more detail below. Examples of DNA-based delivery technologies include “naked DNA”, facilitated (e.g., compositions comprising DNA and polyvinylpyrolidone (“PVP) or bupivicaine polymers or peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, for example, as a vector to express nucleotide sequences that encode the peptides of the invention (e.g., modified vaccinia Ankara (Bavarian-Nordic)). Upon introduction into an acutely or chronically infected host or into a non-infected host, the recombinant vaccinia virus expresses the immunogenic peptide, and thereby elicits a host CTL and/or HTL response. Vaccinia vectors and methods useful in immunization protocols are described in, e.g., U.S. Pat. No. 4,722,848. Another vector is BCG (Bacille Calmette Guerin). BCG vectors are described in Stover, et al., Nature 351:456-460 (1991). A wide variety of other vectors useful for therapeutic administration or immunization of the peptides of the invention, e.g. adeno and adeno-associated virus vectors, retroviral vectors, Salmonella typhi vectors, detoxified anthrax toxin vectors, and the like, will be apparent to those skilled in the art from the description herein.

Furthermore, vaccines in accordance with the invention encompass compositions of one or more of the claimed peptides. A peptide can be present in a vaccine individually. Alternatively, the peptide can exist as a homopolymer comprising multiple copies of the same peptide, or as a heteropolymer of various peptides. Polymers have the advantage of increased immunological reaction and, where different peptide epitopes are used to make up the polymer, the additional ability to induce antibodies and/or CTLs that react with different antigenic determinants of the pathogenic organism or tumor-related peptide targeted for an immune response. The composition can be a naturally occurring region of an antigen or can be prepared, e.g., recombinantly or by chemical synthesis.

Carriers that can be used with vaccines of the invention are well known in the art, and include, e.g., thyroglobulin, albumins such as human serum albumin, tetanus toxoid, polyamino acids such as poly L-lysine, poly L-glutamic acid, influenza, hepatitis B virus core protein, and the like. The vaccines can contain a physiologically tolerable (i.e., acceptable) diluent such as water, or saline, preferably phosphate buffered saline. The vaccines also typically include an adjuvant. Adjuvants such as incomplete Freund's adjuvant, aluminum phosphate, aluminum hydroxide, alum, or Lipid A, MPL and analogues thereof, are examples of materials well known in the art. Additionally, as disclosed herein, CTL responses can be primed by conjugating peptides of the invention to lipids, such as tripalmitoyl-S-glycerylcysteinlyseryl-serine (P₃CSS).

Upon immunization with a peptide composition in accordance with the invention, via injection, aerosol, oral, transdermal, transmucosal, intrapleural, intrathecal, or other suitable routes, the immune system of the host responds to the vaccine by producing large amounts of CTLs and/or HTLs specific for the desired antigen. Consequently, the host becomes at least partially immune to later infection, or at least partially resistant to developing an ongoing chronic infection, or derives at least some therapeutic benefit when the antigen was tumor-associated.

In some embodiments, it may be desirable to combine the class I peptide components with components that induce or facilitate neutralizing antibody and or helper T cell responses to the target antigen of interest. A preferred embodiment of such a composition comprises class I and class II epitopes in accordance with the invention. An alternative embodiment of such a composition comprises a class I and/or class II epitope in accordance with the invention, along with a cross reactive HTL epitope such as PADRE® universal helper T cell epitope (Epimmune, San Diego, Calif.) molecule (described e.g., in U.S. Pat. Nos. 5,679,640, 5,736,142, and 6,413,935).

A vaccine of the invention can also include antigen-presenting cells (APC), such as dendritic cells (DC), as a vehicle to present peptides of the invention. Vaccine compositions can be created in vitro, following dendritic cell mobilization and harvesting, whereby loading of dendritic cells occurs in vitro. For example, dendritic cells are transfected, e.g., with a minigene in accordance with the invention, or are pulsed with peptides. The dendritic cell can then be administered to a patient to elicit immune responses in vivo.

Vaccine compositions, either DNA- or peptide-based, can also be administered in vivo in combination with dendritic cell mobilization whereby loading of dendritic cells occurs in vivo.

Antigenic peptides are used to elicit a CTL and/or HTL response ex vivo, as well. The resulting CTL or HTL cells, can be used to treat chronic infections, or tumors in patients that do not respond to other conventional forms of therapy, or will not respond to a therapeutic vaccine peptide or nucleic acid in accordance with the invention. Ex vivo CTL or HTL responses to a particular antigen (infectious or tumor-associated antigen) are induced by incubating in tissue culture the patient's, or genetically compatible, CTL or HTL precursor cells together with a source of antigen-presenting cells (APC), such as dendritic cells, and the appropriate immunogenic peptide. After an appropriate incubation time (typically about 7-28 days), in which the precursor cells are activated and expanded into effector cells, the cells are infused back into the patient, where they will destroy (CTL) or facilitate destruction (HTL) of their specific target cell (an infected cell or a tumor cell). Transfected dendritic cells may also be used as antigen presenting cells.

The vaccine compositions of the invention may also be used in combination with other procedures to remove warts or treat HPV infections. Such procedures include cryosurgery, application of caustic agents, electrodessication, surgical excision and laser ablation (Fauci, et al. HARRISON'S PRINCIPLES OF INTERNAL MEDICINE, 14th Ed., McGraw-Hill Co., Inc, 1998), as well as treatment with antiviral drugs such as interferon-α (see, e.g., Stellato, G., et al., Clin. Diagn. Virol. 7(3):167-72 (1997)) or interferon-inducing drugs such as imiquimod. Topical antimetabolites such a 5-fluorouracil may also be applied.

In patients with HPV-associated cancer, the vaccine compositions of the invention can also be used in conjunction with other treatments used for cancer, e.g., surgery, chemotherapy, drug therapies, radiation therapies, etc. including use in combination with immune adjuvants such as IL-2, IL-12, GM-CSF, and the like.

Preferably, the following principles are utilized when selecting an array of epitopes for inclusion in a polyepitopic composition for use in a vaccine, or for selecting discrete epitopes to be included in a vaccine and/or to be encoded by nucleic acids such as a minigene. It is preferred that the following principles are balanced in order to make the selection. The multiple epitopes to be incorporated in a given vaccine composition may be, but need not be, contiguous in sequence in the native antigen from which the epitopes are derived.
(a) Epitopes are selected which, upon administration, mimic immune responses that have been observed to be correlated with clearance of HPV infection or tumor clearance. For HLA Class I this includes 1-4 epitopes that come from at least one antigen. For HLA Class II a similar rationale is employed; again 1-4 epitopes are selected from at least one antigen (see, e.g., Rosenberg, et al., Science 278:1447-50). In preferred embodiments, 2-4 CTL and/or 2-4 HTL epitopes are selected from at least one antigen. In more highly preferred embodiments, 3-4 CTL and/or 3-4 HTL epitopes are selected from at least one antigen. Epitopes from one antigen may be used in combination with epitopes from one or more additional antigens to produce a vaccine that targets HPV-infected cells and/or associated tumors with varying expression patterns of frequently-expressed antigens as described, e.g., in Example 15.
(b) Epitopes are selected that have the requisite binding affinity established to be correlated with immunogenicity: for HLA Class I an IC₅₀ of 500 nM or less, often 200 nM or less; and for Class II an IC₅₀ of 1000 nM or less.
(c) Sufficient supermotif bearing-peptides, or a sufficient array of allele-specific motif-bearing peptides, are selected to give broad population coverage. For example, it is preferable to have at least 80% population coverage. A Monte Carlo analysis, a statistical evaluation known in the art, can be employed to assess the breadth, or redundancy of, population coverage.
(d) When selecting epitopes from cancer-related antigens it is often useful to select analogs because the patient may have developed tolerance to the native epitope. When selecting epitopes for infectious disease-related antigens it is preferable to select either native or analoged epitopes or a combination of both native an analoged epitopes.
(e) Of particular relevance are epitopes referred to as “nested epitopes.” Nested epitopes occur where at least two epitopes overlap in a given peptide sequence. A nested peptide sequence can comprise both HLA class I and HLA class II epitopes. When providing nested epitopes, a general objective is to provide the greatest number of epitopes per sequence. Thus, an aspect is to avoid providing a peptide that is any longer than the amino terminus of the amino terminal epitope and the carboxyl terminus of the carboxyl terminal epitope in the peptide. When providing a multi-epitopic sequence, such as a sequence comprising nested epitopes, it is generally important to screen the sequence in order to insure that it does not have pathological or other deleterious biological properties.
(f) If a polyepitopic protein is created, or when creating a minigene, an objective is to generate the smallest peptide that encompasses the epitopes of interest. This principle is similar, if not the same as that employed when selecting a peptide comprising nested epitopes. However, with an artificial polyepitopic peptide, the size minimization objective is balanced against the need to integrate any spacer sequences between epitopes in the polyepitopic protein. Spacer amino acid residues can, for example, be introduced to avoid junctional epitopes (an epitope recognized by the immune system, not present in the target antigen, and only created by the man-made juxtaposition of epitopes), or to facilitate cleavage between epitopes and thereby enhance epitope presentation. Junctional epitopes are generally to be avoided because the recipient may generate an immune response to that non-native epitope. Of particular concern is a junctional epitope that is a “dominant epitope.” A dominant epitope may lead to such a zealous response that immune responses to other epitopes are diminished or suppressed.
(g) In cases where the sequences of multiple variants of the same target protein are available, potential peptide epitopes can also be selected on the basis of their conservancy. For example, a criterion for conservancy may define that the entire sequence of an HLA class I binding peptide or the entire 9-mer core of a class II binding peptide be conserved in a designated percentage of the sequences evaluated for a specific protein antigen.
(h) When selecting an array of epitopes of an infectious agent, it is preferred that at least some of the epitopes are derived from early and late proteins. The early proteins of HPV are expressed when the virus is replicating, either following acute or dormant infection. Therefore, it is particularly preferred to use at least some epitopes from early stage proteins to alleviate disease manifestations at the earliest stage possible.
   Minigene Vaccines

A number of different approaches are available which allow simultaneous delivery of multiple epitopes. Nucleic acids encoding the peptides of the invention are a particularly useful embodiment of the invention. Epitopes for inclusion in a minigene are preferably selected according to the guidelines set forth in the previous section. A preferred means of administering nucleic acids encoding the peptides of the invention uses minigene constructs encoding a peptide comprising one or multiple epitopes of the invention.

The use of multi-epitope minigenes is described below and in, e.g., U.S. Pat. No. 6,534,482; Ishioka, et al., J. Immunol. 162:3915-25, 1999; An, L. and Whitton, J. L., J. Virol. 71:2292, 1997; Thomson, S. A., et al., J. Immunol. 157:822, 1996; Whitton, J. L., et al., J. Virol. 67:348, 1993; Hanke, R., et al., Vaccine 16:426, 1998. For example, a multi-epitope DNA plasmid encoding supermotif- and/or motif-bearing epitopes derived from multiple regions of one or more HPV antigens, a PADRE® universal helper T cell epitope (or multiple HTL epitopes from HPV antigens), and an endoplasmic reticulum-translocating signal sequence can be engineered. A vaccine may also comprise epitopes that are derived from other antigens.

The immunogenicity of a multi-epitopic minigene can be tested in transgenic mice to evaluate the magnitude of CTL induction responses against the epitopes tested. Further, the immunogenicity of DNA-encoded epitopes in vivo can be correlated with the in vitro responses of specific CTL lines against target cells transfected with the DNA plasmid. Thus, these experiments can show that the minigene serves to both: (a) generate a CTL response and (b) that the induced CTLs recognize cells expressing the encoded epitopes.

For example, to create a DNA sequence encoding the selected epitopes (minigene) for expression in human cells, the amino acid sequences of the epitopes may be reverse translated. A human codon usage table can be used to guide the codon choice for each amino acid. These epitope-encoding DNA sequences may be directly adjoined, so that when translated, a continuous polypeptide sequence is created. To optimize expression and/or immunogenicity, additional elements can be incorporated into the minigene design. Examples of amino acid sequences that can be reverse translated and included in the minigene sequence include: HLA class I epitopes, HLA class II epitopes, a ubiquitination signal sequence, and/or an endoplasmic reticulum targeting signal. In addition, HLA presentation of CTL and HTL epitopes may be improved by including synthetic (e.g. poly-alanine) or naturally-occurring flanking sequences adjacent to the CTL or HTL epitopes; these larger peptides comprising the epitope(s) are within the scope of the invention.

In preferred embodiments, spacer sequences are incorporated between one or more of the epitopes in the minigene vaccine. In more preferred embodiments, the epitopes are ordered and/or spacer sequences are incorporated between one or more epitopes so as to minimize the occurrence of junctional epitopes and to promote optimal processing of the individual epitopes as the polyepitopic protein encoded by the minigene is expressed. Details of methods of epitope ordering and incorporating spacer sequences between one or more epitopes to create an optimal polyepitopic minigene sequence are provided, for example, in PCT Publication Nos. WO01/47541 and WO02/083714, each of which is hereby incorporated by reference in its entirety.

The invention provides a method and system for optimizing the efficacy of multi-epitope vaccines so as to minimize the number of junctional epitopes and maximize, or at least increase, the immunogenicity and/or antigenicity of multi-epitope vaccines. In particular, the present invention provides multi-epitope nucleic acid constructs encoding a plurality of CTL and/or HTL epitopes obtained or derived from HPV Types 16, 18, 31, 33, 45, 52, 56, and/or 58.

In one embodiment of the invention, a computerized method for designing a multi-epitope construct having multiple epitopes includes the steps of: storing a plurality of input parameters in a memory of a computer system, the input parameters including a plurality of epitopes, at least one motif for identifying junctional epitopes, a plurality of amino acid insertions and at least one enhancement weight value for each insertion; generating a list of epitope pairs from the plurality of epitopes; determining for each epitope pair at least one optimum combination of amino acid insertions based on the at least one motif, the plurality of insertions and the at least one enhancement weight value for each insertion; and identifying at least one optimum arrangement of the plurality of epitopes, wherein a respective one of the at least one optimum combination of amino acid insertions is inserted at a respective junction of two epitopes, so as to provide an optimized multi-epitope construct. In a preferred embodiment, the step of identifying at least one optimum arrangement of epitopes may be accomplished by performing either an exhaustive search wherein all permutations of arrangements of the plurality of epitopes are evaluated or a stochastic search wherein only a subset of all permutations of arrangements of the plurality of epitopes are evaluated.

In a further embodiment, the method determines for each epitope pair at least one optimum combination of amino acid insertions by calculating a function value (F) for each possible combination of insertions for each epitope pair, wherein the number of insertions in a combination may range from 0 to a maximum number of insertions (MaxInsertions) value input by a user, and the function value is calculated in accordance with the equation F=(C+N)/J, when J>0, and F=2(C+N), when J=0, wherein C equals the enhancement weight value of a C+1 flanking amino acid, N equals the enhancement weight value of an N−1 flanking amino acid, and J equals the number of junctional epitopes detected for each respective combination of insertions in an epitope pair based on said at least one motif.

In another embodiment of the invention, a computer system for designing a multi-epitope construct having multiple epitopes, includes: a memory for storing a plurality of input parameters such as a plurality of epitopes, at least one motif for identifying junctional epitopes, a plurality of amino acid insertions and at least one enhancement weight value for each insertion; a processor for retrieving the input parameters from memory and generating a list of epitope pairs from the plurality of epitopes; wherein the processor further determines for each epitope pair at least one optimum combination of amino acid insertions, based on the at least one motif, the plurality of insertions and the at least one enhancement weight value for each insertion. The processor further identifies at least one optimum arrangement of the plurality of epitopes, wherein a respective one of the optimum combinations of amino acid insertions are inserted at a respective junction of two epitopes, to provide an optimized multi-epitope construct; and a display monitor, coupled to the processor, for displaying at least one optimum arrangement of the plurality of epitopes to a user.

In a further embodiment, the invention provides a data storage device storing a computer program for designing a multi-epitope construct having multiple epitopes, the computer program, when executed by a computer system, performing a process that includes the steps of: retrieving a plurality of input parameters from a memory of a computer system, the input parameters including, for example, a plurality of epitopes, at least one motif for identifying junctional epitopes, a plurality of amino acid insertions and at least one enhancement weight value for each insertion; generating a list of epitope pairs from the plurality of epitopes; determining for each epitope pair at least one optimum combination of amino acid insertions based on the at least one motif, the plurality of insertions and the at least one enhancement weight value for each insertion; and identifying at least one optimum arrangement of the plurality of epitopes, wherein a respective one of the at least one optimum combination of amino acid insertions is inserted at a respective junction of two epitopes, so as to provide an optimized multi-epitope construct.

In another embodiment, the invention provides a method and system for designing a multi-epitope construct that comprises multiple epitopes. The method comprising steps of: (a) sorting the multiple epitopes to minimize the number of junctional epitopes; (b) introducing a flanking amino acid residue at a C+1 position of an epitope to be included within the multi-epitope construct; (c) introducing one or more amino acid spacer residues between two epitopes of the multi-epitope construct, wherein the spacer prevents the occurrence of a junctional epitope; and, (d) selecting one or more multi-epitope constructs that have a minimal number of junctional epitopes, a minimal number of amino acid spacer residues, and a maximum number of flanking amino acid residues at a C+1 position relative to each epitope. In some embodiments, the spacer residues are independently selected from residues that are not known HLA Class II primary anchor residues. In particular embodiments, introducing the spacer residues prevents the occurrence of an HTL epitope. Such a spacer often comprises at least 5 amino acid residues independently selected from the group consisting of G, P, and N. In some embodiments the spacer is GPGPG (SEQ ID NO:______).

In some embodiments, introducing the spacer residues prevents the occurrence of a CTL epitope and further, wherein the spacer is 1, 2, 3, 4, 5, 6, 7 or 8 amino acid residues independently selected from the group consisting of A and G. Often, the flanking residue is introduced at the C+1 position of a CTL epitope and is selected from the group consisting of K, R, N, G, and A. In some embodiments, the flanking residue is adjacent to the spacer sequence. The method of the invention can also include substituting an N-terminal residue of an epitope that is adjacent to a C-terminus of an adjacent epitope within the multi-epitope construct with a residue selected from the group consisting of K, R, N, G, and A.

In some embodiments, the method of the invention can also comprise a step of predicting a structure of the multi-epitope construct, and further, selecting one or more constructs that have a maximal structure, i.e., that are processed by an HLA processing pathway to produce all of the epitopes comprised by the construct. In some embodiments, the multi-epitope construct encodes HPV-64 gene 1 (see Table 38, Panel A), HPV-64 gene 2 (see Table 38, Panel B), HPV-43 gene 3 (see Table 38, Panel C), HPV-43 gene 4 (see Table 38, Panel D), HPV-64 gene 1R (see Table 41, Panel A), HPV-64 gene 2R (see Table 41, Panel B), HPV-43 gene 3R (see Table 41, Panel C), and HPV-43 gene 4R (see Table 41, Panel D); HPV-43 gene 3RC (see Table 44, Panel A); HPV-43 gene 3RN (see Table 44, Panel B); HPV-43 gene 3RNC (see Table 44, Panel C); HPV-43 gene 4R; HPV-43 gene 4RC (see Table 44, Panel D); HPV-43-4RN (see Table 44, Panel E); HPV-43-4RNC (see Table 44, Panel F); HPV-46-5 (see Table 47, Panel A); HPV-46-6 (see Table 47, Panel b); HPV-46-5.2 (see Table 47, Panel C); HPV-47-1 (see Table 52, Panel A); HPV-47-2 (see Table 52, Panel B); HPV E1/E2 HTL constructs 780-21.1, 780-22.1 (see Table 59), 780-21.1 Fix, and 780-22.1 Fix (see Table 60); HPV-47-1 (CTL)/780.21.1 (HTL) (see Table 63, Panel A); HPV-47-1 (CTL)/780.22.1 (HTL) (see Table 63, Panel B); HPV-47-2 (CTL)/780.21.1 (HTL) (see Table 63, Panel C); HPV-47-1 (CTL)/780.22.1 (HTL) (see Table 63, Panel D); or HPV-64-2R (see Table 66); HPV-47-5 (see Table 69 and 83); HPV46 gene 5.2/HTL-20 (see Table 70); HPV46 gene 5.2/GP-HTL-20 (see Table 72C-D); HPV46 gene 5.3/HTL-20 (see Table 71); HPV46 gene 5.3/GP-HTL-20 (see Table 72G-H); HPV46 gene 5.3 optimized A24 (see Table 85); HPV47-3 (E1/E2) (see Table 74); HPV47-4 (E1/E2) (see Table 75); HPV E2/E2 HTL-24 (see Table 78); HPV E1/E2 47-2/HTL-24 (see Table 84); or HPV HTL-30 (see Table 80).

In another embodiment of the invention, a system for optimizing multi-epitope constructs include a computer system having a processor (e.g., central processing unit) and at least one memory coupled to the processor for storing instructions executed by the processor and data to be manipulated (i.e., processed) by the processor. The computer system further includes an input device (e.g., keyboard) coupled to the processor and the at least one memory for allowing a user to input desired parameters and information to be accessed by the processor. The processor may be a single CPU or a plurality of different processing devices/circuits integrated onto a single integrated circuit chip. Alternatively, the processor may be a collection of discrete processing devices/circuits selectively coupled to one another via either direct wire/conductor connections or via a data bus. Similarly, the at least one memory may be one large memory device (e.g., EPROM), or a collection of a plurality of discrete memory devices (e.g., EEPROM, EPROM, RAM, DRAM, SDRAM, Flash, etc.) selectively coupled to one another for selectively storing data and/or program information (i.e., instructions executed by the processor). Those of ordinary skill in the art would easily be able to implement a desired computer system architecture to perform the operations and functions disclosed herein.

In one embodiment, the computer system includes a display monitor for displaying information, instructions, images, graphics, etc. The computer system receives user inputs via a keyboard. These user input parameters may include, for example, the number of insertions (i.e., flanking residues and spacer residues), the peptides to be processed, the C+1 and N−1 weighting values for each amino acid, and the motifs to use for searching for junctional epitopes. Based on these input values/parameters, the computer system executes a “Junctional Analyzer” software program which automatically determines the number of junctional epitope for each peptide pair and also calculates an “enhancement” value for each combination of flanking residues and spacers that may be inserted at the junction of each peptide pair. The results of the junctional analyzer program are then used in either an exhaustive or stochastic search program which determines the “optimal” combination or linkage of the entire set of peptides to create a multi-epitope polypeptide, or nucleic acids, having a minimal number of junctional epitopes and a maximum functional (e.g., immunogenicity) value.

In one embodiment, if the number of peptides to be processed by the computer system is less than fourteen, an exhaustive search program is executed by the computer system which examines all permutations of the peptides making up the polypeptide to find the permutation with the “best” or “optimal” function value. In one embodiment, the function value is calculated using the equation (Ce+Ne)/J when J is greater than zero and 2*(Ce+Ne) when J is equal to zero, where Ce is the enhancement “weight” value of an amino acid at the C+1 position of a peptide, Ne is the enhancement “weight” value of an amino acid at the N−1 position of a peptide, and J is the number of junctional epitopes contained in the polypeptide encoded by multi-epitope nucleic acid sequence. Thus, maximizing this function value will identify the peptide pairs having the least number of junctional epitopes and the maximum enhancement weight value for flanking residues. If the number of peptides to be processed is fourteen or more, the computer system executes a stochastic search program that uses a “Monte Carlo” technique to examine many regions of the permutation space to find the best estimate of the optimum arrangement of peptides (e.g., having the maximum function value).

In a further embodiment, the computer system allows a user to input parameter values which format or limit the output results of the exhaustive or stochastic search program. For example, a user may input the maximum number of results having the same function value (“MaxDuplicateFunctionValue=X”) to limit the number of permutations that are generated as a result of the search. Since it is possible for the search programs to find many arrangements that give the same function value, it may be desirable to prevent the output file from being filled by a large number of equivalent solutions. Once this limit is reached no more results are reported until a larger or “better” function value is found. As another example, the user may input the maximum number of “hits” per probe during a stochastic search process. This parameter prevents the stochastic search program from generating too much output on a single probe. In a preferred embodiment, the number of permutations examined in a single probe is limited by several factors: the amount of time set for each probe in the input text file; the speed of the computer, and the values of the parameters “MaxHitsPerProbe” and “MaxDuplicateFunctionValues.” The algorithms used to generate and select permutations for analysis may be in accordance with well-known recursive algorithms found in many computer science text books. For example, six permutations of three things taken three at a time would be generated in the following sequence: ABC; ACB; BAC; BCA; CBA; CAB. As a further example of an input parameter, a user may input how the stochastic search is performed, e.g., randomly, statistically or other methodology; the maximum time allowed for each probe (e.g., 5 minutes); and the number of probes to perform.

Also disclosed herein are multi-epitope constructs designed by the methods described above and hereafter. The multi-epitope constructs include spacer nucleic acids between a subset of the epitope nucleic acids or all of the epitope nucleic acids. One or more of the spacer nucleic acids may encode amino acid sequences different from amino acid sequences encoded by other spacer nucleic acids to optimize epitope processing and to minimize the presence of junctional epitopes.

The minigene sequence may be converted to DNA by assembling oligonucleotides that encode the plus and minus strands of the minigene. Overlapping oligonucleotides (30-100 bases long) may be synthesized, phosphorylated, purified and annealed under appropriate conditions using well known techniques. The ends of the oligonucleotides can be joined, for example, using T4 DNA ligase. This synthetic minigene, encoding the epitope polypeptide, can then be cloned into a desired expression vector.

Standard regulatory sequences well known to those of skill in the art are preferably included in the vector to ensure expression in the target cells. Several vector elements are desirable: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. Additional suitable transcriptional regulartory sequences are well-known in the art (see, e.g., U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences and sequences for replication in mammalian cells may also be considered for increasing minigene expression.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

In addition, immunostimulatory sequences (ISSs or CpGs) appear to play a role in the immunogenicity of DNA vaccines. These sequences may be included in the vector, outside the minigene coding sequence, if desired to enhance immunogenicity.

In some embodiments, a bi-cistronic expression vector which allows production of both the minigene-encoded epitopes and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF), costimulatory molecules, or for HTL responses, pan-DR binding proteins (i.e., PADRES universal helper T cell epitopes, Epimmune, San Diego, Calif.). Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-β) may be beneficial in certain diseases.

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins supplied by QIAGEN, Inc. (Valencia, Calif.). If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is currently being used for intramuscular (IM) administration in clinical trials. See, e.g., U.S. Pat. Nos. 5,580,859, 5,589,466, 6,214,804, and 6,413,942. To improve the immunotherapeutic effects of minigene DNA vaccines to more therapeutically useful levels, an alternative method for formulating purified plasmid DNA may be desirable. A variety of methods have been described, and new techniques may become available. For example, purified plasmid DNA may be complexed with PVP to improve immunotherapeutic usefulness. Plasmid DNA in such formulations is not considered to be “naked DNA.” See, e.g., U.S. Pat. No. 6,040,295. Cationic lipids, glycolipids, and fusogenic liposomes can also be used in the formulation (see, e.g., as described by PCT Publication No. WO 93/24640; Mannino and Gould-Fogerite, BioTechniques 6(7): 682 (1988); U.S. Pat. No. 5,279,833; PCT Publication No. WO 91/06309; and Feigner, et al., Proc. Nat'l Acad. Sci. USA 84:7413 (1987). In addition, peptides and compounds referred to collectively as protective, interactive, non-condensing compounds (PINC) could also be complexed to purified plasmid DNA to influence variables such as stability, intramuscular dispersion, or trafficking to specific organs or cell types.

Target cell sensitization can be used as a functional assay for expression and HLA class I presentation of minigene-encoded CTL epitopes. For example, the plasmid DNA is introduced into a mammalian cell line that is suitable as a target for standard CTL chromium release or IFN-γ production assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 (⁵¹Cr) labeled and used as target cells for epitope-specific CTL lines; cytolysis, detected by ⁵¹Cr release, indicates both production of, and HLA presentation of, minigene-encoded CTL epitopes. Alternatively, IFN-γ production in response to Epitope presentation may be measured in an ELISPOT or ELISA assay. Expression of HTL epitopes may be evaluated in an analogous manner using assays to assess HTL activity.

In vivo immunogenicity is a second approach for functional testing of minigene DNA formulations. Transgenic mice expressing appropriate human HLA proteins are immunized with the DNA product. The dose and route of administration are formulation dependent (e.g., IM for DNA in PBS, intraperitoneal (“i.p.”) for lipid-complexed DNA). Twenty-one days after immunization, splenocytes are harvested and re-stimulated for one week in the presence of peptides encoding each epitope being tested. Thereafter, for CTL effector cells, assays are conducted for cytolysis of peptide-loaded, ⁵¹Cr-labeled target cells using standard techniques. Lysis of target cells that were sensitized by HLA loaded with peptide epitopes, corresponding to minigene-encoded epitopes, demonstrates DNA vaccine function for in vivo induction of CTLs. Alternatively, IFN-γ production in response to Epitope presentation may be measured in an ELISPOT or ELISA assay. Immunogenicity of HTL epitopes is evaluated in transgenic mice in an analogous manner.

Alternatively, the nucleic acids can be administered using ballistic delivery as described, for instance, in U.S. Pat. No. 5,204,253. Using this technique, particles comprised solely of DNA are administered. In a further alternative embodiment, DNA can be adhered to particles, such as gold particles.

Minigenes can also be delivered using other bacterial or viral delivery systems well known in the art, e.g., an expression construct encoding epitopes of the invention can be incorporated into a viral vector such as vaccinia.

Combinations of CTL Peptides with Helper Peptides

Vaccine compositions comprising CTL peptides of the invention can be modified to provide desired attributes, such as improved serum half life, broadened population coverage or enhanced immunogenicity.

For instance, the ability of a peptide to induce CTL activity can be enhanced by linking the peptide to a sequence which contains at least one epitope that is capable of inducing a T helper cell response. The use of T helper epitopes in conjunction with CTL epitopes to enhance immunogenicity is illustrated, for example, in the U.S. Pat. No. 6,419,931, which is hereby incorporated by reference in its entirety.

Although a CTL peptide can be directly linked to a T helper peptide, often CTL epitope/HTL epitope conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues and sometimes 10 or more residues. The CTL peptide epitope can be linked to the T helper peptide epitope either directly or via a spacer either at the amino or carboxy terminus of the CTL peptide. The amino terminus of either the immunogenic peptide or the T helper peptide may be acylated.

In certain embodiments, the T helper peptide is one that is recognized by T helper cells present in the majority of the population. This can be accomplished by selecting peptides that bind to many, most, or all of the HLA class II molecules. These are known as “loosely HLA-restricted” or “promiscuous” T helper sequences. Examples of amino acid sequences that are promiscuous include sequences from antigens such as tetanus toxoid at positions 830-843 (QYIKANSKFIGITE; SEQ ID NO: ______), Plasmodium falciparum circumsporozoite (CS) protein at positions 378-398 (DIEKKIAKMEKASSVFNVVNS; SEQ ID NO: ______), and Streptococcus 18 kD protein at positions 116 (GAVDSILGGVATYGAA; SEQ ID NO: ______). Other examples include peptides bearing a DR 1-4-7 supermotif, or either of the DR3 motifs.

Alternatively, it is possible to prepare synthetic peptides capable of stimulating T helper lymphocytes, in a loosely HLA-restricted fashion, using amino acid sequences not found in nature. These synthetic compounds called Pan-DR-binding epitopes (e.g., PADRE® universal helper T cell epitopes, Epimmune, Inc., San Diego, Calif.) are designed to most preferrably bind most HLA-DR (human HLA class II) molecules. For instance, a pan-DR-binding epitope peptide having the formula: aKXVAAWTLKAAa, where “X” is either cyclohexylalanine, phenylalanine, or tyrosine, and a is either D-alanine or L-alanine, has been found to bind to most HLA-DR alleles, and to stimulate the response of T helper lymphocytes from most individuals, regardless of their HLA type. An alternative of a pan-DR binding epitope comprises all “L” natural amino acids and can be provided in the form of nucleic acids that encode the epitope. PADRE® Universal T Helper cell epitopes are discussed supra in greater detail.

HTL peptide epitopes can also be modified to alter their biological properties. For example, they can be modified to include D-amino acids to increase their resistance to proteases and thus extend their serum half life, or they can be conjugated to other molecules such as lipids, proteins, carbohydrates, and the like to increase their biological activity. For example, a T helper peptide can be conjugated to one or more palmitic acid chains at either the amino or carboxyl termini.

Combinations of CTL Peptides with T Cell Priming Agents

In some embodiments it may be desirable to include in the pharmaceutical compositions of the invention at least one component which primes cytotoxic T lymphocytes. Lipids have been identified as agents capable of priming CTL in vivo against viral antigens. For example, palmitic acid residues can be attached to the ε- and α-amino groups of a lysine residue and then linked, e.g., via one or more linking residues such as Gly, Gly-Gly-, Ser, Ser-Ser, or the like, to an immunogenic peptide. The lipidated peptide can then be administered either directly in a micelle or particle, incorporated into a liposome, or emulsified in an adjuvant, e.g., incomplete Freund's adjuvant. In a preferred embodiment, a particularly effective immunogenic composition comprises palmitic acid attached to ε- and α-amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P₃CSS) can be used to prime virus specific CTL when covalently attached to an appropriate peptide (see, e.g., Deres, et al., Nature 342:561, 1989). Peptides of the invention can be coupled to P₃CSS, for example, and the lipopeptide administered to an individual to specifically prime a CTL response to the target antigen. Moreover, because the induction of neutralizing antibodies can also be primed with P₃CSS-conjugated epitopes, two such compositions can be combined to more effectively elicit both humoral and cell-mediated responses.

CTL and/or HTL peptides can also be modified by the addition of amino acids to the termini of a peptide to provide for ease of linking peptides one to another, for coupling to a carrier support or larger peptide, for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide, particularly class I peptides. However, it is to be noted that modification at the carboxyl terminus of a CTL epitope may, in some cases, alter binding characteristics of the peptide. In addition, the peptide or oligopeptide sequences can differ from the natural sequence by being modified by terminal-NH₂ acylation, e.g., by alkanoyl (C1-C20) or thioglycolyl acetylation, terminal-carboxylamidation, e.g., ammonia, methylamine, etc. In some instances these modifications may provide sites for linking to a support or other molecule.

Vaccine Compositions Comprising DC Pulsed with CTL and/or HTL Peptides

An embodiment of a vaccine composition in accordance with the invention comprises ex vivo administration of a cocktail of epitope-bearing peptides to PBMC, or isolated DC therefrom, from the patient's blood. A pharmaceutical to facilitate harvesting of DC can be used, such as Progenipoietin (Monsanto, St. Louis, Mo.) or GM-CSF/IL-4. After pulsing the DC with peptides and prior to reinfusion into patients, the DC are washed to remove unbound peptides. In this embodiment, a vaccine comprises peptide-pulsed DCs which present the pulsed peptide epitopes complexed with HLA molecules on their surfaces.

The DC can be pulsed ex vivo with a cocktail of peptides, some of which stimulate CTL responses to one or more HPV antigens of interest. Optionally, a helper T cell (HTL) peptide such as a PADRE® family molecule, can be included to facilitate the CTL response. Thus, a vaccine in accordance with the invention, preferably comprising epitopes from multiple HPV antigens, is used to treat HPV infection or cancer resulting from HPV infection.

Administration of Vaccines for Therapeutic or Prophylactic Purposes

The peptides of the present invention and pharmaceutical and vaccine compositions of the invention are typically used to treat and/or prevent cancer associated with HPV infection. Vaccine compositions containing the peptides of the invention are administered to a patient infected with HPV or to an individual susceptible to, or otherwise at risk for, HPV infection to elicit an immune response against HPV antigens and thus enhance the patient's own immune response capabilities.

As noted above, peptides comprising CTL and/or HTL epitopes of the invention induce immune responses when presented by HLA molecules and contacted with a CTL or HTL specific for an epitope comprised by the peptide. The peptides (or DNA encoding them) can be administered individually, as fusions of one or more peptide sequences or as combinations of individual peptides. The manner in which the peptide is contacted with the CTL or HTL is not critical to the invention. For instance, the peptide can be contacted with the CTL or HTL either in vivo or in vitro. If the contacting occurs in vivo, the peptide itself can be administered to the patient, or other vehicles, e.g., DNA vectors encoding one or more peptides, viral vectors encoding the peptide(s), liposomes and the like, can be used, as described herein.

When the peptide is contacted in vitro, the vaccinating agent can comprise a population of cells, e.g., peptide-pulsed dendritic cells, or HPV-specific CTLs, which have been induced by pulsing antigen-presenting cells in vitro with the peptide or by transfecting antigen-presenting cells with a minigene of the invention. Such a cell population is subsequently administered to a patient in a therapeutically effective dose.

In therapeutic applications, peptide and/or nucleic acid compositions are administered to a patient in an amount sufficient to elicit an effective CTL and/or HTL response to the virus antigen and to cure or at least partially arrest or slow symptoms and/or complications. An amount adequate to accomplish this is defined as “therapeutically effective dose.” Amounts effective for this use will depend on, e.g., the particular composition administered, the manner of administration, the stage and severity of the disease being treated, the weight and general state of health of the patient, and the judgment of the prescribing physician.

For pharmaceutical compositions, the immunogenic peptides of the invention, or DNA encoding them, are generally administered to an individual already infected with HPV. The peptides or DNA encoding them can be administered individually or as fusions of one or more peptide sequences. HPV-infected patients, with or without neoplasia, can be treated with the immunogenic peptides separately or in conjunction with other treatments, such as surgery, as appropriate.

For therapeutic use, administration should generally begin at the first diagnosis of HPV infection or HPV-associated cancer. This is followed by boosting doses until at least symptoms are substantially abated and for a period thereafter. The embodiment of the vaccine composition (i.e., including, but not limited to embodiments such as peptide cocktails, polyepitopic polypeptides, minigenes, or TAA-specific CTLs or pulsed dendritic cells) delivered to the patient may vary according to the stage of the disease or the patient's health status. For example, in a patient with a tumor that expresses HPV antigens, a vaccine comprising HPV-specific CTL may be more efficacious in killing tumor cells in patient with advanced disease than alternative embodiments.

Where susceptible individuals are identified prior to or during infection, the composition can be targeted to them, thus minimizing the need for administration to a larger population. Susceptible populations include those individuals who are sexually active.

The peptide or other compositions used for the treatment or prophylaxis of HPV infection can be used, e.g., in persons who have not manifested symptoms, e.g., genital warts or neoplastic growth. In this context, it is generally important to provide an amount of the peptide epitope delivered by a mode of administration sufficient to effectively stimulate a cytotoxic T cell response; compositions which stimulate helper T cell responses can also be given in accordance with this embodiment of the invention.

The dosage for an initial therapeutic immunization generally occurs in a unit dosage range where the lower value is about 1, 5, 50, 500, or 1,000 μg and the higher value is about 10,000, 20,000, 30,000 or 50,000 μg. Dosage values for a human typically range from about 500 μg to about 50,000 μg per 70 kilogram patient. Boosting dosages of between about 1.0 μg to about 50,000 μg of peptide pursuant to a boosting regimen over weeks to months may be administered depending upon the patient's response and condition as determined by measuring the specific activity of CTL and HTL obtained from the patient's blood. Administration should continue until at least clinical symptoms or laboratory tests indicate that the viral infection, or neoplasia, has been eliminated or reduced and for a period thereafter. The dosages, routes of administration, and dose schedules are adjusted in accordance with methodologies known in the art.

In certain embodiments, the peptides and compositions of the present invention are employed in serious disease states, that is, life-threatening or potentially life threatening situations. In such cases, as a result of the minimal amounts of extraneous substances and the relative nontoxic nature of the peptides in preferred compositions of the invention, it is possible and may be felt desirable by the treating physician to administer substantial excesses of these peptide compositions relative to these stated dosage amounts.

The vaccine compositions of the invention can also be used purely as prophylactic agents. Generally the dosage for an initial prophylactic immunization generally occurs in a unit dosage range where the lower value is about 1, 5, 50, 500, or 1,000 μg and the higher value is about 10,000, 20,000, 30,000 or 50,000 μg. Dosage values for a human typically range from about 500 μg to about 50,000 μl per 70 kilogram patient. This is followed by boosting dosages of between about 1.0 μg to about 50,000 μg of peptide administered at defined intervals from about four weeks to six months after the initial administration of vaccine. The immunogenicity of the vaccine can be assessed by measuring the specific activity of CTL and HTL obtained from a sample of the patient's blood.

The pharmaceutical compositions for therapeutic treatment are intended for parenteral, topical, oral, intrathecal, or local (e.g. as a cream or topical ointment) administration. Preferably, the pharmaceutical compositions are administered parentally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH-adjusting and buffering agents, tonicity adjusting agents, wetting agents, preservatives, and the like, for example, sodium acetate, sodium lactate, sodium chloride, potassium chloride, calcium chloride, sorbitan monolaurate, triethanolamine oleate, etc.

The concentration of peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

A human unit dose form of the peptide composition is typically included in a pharmaceutical composition that comprises a human unit dose of an acceptable carrier, preferably an aqueous carrier, and is administered in a volume of fluid that is known by those of skill in the art to be used for administration of such compositions to humans (see, e.g., Remington's Pharmaceutical Sciences, 17^{th} Edition, A. Gennaro, Ed., Mack Publishing Co., Easton, Pa., 1985).

The peptides of the invention, and/or nucleic acids encoding the peptides, can also be administered via liposomes, which may also serve to target the peptides to a particular tissue, such as lymphoid tissue, or to target selectively to infected cells, as well as to increase the half-life of the peptide composition. Liposomes include emulsions, foams, micelles, insoluble monolayers, liquid crystals, phospholipid dispersions, lamellar layers and the like. In these preparations, the peptide to be delivered is incorporated as part of a liposome, alone or in conjunction with a molecule which binds to a receptor prevalent among lymphoid cells, such as monoclonal antibodies which bind to the CD45 antigen, or with other therapeutic or immunogenic compositions. Thus, liposomes either filled or decorated with a desired peptide of the invention can be directed to the site of lymphoid cells, where the liposomes then deliver the peptide compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), and U.S. Pat. Nos. 4,235,871, 4,501,728, 4,837,028, and 5,019,369.

For targeting cells of the immune system, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

HLA Expression: Implications for T Cell-Based Immunotherapy

Similarly, it is widely recognized that the pathological process by which an individual succumbs to a neoplastic disease is complex. During the course of disease, many changes occur in cancer cells. The tumor accumulates alterations which are in part related to dysfunctional regulation of growth and differentiation, but also related to maximizing its growth potential, escape from drug treatment and/or the body's immunosurveillance. Neoplastic disease results in the accumulation of several different biochemical alterations of cancer cells, as a function of disease progression. It also results in significant levels of intra- and inter-cancer heterogeneity, particularly in the late, metastatic stage.

Familiar examples of cellular alterations affecting treatment outcomes include the outgrowth of radiation or chemotherapy resistant tumors during the course of therapy. These examples parallel the emergence of drug resistant viral strains as a result of aggressive chemotherapy, e.g., of chronic HBV and HIV infection, and the current resurgence of drug resistant organisms that cause Tuberculosis and Malaria. It appears that significant heterogeneity of responses is also associated with other approaches to cancer therapy, including anti-angiogenesis drugs, passive antibody immunotherapy, and active T cell-based immunotherapy. Thus, in view of such phenomena, epitopes from multiple disease-related antigens can be used in vaccines and therapeutics thereby counteracting the ability of diseased cells to mutate and escape treatment.

One of the main factors contributing to the dynamic interplay between host and disease is the immune response mounted against the pathogen, infected cell, or malignant cell. In many conditions such immune responses control the disease. Several animal model systems and prospective studies of natural infection in humans suggest that immune responses against a pathogen can control the pathogen, prevent progression to severe disease and/or eliminate the pathogen. A common theme is the requirement for a multispecific T cell response, and that narrowly focused responses appear to be less effective. These observations guide the skilled artisan as to embodiments of methods and compositions of the present invention that provide for a broad immune response.

In the cancer setting there are several non-limiting findings that indicate that immune responses can impact neoplastic growth:
(a) the demonstration in many different animal models, that anti-tumor T cells, restricted by MHC class I, can prevent or treat tumors.
(b) encouraging results have come from immunotherapy trials.
(c) observations made in the course of natural disease correlated the type and composition of T cell infiltrate within tumors with positive clinical outcomes (Coulie P G, et al. Antitumor immunity at work in a melanoma patient In Advances in Cancer Research, 213-242, 1999).
(d) tumors commonly have the ability to mutate, thereby changing their immunological recognition. For example, the presence of mono-specific CTL was also correlated with control of tumor growth, until antigen loss emerged (Riker, A., et al., Surgery, 126(2):112-20, 1999; Marchand, M., et al., Int. J. Cancer 80(2):219-30, 1999). Similarly, loss of beta 2 microglobulin was detected in 5/13 lines established from melanoma patients after receiving immunotherapy at the National Cancer Institute (Restifo, N. P., et al., Loss of functional Beta2-microglobulin in metastatic melanomas from five patients receiving immunotherapy J. Nat'l Cancer Inst., 88 (2): 100-08, 1996). It has long been recognized that HLA class I is frequently altered in various tumor types. This has led to a hypothesis that this phenomenon might reflect immune pressure exerted on the tumor by means of class I restricted CTL. The extent and degree of alteration in HLA class I expression appears to be reflective of past immune pressures, and may also have prognostic value (van Duinen, S. G., et al., Cancer Res. 48, 1019-25, 1988; Moller, P., et al., Cancer Res. 51, 729-36, 1991).

Taken together, these observations provide a rationale for immunotherapy of cancer and infectious disease, and suggest that effective strategies need to account for the complex series of pathological changes associated with disease.

The level and pattern of expression of HLA class I antigens in tumors has been studied in many different tumor types and alterations have been reported in all types of tumors studied. The molecular mechanisms underlining HLA class I alterations have been demonstrated to be quite heterogeneous. They include alterations in the TAP/processing pathways, mutations of β2-microglobulin and specific HLA heavy chains, alterations in the regulatory elements controlling over class I expression and loss of entire chromosome sections. There are several reviews on this topic, see, e.g., Garrido, F., et al., Immunol. Today 14(10):491-99, 1993; Kaklamanis, L., et al., Int. J. Cancer, 51(3):379-85, 1992. There are three main types of HLA Class I alteration (complete loss, allele-specific loss and decreased expression). The functional significance of each alteration is discussed separately.

Complete loss of HLA expression can result from a variety of different molecular mechanisms, reviewed in (Algarra, I., et al., Human Immunol. 61, 65-73, 2000; Browning, M., et al., Tissue Antigens 47:364-71, 1996; Ferrone, S., et al., Immunol. Today, 16(10): 487-94, 1995; Garrido, F., et al., Immunol. Today 14(10):491-99, 1993; Tait, B. D., Hum. Immunol. 61, 158-65, 2000). In functional terms, this type of alteration has several important implications.

While the complete absence of class I expression will eliminate CTL recognition of those tumor cells, the loss of HLA class I will also render the tumor cells extraordinary sensitive to lysis from NK cells (Ohnmacht, G. A., et al., J. Cell. Phys. 182:332-38, 2000; Liunggren, H. G., et al., J. Exp. Med., 162(6):1745-59, 1985; Maio, M., et al., J. Clin. Invest. 88(1):282-89, 1991; Schrier, P. I., et al., Adv. Cancer Res., 60:181-246, 1993).

The complementary interplay between loss of HLA expression and gain in NK sensitivity is exemplified by the classic studies of Coulie and coworkers (in Advances in Cancer Research, 213-242, 1999) which described the evolution of a patient's immune response over the course of several years. Because of increased sensitivity to NK lysis, it is predicted that approaches leading to stimulation of innate immunity in general and NK activity in particular would be of special significance. An example of such an approach is the induction of large amounts of dendritic cells (DC) by various hematopoietic growth factors, such as Flt3 ligand or ProGP. The rationale for this approach resides in the well known fact that dendritic cells produce large amounts of IL-12, one of the most potent stimulators for innate immunity and NK activity in particular. Alternatively, IL-12 is administered directly, or as nucleic acids that encode it. In this light, it is interesting to note that Flt3 ligand treatment results in transient tumor regression of a class I negative prostate murine cancer model (Ciavarra, R. P., et al., Cancer Res 60:2081-84, 2000). In this context, specific anti-tumor vaccines in accordance with the invention synergize with these types of hematopoietic growth factors to facilitate both CTL and NK cell responses, thereby appreciably impairing a cell's ability to mutate and thereby escape efficacious treatment. Thus, an embodiment of the present invention comprises a composition of the invention together with a method or composition that augments functional activity or numbers of NK cells. Such an embodiment can comprise a protocol that provides a composition of the invention sequentially with an NK-inducing modality, or contemporaneous with an NK-inducing modality.

Secondly, complete loss of HLA frequently occurs only in a fraction of the tumor cells, while the remainder of tumor cells continue to exhibit normal expression. In functional terms, the tumor would still be subject, in part, to direct attack from a CTL response; the portion of cells lacking HLA subject to an NK response. Even if only a CTL response were used, destruction of the HLA expressing fraction of the tumor has dramatic effects on survival times and quality of life.

It should also be noted that in the case of heterogeneous HLA expression, both normal HLA-expressing as well as defective cells are predicted to be susceptible to immune destruction based on “bystander effects.” Such effects were demonstrated, e.g., in the studies of Rosendahl and colleagues that investigated in vivo mechanisms of action of antibody targeted superantigens (J. Immunol. 160(11):5309-13, 1998). The bystander effect is understood to be mediated by cytokines elicited from, e.g., CTLs acting on an HLA-bearing target cell, whereby the cytokines are in the environment of other diseased cells that are concomitantly killed.

One of the most common types of alterations in class I molecules is the selective loss of certain alleles in individuals heterozygous for HLA. Allele-specific alterations might reflect the tumor adaptation to immune pressure, exerted by an immunodominant response restricted by a single HLA restriction element. This type of alteration allows the tumor to retain class I expression and thus escape NK cell recognition, yet still be susceptible to a CTL-based vaccine in accordance with the invention which comprises epitopes corresponding to the remaining HLA type. Thus, a practical solution to overcome the potential hurdle of allele-specific loss relies on the induction of multispecific responses. Just as the inclusion of multiple disease-associated antigens in a vaccine of the invention guards against mutations that yield loss of a specific disease antigens, simultaneously targeting multiple HLA specificities and multiple disease-related antigens prevents disease escape by allele-specific losses.

The sensitivity of effector CTL has long been demonstrated (Brower, R. C., et al., Mol. Immunol., 31; 1285-93, 1994; Chriustnick, E. T., et al., Nature 352:67-70, 1991; Sykulev, Y., et al., Immunity, 4(6):565-71, 1996). Even a single peptide/MHC complex can result in tumor cells lysis and release of anti-tumor lymphokines. The biological significance of decreased HLA expression and possible tumor escape from immune recognition is not fully known. Nevertheless, it has been demonstrated that CTL recognition of as few as one MHC/peptide complex is sufficient to lead to tumor cell lysis.

Further, it is commonly observed that expression of HLA can be upregulated by gamma IFN, commonly secreted by effector CTL. Additionally, HLA class I expression can be induced in vivo by both alpha and beta IFN (Halloran, et al., J. Immunol. 148:3837, 1992; Pestka, S., et al., Annu. Rev. Biochem. 56:727-77, 1987). Conversely, decreased levels of HLA class I expression also render cells more susceptible to NK lysis.

With regard to gamma IFN, Torres, et al. (Tissue Antigens 47:372-81, 1996) note that HLA expression is upregulated by IFN-γ in pancreatic cancer, unless a total loss of haplotype has occurred. Similarly, Rees and Mian note that allelic deletion and loss can be restored, at least partially, by cytokines such as IFN-γ (Cancer Immunol. Immunother. 48:374-81, 1999). It has also been noted that IFN-γ treatment results in upregulation of class I molecules in the majority of the cases studied (Browning, M., et al., Tissue Antigens 47:364-71, 1996). Kaklamakis, et al., also suggested that adjuvant immunotherapy with IFN-γ may be beneficial in the case of HLA class I negative tumors (Kaklamanis, L., Cancer Res. 55:5191-94, 1995). It is important to underline that IFN-gamma production is induced and self-amplified by local inflammation/immunization (Halloran, et al., J. Immunol. 148:3837, 1992), resulting in large increases in MHC expressions even in sites distant from the inflammatory site.

Finally, studies have demonstrated that decreased HLA expression can render tumor cells more susceptible to NK lysis (Ohnmacht, G. A., et al., J. Cell. Phys. 182:332-38, 2000; Liunggren, H. G., et al., J. Exp. Med., 162(6):1745-59, 1985; Maio, M., et al., J. Clin. Invest. 88(1):282-89, 1991; Schrier, P. I., et al., Adv. Cancer Res., 60:181-246, 1993). If decreases in HLA expression benefit a tumor because it facilitates CTL escape, but render the tumor susceptible to NK lysis, then a minimal level of HLA expression that allows for resistance to NK activity would be selected for (Garrido, F., et al., Immunol Today 18(2):89-96, 1997). Therefore, a therapeutic compositions or methods in accordance with the invention together with a treatment to upregulate HLA expression and/or treatment with high affinity T-cells renders the tumor sensitive to CTL destruction.

The frequency of alterations in class I expression is the subject of numerous studies (Algarra, I., et al., Human Immunol. 61, 65-73, 2000). Rees and Mian estimate allelic loss to occur overall in 3-20% of tumors, and allelic deletion to occur in 15-50% of tumors. It should be noted that each cell carries two separate sets of class I genes, each gene carrying one HLA-A and one HLA-B locus. Thus, fully heterozygous individuals carry two different HLA-A molecules and two different HLA-B molecules. Accordingly, the actual frequency of losses for any specific allele could be as little as one quarter of the overall frequency. They also note that, in general, a gradient of expression exists between normal cells, primary tumors and tumor metastasis. In a study from Natali and coworkers (Proc. Natl. Acad. Sci. U.S.A. 86:6719-23, 1989), solid tumors were investigated for total HLA expression, using W6/32 antibody, and for allele-specific expression of the A2 antigen, as evaluated by use of the BB7.2 antibody. Tumor samples were derived from primary cancers or metastasis, for 13 different tumor types, and scored as negative if less than 20%, reduced if in the 30-80% range, and normal above 80%. All tumors, both primary and metastatic, were HLA positive with W6/32. In terms of A2 expression, a reduction was noted in 16.1% of the cases, and A2 was scored as undetectable in 39.4% of the cases. Garrido and coworkers (Immunol. Today 14(10):491-99, 1993) emphasize that HLA changes appear to occur at a particular step in the progression from benign to most aggressive. Jiminez et al (Cancer Immunol. Immunother. 48:684-90, 2000) have analyzed 118 different tumors (68 colorectal, 34 laryngeal and 16 melanomas). The frequencies reported for total loss of HLA expression were 11% for colon, 18% for melanoma and 13% for larynx. Thus, HLA class I expression is altered in a significant fraction of the tumor types, possibly as a reflection of immune pressure, or simply a reflection of the accumulation of pathological changes and alterations in diseased cells.

A majority of the tumors express HLA class I, with a general tendency for the more severe alterations to be found in later stage and less differentiated tumors. This pattern is encouraging in the context of immunotherapy, especially considering that: 1) the relatively low sensitivity of immunohistochemical techniques might underestimate HLA expression in tumors; 2) class I expression can be induced in tumor cells as a result of local inflammation and lymphokine release; and, 3) class I negative cells are sensitive to lysis by NK cells.

Accordingly, various embodiments of the present invention can be selected in view of the fact that there can be a degree of loss of HLA molecules, particularly in the context of neoplastic disease. For example, the treating physician can assay a patient's tumor to ascertain whether HLA is being expressed. If a percentage of tumor cells express no class I HLA, then embodiments of the present invention that comprise methods or compositions that elicit NK cell responses can be employed. As noted herein, such NK-inducing methods or composition can comprise a Flt3 ligand or ProGP which facilitate mobilization of dendritic cells, the rationale being that dendritic cells produce large amounts of IL-12. IL-12 can also be administered directly in either amino acid or nucleic acid form. It should be noted that compositions in accordance with the invention can be administered concurrently with NK cell-inducing compositions, or these compositions can be administered sequentially.

In the context of allele-specific HLA loss, a tumor retains class I expression and may thus escape NK cell recognition, yet still be susceptible to a CTL-based vaccine in accordance with the invention which comprises epitopes corresponding to the remaining HLA type. The concept here is analogous to embodiments of the invention that include multiple disease antigens to guard against mutations that yield loss of a specific antigen. Thus, one can simultaneously target multiple HLA specificities and epitopes from multiple disease-related antigens to prevent tumor escape by allele-specific loss as well as disease-related antigen loss. In addition, embodiments of the present invention can be combined with alternative therapeutic compositions and methods. Such alternative compositions and methods comprise, without limitation, radiation, cytotoxic pharmaceuticals, and/or compositions/methods that induce humoral antibody responses.

Moreover, it has been observed that expression of HLA can be upregulated by gamma IFN, which is commonly secreted by effector CTL, and that HLA class I expression can be induced in vivo by both alpha and beta IFN. Thus, embodiments of the invention can also comprise alpha, beta and/or gamma IFN to facilitate upregualtion of HLA.

Reprieve Periods from Therapies that Induce Side Effects: “Scheduled Treatment Interruptions or Drug Holidays”

Recent evidence has shown that certain patients infected with a pathogen, whom are initially treated with a therapeutic regimen to reduce pathogen load, have been able to maintain decreased pathogen load when removed from the therapeutic regimen, i.e., during a “drug holiday” (Rosenberg, E., et al., Nature 407:523-26, Sep. 28, 2000). As appreciated by those skilled in the art, many therapeutic regimens for both pathogens and cancer have numerous, often severe, side effects. During the drug holiday, the patient's immune system is keeping the disease in check. Methods for using compositions of the invention are used in the context of drug holidays for cancer and pathogenic infection.

For treatment of an infection, where therapies are not particularly immunosuppressive, compositions of the invention are administered concurrently with the standard therapy. During this period, the patient's immune system is directed to induce responses against the epitopes comprised by the present inventive compositions. Upon removal from the treatment having side effects, the patient is primed to respond to the infectious pathogen should the pathogen load begin to increase. Composition of the invention can be provided during the drug holiday as well.

For patients with cancer, many therapies are immunosuppressive. Thus, upon achievement of a remission or identification that the patient is refractory to standard treatment, then upon removal from the immunosuppressive therapy, a composition in accordance with the invention is administered. Accordingly, as the patient's immune system reconstitutes, precious immune resources are simultaneously directed against the cancer. Composition of the invention can also be administered concurrently with an immunosuppressive regimen if desired.

Kits

The peptide and nucleic acid compositions of this invention can be provided in kit form together with instructions for vaccine administration. Typically the kit would include desired peptide compositions in a container, preferably in unit dosage form and instructions for administration. An alternative kit would include a minigene construct with desired polynucleotides of the invention in a container, preferably in unit dosage form together with instructions for administration. Lymphokines or polynucleotides encoding them such as IL-2 or IL-12 may also be included in the kit. Other kit components that may also be desirable include, for example, a sterile syringe, booster dosages, and other desired excipients.

Overview

Epitopes in accordance with the present invention were successfully used to induce an immune response. Immune responses with these epitopes have been induced by administering the epitopes in various forms. The epitopes have been administered as peptides, as polynucleotides, and as viral vectors comprising nucleic acids that encode the epitope(s) of the invention. Upon administration of peptide-based epitope forms, immune responses have been induced by direct loading of an epitope onto an empty HLA molecule that is expressed on a cell, and via internalization of the epitope and processing via the HLA class I pathway; in either event, the HLA molecule expressing the epitope was then able to interact with and induce a CTL response. Peptides can be delivered directly or using such agents as liposomes. They can additionally be delivered using ballistic delivery, in which the peptides are typically in a crystalline form. When DNA is used to induce, an immune response, it is administered either as naked DNA or as DNA complexed to a polymer (e.g., PVP) or with a lipid, generally in a dose range of approximately 1-5 mg, or via the ballistic “gene gun” delivery, typically in a dose range of approximately 10-100 μg. The DNA can be delivered in a variety of conformations, e.g., linear, circular etc. Various viral vectors have also successfully been used that comprise nucleic acids which encode epitopes in accordance with the invention.

Accordingly compositions in accordance with the invention exist in several forms. Embodiments of each of these composition forms in accordance with the invention have been successfully used to induce an immune response.

One composition in accordance with the invention comprises a plurality of peptides. This plurality or cocktail of peptides is generally admixed with one or more pharmaceutically acceptable excipients. The peptide cocktail can comprise multiple copies of the same peptide or can comprise a mixture of peptides. One or more of the peptides can be analogs of naturally occurring epitopes. The peptides can comprise artificial amino acids and/or chemical modifications such as addition of a surface active molecule, e.g., lipidation; acetylation, glycosylation, biotinylation, phosphorylation etc. The peptides can be CTL or HTL epitopes. In a preferred embodiment the peptide cocktail comprises a plurality of different CTL epitopes and at least one HTL epitope. The HTL epitope can be naturally or non-naturally occurring (e.g., the PADRE® universal HTL epitope, Epimmune Inc., San Diego, Calif.). The number of distinct epitopes in an embodiment of the invention is generally a whole unit integer from one through one hundred fifty (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or 150).

An additional embodiment of a composition in accordance with the invention comprises a polypeptide multi-epitope construct, i.e., a polyepitopic peptide. Polyepitopic peptides in accordance with the invention are prepared by use of technologies well-known in the art. By use of these known technologies, epitopes in accordance with the invention are connected one to another. The polyepitopic peptides can be linear or non-linear, e.g., multivalent. These polyepitopic constructs can comprise artificial amino acid residue, spacing or spacer amino acid residues, flanking amino acid residues, or chemical modifications between adjacent epitope units. The polyepitopic construct can be a heteropolymer or a homopolymer. The polyepitopic constructs generally comprise epitopes in a quantity of any whole unit integer between 2-150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or 150). In a preferred embodiment, the polyepitopic construct can comprise CTL and/or HTL epitopes. The HTL epitope can be naturally or non-naturally (e.g., the PADRE® Universal HTL epitope, Epimmune Inc., San Diego, Calif.). One or more of the epitopes in the construct can be modified, e.g., by addition of a surface active material, e.g. a lipid, or chemically modified, e.g., acetylation, etc. Moreover, bonds in the multi-epitopic construct can be other than peptide bonds, e.g., covalent bonds, ester or ether bonds, disulfide bonds, hydrogen bonds, ionic bonds etc.

Alternatively, a composition in accordance with the invention comprises a construct which comprises a series, sequence, stretch, etc., of amino acids that have homology to or identity with (i.e., corresponds to or is contiguous with) to a native sequence. This stretch of amino acids comprises at least one subsequence of amino acids that, if cleaved or isolated from the longer series of amino acids, functions as an HLA class I or HLA class II epitope in accordance with the invention. In this embodiment, the peptide sequence is modified, so as to become a construct as defined herein, by use of any number of techniques known or to be provided in the art. The polyepitopic constructs can contain homology to or exhibit identity with a naturally occurring sequence in any whole unit integer increment from 70-100%, e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or, 100 percent.

A further embodiment of a composition in accordance with the invention is an antigen presenting cell that comprises one or more epitopes in accordance with the invention. The antigen presenting cell can be a “professional” antigen presenting cell, such as a dendritic cell. The antigen presenting cell can comprise the epitope of the invention by any means known or to be determined in the art. Such means include pulsing of dendritic cells with one or more individual epitopes or with one or more peptides that comprise multiple epitopes, by polynucleotide administration such as ballistic DNA or by other techniques in the art for administration of nucleic acids, including vector-based, e.g. viral vector, delivery of polynucleotide.

Further embodiments of compositions in accordance with the invention comprise polynucleotides that encode one or more peptides of the invention, or polynucleotides that encode a polyepitopic peptide in accordance with the invention. As appreciated by one of ordinary skill in the art, various polynucleotide compositions will encode the same peptide due to the redundancy of the genetic code. Each of these polynucleotide compositions falls within the scope of the present invention. This embodiment of the invention comprises DNA or RNA, and in certain embodiments a combination of DNA and RNA. It is to be appreciated that any composition comprising polynucleotides that will encode a peptide in accordance with the invention or any other peptide based composition in accordance with the invention, falls within the scope of this invention.

It is to be appreciated that peptide-based forms of the invention (as well as the polynucleotides that encode them) can comprise analogs of epitopes of the invention generated using principles already known, or to be known, in the art. Principles related to analoging are now known in the art, and are disclosed herein; moreover, analoging principles (heteroclitic analoging) are disclosed in co-pending application serial number U.S. Ser. No. 09/226,775 filed 6 Jan. 1999. Generally the compositions of the invention are isolated or purified.

The invention will be described in greater detail by way of specific examples. The following examples are offered for illustrative purposes, and are not intended to limit the invention in any manner. Those of skill in the art will readily recognize a variety of non-critical parameters that can be changed or modified to yield alternative embodiments in accordance with the invention.

### EXAMPLES

### Example 1

### HLA Class I and Class II Binding Assays

The following example of peptide binding to HLA molecules demonstrates quantification of binding affinities of HLA class I and class II peptides. Binding assays can be performed with peptides that are either motif-bearing or not motif-bearing.

HLA class I and class II binding assays using purified HLA molecules were performed in accordance with disclosed protocols (e.g., PCT publications WO 94/20127 and WO 94/03205; Sidney, et al., Current Protocols in Immunology 18.3.1 (1998); Sidney, et al., J. Immunol. 154:247 (1995); Sette, et al., Mol. Immunol. 31:813 (1994)). Briefly, purified MHC molecules (5 to 500 nM) were incubated with various unlabeled peptide inhibitors and 1-10 nM ¹²⁵I-radiolabeled probe peptides as described. Following incubation, MHC-peptide complexes were separated from free peptide by gel filtration and the fraction of peptide bound was determined. Typically, in preliminary experiments, each MHC preparation was titered in the presence of fixed amounts of radiolabeled peptides to determine the concentration of HLA molecules necessary to bind 10-20% of the total radioactivity. All subsequent inhibition and direct binding assays were performed using these HLA concentrations.

Since under these conditions [label]<[HLA] and IC₅₀≧[HLA], the measured IC₅₀ values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is calculated for each peptide by dividing the IC₅₀ of a positive control for inhibition by the IC₅₀ for each tested peptide (typically unlabeled versions of the radiolabeled probe peptide). For database purposes, and inter-experiment comparisons, relative binding values are compiled. These values can subsequently be converted back into IC₅₀ nM values by dividing the IC₅₀ nM of the positive controls for inhibition by the relative binding of the peptide of interest. This method of data compilation has proven to be the most accurate and consistent for comparing peptides that have been tested on different days, or with different lots of purified MHC.

Binding assays as outlined above may be used to analyze supermotif and/or motif-bearing epitopes as, for example, described in Example 2.

### Example 2

### Identification of HPV HLA Supermotif- and Motif-Bearing CTL Candidate Epitopes

Vaccine compositions of the invention can include multiple epitopes that comprise multiple HLA supermotifs or motifs to achieve broad population coverage. This example illustrates the identification of supermotif- and motif-bearing epitopes for the inclusion in such a vaccine composition. Calculation of population coverage was performed using the strategy described below.

Computer Searches and Algorithms for Identification of Supermotif and/or Motif-Bearing Epitopes

The searches performed to identify the motif-bearing peptide sequences in Examples 2 and 5 employed the protein sequence data from seven proteins (E1, E2, E5, E6, E7, L1 and L2) (see, Table 11, below) obtained from HPV types 6a, 6b, 11a, 16, 18, 31, 33, 45, 52, 56, and 58 (see, Table 12, below).

TABLE 11
Accession Nos. for Individual Proteins According to HPV Type

| | E1 | E2 | E4 | E5 | E5a | E5b | E6 | E7 | L1 | L2 |
|---|---|---|---|---|---|---|---|---|---|---|
| 6a | Q84293 | Q84294 | Q84295 | N/A | Q84296 | N/A | Q84291 | Q84929 | P03100 | Q84297 |
| | AAA74213 | AAA74214 | AAA74215 | | AAA74216 | | AAA74211 | AAA74212 | AAA74218 | |
| 6b | P03113 | P03119 | CAA25022 | N/A | P06460 | P06461 | P06462 | P06464 | P03100 | P03106 |
| | CAA25020 | CAA25021 | W4WL6 | | CAA25023 | CAA25024 | CAA25018 | CAA25019 | CAA25026 | CAA25025 |
| | W1WL6 | W2WL6 | | | W5WL6A | W5WLB | W6WL6 | W7WL6 | P1WL6 | P2WL6 |
| 11 | W1WL11 | AAA46930 | P04016 | N/A | W5WL11 | W5WL1B | W6WL11 | AAA46928 | P04012 | P2WL11 |
| | P04014 | W2WLI1 | W4WL11 | | P04017 | P04018 | P04019 | AAA21704 | P1WL11 | AAA46934 |
| | AAA46929 | P04015 | AAA46931 | | AAA46932 | AAA46933 | AAA21703 | W7WL11 | AAA4635 | P040I3 |
| | | | | | | | AAA46927 | P04020 | | |
| 16 | W1SLHS | W2WLHS | N/A | W5WLHS | N/A | N/A | W6WLHS | W7WLHS | AAD33259 | AAD33258 |
| 18 | W1WL18 | WL18 | N/A | W5WL18 | N/A | N/A | W6WL18 | PO6788 | CAA28671 | P2WL18 |
| 31 | W1WL31 | W2WL3 | N/A | W5WL31 | N/A | N/A | W6WL31 | W7WL31 | P1WL31 | P2WL31 |
| 33 | W1WL33 | W2WL33 | N/A | W5WL33 | N/A | N/A | W6WL33 | W7WL33 | P1WL33 | P2WL33 |
| 45 | S36563 | S36564 | N/A | N/A | N/A | N/A | CAB44706 | CAB44707 | CAB44705 | S36565 |
| 56 | N/A | S36581 | N/A | N/A | N/A | N/A | W6WL56 | S36580 | S38563 | S36582 |

TABLE 12
Accession Nos. for Entire HPV Sequence According to HPV Type

| HPV Type | Accession No. |
|---|---|
| 6a | X00203 |
| 6b | X00203 |
| 11a | M14119 |
| 16 | K02718 |
| 18 | X05015 |
| 31 | J04353 |
| 33 | M12732 |
| 45 | X74479 |
| 52 | X74481 |
| 56 | X74483 |
| 58 | D90400 |

Computer searches for epitopes bearing HLA Class I or Class II supermotifs or motifs were performed as follows. All translated HPV protein sequences were analyzed using a text string search software program, e.g., MotifSearch 1.4 (D. Brown, San Diego) to identify potential peptide sequences containing appropriate HLA binding motifs; alternative programs are readily produced in accordance with information in the art in view of the motif/supermotif disclosure herein. Furthermore, such calculations can be made mentally.

Identified HLA-A1, -A2, -A3, -A11, A24, -B7, -B44, and -DR supermotif sequences were scored using polynomial algorithms to predict their capacity to bind to specific HLA-Class I or Class II molecules. These polynomial algorithms take into account both extended and refined motifs (that is, to account for the impact of different amino acids at different positions), and are essentially based on the premise that the overall affinity (or ΔG) of peptide-HLA molecule interactions can be approximated as a linear polynomial function of the type:
“ΔG”=aₗᵢ×a₂ᵢ×a₃ᵢ . . . ×aₙᵢ

where aⱼᵢ is a coefficient which represents the effect of the presence of a given amino acid (i) at a given position (i) along the sequence of a peptide of n amino acids. The crucial assumption of this method is that the effects at each position are essentially independent of each other (i.e., independent binding of individual side-chains). When residue j occurs at position i in the peptide, it is assumed to contribute a constant amount jᵢ to the free energy of binding of the peptide irrespective of the sequence of the rest of the peptide. This assumption is justified by studies from our laboratories that demonstrated that peptides are bound to MHC and recognized by T cells in essentially an extended conformation.

The method of derivation of specific algorithm coefficients has been described in Gulukota, et al., J. Mol. Biol. 267:1258-67, 1997; (see also Sidney, J., et al., Human Immunol. 45:79-93, 1996; and Southwood, S., et al., J. Immunol. 160:3363-3373 (1998)). Briefly, for all i positions, anchor and non-anchor alike, the geometric mean of the average relative binding (ARB) of all peptides carrying j is calculated relative to the remainder of the group, and used as the estimate of jᵢ. For Class II peptides, if multiple alignments are possible, only the highest scoring alignment is utilized, following an iterative procedure. To calculate an algorithm score of a given peptide in a test set, the ARB values corresponding to the sequence of the peptide are multiplied. If this product exceeds a chosen threshold, the peptide is predicted to bind. Appropriate thresholds are chosen as a function of the degree of stringency of prediction desired.

Selection of HLA-A2 Supertype Cross-Reactive Peptides

Complete protein sequences from the seven HPV structural and regulatory proteins of the HPV strains listed above were aligned, then scanned, utilizing motif identification software, to identify 9- and 10-mer sequences containing the HLA-A2-supermotif main anchor specificity.

HLA-A2 supermotif-bearing sequences are shown in Tables 15 and 16. Typically, these sequences are then scored using the A2 algorithm and the peptides corresponding to the positive-scoring sequences are synthesized and tested for their capacity to bind purified HLA-A*0201 molecules in vitro (HLA-A*0201 is considered a prototype A2 supertype molecule).

Examples of peptides that bind to HLA-A*0201 with IC₅₀ values ≦500 nM are shown in Tables 15-16. Peptides that bind to at least three of the five A2-supertype alleles tested are typically deemed A2-supertype cross-reactive binders. Preferred peptides bind at an affinity equal to or less than 500 nM to three or more HLA-A2 supertype molecules.

Selection of HLA-A3 Supermotif-Bearing Epitopes

The HPV protein sequences scanned above were also examined for the presence of peptides with the HLA-A3-supermotif primary anchors. Peptides corresponding to the supermotif-bearing sequences are then synthesized and tested for binding to HLA-A*0301 and HLA-A*1101 molecules, the two most prevalent A3-supertype alleles. The peptides that are found to bind one of the two alleles with binding affinities of ≦500 nM, often ≦200 nM, are then tested for binding cross-reactivity to the other common A3-supertype alleles (e.g., A*3101, A*3301, and A*6801) to identify those that can bind at least three of the five HLA-A3-supertype molecules tested.

Selection of HLA-B7 Supermotif Bearing Epitopes

The same HPV target antigen protein sequences were also analyzed for the presence of 9- or 10-mer peptides with the HLA-B7-supermotif. Corresponding peptides are synthesized and tested for binding to HLA-B*0702, the most common B7-supertype allele (i.e., the prototype B7 supertype allele). Peptides binding B*0702 with IC₅₀ of ≦500 nM are identified using standard methods. These peptides are then tested for binding to other common B7-supertype molecules (e.g., B*3501, B*5101, B*5301, and B*5401). Peptides capable of binding to three or more of the five B7-supertype alleles tested are thereby identified.

Selection of A1 and A24 Motif-Bearing Epitopes

To further increase population coverage, HLA-A1 and -A24 epitopes can, for example, also be incorporated into potential vaccine constructs. An analysis of the protein sequence data from the HPV target antigens utilized above can also be performed to identify HLA-A1- and A24-motif-containing sequences.

High affinity and/or cross-reactive binding epitopes that bear other motif and/or supermotifs are identified using analogous methodology.

### Example 3

### Confirmation of Immunogenicity

Cross-reactive candidate CTL A2-supermotif-bearing peptides that are identified as described in Example 2 were selected for in vitro immunogenicity testing. Testing was performed using the following methodology.

Target Cell Lines for Cellular Screening:

The .221A2.1 cell line, produced by transferring the HLA-A2.1 gene into the HLA-A, -B, -C null mutant human B-lymphoblastoid cell line 721.221, is used as the peptide-loaded target to measure activity of HLA-A2.1-restricted CTL. This cell line is grown in RPMI-1640 medium supplemented with antibiotics, sodium pyruvate, nonessential amino acids and 10% (v/v) heat inactivated FCS. Cells that express an antigen of interest, or transfectants comprising the gene encoding the antigen of interest, can be used as target cells to test the ability of peptide-specific CTLs to recognize endogenous antigen.

Primary CTL Induction Cultures:

Generation of Dendritic Cells (DC): PBMCs are thawed in RPMI with 30 μg/ml DNAse, washed twice and resuspended in complete medium (RPMI-1640 plus 5% AB human serum, non-essential amino acids, sodium pyruvate, L-glutamine and penicillin/strpetomycin). The monocytes are purified by plating 10×10⁶ PBMC/well in a 6-well plate. After 2 hours at 37° C., the non-adherent cells are removed by gently shaking the plates and aspirating the supernatants. The wells are washed a total of three times with 3 ml RPMI to remove most of the non-adherent and loosely adherent cells. Three ml of complete medium containing 50 ng/ml of GM-CSF and 1,000 U/ml of IL-4 are then added to each well. TNFα is added to the DCs on day 6 at 75 ng/ml and the cells are used for CTL induction cultures on day 7.

Induction of CTL with DC and Peptide: CD8⁺ T-cells are isolated by positive selection with Dynal immunomagnetic beads (Dynabeads® M-450) and the detacha-bead® reagent. Typically about 200-250×10⁶ PBMC are processed to obtain 24×10⁶ CD8⁺ T-cells (enough for a 48-well plate culture). Briefly, the PBMCs are thawed in RPMI with 30 μg/ml DNAse, washed once with PBS containing 1% human AB serum and resuspended in PBS/1% AB serum at a concentration of 20×10⁶ cells/ml. The magnetic beads are washed 3 times with PBS/AB serum, added to the cells (140 μl beads/20×10⁶ cells) and incubated for 1 hour at 4° C. with continuous mixing. The beads and cells are washed 4× with PBS/AB serum to remove the non-adherent cells and resuspended at 100×10⁶ cells/ml (based on the original cell number) in PBS/AB serum containing 100 μl/ml detacha-bead® reagent and 30 kg/ml DNAse. The mixture is incubated for 1 hour at room temperature with continuous mixing. The beads are washed again with PBS/AB/DNAse to collect the CD8⁺ T-cells. The DC are collected and centrifuged at 1300 rpm for 5-7 minutes, washed once with PBS with 1% BSA, counted and pulsed with 40 μg/ml of peptide at a cell concentration of 1-2×10⁶/ml in the presence of 3 μg/ml β₂-microglobulin for 4 hours at 20° C. The DC are then irradiated (4,200 rads), washed 1 time with medium and counted again.

Setting up induction cultures: 0.25 ml cytokine-generated DC (at 1×10⁵ cells/ml) are co-cultured with 0.25 ml of CD8⁺ T-cells (at 2×10⁶ cell/ml) in each well of a 48-well plate in the presence of 10 ng/ml of IL-7. Recombinant human IL-10 is added the next day at a final concentration of 10 ng/ml and rhuman IL-2 is added 48 hours later at 10 IU/ml.

Restimulation of the induction cultures with peptide-pulsed adherent cells: Seven and fourteen days after the primary induction the cells are re-stimulated with peptide-pulsed adherent cells. The PBMCS are thawed and washed twice with RPMI and DNAse. The cells are resuspended at 5×10⁶ cells/ml and irradiated at approximately 4200 rads. The PBMCs are plated at 2×10⁶ in 0.5 ml complete medium per well and incubated for 2 hours at 37° C. The plates are washed twice with RPMI by tapping the plate gently to remove the non-adherent cells and the adherent cells pulsed with 10 μg/ml of peptide in the presence of 3 μg/ml 12 microglobulin in 0.25 ml RPMI/5% AB per well for 2 hours at 37° C. Peptide solution from each well is aspirated and the wells are washed once with RPMI. Most of the media is aspirated from the induction cultures (CD8⁺ cells) and brought to 0.5 ml with fresh media. The cells are then transferred to the wells containing the peptide-pulsed adherent cells. Twenty four hours later rhuman IL-10 is added at a final concentration of 10 ng/ml and rhuman IL-2 is added the next day and again 2-3 days later at 50 IU/ml (Tsai, et al., Crit. Rev. Immunol. 18(1-2):65-75, 1998). Seven days later the cultures are assayed for CTL activity in a ⁵¹Cr release assay. In some experiments the cultures are assayed for peptide-specific recognition in the in situ IFNγ ELISA at the time of the second restimulation followed by assay of endogenous recognition 7 days later. After expansion, activity is measured in both assays for a side by side comparison.

Measurement of CTL Lytic Activity by ⁵¹Cr Release:

Seven days after the second restimulation, cytotoxicity is determined in a standard (5 hr) ⁵¹Cr release assay by assaying individual wells at a single E:T. Peptide-pulsed targets are prepared by incubating the cells with 10 μg/ml peptide overnight at 37° C.

Adherent target cells are removed from culture flasks with trypsin-EDTA. Target cells are labeled with 200 μCi of ⁵¹Cr sodium chromate (Dupont, Wilmington, Del.) for 1 hour at 37° C. Labeled target cells are resuspended at 106 per ml and diluted 1:10 with K562 cells at a concentration of 3.3×10⁶/ml (an NK-sensitive erythroblastoma cell line used to reduce non-specific lysis). Target cells (100 μl) and 100 μl of effectors are plated in 96 well round-bottom plates and incubated for 5 hours at 37° C. At that time, 100 μl of supernatant are collected from each well and percent lysis is determined according to the formula: [(cpm of the test sample−cpm of the spontaneous ⁵¹Cr release sample)/(cpm of the maximal ⁵¹Cr release sample−cpm of the spontaneous ⁵¹Cr release sample)]×100. Maximum and spontaneous release are determined by incubating the labeled targets with 1% Triton X-100 and media alone, respectively. A positive culture is defined as one in which the specific lysis (sample-background) is 10% or higher in the case of individual wells and is 15% or more at the 2 highest E:T ratios when expanded cultures are assayed.

In Situ Measurement of Human IFNγ Production as an Indicator of Peptide-Specific and Endogenous Recognition:

Immulon 2 plates are coated with mouse anti-human IFNγ monoclonal antibody (4 μg/ml 0.1M NaHCO₃, pH8.2) overnight at 4° C. The plates are washed with Ca²⁺, Mg²⁺-free PBS/0.05% Tween 20 and blocked with PBS/10% FCS for 2 hours, after which the CTLs (100 μl/well) and targets (100 μl/well) are added to each well, leaving empty wells for the standards and blanks (which received media only). The target cells, either peptide-pulsed or endogenous targets, are used at a concentration of 1×10⁶ cells/ml. The plates are incubated for 48 hours at 37° C. with 5% CO₂.

Recombinant human IFNγ is added to the standard wells starting at 400 pg or 1200 pg/100 μl/well and the plate incubated for 2 hours at 37° C. The plates are washed and 100 μl of biotinylated mouse anti-human IFNγ monoclonal antibody (2 μg/ml in PBS/3% FCS/0.05% Tween 20) are added and incubated for 2 hours at room temperature. After washing again, 100 μl HRP-streptavidin (1:4000) are added and the plates incubated for 1 hour at room temperature. The plates are then washed 6 times with wash buffer, 100 μl/well developing solution (TMB 1:1) are added, and the plates allowed to develop for 5-15 minutes. The reaction is stopped with 50 μl/well 1M H₃PO₄ and read at OD₄₅₀. A culture is considered positive if it measured at least 50 pg of IFNγ/well above background and is twice the background level of expression.

Those cultures that demonstrate specific lytic activity against peptide-pulsed targets and/or tumor targets are expanded over a two week period with anti-CD3. Briefly, 5×10⁴ CD8⁺ cells are added to a T25 flask containing the following: 1×10⁶ irradiated (4,200 rad) PBMC (autologous or allogeneic) per ml, 2×10⁵ irradiated (8,000 rad) EBV-transformed cells per ml, and OKT3 (anti-CD3) at 30 ng per ml in RPMI-1640 containing 10% (v/v) human AB serum, non-essential amino acids, sodium pyruvate, 25 μM 2-mercaptoethanol, L-glutamine and penicillin/streptomycin. Rhuman IL2 is added 24 hours later at a final concentration of 200 IU/ml and every 3 days thereafter with fresh media at 50 IU/ml. The cells are split if the cell concentration exceeded 1×10⁶/ml and the cultures are assayed between days 13 and 15 at E:T ratios of 30, 10, 3 and 1:1 in the ⁵¹Cr release assay or at 1×10⁶/ml in the in situ IFNγ assay using the same targets as before the expansion.

Cultures are expanded in the absence of anti-CD3⁺ as follows. Those cultures that demonstrate specific lytic activity against peptide and endogenous targets are selected and 5×10⁴ CD8⁺ cells are added to a T25 flask containing the following: 1×10⁶ autologous PBMC per ml which have been peptide-pulsed with 10 μg/ml peptide for 2 hours at 37° C. and irradiated (4,200 rad); 2×10⁵ irradiated (8,000 rad) EBV-transformed cells per ml RPMI-1640 containing 10% (v/v) human AB serum, non-essential AA, sodium pyruvate, 25 mM 2-mercaptoethanol, L-glutamine and gentamicin.

Evaluation of Immunogenicity:

Immunogenicity of HLA-A1 Motif-Bearing Peptides

HLA-A1 motif cross-reactive binding peptides are tested in the cellular assay for the ability to induce peptide-specific CTL in normal individuals. In this analysis, a peptide is typically considered to be an epitope if it induces peptide-specific CTLs in at least 2 donors (unless otherwise noted) and preferably, also recognizes the endogenously expressed peptide. See, Table 31. The data presented in Table 31 summarize such an analysis of the recognition of HLA-A1-restricted peptides by PBL isolated from HLA-A1 positive individuals. In the Table, the sequence of each peptide analyzed is presented in the first column (labeled “Sequence”). The unique sequence identifier assigned to each peptide is presented in the second column (labeled “SEQ ID NO”). The viral type and antigenic origin of each peptide is provided in the third column (labeled “Source”). In this column, the viral type is provided as the first component of each entry and the antigenic origin is provided as the second component of each entry. The third component of each entry indicates the position within the antigen of the N-terminal amino acid residue of the peptide epitope. A fourth component is present for analog peptide epitopes. If present, this component of each entry indicates the position and substituted amino acid residue for each analog peptide epitope. The fourth and fifth columns are collectively labeled “+donors/total.” Column four provides the data for the peptide being examined. If the peptide is an analog, then column five provides the data for the corresponding wild type (i.e., naturally occurring or non-analoged) peptide. In each column, the number to the left of the slash represents the number of donors for which an immunogenic response was observed, while the number to the right of the slash represents the number of donors tested. The sixth and seventh columns are collectively labeled “Positive wells/total tested.” In each column, the number to the left of the slash represents the number of positive wells in the immunogenicity assay described above, while the number to the right of the slash represents total number of wells tested. The eighth and ninth columns are collectively labeled “Stimulation index.” In each column, the amount of IFNγ released in the positive well is compared to the amount released in a control well. In cases where multiple wells are positive, the mean value of the positive wells is calculated. The amount of IFNγ released in the positive well is expressed as the number of times over the background level of γ released (i.e., in the control well). Values of the actual peptides recited in the Table are provided in the column labeled “Peptide,” whereas values of the wild type peptides corresponding to analog peptides recited in the Table are provided in the column labeled “WT.” The tenth and eleventh columns are collectively labeled “Net IFNγ release (pg/well).” Values of IFNγ released in each positive well for each peptide recited in the Table are provided in the column labeled “Peptide.” In cases where multiple wells are positive, the mean value of the positive wells is calculated. Values of the actual peptides recited in the Table are provided in the column labeled “Peptide,” whereas values of the wild type peptides corresponding to analog peptides recited in the Table are provided in the column labeled “WT.”

Thus, for example, the first entry on Table 31 indicates that the peptide comprising the sequence ITDIILECVY (first column) (SEQ ID NO:______; second column): (third column) was obtained from the E6 protein of HPV-16 beginning at position 30; (third column) is an analog peptide with a threonine substitution at position 2; (fourth column) exhibited a positive immunogenic response in PBL isolated from 1 out of 5 HLA-A1 positive donors; (fifth column) whereas the wild type peptide corresponding to the peptide recited in the Table failed to exhibit a positive immunogenic response in PBL isolated from any of 5 HLA-A1 positive donors; (sixth column) exhibited a positive response in 1 out of 234 wells tested in the immunogenicity assay described above; (seventh column) whereas the corresponding wild type peptide exhibited a positive response in zero out of one wells tested; (eighth column) the amount of IFNγ detected was 8 times that detected in a control well; (ninth column) whereas the stimulation index of the corresponding wild type peptide was not tested; (tenth column) the positive well produced 103 pg of IFNγ; (eleventh column) whereas there was no IFNγ produced in the well of the corresponding wild type peptide.

Immunogenicity is additionally confirmed using PBMCs isolated from HPV-infected patients. Briefly, PBMCs are isolated from patients, re-stimulated with peptide-pulsed monocytes and assayed for the ability to recognize peptide-pulsed target cells as well as transfected cells endogenously expressing the antigen.

Immunogenicity of HLA-A2 Supermotif-Bearing Peptides

A2-supermotif cross-reactive binding peptides are tested in the cellular assay for the ability to induce peptide-specific CTL in normal individuals. In this analysis, a peptide is typically considered to be an epitope if it induces peptide-specific CTLs in at least 2 donors (unless otherwise noted) and preferably, also recognizes the endogenously expressed peptide.

Immunogenicity is additionally confirmed using PBMCs isolated from HPV-infected patients. Briefly, PBMCs are isolated from patients, re-stimulated with peptide-pulsed monocytes and assayed for the ability to recognize peptide-pulsed target cells as well as transfected cells endogenously expressing the antigen.

Immunogenicity of HLA-A*03/A11 Supermotif-Bearing Peptides

HLA-A3 supermotif-bearing cross-reactive binding peptides are also evaluated for immunogenicity using methodology analogous for that used to evaluate the immunogenicity of the HLA-A2 supermotif peptides. See, Table 32. The data presented in Table 32 summarize such an analysis of the recognition of HLA-A3-restricted peptides by PBL isolated from HLA-A3 positive individuals. The contents of each column are as described above for the HLA-A1 analysis, with the exception that, in Table 32, the first column (labeled “Epimmune ID”) refers to a peptide identification system utilized by the inventors.

Immunogenicity of HLA-A24 Supermotif-Bearing Peptides

HLA-A24 motif-bearing cross-reactive binding peptides are also evaluated for immunogenicity using methodology analogous for that used to evaluate the immunogenicity of the HLA-A24 motif peptides. See, Table 33. The data presented in Table 33 summarize such an analysis of the recognition of HLA-A24-restricted peptides by PBL isolated from HLA-A24 positive individuals. The contents of each column are as described above for the HLA-A24 analysis.

Immunogenicity of HLA-B7 Supermotif-Bearing Peptides

Immunogenicity screening of the B7-supertype cross-reactive binding peptides identified in Example 2 are evaluated in a manner analogous to the evaluation of HLA-A2- and A3-supermotif-bearing peptides.

### Example 4

### Implementation of the Extended Supermotif to Improve the Binding Capacity of Native Epitopes by Creating Analogs

HLA motifs and supermotifs (comprising primary and/or secondary residues) are useful in the identification and preparation of highly cross-reactive native peptides, as demonstrated herein. Moreover, the definition of HLA motifs and supermotifs also allows one to engineer highly cross-reactive epitopes by identifying residues within a native peptide sequence which can be analoged, or “fixed” to confer upon the peptide certain characteristics, e.g. greater cross-reactivity within the group of HLA molecules that comprise a supertype, and/or greater binding affinity for some or all of those HLA molecules. Examples of analoging peptides to exhibit modulated binding affinity are set forth in this example.

Analoging at Primary Anchor Residues

Peptide engineering strategies are implemented to further increase the cross-reactivity of the epitopes. For example, on the basis of the data disclosed, e.g., in related and co-pending U.S. patent application Ser. No. 09/226,775, the main anchors of A2-supermotif-bearing peptides are altered, for example, to introduce a preferred L, I, V, or M at position 2, and I or V at the C-terminus.

To analyze the cross-reactivity of the analog peptides, each engineered analog is initially tested for binding to the prototype A2 supertype allele A*0201, then, if A*0201 binding capacity is maintained, for A2-supertype cross-reactivity.

Alternatively, a peptide is tested for binding to one or all supertype members and then analoged to modulate binding affinity to any one (or more) of the supertype members to add population coverage.

The selection of analogs for immunogenicity in a cellular screening analysis is typically further restricted by the capacity of the parent peptide to bind at least weakly, i.e., bind at an IC₅₀ of 5000 nM or less, to three of more A2 supertype alleles. The rationale for this requirement is that the naturally-occurring peptides must be present endogenously in sufficient quantity to be biologically relevant. Analoged peptides have been shown to have increased immunogenicity and cross-reactivity by T cells specific for the parent epitope (see, e.g., Parkhurst, et al., J. Immunol. 157:2539, 1996; and Pogue, et al., Proc. Natl. Acad. Sci. U.S.A. 92:8166, 1995).

In the cellular screening of these peptide analogs, it is important to demonstrate that analog-specific CTLs are also able to recognize the wild-type peptide and, when possible, target cells that endogenously express the epitope.

Analoging of HLA-A3 and B7-Supermotif-Bearing Peptides

Analogs of HLA-A3 supermotif-bearing epitopes are generated using strategies similar to those employed in analoging HLA-A2 supermotif-bearing peptides. For example, peptides binding to 3/5 of the A3-supertype molecules are engineered at primary anchor residues to possess a preferred residue (V, S, M, or A) at position 2.

The analog peptides are then tested for the ability to bind A*03 and A*11 (prototype A3 supertype alleles). Those peptides that demonstrate ≦500 nM binding capacity are then tested for A3-supertype cross-reactivity.

Similarly to the A2- and A3-motif bearing peptides, peptides binding 3 or more B7-supertype alleles can be improved, where possible, to achieve increased cross-reactive binding. B7 supermotif-bearing peptides are, for example, engineered to possess a preferred residue (V, I, L, or F) at the C-terminal primary anchor position, as demonstrated by Sidney, J., et al. (J. Immunol. 157:3480-3490, 1996).

Analoging at primary anchor residues of other motif and/or supermotif-bearing epitopes is performed in a like manner.

The analog peptides are then be tested for immunogenicity, typically in a cellular screening assay. Again, it is generally important to demonstrate that analog-specific CTLs are also able to recognize the wild-type peptide and, when possible, targets that endogenously express the epitope.

Analoging at Secondary Anchor Residues

Moreover, HLA supermotifs are of value in engineering highly cross-reactive peptides and/or peptides that bind HLA molecules with increased affinity by identifying particular residues at secondary anchor positions that are associated with such properties. For example, the binding capacity of a B7 supermotif-bearing peptide with an F residue at postion 1 is analyzed. The peptide is then analoged to, for example, substitute L for F at position 1. The analoged peptide is evaluated for increased binding affinity/and or increased cross-reactivity. Such a procedure identifies analoged peptides with modulated binding affinity.

Engineered analogs with sufficiently improved binding capacity or cross-reactivity can also be tested for immunogenicity in HLA-B7-transgenic mice, following for example, IFA immunization or lipopeptide immunization. Analoged peptides are additionally tested for the ability to stimulate a recall response using PBMC from HPV-infected patients.

Other Analoging Strategies

Another form of peptide analoging, unrelated to the anchor positions, involves the substitution of a cysteine with α-amino butyric acid. Due to its chemical nature, cysteine has the propensity to form disulfide bridges and sufficiently alter the peptide structurally so as to reduce binding capacity. Substitution of α-amino-butyric acid for cysteine not only alleviates this problem, but has been shown to improve binding and crossbinding capabilities in some instances (see, e.g., the review by Sette, et al., In: Persistent Viral Infections, Eds. R. Ahmed and I. Chen, John Wiley & Sons, England, 1999).

Thus, by the use of even single amino acid substitutions, the binding affinity and/or cross-reactivity of peptide ligands for HLA supertype molecules can be modulated.

### Example 5

### Identification of HPV-Derived Sequences with HLA-DR Binding Motifs

Peptide epitopes bearing an HLA class II supermotif or motif are identified as outlined below using methodology similar to that described in Examples 1-3.

Selection of HLA-DR-Supermotif-Bearing Epitopes.

To identify HPV-derived, HLA class II HTL epitopes, the protein sequences from the same HPV antigens used for the identification of HLA Class I supermotif/motif sequences were analyzed for the presence of sequences bearing an HLA-DR-motif or supermotif. Specifically, 15-mer sequences were selected comprising a DR-supermotif, further comprising a 9-mer core, and three-residue N- and C-terminal flanking regions (15 amino acids total).

Protocols for predicting peptide binding to DR molecules have been developed (Southwood, et al. J. Immunology 160:3363-3373 (1998)). These protocols, specific for individual DR molecules, allow the scoring, and ranking, of 9-mer core regions. Each protocol not only scores peptide sequences for the presence of DR-supermotif primary anchors (i.e., at position 1 and position 6) within a 9-mer core, but additionally evaluates sequences for the presence of secondary anchors. Using allele specific selection tables (see, e.g., Southwood, et al. J. Immunology 160:3363-3373 (1998)), it has been found that the same protocols efficiently select peptide sequences with a high probability of binding a particular DR molecule. Additionally, it has been found that performing these protocols in tandem, specifically those for DR1, DR4w4, and DR7, can efficiently select DR cross-reactive peptides.

The HPV-derived peptides identified above are tested for their binding capacity for various common HLA-DR molecules. All peptides are initially tested for binding to the DR molecules in the primary panel: DR1, DR4w4, and DR7. Peptides binding at least 2 of these 3 DR molecules are then tested for binding to DR2w2 β1, DR2w2 β2, DR6w19, and DR9 molecules in secondary assays. Finally, peptides binding at least 2 of the 4 secondary panel DR molecules, and thus cumulatively at least 4 of 7 different DR molecules, are screened for binding to DR4w15, DR5w11, and DR8w2 molecules in tertiary assays. Peptides binding at least 7 of the 10 DR molecules comprising the primary, secondary, and tertiary screening assays are considered cross-reactive DR binders. HPV-derived peptides found to bind common HLA-DR alleles are of particular interest.

Selection of DR3 Motif Peptides

Because HLA-DR3 is an allele that is prevalent in Caucasian, Black, and Hispanic populations, DR3 binding capacity is an important criterion in the selection of HTL epitopes. However, data generated previously indicated that DR3 only rarely cross-reacts with other DR alleles (Sidney, J., et al., J. Immunol. 149:2634-2640, 1992; Geluk, et al., J. Immunol. 152:5742-48, 1994; Southwood, et al. J. Immunology 160:3363-3373 (1998)). This is not entirely surprising in that the DR3 peptide-binding motif appears to be distinct from the specificity of most other DR alleles. For maximum efficiency in developing vaccine candidates it would be desirable for DR3 motifs to be clustered in proximity with DR supermotif regions. Thus, peptides shown to be candidates may also be assayed for their DR3 binding capacity. However, in view of the distinct binding specificity of the DR3 motif, peptides binding only to DR3 can also be considered as candidates for inclusion in a vaccine formulation.

To efficiently identify peptides that bind DR3, target HPV antigens are analyzed for sequences carrying one of the two DR3 specific binding motifs reported by Geluk, et al. (J. Immunol. 152:5742-48, 1994). The corresponding peptides are then synthesized and tested for the ability to bind DR3 with an affinity of 1 μM or better, i.e., less than 1 μM. Peptides are found that meet this binding criterion and qualify as HLA class II high affinity binders.

DR3 binding epitopes identified in this manner are included in vaccine compositions with DR supermotif-bearing peptide epitopes.

Similarly to the case of HLA class I motif-bearing peptides, the class II motif-bearing peptides are analoged to improve affinity or cross-reactivity. For example, aspartic acid at position 4 of the 9-mer core sequence is an optimal residue for DR3 binding, and substitution for that residue often improves DR 3 binding.

### Example 6

### Immunogenicity of HPV-Derived HTL Epitopes

This example determines immunogenic DR supermotif- and DR3 motif-bearing epitopes among those identified using the methodology in Example 5.

Immunogenicity of HTL epitopes are evaluated in a manner analogous to the determination of immunogenicity of CTL epitopes by assessing the ability to stimulate HTL responses and/or by using appropriate transgenic mouse models. Immunogenicity is determined by screening for: 1.) in vitro primary induction using normal PBMC or 2.) recall responses from human PBMCs.

### Example 7

### Calculation of Phenotypic Frequencies of HLA-Supertypes in Various Ethnic Backgrounds to Determine Breadth of Population Coverage

This example illustrates the assessment of the breadth of population coverage of a vaccine composition comprised of multiple epitopes comprising multiple supermotifs and/or motifs.

In order to analyze population coverage, gene frequencies of HLA alleles were determined. Gene frequencies for each HLA allele were calculated from antigen or allele frequencies utilizing the binomial distribution formulae gf=1−(SQRT(1−af)) (see, e.g., Sidney, J., et al., Human Immunol. 45:79-93, 1996). To obtain overall phenotypic frequencies, cumulative gene frequencies were calculated, and the cumulative antigen frequencies derived by the use of the inverse formula [af=1−(1−Cgf)²].

Where frequency data was not available at the level of DNA typing, correspondence to the serologically defined antigen frequencies was assumed. To obtain total potential supertype population coverage no linkage disequilibrium was assumed, and only alleles confirmed to belong to each of the supertypes were included (minimal estimates). Estimates of total potential coverage achieved by inter-loci combinations were made by adding to the A coverage the proportion of the non-A covered population that could be expected to be covered by the B alleles considered (e.g., total=A+B*(1−A)). Confirmed members of the A3-like supertype are A3, A11, A31, A*3301, and A*6801. Although the A3-like supertype may also include A34, A66, and A*7401, these alleles were not included in overall frequency calculations. Likewise, confirmed members of the A2-like supertype family are A*0201, A*0202, A*0203, A*0204, A*0205, A*0206, A*0207, A*6802, and A*6901. Finally, the B7-like supertype-confirmed alleles are: B7, B*3501-03, B51, B*5301, B*5401, B*5501-2, B*5601, B*6701, and B*7801 (potentially also B*1401, B*3504-06, B*4201, and B*5602).

Population coverage achieved by combining the A2-, A3- and B7-supertypes is approximately 86% in five major ethnic groups, supra. Coverage may be extended by including peptides bearing the A1 and A24 motifs. On average, A1 is present in 12% and A24 in 29% of the population across five different major ethnic groups (Caucasian, North American Black, Chinese, Japanese, and Hispanic). Together, these alleles are represented with an average frequency of 39% in these same ethnic populations. The total coverage across the major ethnicities when A1 and A24 are combined with the coverage of the A2-, A3- and B7-supertype alleles is >95%. An analogous approach can be used to estimate population coverage achieved with combinations of class II motif-bearing epitopes.

Immunogenicity studies in humans (e.g., Bertoni, et al., J. Clin. Invest. 100:503, 1997; Doolan, et al., Immunity 7:97, 1997; and Threlkeld, et al., J. Immunol. 159:1648, 1997) have shown that highly cross-reactive binding peptides are almost always recognized as epitopes. The use of highly cross-reactive binding peptides is an important selection criterion in identifying candidate epitopes for inclusion in a vaccine that is immunogenic in a diverse population.

With a sufficient number of epitopes (as disclosed herein and from the art), an average population coverage is predicted to be greater than 95% in each of five major ethnic populations. The game theory Monte Carlo simulation analysis, which is known in the art (see, e.g., Osborne, M. J. and Rubinstein, A., A course in game theory, MIT Press, 1994), can be used to estimate what percentage of the individuals in a population comprised of the Caucasian, North American Black, Japanese, Chinese, and Hispanic ethnic groups would recognize the vaccine epitopes described herein. A preferred percentage is 90%. A more preferred percentage is 90%, 91%, 92%, 93%, 94%, 95%, 96%, 97%, 98%, 99% or 100%.

### Example 8

### CTL Recognition of Endogenous Processed Antigens after Priming

This example determines that CTL induced by native or analoged peptide epitopes identified and selected as described in Examples 1-5 recognize endogenously synthesized, i.e., native antigens.

Effector cells isolated from transgenic mice that are immunized with peptide epitopes as in Example 3, for example HLA-A2 supermotif-bearing epitopes, are re-stimulated in vitro using peptide-coated stimulator cells. Six days later, effector cells are assayed for cytotoxicity and the cell lines that contain peptide-specific cytotoxic activity are further re-stimulated. An additional six days later, these cell lines are tested for cytotoxic activity on ⁵¹Cr labeled Jurkat-A2.1/K^{b} target cells in the absence or presence of peptide, and also tested on ⁵¹Cr labeled target cells bearing the endogenously synthesized antigen, i.e. cells that are stably transfected with HPV expression vectors.

Alternatively, appropriate processing and presentation of epitopes derived from either the full-length HPV genes may be demonstrated using an in vitro assay. Jurkat cells expressing the HLA-A*0201 are transfected by lipofection with a construct encoding the HPV gene of interest. The coding regions may be subcloned into the replicating pCEI episomal vector. For transfection, 200 μl of cells are incubated for 4 hours at 37 degrees C. with a mixture of 4 μg of DNA and 6 μg of DMRIE-C (Invitrogen, Carlsbad, Calif.). Lipofected cells are then grown in RPMI-1640 containing 15% FBS, 1 μg/ml PHA, and 50 ng/ml PMA. Transient transfectants are assayed 24 to 48 hours after transfection.

High-affinity peptide epitope-specific CTL lines are generated from splenocytes of HLA-A*0201/K^{b} or HLA-A*1101/K^{b} transgenic mice previously immunized with peptide epitopes or DNA encoding them. Splenocytes are stimulated in vitro with 0.1 μg/ml peptide using LPS blasts as feeders and antigen-presenting cells (APC). Ten days after the initial stimulation, and weekly thereafter, cells are restimulated with LPS blasts pulsed for 1 hour with 0.1 μg/ml peptide. CTL lines are then used in assays 5 days following restimulation.

Epitope peptide-pulsed Jurkat target cells are used to establish the activity of CTL lines. Set numbers of CTLs (1-4×10⁵) are incubated with 10⁵ Jurkat cells pulsed with decreasing concentrations of peptide, 1-10 μg/ml. The amount of IFN-γ generated by the CTL lines upon recognition of the target cells pulsed with peptide is measured using the in situ ELISA and, when needed, to establish a standard curve. The same CTL lines are used to demonstrate processing and presentation of selected epitopes by the transfected cells.

The results of either approach will demonstrate that CTL lines obtained from animals primed with peptide epitope recognize endogenously synthesized HPV antigen. The choice of transgenic mouse model to be used for such an analysis depends upon the epitope(s) that is being evaluated. In addition to HLA-A*0201/Kb transgenic mice, several other transgenic mouse models including mice with human A11, which may also be used to evaluate A3 epitopes, and B7 alleles have been characterized and others (e.g., transgenic mice for HLA-A1 and A24) are being developed. HLA-DR1 and HLA-DR3 mouse models have also been developed, which may be used to evaluate HTL epitopes.

### Example 9

### Activity of CTL-HTL Conjugated Epitopes in Transgenic Mice

This example illustrates the induction of CTLs and HTLs in transgenic mice by use of an HPV antigen CTL/HTL peptide conjugate whereby the vaccine composition comprises peptides to be administered to an HPV-infected patient. The peptide composition can comprise multiple CTL and/or HTL epitopes and further, can comprise epitopes selected from multiple HPV target antigens. The epitopes are identified using methodology as described in Examples 1-5. The analysis demonstrates the enhanced immunogenicity that can be achieved by inclusion of one or more HTL epitopes in a vaccine composition. Such a peptide composition can comprise an HTL epitope conjugated to a preferred CTL epitope containing, for example, at least one CTL epitope that binds to multiple HLA family members at an affinity of 500 nM or less, or analogs of that epitope. The peptides may be lipidated, if desired.

Immunization procedures: Immunization of transgenic mice is performed as described (Alexander, et al., J. Immunol. 159:4753-4761, 1997). For example, A2/K^{b} mice, which are transgenic for the human HLA A2.1 allele and are useful for the assessment of the immunogenicity of HLA-A*0201 motif- or HLA-A2 supermotif-bearing epitopes, are primed subcutaneously (base of the tail) with a 0.1 ml of peptide in Incomplete Freund's Adjuvant, or if the peptide composition is a lipidated CTL/HTL conjugate, in DMSO/saline or if the peptide composition is a polypeptide, in PBS or Incomplete Freund's Adjuvant. Seven days after priming, splenocytes obtained from these animals are re-stimulated with syngenic irradiated LPS-activated lymphoblasts coated with peptide.

Cell lines: Target cells for peptide-specific cytotoxicity assays are Jurkat cells transfected with the HLA-A2.1/K^{b} chimeric gene (e.g., Vitiello, et al., J. Exp. Med. 173:1007, 1991)

In vitro CTL activation: One week after priming, spleen cells (30×10⁶ cells/flask) are co-cultured at 37° C. with syngeneic, irradiated (3000 rads), peptide coated lymphoblasts (10×10⁶ cells/flask) in 10 ml of culture medium/T25 flask. After six days, effector cells are harvested and assayed for cytotoxic activity.

Assays for Cytotoxic Activity:

Assay 1: Target cells (1.0 to 1.5×10⁶) are incubated at 37° C. in the presence of 200 μl of ⁵¹Cr. After 60 minutes, cells are washed three times and re-suspended in R10 medium. Peptide is added where required at a concentration of 1 μg/ml. For the assay, 10^{4 51}Cr-labeled target cells are added to different concentrations of effector cells (final volume of 200 μl) in U-bottom 96-well plates. After a 6 hour incubation period at 37° C., a 0.1 ml aliquot of supernatant is removed from each well and radioactivity is determined in a Micromedic automatic gamma counter. The percent specific lysis is determined by the formula: percent specific release=100×(experimental release−spontaneous release)/(maximum release−spontaneous release). To facilitate comparison between separate CTL assays run under the same conditions, % ⁵¹Cr release data is expressed as lytic units/10⁶ cells. One lytic unit is arbitrarily defined as the number of effector cells required to achieve 30% lysis of 10,000 target cells in a 6 hour ⁵¹Cr release assay. To obtain specific lytic units/10⁶, the lytic units/10⁶ obtained in the absence of peptide is subtracted from the lytic units/10⁶ obtained in the presence of peptide. For example, if 30% ⁵¹Cr release is obtained at the effector (E): target (T) ratio of 50:1 (i.e., 5×10⁵ effector cells for 10,000 targets) in the absence of peptide and 5:1 (i.e., 5×10⁴ effector cells for 10,000 targets) in the presence of peptide, the specific lytic units would be: [( 1/50,000)-( 1/500,000)]×10⁶⁼¹⁸ LU.

Assay 2: One to three days prior to the assay, 96-well ELISA plates (Costar, Corning, N.Y.) are coated with 50 μl per well of rat monoclonal antibody specific for murine IFN-γ (Clone RA-6A2, BD Biosciences/Pharmingen, San Diego, Calif.) at a concentration of 4 μg/ml in coating buffer (100 mM NaHCO₃, pH 8.2). The plates are then stored at 4-10 degrees C. until the day of the assay.

On the day of the assay, the plates are washed and blocked for 2 hours with 10% FBS in PBS. Cells from each 25 cm² flask are treated as an independent group. Duplicate wells of serially diluted splenocytes are cultured for 20 hours with and without peptide (1 μg/ml) and 10⁵ Jurkat A2.1/K^{b} cells per well at 37 degrees C. in 5% CO₂. The following day, the cells are removed by washing the plates with PBS and Tween 20 and the amount of IFN-γ that was secreted and captured by the bound Clone RA-6A2 monoclonal antibody is measured using a sandwich format ELISA. In this assay, a biotinylated rat monoclonal antibody specific for murine IFN-γ (Clone XMG1.2, BD Biosciences/Pharmingen) is used to detect the secreted IFN-γ. Horseradish peroxidase-coupled streptavidin (Zymed, South San Francisco, Calif.) and 3,3′,5,5′ tetramethylbenzidine and H₂O₂ (IMMUNOPURE® TMB Substrate Kit, Pierce, Rockford, Ill.) are used according to the manufacturer's directions for color development. The absorbance is read at 450 nm on a Labsystems Multiskan RC ELISA plate reader (Helsinki, Finland).

In situ IFN-γ ELISA data is then converted to secretory units (“SU”) for evaluation. The SU calculation is based on the number of cells that secrete 100 pg of IFN-γ in response to a particular peptide, corrected for the background amount of IFN-γ produced in the absence of peptide. To calculate the number of cells that secrete 100 pg of IFN-γ per well, a graph of the effector cell number (X axis) versus the pg/well of IFN-γ secreted (Y axis) is plotted. The slope (m) and y intercept (b) are calculated using the formula [(100−b)/m]. Because the number of cells needed to secrete 100 pg of IFN-γ in response to peptide will be lower than the cell number required for 100 pg of spontaneous release, the reciprocal values are calculated. The value obtained for the spontaneous release is then subtracted from the value obtained for specific peptide stimulation [(i/peptide stimulation)−(1/spontaneous release)]. The resulting number is multiplied by a constant of 10⁶, and this final number is designated the SU.

Results from the analysis of a subset of HLA-A2 and HLA-A3 supertype peptides obtained from Tables 16 and 18 are shown in Tables 29 and 30, respectively. In the Table, the sequence of each peptide is provided in the column labeled “Sequence.” The viral type and antigenic origin of each peptide is provided in the column labeled “Source.” In this column, the viral type is provided as the first component of each entry and the antigenic origin is provided as the second component of each entry. The third component of each entry indicates the position within the antigen of the N-terminal amino acid residue of the peptide epitope. A fourth component is present for analog peptide epitopes. If present, this component of each entry indicates the position and substituted amino acid residue for each analog peptide epitope. The final column of the Table provides a measurement of immunogenicity in secretory units (“SU;” as described above). The final column provides the SEQ ID NO for the peptide epitope. Thus, for example, the first entry on Table 29 indicates that the peptide comprising the sequence KLPQLCTEV (SEQ ID NO:______): (a) was obtained from the E6 protein of HPV-16 beginning at position 18; (b) is an analog peptide with a valine substitution at position 9; and (c) has an immunogenicity of 0.0 SU in the assay.

In situ ELISA assays for human cells are performed using a similar protocol, using mouse anti-human IFN-γ monoclonal antibody (Clone NIB42; BD Biosciences/Pharmingen) for coating, recombinant human IFN-γ (BD Biosciences/Pharmingen) for standards, and biotinylated mouse anti-human IFN-γ (Clone 4S.B3, BD Biosciences/Pharmingen) for detection. The plates are incubated for 48 hours with standards added after 24 hours. A culture was considered positive if it measured at least 50 pg of IFN-γ per well above background and is twice the background level of expression.

The results of either assay are analyzed to assess the magnitude of the CTL responses of animals injected with the immunogenic CTL/HTL conjugate vaccine preparation and are compared to the magnitude of the CTL response achieved using the CTL epitope as outlined in Example 3. Analyses similar to this may be performed to evaluate the immunogenicity of peptide conjugates containing multiple CTL epitopes and/or multiple HTL epitopes. In accordance with these procedures it is found that a CTL response is induced, and concomitantly that an HTL response is induced upon administration of such compositions.

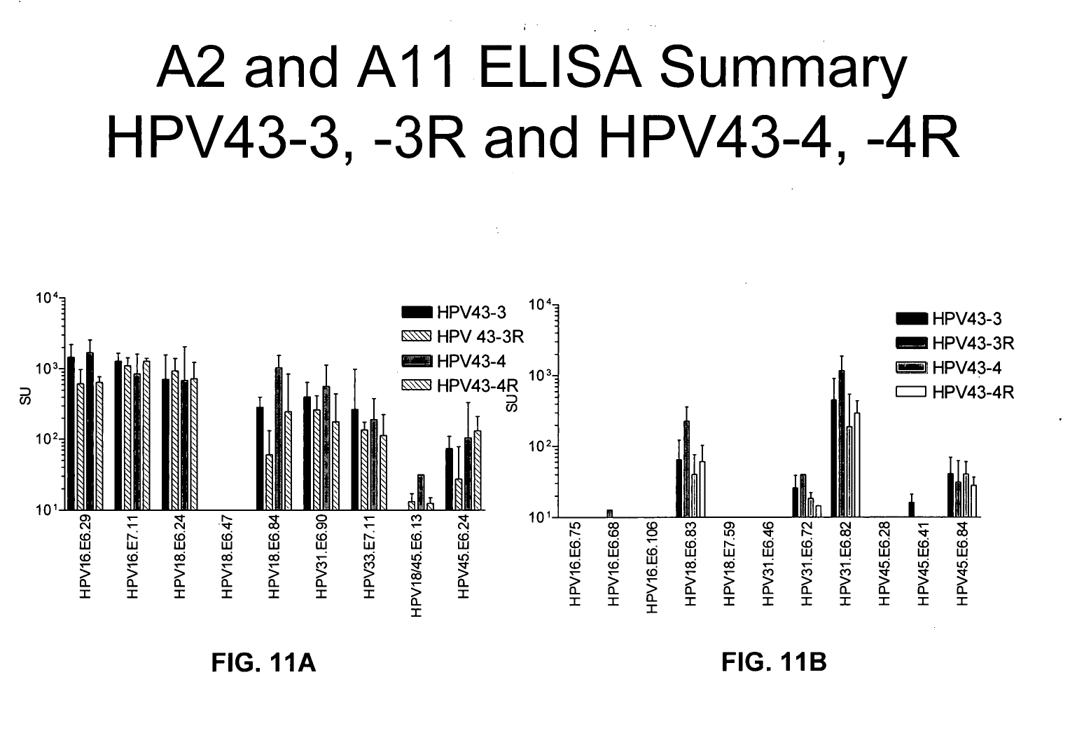

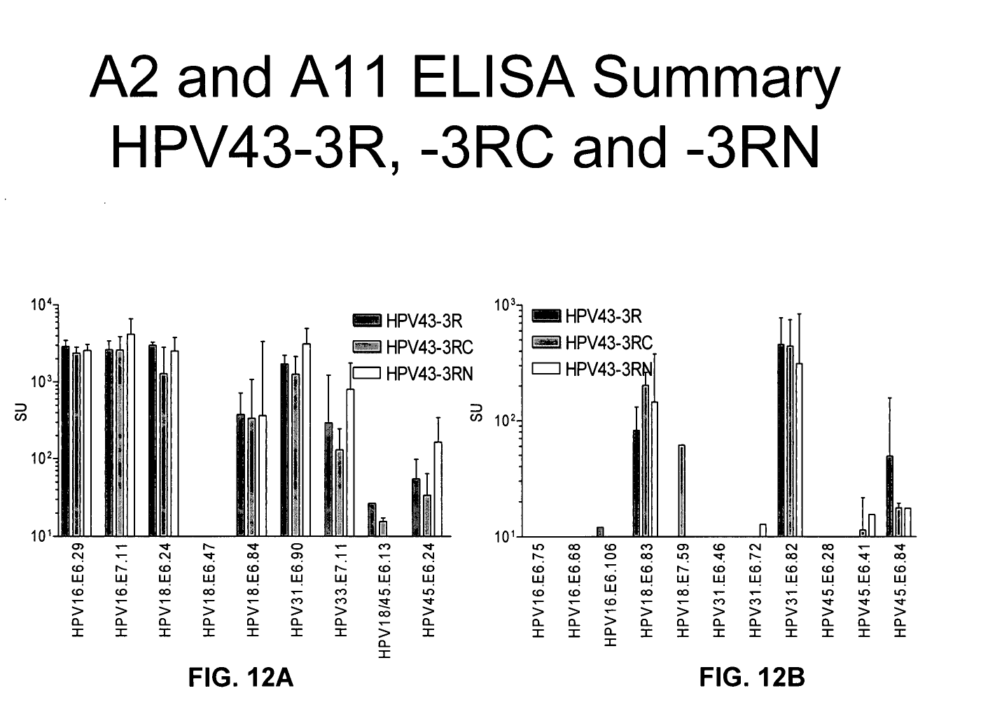

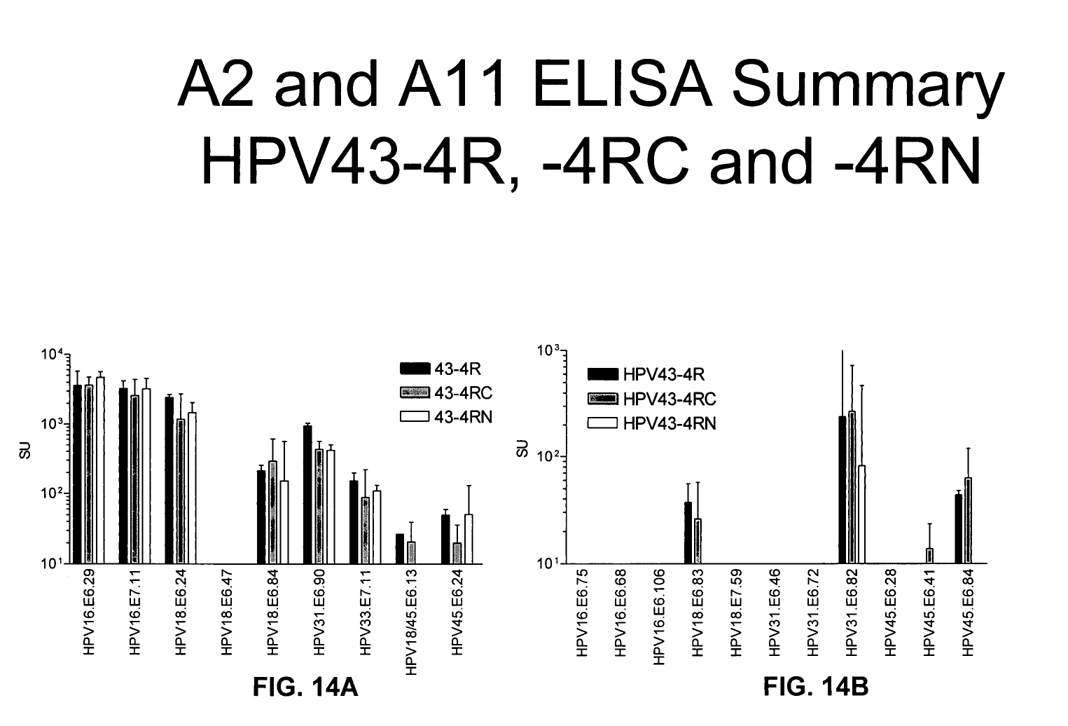

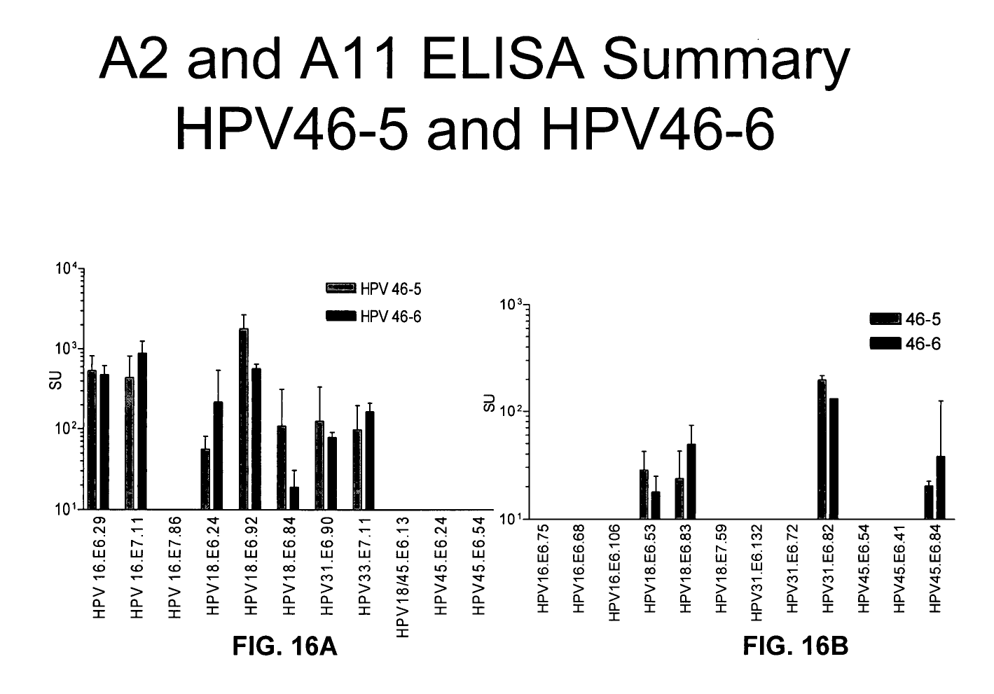

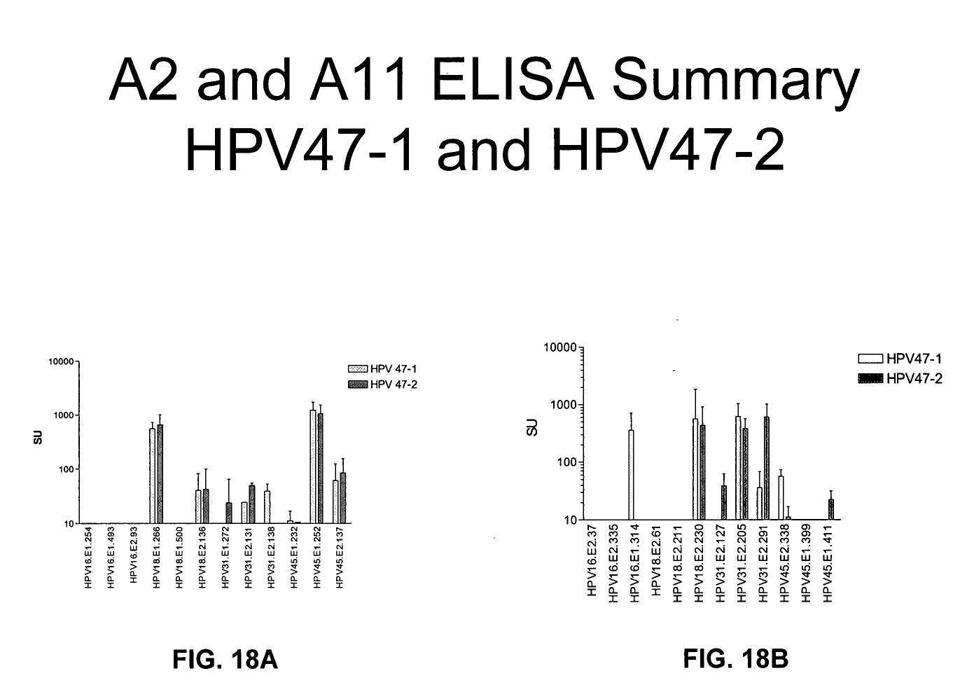

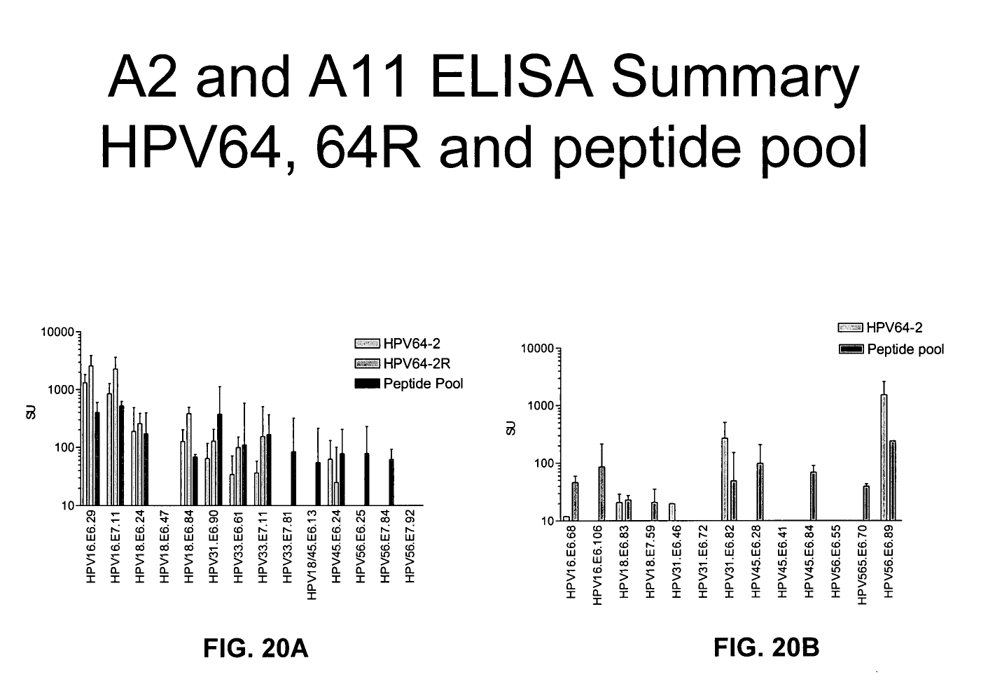

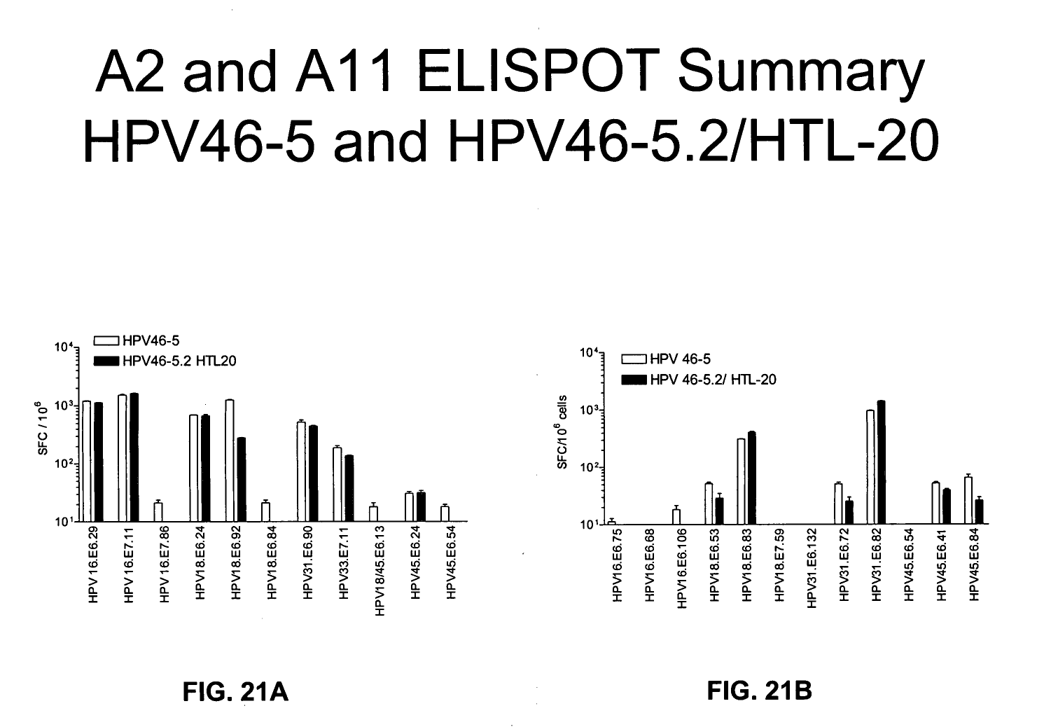

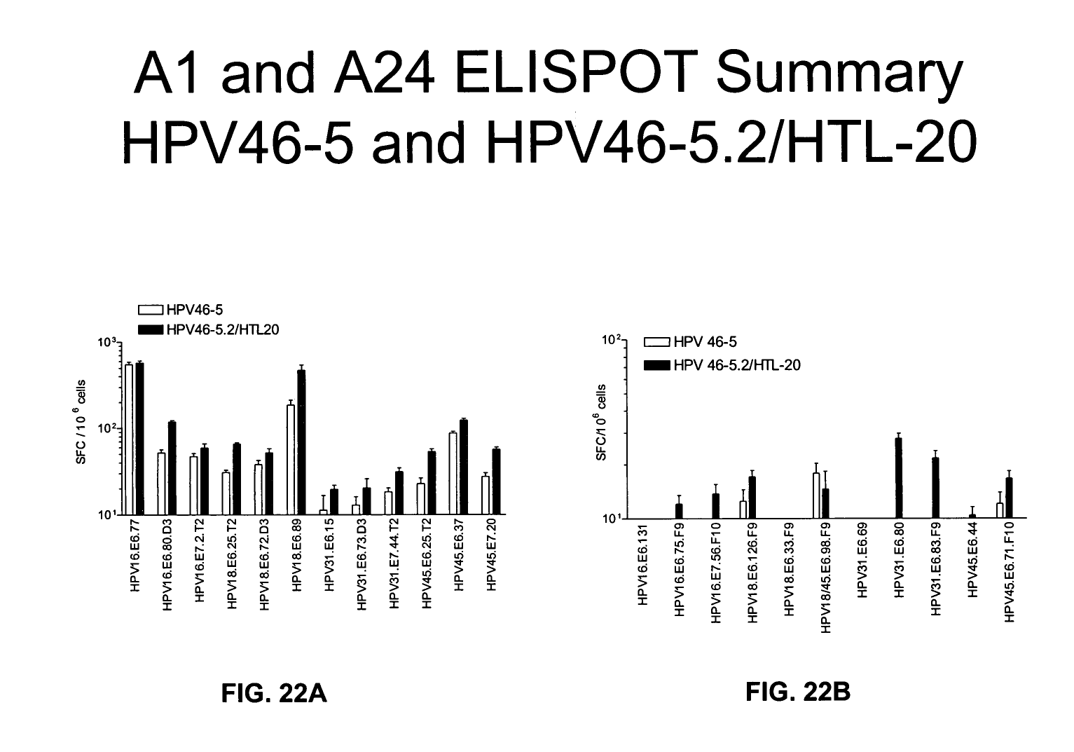

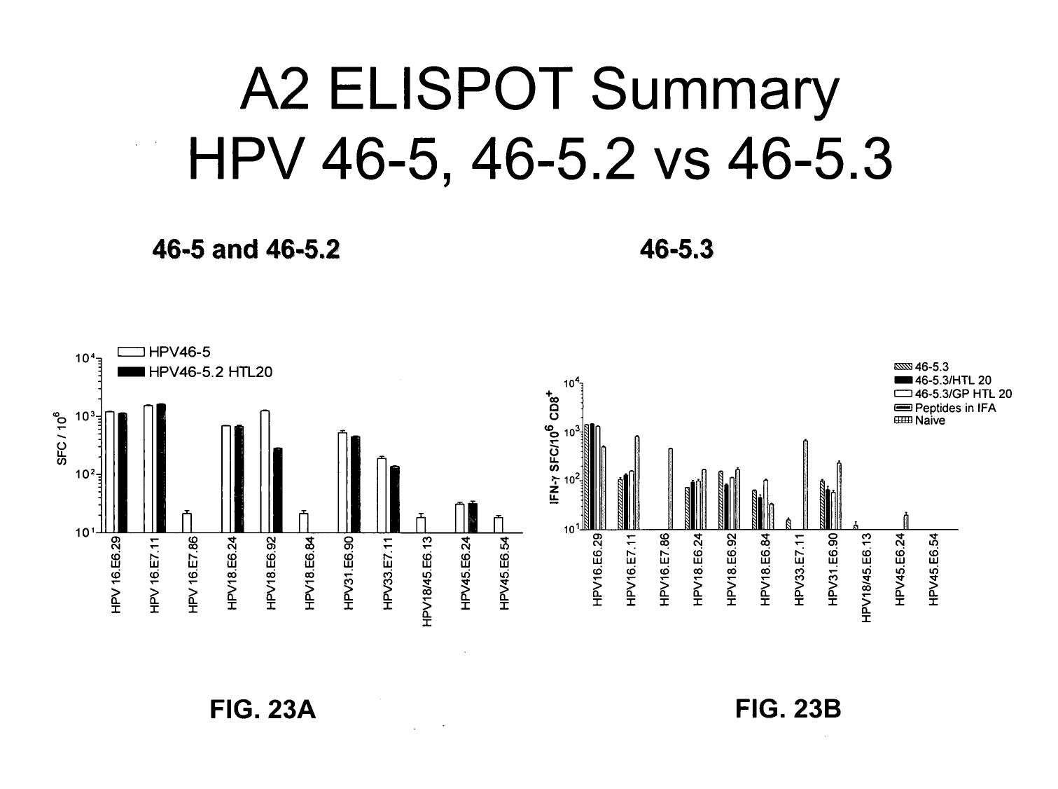

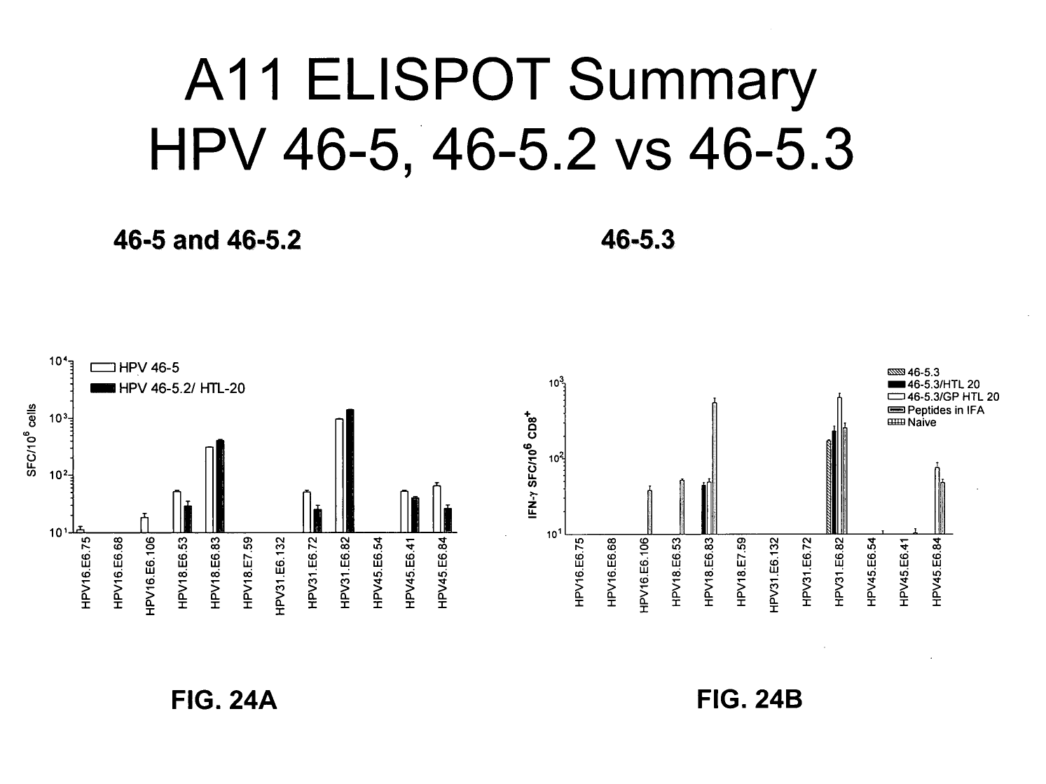

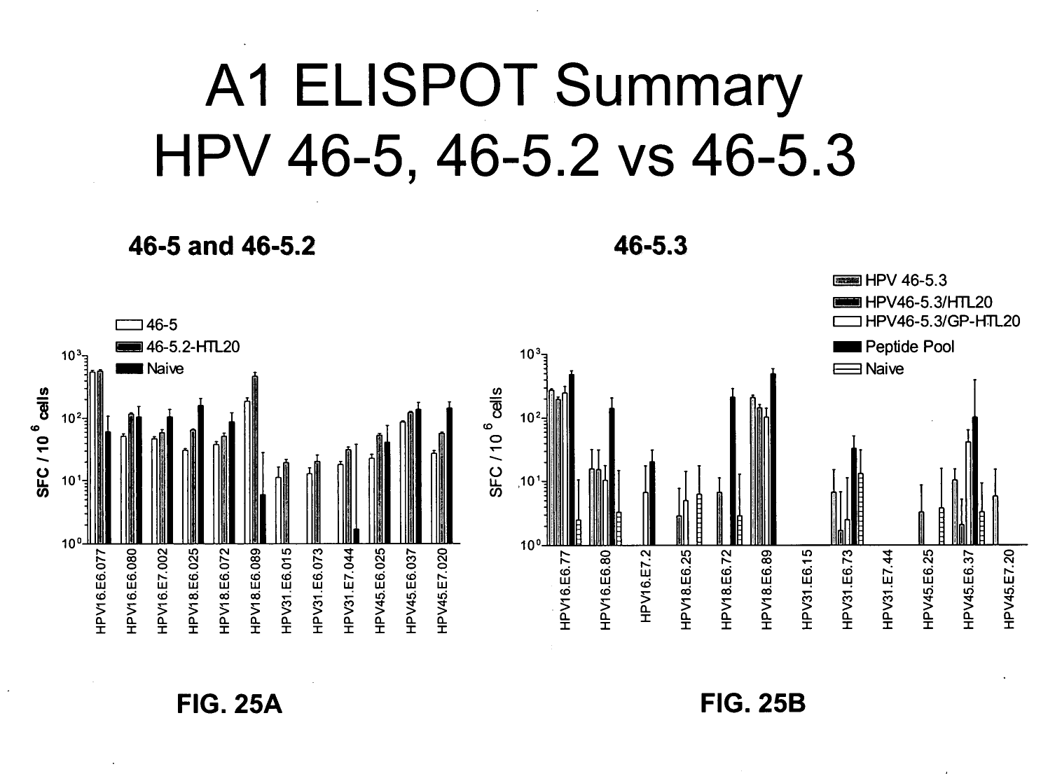

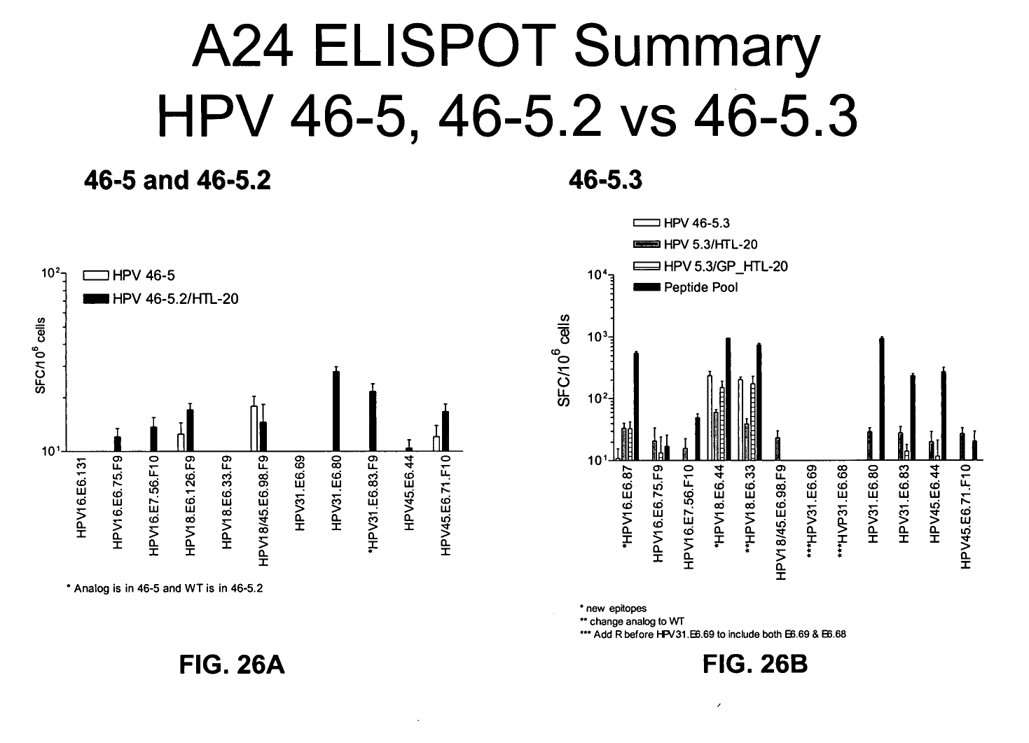

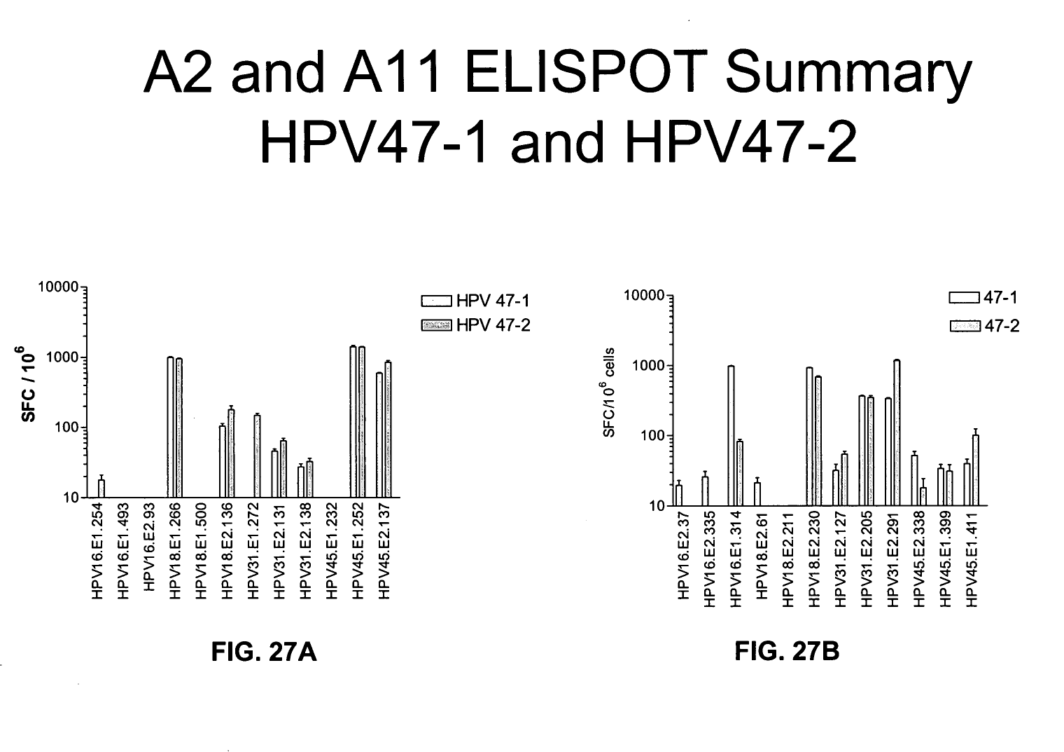

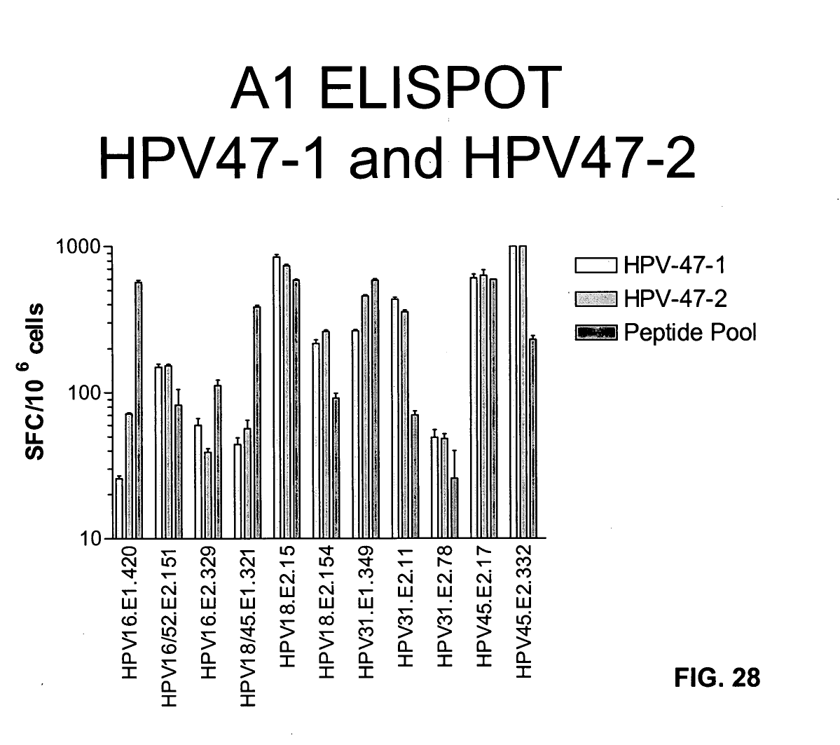

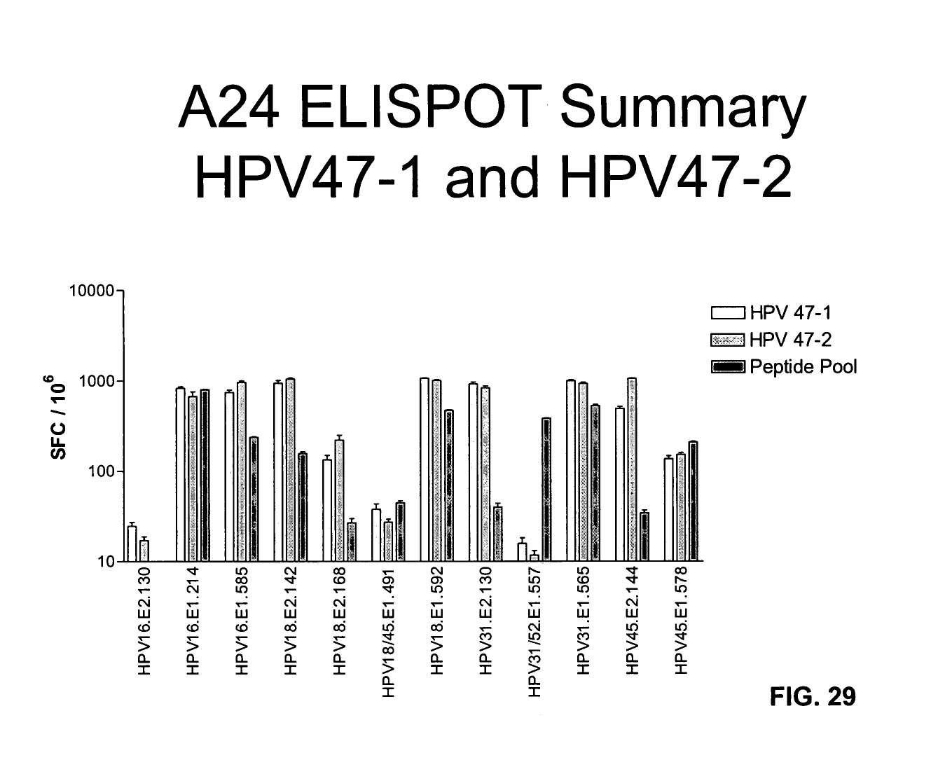

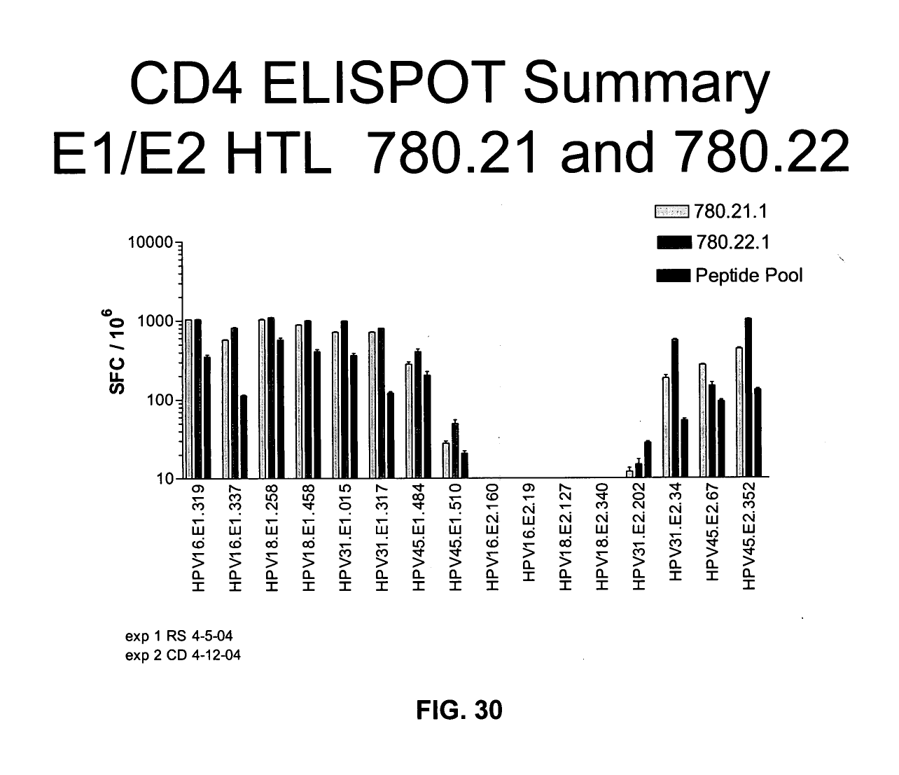

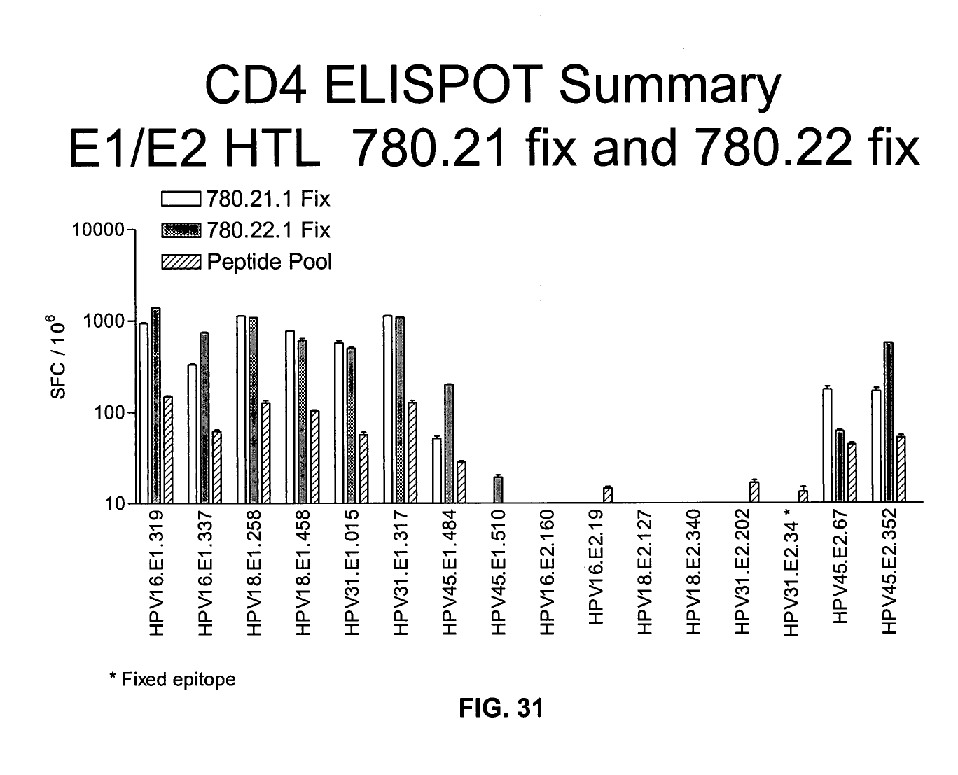

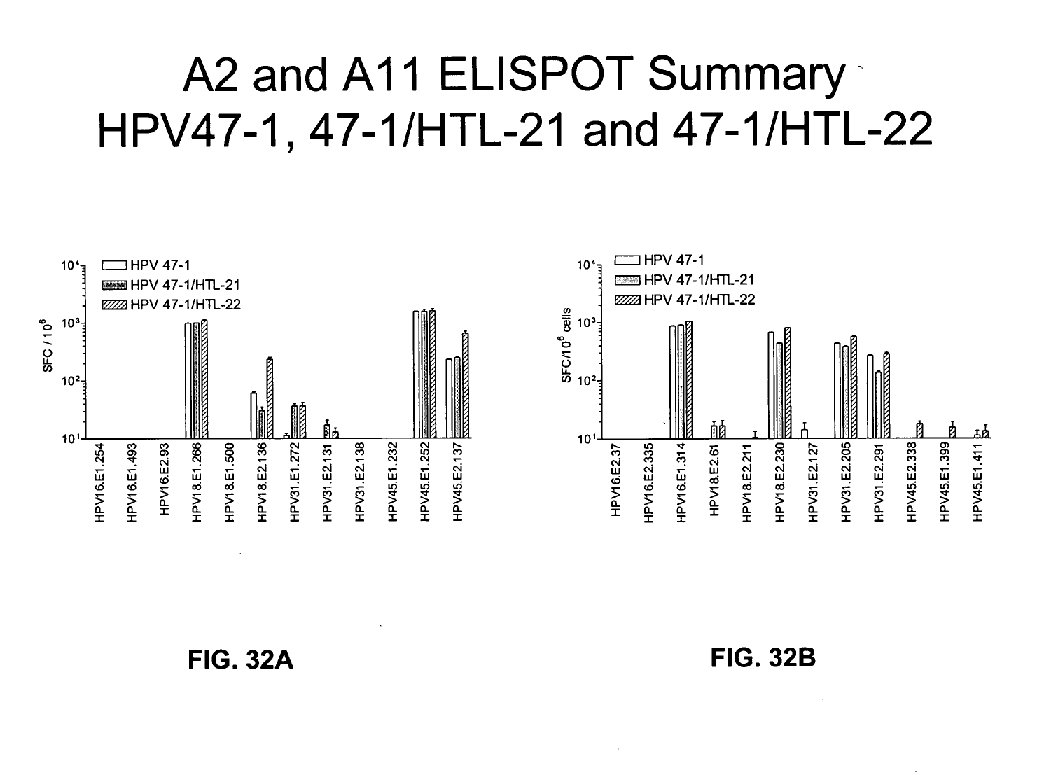

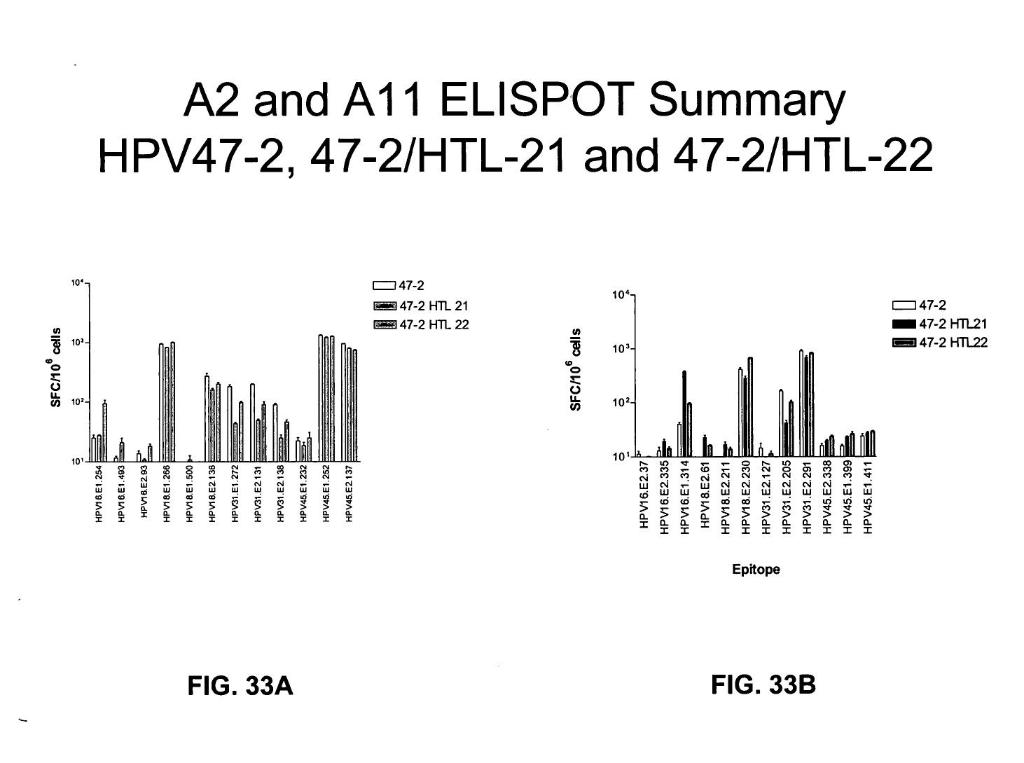

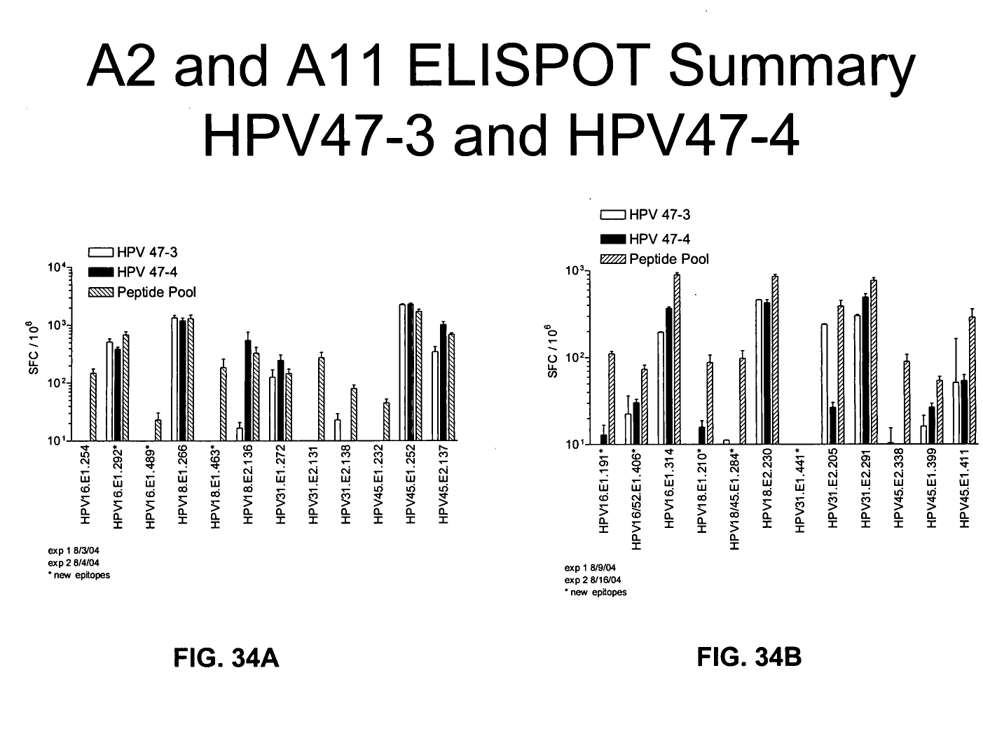

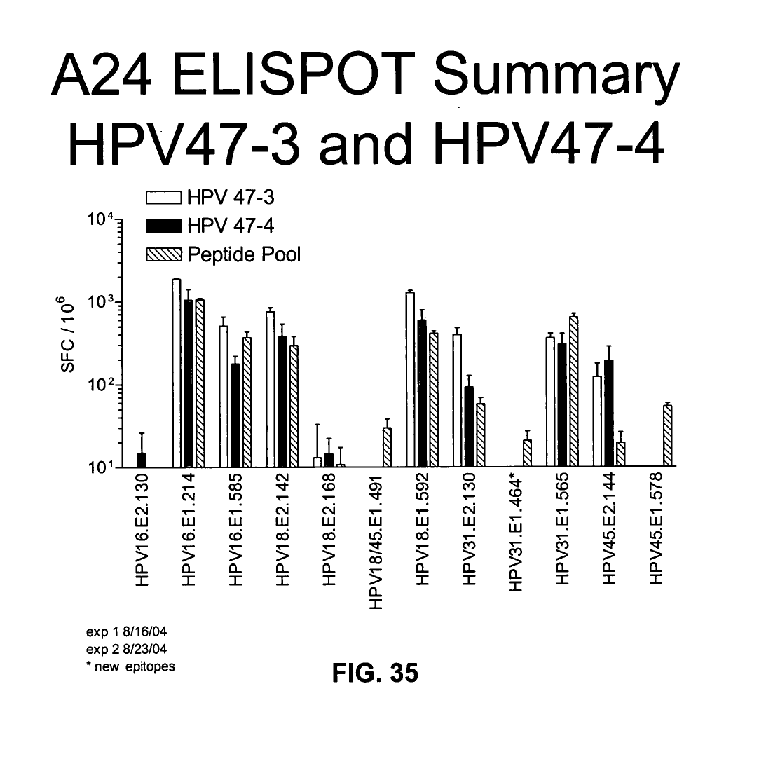

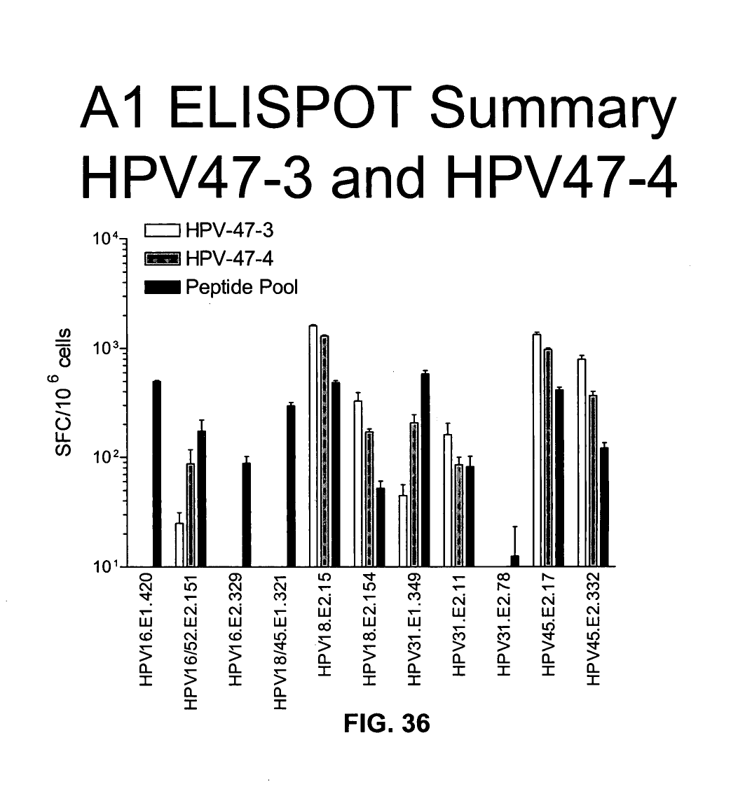

Results from experiments described in this Example are shown in FIGS. 11a, 11b, 12a, 12b, 14a, 14b, 16a, 16b, 18a, 18b, 20a and 20b.

### Example 10

### Analysis of Cross-Type Immunogenicity of HPV Peptides

This example illustrates the procedure for the analysis of peptide epitope immunogenicity across HPV types. Peptide epitope candidates are selected for analysis on the basis of immunogenicity (see e.g., Example 3) and sequence conservation across multiple HPV types (as discussed above in the specification). In the present example, peptide epitope candidates are analyzed for immunogenicity across HPV Types 16, 18, 31, 33, 45, 52, 56, and 58 are analyzed, but in practice, these types and/or any other HPV Types may be analyzed in the same manner. Although in the present study, peptide epitope candidates comprise both naturally occurring HPV amino acid sequences and analog sequences, this example may be exploited for either naturally occurring peptide epitope candidates (i.e., “wild type” peptide epitopes) or analog sequences alone.

A set of peptide epitope candidates is selected on the basis of immunogenicity as described above in Example 3. Each of the peptide epitope candidates is then analyzed according to sequence alignments of selected HPV proteins (e.g., alignments of the HPV E1, E2, E6, and E7 protein sequences of HPV Types 16, 18, 31, 33, 45, 52, 56, and 58 are provided in Tables 25, 26, 27, and 28, respectively) to determine the level of conservation of each peptide epitope candidate across multiple HPV Types.

Peptide epitope candidates that are conserved across multiple HPV types are selected for analysis of immunogenicity across each of the HPV types considered in this example. Each conserved peptide epitope candidate is then analyzed according to the transgenic mouse immunogenicity analysis provided hereinabove in Example 9. Briefly, each conserved peptide epitope candidate is synthesized and used to inoculate the appropriate strain of HLA transgenic mouse. Splenocytes are then isolated and re-stimulated for one week with the conserved peptide epitope candidate. The cultures are then tested with the corresponding peptide epitope from each HPV type tested.

Results of this analysis are provided in Tables 34 (HLA-A2-restricted peptide epitope candidates), 35 (HLA-A11-restricted peptide epitope candidates), and 48 (HLA-A2-restricted and HLA-A3-restricted peptide epitope candidates). In each Table, the amino acid sequence of each peptide epitope candidate considered is provided in the first column (labeled “Sequence”). The individual sequence identifier is provided in the second column (labeled “SEQ ID NO”). The HPV type and antigenic source are provided in the third column (labeled “Source”). The fourth through the eleventh columns are collectively labeled “Immunogenicity (cross-reactivity on HPV Strain)” and provide a measure of the immunogenicity (in secretory units) of each peptide epitope candidate as measured against the corresponding peptide epitope in each of HPV Types 16, 18, 31, 33, 45, 52, 56, and 58.

Thus, for example, the first entry on Table 34 provides the data for the peptide epitope candidate TIHDIILECV (first column) (SEQ ID NO:______; second column). The immunogenicity of this peptide epitope candidate as challenged by the corresponding peptide epitope synthesized according to the naturally occurring amino acid sequence of HPV Types 16 (fourth column), 18 (fifth column), 31 (sixth column), 33 (seventh column), 45 (eighth column), 52 (ninth column), 56 (tenth column), and 58 (eleventh column) is provided.

### Example 11

### Selection of CTL and HTL Epitopes for Inclusion in an HPV-Specific Vaccine

This example illustrates the procedure for the selection of peptide epitopes for vaccine compositions of the invention. The peptides in the composition can be in the form of a polynucleotide sequence, either single or one or more sequences (i.e., minigene) that encodes peptide(s), or can be single and/or polyepitopic peptides.

The following principles are utilized when selecting an array of epitopes for inclusion in a vaccine composition. Each of the following principles is balanced in order to make the selection.

Epitopes are selected which, upon administration, mimic immune responses that have been observed to be correlated with HPV clearance. The number of epitopes used depends on observations of patients who spontaneously clear HPV. For example, if it has been observed that patients who spontaneously clear HPV generate an immune response to at least 3 epitopes on at least one HPV antigen, then 3-4 epitopes should be included for HLA class I. A similar rationale is used to determine HLA class II epitopes.

When selecting an array of HPV epitopes, it is preferred that at least some of the epitopes are derived from early proteins. The early proteins of HPV are expressed when the virus is replicating, either following acute or dormant infection. Therefore, it is particularly preferred to use epitopes from early stage proteins to alleviate disease manifestations at the earliest stage possible.

Epitopes are often selected that have a binding affinity of an IC₅₀ of 500 nM or less for an HLA class I molecule, or for class II, an IC₅₀ of 1000 nM or less. See e.g., Tables 36A-B, 37A-B, and 48. Tables 36A-B, 37A-B, and 48 provide binding and immunogenicity data for peptide selections chosen to comprise first and second generation HPV vaccines, respectively. Each Table provides data for peptides analyzed to generate a 6 strain HPV vaccine (Tables 36A, 37A, and 48) and a 4 strain HPV vaccine (Tables 36B and 37B). Within each Table, data are provided for HLA-A2, -A3, -A1, and -A24 peptides.

With respect to Tables 36A, 37A, and 48: For the HLA-A2 peptides, data are provided to illustrate: (a) the binding affinity to purified HLA molecules and (b) the cross-strain immunogenicity of each peptide. These experiments were done as described herein. For the HLA-A3 peptides, data are provided to illustrate: (a) the binding affinity to purified HLA molecules, (b) the cross-strain immunogenicity of each peptide, and, in some cases, (c) the recognition of HLA-A3-restricted peptides by PBL from HLA-A3 positive donors. These experiments were done as described herein. For the HLA-A1 and -A24 peptides, data are provided to illustrate: (a) the binding affinity to purified HLA molecules and (b) the recognition of HLA-A1- and HLA-A24-restricted peptides by PBL from HLA-A1- and HLA-A24 positive donors, respectively. These experiments were done as described herein.

With respect to Tables 36B and 37B: For HLA-A2 and -A3 peptides, data are provided to illustrate: (a) the binding affinity to purified HLA molecules and (b) the cross-strain immunogenicity of each peptide. The first entry for HLA-A3 on Table 37B also provides data for the recognition of HLA-A3-restricted peptides by PBL from HLA-A3 positive donors. These experiments were done as described herein. For the HLA-A1 and -A24 peptides, data are provided to illustrate: (a) the binding affinity to purified HLA molecules and (b) the recognition of HLA-A1- and HLA-A24-restricted peptides by PBL from HLA-A1- and HLA-A24 positive donors, respectively. These experiments were done as described herein.

Sufficient supermotif bearing peptides, or a sufficient array of allele-specific motif bearing peptides, are selected to give broad population coverage. For example, epitopes are selected to provide at least 80% population coverage. A Monte Carlo analysis, a statistical evaluation known in the art, can be employed to assess breadth, or redundancy, of population coverage.

When creating polyepitopic compositions, e.g. a minigene, it is typically desirable to generate the smallest peptide possible that encompasses the epitopes of interest. The principles employed are similar, if not the same, as those employed when selecting a peptide comprising nested epitopes.

In cases where the sequences of multiple variants of the same target protein are available, potential peptide epitopes can also be selected on the basis of their conservancy. For example, a criterion for conservancy may define that the entire sequence of an HLA class I binding peptide or the entire 9-mer core of a class II binding peptide be conserved in a designated percentage of the sequences evaluated for a specific protein antigen.

A vaccine composition comprised of selected peptides, when administered, is safe, efficacious, and elicits an immune response similar in magnitude to an immune response that controls or clears an acute HPV infection.

### Example 12

### Construction of Minigene Multi-Epitope DNA Plasmids

This example provides general guidance for the construction of a minigene expression plasmid. Minigene plasmids may, of course, contain various configurations of CTL and/or HTL epitopes or epitope analogs as described herein. Examples of the construction and evaluation of expression plasmids are described, for example, in U.S. Pat. No. 6,534,482.

A minigene expression plasmid typically includes multiple CTL and HTL peptide epitopes. In the present example, HLA-A2, -A3, -A1 and -A24 supermotif-bearing peptide epitopes are used in conjunction with DR supermotif-bearing epitopes and/or DR3 epitopes. HLA class I supermotif or motif-bearing peptide epitopes derived from multiple HPV antigens, preferably including both early and late phase antigens, are selected such that multiple supermotifs/motifs are represented to ensure broad population coverage. Similarly, HLA class II epitopes are selected from multiple HPV antigens to provide broad population coverage, i.e. both HLA DR-1-4-7 supermotif-bearing epitopes and HLA DR-3 motif-bearing epitopes are selected for inclusion in the minigene construct. The selected CTL and HTL epitopes are then incorporated into a minigene for expression in an expression vector.

Such a construct may additionally include sequences that direct the HTL epitopes to the endocytic compartment. For example, the Ii protein may be fused to one or more HTL epitopes as described in U.S. Pat. No. 6,534,482, wherein the CLIP sequence of the Ii protein is removed and replaced with an HLA class II epitope sequence so that HLA class II epitope is directed to the endocytic compartment, where the epitope binds to an HLA class II molecules.

This example illustrates the methods to be used for construction of a minigene-bearing expression plasmid. Other expression vectors that may be used for minigene compositions are available and known to those of skill in the art.

The minigene DNA plasmid of this example contains a consensus Kozak sequence and a consensus murine kappa Ig-light chain signal sequence followed by CTL and/or HTL epitopes selected in accordance with principles disclosed herein. Overlapping oligonucleotides that can, for example, average about 70 nucleotides in length with 15 nucleotide overlaps, are synthesized and HPLC-purified. The oligonucleotides encode the selected peptide epitopes as well as appropriate linker nucleotides, Kozak sequence, and signal sequence. The final multiepitope minigene is assembled by extending the overlapping oligonucleotides in three sets of reactions using PCR. A Perkin/Elmer 2400 PCR machine is used and a total of 30 cycles are performed using the following conditions: 95° C. for 15 sec, annealing temperature (5° below the lowest calculated Tm of each primer pair) for 30 sec, and 72° C. for 1 min.

For example, a minigene can be prepared as follows. For a first PCR reaction, 5 μg of each of two oligonucleotides are annealed and extended: In an example using eight oligonucleotides, i.e., four pairs of primers, oligonucleotides 1+2, 3+4, 5+6, and 7+8 are combined in 100 μl reactions containing Pfu polymerase buffer (1×=10 mM KCL, 10 mM (NH4)₂SO₄, 20 mM Tris-chloride, pH 8.75, 2 mM MgSO₄, 0.1% Triton X-100, 100 μg/ml BSA), 0.25 mM each dNTP, and 2.5 U of Pfu polymerase. The full-length dimer products are gel-purified, and two reactions containing the product of 1+2 and 3+4, and the product of 5+6 and 7+8 are mixed, annealed, and extended for 10 cycles. Half of the two reactions are then mixed, and 5 cycles of annealing and extension carried out before flanking primers are added to amplify the full length product. The full-length product is gel-purified and cloned into pCR-blunt (Invitrogen) and individual clones are screened by sequencing.

This method has been used to generate several HPV minigene vaccine constructs. For example, a subset of the peptides shown in Tables 13-24 were analyzed according to the methods described herein (e.g., section IV.L. of the specification) to determine the optimal arrangement of the epitopes in the minigenes disclosed herein. The peptides were then linked together using the method described in this Example to create numerous HPV minigene vaccine constructs. See e.g., Tables 38A-B, 41, 46-47, 52, 58, 63, and 66. In addition, the peptides were also analyzed according to the methods described herein (e.g., section IV.L. of the specification) to determine the optimal arrangement of the epitopes in the minigenes disclosed herein. The peptides were then also linked together using the method described in this Example to create two additional HPV minigene vaccine constructs. See e.g., Table 38C-D. The polynucleotide and amino acid sequences encoding these constructs are provided in Tables 39A-D, 40A-D, 42-45, 49-50, 53-54, 59, 60-62, 64-65, and 67-68.

Following additional analyses of the immunogenicity of the individual peptides included in the minigenes shown in Tables 38A-D, several of the peptide epitopes were replaced with other peptide epitopes of the invention that exhibited superior immunogenicity characteristics. In addition, the order and spacer characteristics of the revised minigenes were reanalyzed according to the methods described herein, e.g., in section IV.L. of the specification. The resulting minigenes are designated “second generation” and are provided in Tables 41A-D. The polynucleotide and amino acid sequences encoding these constructs are provided in Tables 42A-D and 43A-D.

Following additional analyses of the immunogenicity of the individual peptides included in the “first” and “second” generation minigenes described herein, several of the peptide epitopes were replaced with other peptide epitopes of the invention that exhibited superior immunogenicity characteristics. Alternatively, or in addition to, several of the peptide epitopes were modified so as to exhibit superior immunogenicity characteristics. Alternatively, or in addition to, additional peptide epitopes of the invention that exhibited superior immunogenicity characteristics were added to existing minigenes of the invention. The order and spacer characteristics of the revised minigenes were then reanalyzed according to the methods described herein, e.g., in section IV.L. of the specification. The resulting minigenes are designated “third” generation minigenes. Schematic diagrams, nucleotide and amino acid sequences, and data are provided and described in Tables 44-68. nucleotide and amino acid sequences, and data are provided and described in Tables 44-85.

### Example 13

### The Plasmid Construct and the Degree to which it Induces Immunogenicity

The degree to which a plasmid construct, for example a plasmid constructed in accordance with Example 11, is able to induce immunogenicity can be evaluated in vitro by testing for epitope presentation by APC following transduction or transfection of the APC with an epitope-expressing nucleic-acid construct. Such a study determines “antigenicity” and allows the use of human APC. The assay determines the ability of the epitope to be presented by the APC in a context that is recognized by a T cell by quantifying the density of epitope-HLA class I complexes on the cell surface. Quantitation can be performed by directly measuring the amount of peptide eluted from the APC (see, e.g., Sijts, et al., J. Immunol. 156:683-92, 1996; Demotz, et al., Nature 342:682-84, 1989); or the number of peptide-HLA class I complexes can be estimated by measuring the amount of lysis or lymphokine release induced by infected or transfected target cells, and then determining the concentration of peptide necessary to obtained equivalent levels of lysis or lymphokine release (see, e.g., Kageyama, et al., J. Immunol. 154:567-76, 1995).

Atlernatively, immunogenicity can be evaluated through in vivo injections into mice and subsequent in vitro assessment of CTL and HTL activity, which are analysed using cytotoxicity and proliferation assays, respectively, as detailed e.g., in U.S. Pat. No. 6,534,482 and Alexander, et al., Immunity 1:751-61, 1994.

For example, to assess the capacity of a DNA minigene construct (e.g., a pMin minigene construct generated as described in U.S. Pat. No. 6,534,482) containing at least one HLA-A2 supermotif peptide to induce CTLs in vivo, HLA-A2.1/K^{b} transgenic mice, for example, are immunized intramuscularly with 100 μg of naked cDNA. As a means of comparing the level of CTLs induced by cDNA immunization, a control group of animals is also immunized with an actual peptide composition that comprises multiple epitopes synthesized as a single polypeptide as they would be encoded by the minigene.

Splenocytes from immunized animals are subsequently stimulated with each of the respective compositions (peptide epitopes encoded in the minigene or the polyepitopic peptide), then assayed for peptide-specific cytotoxic activity in a ⁵¹Cr release assay. The results indicate the magnitude of the CTL response directed against the A2-restricted epitope, thus indicating the in vivo immunogenicity of the minigene vaccine and polyepitopic vaccine. It is, therefore, found that the minigene elicits immune responses directed toward the HLA-A2 supermotif peptide epitopes as does the polyepitopic peptide vaccine. A similar analysis is also performed using other HLA-A3 and HLA-B7 transgenic mouse models to assess CTL induction by HLA-A3 and HLA-B7 motif or supermotif epitopes.

Alternatively, an in situ ELISA assay may be used to evaluate immunogenicity. The assay is performed as described in Example 9.

To assess the capacity of a class II epitope encoding minigene to induce HTLs in vivo, DR transgenic mice, or for those epitope that cross react with the appropriate mouse MHC molecule, I-A^{b}-restricted mice, for example, are immunized intramuscularly with 100 μg of plasmid DNA. As a means of comparing the level of HTLs induced by DNA immunization, a group of control animals is also immunized with an actual peptide composition emulsified in complete Freund's adjuvant. CD4⁺ T cells, i.e. HTLs, are purified from splenocytes of immunized animals and stimulated with each of the respective compositions (peptides encoded in the minigene). The HTL response is measured by using a ³H-thymidine incorporation proliferation assay, (see, e.g., Alexander et al. Immunity 1:751-761, 1994) or by ELISPOT. The results of either assay indicate the magnitude of the HTL response, thus demonstrating the in vivo immunogenicity of the minigene.

Mouse CD4⁺ ELISPOT Assay

MHC class II restricted responses are measured using an IFN-γ ELISPOT assay. Purified splenic CD4⁺ cells (4×10⁵/well), isolated using MACS columns (Milteny), and irradiated splenocytes (1×10⁵ cells/well) are added to membrane-backed 96 well ELISA plates (Millipore) pre-coated with monoclonal antibody specific for murine IFN-γ (Mabtech). Cells are cultured with 10 μg/ml peptide for 20 hours at 37 degrees C. The IFN-γ secreting cells are detected by incubation with biotinylated anti-mouse IFN-γ antibody (Mabtech), followed by incubation with Avidin-Peroxidase Complex (Vectastain). The plates are developed using AEC (3-amino-9-ethyl-carbazole; Sigma), washed and dried. Spots are counted using the Zeiss KS ELISPOT reader and the results are presented as the number of IFN-γ spot forming cells (“SFC”) per 10⁶ CD4⁺ T cells.

Mouse CD8⁺ ELISPOT Assay

MHC class II restricted responses are measured using an IFN-γ ELISPOT assay. Purified splenic CD4⁺ cells (4×10⁵/well), isolated using MACS columns (Milteny), and irradiated splenocytes (1×10⁵ cells/well) are added to membrane-backed 96 well ELISA plates (Millipore) pre-coated with monoclonal antibody specific for murine IFN-γ (Mabtech). Cells are cultured with 10 μg/ml peptide and target cells for 20 hours at 37 degrees C. The IFN-γ secreting cells are detected by incubation with biotinylated anti-mouse IFN-γ antibody (Mabtech), followed by incubation with Avidin-Peroxidase Complex (Vectastain). The plates are developed using AEC (3-amino-9-ethyl-carbazole; Sigma), washed and dried. Spots are counted using the Zeiss KS ELISPOT reader and the results are presented as the number of IFN-γ spot forming cells (“SFC”) per 106 CD4⁺ T cells.

Human IFN-γ ELISPOT Assay

PBMC responses to the panel of CTL or HTL epitope peptides are evaluated using an IFN-γ ELISPOT assay. Briefly, membrane-based 96 well plates (Millipore, Bedford, Mass.) are coated overnight at 4 degrees C. with the murine monoclonal antibody specific for human IFN-γ (Clone 1-D1k, Mabtech Inc., Cincinnati, Ohio) at the concentration of 5 μg/ml. After washing with PBS, RPMI+10% heat-inactivated human AB serum is added to each well and incubated at 37 degrees C. for at least 1 hour to block membranes. The CTL or HTL epitope peptides are diluted in AIM-V media and added to triplicate wells in a volume of 100 μl at a final concentration of 10 γg/ml. Cryopreserved PBMC are thawed, resuspended in AIM-V at a concentration of 1×10⁶ PBMC/ml and dispensed in 100 μl volumes into test wells. The assay plates are incubated at 37 degrees C. for 40 hours after which they are washed with PBS+0.05% Tween 20. To each well, 100 μl of biotinylated monoclonal antibody specific for human IFN-γ (Clone 7-B6-1, Mabtech) at a concentration of 2 μg/ml is added and plates are incubated at 37 degrees C. for 2 hours. The plates are again washed avidin-peroxidase complex (Vectastain Elite kit) is added to each well, and the plates are incubated at room temperature for 1 hour. The plates are then developed and read as described above.

DNA minigenes, constructed as describe in Example 11, may also be evaluated as a vaccine in combination with a boosting agent using a prime boost protocol. The boosting agent can consist of recombinant protein (e.g., Barnett, et al., Aids Res. and Human Retroviruses 14, Suppl. 3:S299-S309, 1998) or recombinant vaccinia, for example, expressing a minigene or DNA encoding the complete protein of interest (see, e.g., Hanke, et al., Vaccine 16:439-45, 1998; Sedegah, et al., Proc. Natl. Acad. Sci U.S.A. 95:7648-53, 1998; Hanke and McMichael, Immunol. Lett. 66:177-81, 1999; and Robinson, et al., Nature Med. 5:526-34, 1999).

For example, the efficacy of the DNA minigene used in a prime boost protocol is initially evaluated in transgenic mice. In this example, A2.1/K^{b} transgenic mice are immunized IM with 100 μg of a DNA minigene encoding the immunogenic peptides including at least one HLA-A2 supermotif-bearing peptide. After an incubation period (ranging from 3-9 weeks), the mice are boosted IP with 10⁷ pfu/mouse of a recombinant vaccinia virus expressing the same sequence encoded by the DNA minigene. Control mice are immunized with 100 μg of DNA or recombinant vaccinia without the minigene sequence, or with DNA encoding the minigene, but without the vaccinia boost. After an additional incubation period of two weeks, splenocytes from the mice are immediately assayed for peptide-specific activity in an ELISPOT assay. Additionally, splenocytes are stimulated in vitro with the A2-restricted peptide epitopes encoded in the minigene and recombinant vaccinia, then assayed for peptide-specific activity in an in situ IFN-γ ELISA.

It is found that the minigene utilized in a prime-boost protocol elicits greater immune responses toward the HLA-A2 supermotif peptides than with DNA alone. Such an analysis can also be performed using HLA-A11 or HLA-B7 transgenic mouse models to assess CTL induction by HLA-A3 or HLA-B7 motif or supermotif epitopes.

The use of prime boost protocols in humans is described in Example 20.

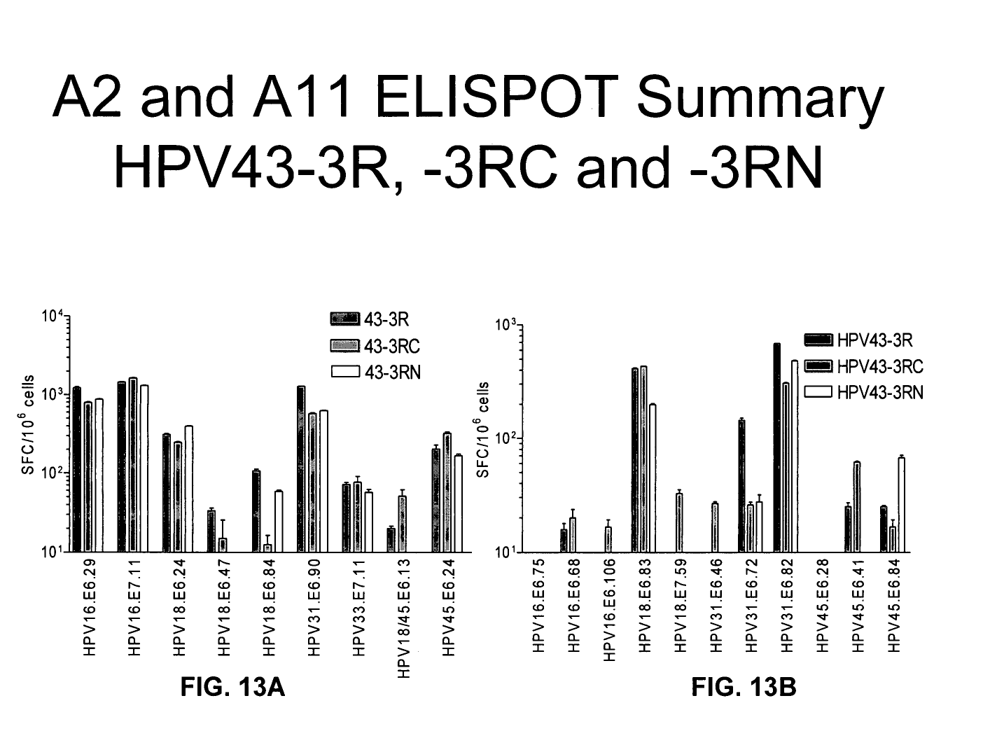

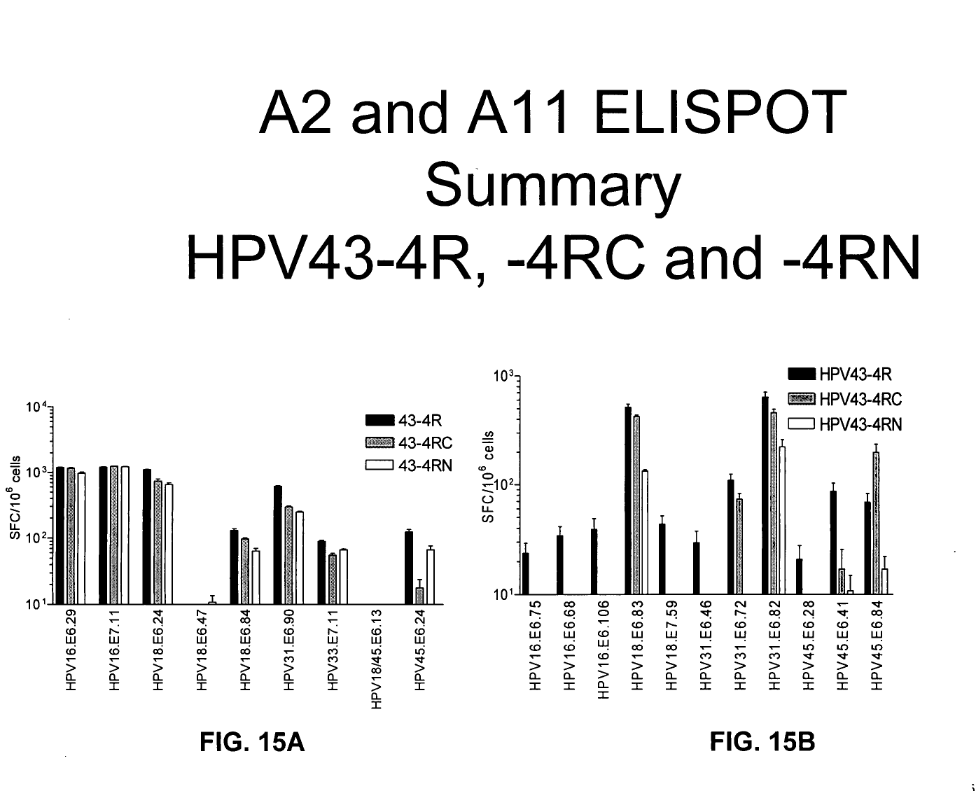

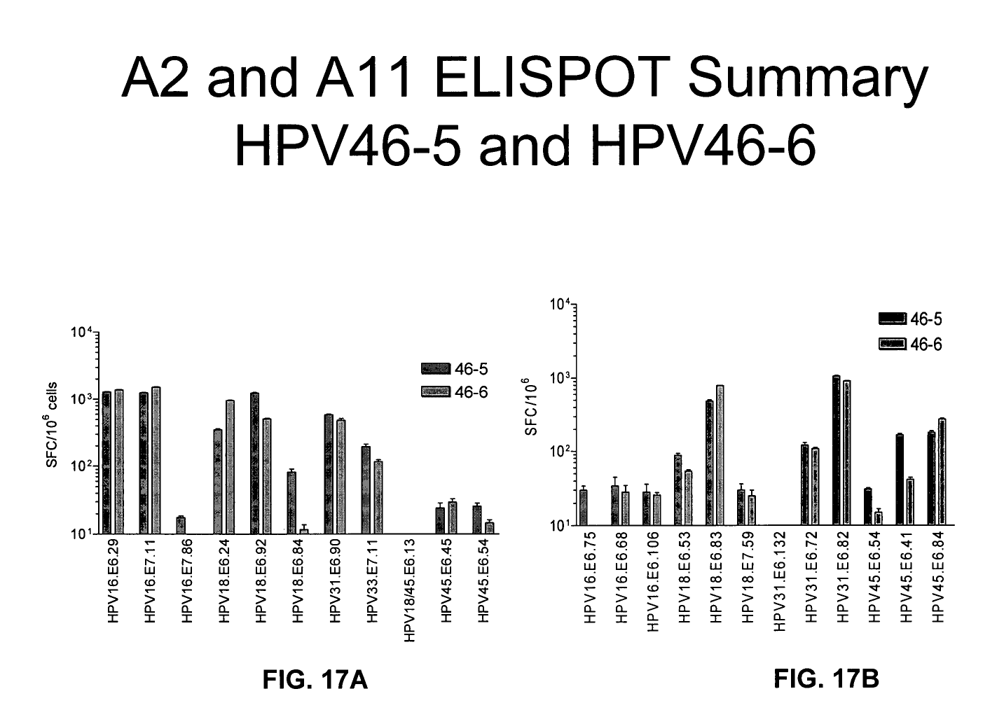

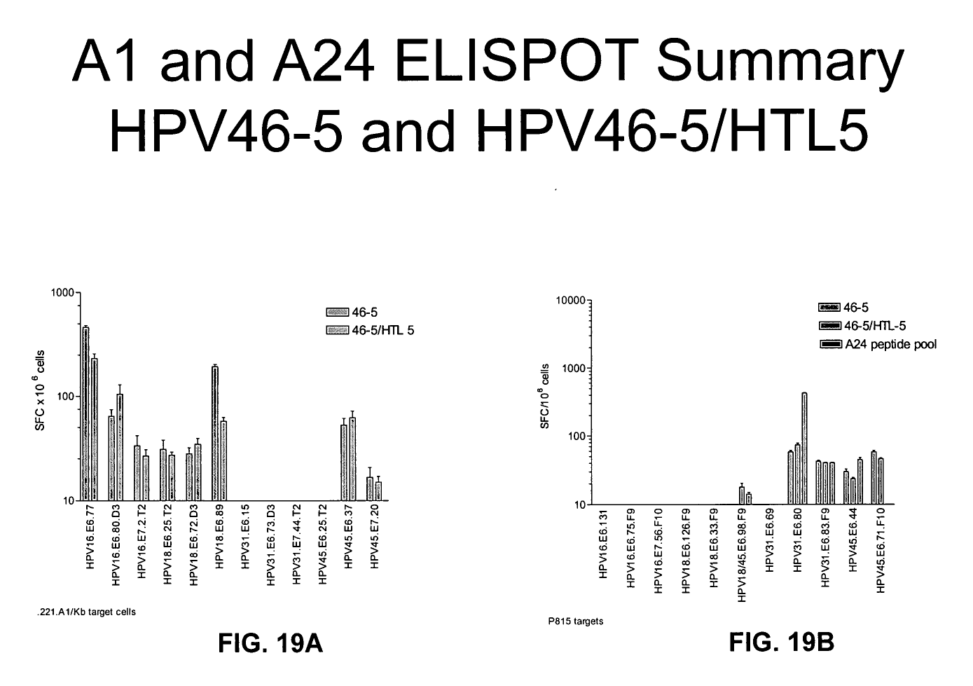

Results from experiments described in this Example can be seen in FIGS. 13a, 13b, 15a, 15b, 17a, 17b, 19a and 19b.

### Example 14

### Peptide Composition for Prophylactic Uses

Vaccine compositions of the present invention can be used to prevent HPV infection in persons who are at risk for such infection. For example, a polyepitopic peptide epitope composition (or a nucleic acid comprising the same) containing multiple CTL and HTL epitopes such as those selected in Examples 9 and/or 10, which are also selected to target greater than 80% of the population, is administered to individuals at risk for HPV infection.

For example, a peptide-based composition can be provided as a single polypeptide that encompasses multiple epitopes. The vaccine is typically administered in a physiological solution that comprises an adjuvant, such as Incomplete Freunds Adjuvant (“IFA”). The dose of peptide for the initial immunization is from about 1 to about 50,000 μg, generally 100-5,000 μg, for a 70 kg patient. The initial administration of vaccine is followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious as a prophylaxis against HPV infection.

Alternatively, a composition typically comprising transfecting agents can be used for the administration of a nucleic acid-based vaccine in accordance with methodologies known in the art and disclosed herein.

### Example 15

### Polyepitopic Vaccine Compositions Derived from Native HPV Sequences

A native HPV polyprotein sequence is screened, preferably using computer algorithms defined for each class I and/or class II supermotif or motif, to identify “relatively short” regions of the polyprotein that comprise multiple epitopes and is preferably less in length than an entire native antigen. This relatively short sequence that contains multiple distinct, even overlapping, epitopes is selected and used to generate a minigene construct. The construct is engineered to express the peptide, which corresponds to the native protein sequence. The “relatively short” peptide is generally less than 250 amino acids in length, often less than 100 amino acids in length, preferably less than 75 amino acids in length, and more preferably less than 50 amino acids in length. The protein sequence of the vaccine composition is selected because it has maximal number of epitopes contained within the sequence, i.e., it has a high concentration of epitopes. As noted herein, epitope motifs may be nested or overlapping (i.e., frame shifted relative to one another). For example, with overlapping epitopes, two 9-mer epitopes and one 10-mer epitope can be present in a 10 amino acid peptide. Such a vaccine composition is administered for therapeutic or prophylactic purposes.

The vaccine composition will include, for example, three CTL epitopes from at least one HPV target antigen and at least one HTL epitope. This polyepitopic native sequence is administered either as a peptide or as a nucleic acid sequence which encodes the peptide. Alternatively, an analog can be made of this native sequence, whereby one or more of the epitopes comprise substitutions that alter the cross-reactivity and/or binding affinity properties of the polyepitopic peptide.

The embodiment of this example provides for the possibility that an as yet undiscovered aspect of immune system processing will apply to the native nested sequence and thereby facilitate the production of therapeutic or prophylactic immune response-inducing vaccine compositions. Additionally such an embodiment provides for the possibility of motif-bearing epitopes for an HLA makeup that is presently unknown. Furthermore, this embodiment (absent analogs) directs the immune response to multiple peptide sequences that are actually present in native HPV antigens thus avoiding the need to evaluate any junctional epitopes. Lastly, the embodiment provides an economy of scale when producing nucleic acid vaccine compositions.

Related to this embodiment, computer programs can be derived in accordance with principles in the art, which identify in a target sequence, the greatest number of epitopes per sequence length.

### Example 16

### Polyepitopic Vaccine Compositions from Multiple Antigens

The HPV peptide epitopes of the present invention are used in conjunction with peptide epitopes from other target tumor-associated antigens to create a vaccine composition that is useful for the prevention or treatment of cancer resulting from HPV infection in multiple patients.

For example, a vaccine composition can be provided as a single polypeptide that incorporates multiple epitopes from HPV antigens as well as tumor-associated antigens that are often expressed with a target cancer, e.g., cervical cancer, associated with HPV infection, or can be administered as a composition comprising one or more discrete epitopes. Alternatively, the vaccine can be administered as a minigene construct or as dendritic cells which have been loaded with the peptide epitopes in vitro.

### Example 17

### Use of Peptides to Evaluate an Immune Response

Peptides of the invention may be used to analyze an immune response for the presence of specific CTL or HTL populations directed to HPV. Such an analysis may be performed in a manner as that described by Ogg, et al., Science 279:2103-06, 1998. In the following example, peptides in accordance with the invention are used as a reagent for diagnostic or prognostic purposes, not as an immunogen.

In this example highly sensitive human leukocyte antigen tetrameric complexes (“tetramers”) are used for a cross-sectional analysis of, for example, HPV HLA-A*0201-specific CTL frequencies from HLA A*0201-positive individuals at different stages of infection or following immunization using an HPV peptide containing an A*0201 motif. Tetrameric complexes are synthesized as described (Musey, et al., N. Engl. J. Med. 337:1267, 1997). Briefly, purified HLA heavy chain (A*0201 in this example) and β2-microglobulin are synthesized by means of a prokaryotic expression system. The heavy chain is modified by deletion of the transmembrane-cytosolic tail and COOH-terminal addition of a sequence containing a BirA enzymatic biotinylation site. The heavy chain, β2-microglobulin, and peptide are refolded by dilution. The 45-kD refolded product is isolated by fast protein liquid chromatography and then biotinylated by BirA in the presence of biotin (Sigma, St. Louis, Mo.), adenosine 5′triphosphate and magnesium. Streptavidin-phycoerythrin conjugate is added in a 1:4 molar ratio, and the tetrameric product is concentrated to 1 mg/ml. The resulting product is referred to as tetramer-phycoerythrin.

For the analysis of patient blood samples, approximately one million PBMCs are centrifuged at 300 g for 5 minutes and resuspended in 50 μl of cold phosphate-buffered saline. Tri-color analysis is performed with the tetramer-phycoerythrin, along with anti-CD8-Tricolor, and anti-CD38. The PBMCs are incubated with tetramer and antibodies on ice for 30 to 60 min and then washed twice before formaldehyde fixation. Gates are applied to contain >99.98% of control samples. Controls for the tetramers include both A*0201-negative individuals and A*0201-positive uninfected donors. The percentage of cells stained with the tetramer is then determined by flow cytometry. The results indicate the number of cells in the PBMC sample that contain epitope-restricted CTLs, thereby readily indicating the extent of immune response to the HPV epitope, and thus the stage of infection with HPV, the status of exposure to HPV, or exposure to a vaccine that elicits a protective or therapeutic response.

### Example 18

### Use of Peptide Epitopes to Evaluate Recall Responses

The peptide epitopes of the invention are used as reagents to evaluate T cell responses, such as acute or recall responses, in patients. Such an analysis may be performed on patients who have recovered from infection, who are chronically infected with HPV, or who have been vaccinated with an HPV vaccine.

For example, the class I restricted CTL response of persons who have been vaccinated may be analyzed. The vaccine may be any HPV vaccine. PBMC are collected from vaccinated individuals and HLA typed. Appropriate peptide epitopes of the invention that, optimally, bear supermotifs to provide cross-reactivity with multiple HLA supertype family members, are then used for analysis of samples derived from individuals who bear that HLA type.

PBMC from vaccinated individuals are separated on Ficoll-Histopaque density gradients (Sigma Chemical Co., St. Louis, Mo.), washed three times in HBSS (Invitrogen Life Technologies, Carlsbad, Calif.), resuspended in RPMI-1640 (Invitrogen Life Technologies, Carlsbad, Calif.) supplemented with L-glutamine (2 mM), penicillin (50 U/ml), streptomycin (50 μg/ml), and Hepes (10 mM) containing 10% heat-inactivated human AB serum (complete RPMI) and plated using microculture formats. A synthetic peptide comprising an epitope of the invention is added to each well at a concentration of 10 μg/ml and HBV core 128-140 epitope is added at 1 μg/ml to each well as a source of T cell help during the first week of stimulation.

Cytotoxicity assays may be performed in several ways well known in the art. Several non-limiting examples follow.

A Direct Cellular Cytotoxicity Assay

In the microculture format, 4×10⁵ PBMC are stimulated with peptide in 8 replicate cultures in 96-well round bottom plate in 100 μl/well of complete RPMI. On days 3 and 10, 100 μl of complete RPMI and 20 U/ml final concentration of rIL-2 are added to each well. On day 7 the cultures are transferred into a 96-well flat-bottom plate and restimulated with peptide, rIL-2 and 10⁵ irradiated (3,000 rad) autologous feeder cells. The cultures are tested for cytotoxic activity on day 14. A positive CTL response requires two or more of the eight replicate cultures to display greater than 10% specific ⁵¹Cr release, based on comparison with uninfected control subjects as previously described (Rehermann, et al., Nature Med. 2:1104, 1996; Rehermann, et al., J. Clin. Invest. 97:1655-65, 1996; and Rehermann, et al., J. Clin. Invest. 98:1432-40, 1996).

Target cell lines are autologous and allogeneic EBV-transformed B-LCL that are either purchased from the American Society for Histocompatibility and Immunogenetics (ASHI, Boston, Mass.) or established from the pool of patients as described (Guilhot, et al. J. Virol. 66:2670-78, 1992).

Target cells consist of either allogeneic HLA-matched or autologous EBV-transformed B lymphoblastoid cell line that are incubated overnight with the synthetic peptide epitope of the invention at 10 μM, and labeled with 100 μCi of ⁵¹Cr (Amersham Corp., Arlington Heights, Ill.) for 1 hour after which they are washed four times with HBSS.

Cytolytic activity is determined in a standard 4-h, split well ⁵¹Cr release assay using U-bottomed 96 well plates containing 3,000 targets/well. Stimulated PBMC are tested at effector/target (E/T) ratios of 20-50:1 on day 14. Percent cytotoxicity is determined from the formula: 100×[(experimental release-spontaneous release)/maximum release-spontaneous release)]. Maximum release is determined by lysis of targets by detergent (2% Triton X-100; Sigma Chemical Co., St. Louis, Mo.). Spontaneous release is <25% of maximum release for all experiments.

ELISPOT Assay

An ELISPOT assay may be performed essentially as described in Example 13.

The results of either analysis indicate the extent to which HLA-restricted CTL populations have been stimulated by previous exposure to HPV or an HPV vaccine.

The class II restricted HTL responses may also be analyzed in several ways that are well known in the art.

A Direct Cellular Antigen-Specific T Cell Proliferation Assay

Purified PBMC are cultured in a 96-well flat bottom plate at a density of 1.5×10⁵ cells/well and are stimulated with 10 μg/ml synthetic peptide, whole antigen, or PHA. Cells are routinely plated in replicates of 4-6 wells for each condition. After seven days of culture, the medium is removed and replaced with fresh medium containing 10 U/ml IL-2. Two days later, 1 μCi ³H-thymidine is added to each well and incubation is continued for an additional 18 hours. Cellular DNA is then harvested on glass fiber mats and analyzed for ³H-thymidine incorporation. Antigen-specific T cell proliferation is calculated as the ratio of ³H-thymidine incorporation in the presence of antigen divided by the ³H-thymidine incorporation in the absence of antigen.

ELISPOT Antigen-Specific T Cell Proliferation Assay

An ELISPOT antigen-specific T cell proliferation assay may be performed to analyze a class II restricted helper T cell response. The assay is performed essentially as described in Example 13.

### Example 19

### Induction of Specific CTL Response in Humans

A human clinical trial for an immunogenic composition comprising CTL and HTL epitopes of the invention is set up as an IND Phase I, dose escalation study and carried out as a randomized, double-blind, placebo-controlled trial. Such a trial is designed, for example, as follows:

A total of about 27 individuals are enrolled and divided into 3 groups:

Group I: 3 subjects are injected with placebo and 6 subjects are injected with 5 μg of peptide composition;

Group II: 3 subjects are injected with placebo and 6 subjects are injected with 50 μg peptide composition;

Group III: 3 subjects are injected with placebo and 6 subjects are injected with 500 μg of peptide composition.

After 4 weeks following the first injection, all subjects receive a booster inoculation at the same dosage.

The endpoints measured in this study relate to the safety and tolerability of the peptide composition as well as its immunogenicity. Cellular immune responses to the peptide composition are an index of the intrinsic activity of this the peptide composition, and can therefore be viewed as a measure of biological efficacy. The following summarize the clinical and laboratory data that relate to safety and efficacy endpoints.

Safety: The incidence of adverse events is monitored in the placebo and drug treatment group and assessed in terms of degree and reversibility.

Evaluation of Vaccine Efficacy: For evaluation of vaccine efficacy, subjects are bled before and after injection. Peripheral blood mononuclear cells are isolated from fresh heparinized blood by Ficoll-Hypaque density gradient centrifugation, aliquoted in freezing media and stored frozen. Samples are assayed for CTL and HTL activity.

An acceptable vaccine is found to be both safe and efficacious.

### Example 20

### Phase II Trials in Patients Infected with HPV

Phase II trials are performed to study the effect of administering the CTL-HTL peptide compositions to patients having cancer associated with HPV infection. The main objectives of the trials are to determine an effective dose and regimen for inducing CTLs in HPV-infected patients with cancer, to establish the safety of inducing a CTL and HTL response in these patients, and to see to what extent activation of CTLs improves the clinical picture of chronically infected HPV patients, as manifested by a reduction in viral load, e.g., the reduction and/or shrinking of lesions. Such a study is designed, for example, as follows.

The studies are performed in multiple centers. The trial design is an open-label, uncontrolled, dose escalation protocol wherein the peptide composition is administered as a single dose followed six weeks later by a single booster shot of the same dose. The dosages are 50, 500 and 5,000 micrograms per injection. Drug-associated adverse effects (severity and reversibility) are recorded.

There are three patient groupings. The first group is injected with 50 micrograms of the peptide composition and the second and third groups with 500 and 5,000 micrograms of peptide composition, respectively. The patients within each group range in age from 21-65 and represent diverse ethnic backgrounds. All of them are infected with HPV and are HIV, HCV, HBV and delta hepatitis virus (HDV) negative, but are positive for HPV DNA as monitered by PCR.

Clinical manifestations or antigen-specific T-cell responses are monitored to assess the effects of administering the peptide compositions. An acceptable vaccine composition is found to be both safe and efficacious in the treatment of HPV infection.

### Example 21

### Induction of CTL Responses Using a Prime Boost Protocol

A prime boost protocol similar in its underlying principle to that used to evaluate the efficacy of a DNA vaccine in transgenic mice, such as described in Example 12, can also be used for the administration of the vaccine to humans. Such a vaccine regimen can include an initial administration of, for example, naked DNA followed by a boost using recombinant virus encoding the vaccine, or recombinant protein/polypeptide or a peptide mixture administered in an adjuvant.

For example, the initial immunization may be performed using an expression vector, such as that constructed in Example 11, in the form of naked polynucleotide administered IM (or SC or ID) in the amounts of 0.5-5 mg at multiple sites. The polynucleotide (0.1 to 1000 μg) can also be administered using a gene gun. Following an incubation period of 3-4 weeks, a booster dose is then administered. The booster can be recombinant fowlpox virus administered at a dose of 5×10⁷ to 5×10⁹ pfu. An alternative recombinant virus, such as an MVA (for example, modified Vaccinia Virus Ankara (“MVA-BN,” Bavarian-Nordic)), canarypox, adenovirus, or adeno-associated virus, can also be used for the booster, or the polyepitopic protein or a mixture of the peptides can be administered. For evaluation of vaccine efficacy, patient blood samples will be obtained before immunization as well as at intervals following administration of the initial vaccine and booster doses of the vaccine. Peripheral blood mononuclear cells are isolated from fresh heparinized blood by Ficoll-Hypaque density gradient centrifugation, aliquoted in freezing media and stored frozen. Samples are assayed for CTL and HTL activity.

Analysis of the results indicates that a magnitude of response sufficient to achieve protective immunity against HPV is generated.

### Example 22

### Administration of Vaccine Compositions Using Dendritic Cells (DC)

Vaccines comprising peptide epitopes of the invention can be administered using APCs, or “professional” APCs such as DC. In this example, the peptide-pulsed DC are administered to a patient to stimulate a CTL response in vivo. In this method, dendritic cells are isolated, expanded, and pulsed with a vaccine comprising peptide CTL and HTL epitopes of the invention. The dendritic cells are infused back into the patient to elicit CTL and HTL responses in vivo. The induced CTL and HTL then destroy or facilitate destruction of the specific target cells that bear the proteins from which the epitopes in the vaccine are derived.

For example, a cocktail of epitope-bearing peptides is administered ex vivo to PBMC, or isolated DC therefrom. A pharmaceutical to facilitate harvesting of DC can be used, such as Progenipoietin (Monsanto, St. Louis, Mo.) or GM-CSF/IL-4. After pulsing the DC with peptides and prior to reinfusion into patients, the DC are washed to remove unbound peptides.

As appreciated clinically, and readily determined by one of skill based on clinical outcomes, the number of DC reinfused into the patient can vary (see, e.g., Nature Med. 4:328, 1998; Nature Med. 2:52, 1996 and Prostate 32:272, 1997). Although 2-50×10⁶ DC per patient are typically administered, larger number of DC, such as 10⁷ or 10⁸ can also be provided. Such cell populations typically contain between 50-90% DC.

In some embodiments, peptide-loaded PBMC are injected into patients without purification of the DC. For example, PBMC containing DC generated after treatment with an agent such as Progenipoietin are injected into patients without purification of the DC. The total number of PBMC that are administered often ranges from 10⁸ to 10¹⁰. Generally, the cell doses injected into patients is based on the percentage of DC in the blood of each patient, as determined, for example, by immunofluorescence analysis with specific anti-DC antibodies. Thus, for example, if Progenipoietin™ mobilizes 2% DC in the peripheral blood of a given patient, and that patient is to receive 5×10⁶ DC, then the patient will be injected with a total of 2.5×10⁸ peptide-loaded PBMC. The percent DC mobilized by an agent such as Progenipoietin is typically estimated to be between 2-10%, but can vary as appreciated by one of skill in the art.

Ex Vivo Activation of CTL/HTL Responses

Alternatively, ex vivo CTL or HTL responses to HPV antigens can be induced by incubating in tissue culture the patient's, or genetically compatible, CTL or HTL precursor cells together with a source of APC, such as DC, and the appropriate immunogenic peptides. After an appropriate incubation time (typically about 7-28 days), in which the precursor cells are activated and expanded into effector cells, the cells are infused back into the patient, where they will destroy (CTL) or facilitate destruction (HTL) of their specific target cells, i.e., tumor cells.

### Example 23

### Alternative Method of Identifying Motif-Bearing Peptides

Another method of identifying motif-bearing peptides is to elute them from cells bearing defined MHC molecules. For example, EBV transformed B cell lines used for tissue typing have been extensively characterized to determine which HLA molecules they express. In certain cases these cells express only a single type of HLA molecule. These cells can be infected with a pathogenic organism or transfected with nucleic acids that express the antigen of interest, e.g. HPV regulatory or structural proteins. Peptides produced by endogenous antigen processing of peptides produced consequent to infection (or as a result of transfection) will then bind to HLA molecules within the cell and be transported and displayed on the cell surface. Peptides are then eluted from the HLA molecules by exposure to mild acid conditions and their amino acid sequence determined, e.g., by mass spectral analysis (e.g., Kubo, et al., J. Immunol. 152:3913, 1994). Because the majority of peptides that bind a particular HLA molecule are motif-bearing, this is an alternative modality for obtaining the motif-bearing peptides correlated with the particular HLA molecule expressed on the cell.

Alternatively, cell lines that do not express endogenous HLA molecules can be transfected with an expression construct encoding a single HLA allele. These cells can then be used as described, i.e., they can be infected with a pathogen or transfected with nucleic acid encoding an antigen of interest to isolate peptides corresponding to the pathogen or antigen of interest that have been presented on the cell surface. Peptides obtained from such an analysis will bear motif(s) that correspond to binding to the single HLA allele that is expressed in the cell.

As appreciated by one in the art, one can perform a similar analysis on a cell bearing more than one HLA allele and subsequently determine peptides specific for each HLA allele expressed. Moreover, one of skill would also recognize that means other than infection or transfection, such as loading with a protein antigen, can be used to provide a source of antigen to the cell.

The above examples are provided to illustrate the invention but not to limit its scope. For example, the human terminology for the Major Histocompatibility Complex, namely HLA, is used throughout this document. It is to be appreciated that these principles can be extended to other species as well. Thus, other variants of the invention will be readily apparent to one of ordinary skill in the art and are encompassed by the appended claims. All publications, patents, and patent applications, and all figures, drawings, and sequence listings associated therewith, cited herein are hereby incorporated by reference for all purposes.

LENGTHY TABLE REFERENCED HERE
US20070014810A1-20070118-T00001
Please refer to the end of the specification for access instructions.

LENGTHY TABLE REFERENCED HERE
US20070014810A1-20070118-T00002
Please refer to the end of the specification for access instructions.

LENGTHY TABLE REFERENCED HERE
US20070014810A1-20070118-T00003
Please refer to the end of the specification for access instructions.

LENGTHY TABLE REFERENCED HERE
US20070014810A1-20070118-T00004
Please refer to the end of the specification for access instructions.

LENGTHY TABLE REFERENCED HERE
US20070014810A1-20070118-T00005
Please refer to the end of the specification for access instructions.

LENGTHY TABLE REFERENCED HERE
US20070014810A1-20070118-T00006
Please refer to the end of the specification for access instructions.

LENGTHY TABLE REFERENCED HERE
US20070014810A1-20070118-T00007
Please refer to the end of the specification for access instructions.

LENGTHY TABLE REFERENCED HERE
US20070014810A1-20070118-T00008
Please refer to the end of the specification for access instructions.

LENGTHY TABLE REFERENCED HERE
US20070014810A1-20070118-T00009
Please refer to the end of the specification for access instructions.

LENGTHY TABLE REFERENCED HERE
US20070014810A1-20070118-T00010
Please refer to the end of the specification for access instructions.

LENGTHY TABLE REFERENCED HERE
US20070014810A1-20070118-T00011
Please refer to the end of the specification for access instructions.

LENGTHY TABLE REFERENCED HERE
US20070014810A1-20070118-T00012
Please refer to the end of the specification for access instructions.

LENGTHY TABLE REFERENCED HERE
US20070014810A1-20070118-T00013
Please refer to the end of the specification for access instructions.

LENGTHY TABLE REFERENCED HERE
US20070014810A1-20070118-T00014
Please refer to the end of the specification for access instructions.

LENGTHY TABLE REFERENCED HERE
US20070014810A1-20070118-T00015
Please refer to the end of the specification for access instructions.

LENGTHY TABLE REFERENCED HERE
US20070014810A1-20070118-T00016
Please refer to the end of the specification for access instructions.

LENGTHY TABLE REFERENCED HERE
US20070014810A1-20070118-T00017
Please refer to the end of the specification for access instructions.

LENGTHY TABLE REFERENCED HERE
US20070014810A1-20070118-T00018
Please refer to the end of the specification for access instructions.

LENGTHY TABLE REFERENCED HERE
US20070014810A1-20070118-T00019
Please refer to the end of the specification for access instructions.

LENGTHY TABLE REFERENCED HERE
US20070014810A1-20070118-T00020
Please refer to the end of the specification for access instructions.

LENGTHY TABLE REFERENCED HERE
US20070014810A1-20070118-T00021
Please refer to the end of the specification for access instructions.

LENGTHY TABLE REFERENCED HERE
US20070014810A1-20070118-T00022
Please refer to the end of the specification for access instructions.

LENGTHY TABLE REFERENCED HERE
US20070014810A1-20070118-T00023
Please refer to the end of the specification for access instructions.

LENGTHY TABLE REFERENCED HERE
US20070014810A1-20070118-T00024
Please refer to the end of the specification for access instructions.

LENGTHY TABLE REFERENCED HERE
US20070014810A1-20070118-T00025
Please refer to the end of the specification for access instructions.

LENGTHY TABLE REFERENCED HERE
US20070014810A1-20070118-T00026
Please refer to the end of the specification for access instructions.

LENGTHY TABLE REFERENCED HERE
US20070014810A1-20070118-T00027
Please refer to the end of the specification for access instructions.

LENGTHY TABLE REFERENCED HERE
US20070014810A1-20070118-T00028
Please refer to the end of the specification for access instructions.

LENGTHY TABLE REFERENCED HERE
US20070014810A1-20070118-T00029
Please refer to the end of the specification for access instructions.

LENGTHY TABLE REFERENCED HERE
US20070014810A1-20070118-T00030
Please refer to the end of the specification for access instructions.

LENGTHY TABLE REFERENCED HERE
US20070014810A1-20070118-T00031
Please refer to the end of the specification for access instructions.

LENGTHY TABLE REFERENCED HERE
US20070014810A1-20070118-T00032
Please refer to the end of the specification for access instructions.

LENGTHY TABLE REFERENCED HERE
US20070014810A1-20070118-T00033
Please refer to the end of the specification for access instructions.

LENGTHY TABLE REFERENCED HERE
US20070014810A1-20070118-T00034
Please refer to the end of the specification for access instructions.

LENGTHY TABLE REFERENCED HERE
US20070014810A1-20070118-T00035
Please refer to the end of the specification for access instructions.

LENGTHY TABLE REFERENCED HERE
US20070014810A1-20070118-T00036
Please refer to the end of the specification for access instructions.

LENGTHY TABLE REFERENCED HERE
US20070014810A1-20070118-T00037
Please refer to the end of the specification for access instructions.

LENGTHY TABLE REFERENCED HERE
US20070014810A1-20070118-T00038
Please refer to the end of the specification for access instructions.

LENGTHY TABLE REFERENCED HERE
US20070014810A1-20070118-T00039
Please refer to the end of the specification for access instructions.

LENGTHY TABLE REFERENCED HERE
US20070014810A1-20070118-T00040
Please refer to the end of the specification for access instructions.

LENGTHY TABLE REFERENCED HERE
US20070014810A1-20070118-T00041
Please refer to the end of the specification for access instructions.

LENGTHY TABLE REFERENCED HERE
US20070014810A1-20070118-T00042
Please refer to the end of the specification for access instructions.

LENGTHY TABLE REFERENCED HERE
US20070014810A1-20070118-T00043
Please refer to the end of the specification for access instructions.

LENGTHY TABLE REFERENCED HERE
US20070014810A1-20070118-T00044
Please refer to the end of the specification for access instructions.

LENGTHY TABLE REFERENCED HERE
US20070014810A1-20070118-T00045
Please refer to the end of the specification for access instructions.

LENGTHY TABLE REFERENCED HERE
US20070014810A1-20070118-T00046
Please refer to the end of the specification for access instructions.

LENGTHY TABLE REFERENCED HERE
US20070014810A1-20070118-T00047
Please refer to the end of the specification for access instructions.

LENGTHY TABLE REFERENCED HERE
US20070014810A1-20070118-T00048
Please refer to the end of the specification for access instructions.

LENGTHY TABLE REFERENCED HERE
US20070014810A1-20070118-T00049
Please refer to the end of the specification for access instructions.

LENGTHY TABLE REFERENCED HERE
US20070014810A1-20070118-T00050
Please refer to the end of the specification for access instructions.

LENGTHY TABLE REFERENCED HERE
US20070014810A1-20070118-T00051
Please refer to the end of the specification for access instructions.

LENGTHY TABLE REFERENCED HERE
US20070014810A1-20070118-T00052
Please refer to the end of the specification for access instructions.

LENGTHY TABLE REFERENCED HERE
US20070014810A1-20070118-T00053
Please refer to the end of the specification for access instructions.

LENGTHY TABLE REFERENCED HERE
US20070014810A1-20070118-T00054
Please refer to the end of the specification for access instructions.

LENGTHY TABLE REFERENCED HERE
US20070014810A1-20070118-T00055
Please refer to the end of the specification for access instructions.

LENGTHY TABLE REFERENCED HERE
US20070014810A1-20070118-T00056
Please refer to the end of the specification for access instructions.

LENGTHY TABLE REFERENCED HERE
US20070014810A1-20070118-T00057
Please refer to the end of the specification for access instructions.

LENGTHY TABLE REFERENCED HERE
US20070014810A1-20070118-T00058
Please refer to the end of the specification for access instructions.

LENGTHY TABLE REFERENCED HERE
US20070014810A1-20070118-T00059
Please refer to the end of the specification for access instructions.

LENGTHY TABLE REFERENCED HERE
US20070014810A1-20070118-T00060
Please refer to the end of the specification for access instructions.

LENGTHY TABLE REFERENCED HERE
US20070014810A1-20070118-T00061
Please refer to the end of the specification for access instructions.

LENGTHY TABLE REFERENCED HERE
US20070014810A1-20070118-T00062
Please refer to the end of the specification for access instructions.

LENGTHY TABLE REFERENCED HERE
US20070014810A1-20070118-T00063
Please refer to the end of the specification for access instructions.

LENGTHY TABLE REFERENCED HERE
US20070014810A1-20070118-T00064
Please refer to the end of the specification for access instructions.

LENGTHY TABLE REFERENCED HERE
US20070014810A1-20070118-T00065
Please refer to the end of the specification for access instructions.

LENGTHY TABLE REFERENCED HERE
US20070014810A1-20070118-T00066
Please refer to the end of the specification for access instructions.

LENGTHY TABLE REFERENCED HERE
US20070014810A1-20070118-T00067
Please refer to the end of the specification for access instructions.

LENGTHY TABLE REFERENCED HERE
US20070014810A1-20070118-T00068
Please refer to the end of the specification for access instructions.

LENGTHY TABLE REFERENCED HERE
US20070014810A1-20070118-T00069
Please refer to the end of the specification for access instructions.

LENGTHY TABLE REFERENCED HERE
US20070014810A1-20070118-T00070
Please refer to the end of the specification for access instructions.

LENGTHY TABLE REFERENCED HERE
US20070014810A1-20070118-T00071
Please refer to the end of the specification for access instructions.

LENGTHY TABLE REFERENCED HERE
US20070014810A1-20070118-T00072
Please refer to the end of the specification for access instructions.

LENGTHY TABLE REFERENCED HERE
US20070014810A1-20070118-T00073
Please refer to the end of the specification for access instructions.

LENGTHY TABLE REFERENCED HERE
US20070014810A1-20070118-T00074
Please refer to the end of the specification for access instructions.

LENGTHY TABLE REFERENCED HERE
US20070014810A1-20070118-T00075
Please refer to the end of the specification for access instructions.

In other embodiments, the invention provides a polynucleotide selected from the following polynucleotides (a)-(t), each encoding the human papillomavirus (HPV) helper T lymphocyte (HTL) epitopes of Core Group HTL780-21.1/22.1/24.

(a) A multi-epitope polynucleotide construct comprising nucleic acids encoding the human papillomavirus (HPV) helper T lymphocyte (HTL) epitopes of Core Group HTL780-21.1/22.1/24. These epitopes are: HPV16.E1.319, HPV16.E1.337, HPV18.E1.258, HPV18.E1.458, HPV18.E2.140, HPV31.E1.015, HPV31.E1.317, HPV45.E1.484, HPV45.E1.510, HPV45.E2.352 and HPV45.E2.67, wherein the nucleic acids are directly or indirectly joined to one another in the same reading frame. Note that the nucleic acids encoding the epitopes listed above may be arranged in any order.

(b) A multi-epitope polynucleotide construct comprising nucleic acids encoding the human papillomavirus (HPV) cytotoxic T lymphocyte (CTL) epitopes of Core Group HTL780-21.1/22.1/24. (hereinafter “the HTL780-21.1/22.1/24. core construct”), and also encoding one or more additional CTL and/or HTL epitopes.

(c) The HTL780-21.1/22.1/24 core construct as in (a)-(b), where the nucleic acids encoding the epitopes listed above are arranged in a specified order, but may have additional nucleic acids encoding additional epitopes and/or spacer amino acids dispersed therein.

(d) The HTL780-21.1/22.1/24 core construct as in (a)-(c), where one or more epitope-encoding nucleic acids are flanked by spacer nucleotides, and/or other polynucleotide sequences as described herein or otherwise known in the art. Such spacer nucleotides encode one or more spacer amino acids so as to keep the multi-epitope construct in frame.

(e) The HTL780-21.1/22.1/24 core construct as in (a)-(d), where the multi-epitopeconstruct is distinguished from other multi-epitopeconstructs according to whether the spacer nucleotides in one construct encode spacer amino acids which optimize epitope processing and/or minimize junctional epitopes with respect to other constructs as described herein or elsewhere.

(f) The HTL780-21.1/22.1/24 core construct as in (a)-(e), where the multi-epitope construct encodes a polypeptide which is concomitantly optimized for epitope processing and junctional epitopes with respect to one or more other constructs as described herein.

(g) The HTL780-21.1/22.1/24 core construct as in (a)-(f), where the multi-epitope-construct further comprises a PADRE HTL epitope, as described herein.

(h) The HTL780-21.1/22.1/24 core construct as in (a)-(g), further comprising nucleic acids encoding HPV HTL epitopes HPV16.E2.156, HPV16.E2.7, HPV31.E2.354, HPV31.E2.67 and HPV18.E2.277.

(i) The HTL780-21.1/22.1/24 core construct as in (a)-(h), further comprising nucleic acids encoding HPV HTL epitopes HPV16.E2.160, HPV16.E2.19, HPV18.E2.127, HPV18.E2.340 and HPV31.E2.202.

(j) The HTL780-21.1/22.1/24 core construct as in (h), comprising or alternatively consisting of the multi-epitope construct HTL 780-24 (See Tables 78 and 79).

(k) The HTL780-21.1/22.1/24 core construct as in (i), comprising or alternatively consisting of the multi-epitope construct HTL 780-21.1 (See Tables 58A and 59).

(l) The HTL780-21.1/22.1/24 core construct as in (i), comprising or alternatively consisting of the multi-epitope construct HTL 780-22.1 (See Tables 58B and 61).

(m) The HTL780-21.1/22.1/24 core construct as in (a)-(1), further comprising further comprising any of the HPV 46 core constructs (a)-(m) as described above.

(n) The HTL780-21.1/22.1/24 core construct as in (a)-(m), further comprising nucleic acids encoding HPV CTL epitopes HPV16.E1.493, HPV31/52.E1.557, HPV31.E2.131, HPV31.E2.127, HPV16.E2.335, HPV16.E2.37, HPV16.E2.93, HPV18.E2.211, HPV18.E2.61, HPV18.E1.266 and HPV18.E1.500.

(o) The HTL780-21.1/22.1/24 core construct as in (a)-(n), further comprising nucleic acids encoding HPV CTL epitopes HPV16.E1.191, HPV16.E1.292, HPV16.E1.489, HPV16.E1.489, HPV16/52.E1.406, HPV18.E1.210, HPV18.E1.266, HPV18.E1.463, HPV31.E1.464, HPV18/45.E1.284 and HPV31.E1.441.

(p) The HTL780-21.1/22.1/24 core construct as in (n), comprising or alternatively consisting of the multi-epitope construct HPV 47-1/HTL780.21.1 (See Tables 63A, 64A and 65A).

(q) The HTL780-21.1/22.1/24 core construct as in (n), comprising or alternatively consisting of the multi-epitope construct HPV 47-1/HTL780.22.1 (See Tables 63B, 64B and 65B).

(r) The HTL780-21.1/22.1/24 core construct as in (n), comprising or alternatively consisting of the multi-epitope construct HPV 47-2/HTL780.21.1 (See Tables 63C, 64C and 65C).

(s) The HTL780-21.1/22.1/24 core construct as in (n), comprising or alternatively consisting of the multi-epitope construct HPV 47-2/HTL780.22.1 (See Tables 63D, 64D and 65D).

(t) The HTL780-21.1/22.1/24 core construct as in (o), comprising or alternatively consisting of the multi-epitope construct HPV 47-3/HTL780.24 (See Tables.

In other embodiments, the invention provides a polypeptide comprising HTL780-21.1/22.1/24 HTL epitopes encoded by any of polynucleotides (a)-(t) listed above.

LENGTHY TABLE
The patent application contains a lengthy table section. A copy of the table is available in electronic form from the USPTO web site (http://seqdata.uspto.gov/?pageRequest=docDetail&DocID=US20070014810A1) An electronic copy of the table will also be available from the USPTO upon request and payment of the fee set forth in 37 CFR 1.19(b)(3).

1. A polynucleotide selected from the group consisting of:
(a) a multi-epitope construct comprising nucleic acids encoding the human papillomavirus (HPV) cytotoxic T lymphocyte (CTL) epitopes HPV16.E1.214, HPV16.E1.254, HPV16.E1.314, HPV16.E1.420, HPV16.E1.585, HPV16.E2.130, HPV16.E2.329, HPV16/52.E2.151, HPV18.E1.592, HPV18.E2.136, HPV18.E2.142, HPV18.E2.15, HPV18.E2.154, HPV18.E2.168, HPV18.E2.230, HPV18/45.E1.321, HPV18/45.E1.491, HPV31.E1.272, HPV31.E1.349, HPV31.E1.565, HPV31.E2.11, HPV31.E2.130, HPV31.E2.138, HPV31.E2.205, HPV31.E2.291, HPV31.E2.78, HPV45.E1.232, HPV45.E1.252, HPV45.E1.399, HPV45.E1.411, HPV45.E1.578, HPV45.E2.137, HPV45.E2.144, HPV45.E2.17, HPV45.E2.332, and HPV45.E2.338, wherein the nucleic acids are directly or indirectly joined to one another in the same reading frame;
(b) the multi-epitope construct of (a), further comprising nucleic acids encoding the human papillomavirus (HPV) cytotoxic T lymphocyte (CTL) epitopes HPV16.E1.493, HPV31/52.E1.557, HPV31.E2.131, HPV31.E2.127, HPV16.E2.335, HPV16.E2.37, HPV16.E2.93, HPV18.E2.211, HPV18.E2.61, HPV18.E1.266, and HPV18.E1.500, directly or indirectly joined in the same reading frame to said CTL epitope nucleic acids of (a);
(c) the multi-epitope construct of (a), further comprising nucleic acids encoding the human papillomavirus (HPV) cytotoxic T lymphocyte (CTL) epitopes HPV16.E1.191, HPV16.E1.292, HPV16.E1.489, HPV16.E1.489, HPV16/52.E1.406, HPV18.E1.210, HPV18.E1.266, HPV18.E1.463, HPV31.E1.464, HPV18/45.E1.284, and HPV31.E1.441 directly or indirectly joined in the same reading frame to said CTL epitope nucleic acids of (a);
(d) the multi-epitope construct of (a), further comprising nucleic acids encoding the human papillomavirus (HPV) cytotoxic T lymphocyte (CTL) epitopes HPV16.E1.191, HPV16.E1.292, HPV16.E1.489, HPV16.E1.489, HPV16/52.E1.406, HPV18.E1.210, HPV18.E1.266, HPV18.E1.463, HPV31.E1.464, HPV18/45.E1.284, and HPV31.E1.441 directly or indirectly joined in the same reading frame to said CTL epitope nucleic acids of (a);
(e) a multi-epitope construct comprising nucleic acids encoding the human papillomavirus (HPV) cytotoxic T lymphocyte (CTL) epitopes HPV16.E6.106, HPV16.E6.29.L2, HPV16.E6.68.R10, HPV16.E6.75.F9, HPV16.E6.75.L2, HPV16.E6.77, HPV16.E6.80.D3, HPV16.E7.11.V10, HPV16.E7.2.T2, HPV16.E7.56.F10, HPV16.E7.86.V8, HPV18.E6.24, HPV18.E6.25.T2, HPV18.E6.53.K10, HPV18.E6.72.D3, HPV18.E6.83.R10, HPV18.E6.84.V10, HPV18.E6.89, HPV18.E6.92.V10, HPV18.E7.59.R9, HPV18/45.E6.13, HPV18/45.E6.98.F9, HPV31.E6.132.K10, HPV31.E6.15, HPV31.E6.72, HPV31.E6.73 D3, HPV31.E6.80, HPV31.E6.82R9, HPV31.E6.83, HPV31.E6.90, HPV31.E7.44.T2, HPV33.E7.11 V10, HPV45.E6.24, HPV45.E6.25 T2, HPV45.E6.37, HPV45.E6.41.R10, HPV45.E6.44, HPV45.E6.54, HPV45.E6.54. V10, HPV45.E6.71.F10, HPV45.E6.84.R9 and HPV45.E7.20, wherein the nucleic acids are directly or indirectly joined to one another in the same reading frame;
(f) the multi-epitope construct of (e), further comprising nucleic acids encoding the human papillomavirus (HPV) cytotoxic T lymphocyte (CTL) epitopes HPV16.E6.131, HPV18.E6.126.F9, HPV31.E6.69, HPV18.E6.33.F9, directly or indirectly joined in the same reading frame to said CTL epitope nucleic acids of (d);
(g) the the multi-epitope construct of (e), further comprising nucleic acids encoding the human papillomavirus (HPV) cytotoxic T lymphocyte (CTL) epitopes HPV18.E6.33, HPV16.E6.87, HPV18.E6.44, HPV31.E6.69+R@68, directly or indirectly joined in the same reading frame to said CTL epitope nucleic acids of (d);
(h) the multi-epitope construct of (a) or (b) or (c) or (d) or (e) or (f) or (g), further comprising one or more spacer nucleic acids encoding one or more spacer amino acids, directly or indirectly joined in the same reading frame to said CTL epitope nucleic acids;
(i) the multi-epitope construct of (h), wherein said one or more spacer nucleic acids are positioned between the CTL epitope nucleic acids of (a), between the CTL epitope nucleic acids of (b), between the CTL epitope nucleic acids of (c), between the CTL epitope nucleic acids of (d), between the CTL epitope nucleic acids of (a) and (b), between the CTL epitope nucleic acids of (a) and (c), between the CTL epitope nucleic acids of (a) and (d), between the CTL epitope nucleic acids of (e), between the CTL epitope nucleic acids of (f), between the CTL epitope nucleic acids of (g), between the CTL epitope nucleic acids of (e) and (f), or between the CTL epitope nucleic acids of (e) and (g);
(j) the multi-epitope construct of (h) or (i), wherein said one or more spacer nucleic acids each encode 1 to 8 amino acids;
(k) the multi-epitope construct of any of (h) to (j), wherein two or more of said spacer nucleic acids encode different (i.e., non-identical) amino acid sequences;
(l) the multi-epitope construct of any of (h) to (k), wherein two or more of said spacer nucleic acids encode an amino acid sequence different from an amino acid sequence encoded by one or more other spacer nucleic acids;
(m) the multi-epitope construct of any of (h) to (l), wherein two or more of the spacer nucleic acids encodes the identical amino acid sequence;
(n) the multi-epitope construct of any of (h) to (m), wherein one or more of said spacer nucleic acids encode an amino acid sequence comprising or consisting of three consecutive alanine (Ala) residues;
(o) the multi-epitope construct of any of (a) to (n), further comprising one or more nucleic acids encoding one or more HTL epitopes, directly or indirectly joined in the same reading frame to said CTL epitope nucleic acids and/or said spacer nucleic acids;
(p) the multi-epitope construct of (o), wherein said one or more HTL epitopes comprises a PADRE epitope;
(q) the multi-epitope construct of (o) or (p), wherein said one or more HTL epitopes comprise one or more HPV HTL epitopes;
(r) the multi-epitope construct of (q), wherein said one or more HPV HTL epitopes comprise HPV16.E1.319,HPV16.E1.337, HPV18.E1.258, HPV18.E1.458, HPV18.E2.140, HPV31.E1.015, HPV31.E1.317, HPV31.E2.67, HPV45.E1.484, HPV45.E1.510, and HPV45.E2.352;
(s) the multi-epitope construct of (r), wherein said one or more HPV HTL epitopes further comprise HPV16.E2.156, HPV16.E2.7, HPV18.E2.277, HPV31.E2.354, and HPV45.E2.67;
(t) the multi-epitope construct of (r), wherein said one or more HPV HTL epitopes further comprise HPV16.E2.160, HPV16.E2.19, HPV18.E2.127, HPV18.E2.340, and HPV31.E2.202;
(u) the multi-epitope construct of (q), wherein said one or more HPV HTL epitopes comprise HPV16.E6.13, HPV16.E6.130, HPV16.E7.13, HPV16.E7.46, HPV16.E7.76, HPV18.E6.43, HPV31.E6.132, HPV31.E6.42, HPV31.E6.78, HPV45.E6.127, and HPV45.E7.10;
(v) the multi-epitope construct of (u), wherein said one or more HPV HTL epitopes further comprise HPV18.E6.94, HPV18.E7.78, HPV31.E6.1, HPV31.E7.36, and HPV45.E7.82;
(w) the multi-epitope construct of (u), wherein said one or more HPV HTL epitopes further comprise HPV18.E6.52 and 53, HPV18.E6.94+Q, HPV18.E7.86, HPV31.E7.76, and HPV45.E6.52;
(x) the multi-epitope construct of any of (o) to (w), further comprising one or more spacer nucleic acids encoding one or more spacer amino acids directly or indirectly joined in the same reading frame between a CTL epitope and an HTL epitope or between HTL epitopes;
(y) the multi-epitope construct of (x), wherein said spacer nucleic acid encodes an amino acid sequence selected from the group consisting of: an amino acid sequence comprising or consisting of GPGPG (SEQ ID NO: 1), an amino acid sequence comprising or consisting of PGPGP (SEQ ID NO: 2), an amino acid sequence comprising or consisting of (GP)n (SEQ ID NO: 3), an amino acid sequence comprising or consisting of (PG)n (SEQ ID NO: 4), an amino acid sequence comprising or consisting of (GP)nG (SEQ ID NO: 5), and an amino acid sequence comprising or consisting of (PG)Np (SEQ ID NO: 6), where n is an integer between zero and eleven;
(z) the multi-epitope construct of any of (a) to (y), further comprising one or more MHC Class I and/or MHC Class II targeting nucleic acids;
(aa) the multi-epitope construct of (z), wherein said one or more targeting nucleic acids encode one or more targeting sequences selected from the group consisting of: an Ig kappa signal sequence, a tissue plasminogen activator signal sequence, an insulin signal sequence, an endoplasmic reticulum signal sequence, a LAMP-1 lysosomal targeting sequence, a LAMP-2 lysosomal targeting sequence, an HLA-DM lysosomal targeting sequence, an HLA-DM-association sequence of HLA-DO, an Ig-a cytoplasmic domain, Ig-ss cytoplasmic domain, a Ii protein, an influenza matrix protein, an HCV antigen, and a yeast Ty protein;
(bb) the multi-epitope construct of any of (a) to (aa), which is optimized for CTL and/or HTL epitope processing;
(cc) the multi-epitope construct of any of (a) to (bb), wherein said CTL nucleic acids are sorted to minimize the number of CTL and/or HTL junctional epitopes encoded therein;
(dd) the multi-epitope construct of any of (q) to (cc), wherein said HTL nucleic acids are sorted to minimize the number of CTL and/or HTL junctional epitopes encoded therein;
(ee) the multi-epitope construct of any of (a) to (dd) further comprising one or more nucleic acids encoding one or more flanking amino acid residues;
(ff) the multi-epitope construct of (ee), wherein said one or more flanking amino acid residues are selected from the group consisting of: K, R, N, Q, G, A, S, C, and T at a C+1 position of one of said CTL epitopes;
(gg) the multi-epitope construct of any of (e), (f), (h)-(n), (z)-(cc), (ee) or (ff), wherein said HPV CTL epitopes are directly or indirectly joined in the order shown in Table 47C;
(hh) the multi-epitope construct of any of (e), (g), (h)-(n), (z)-(cc), (ee) or (ff), wherein the HPV CTL epitopes are directly or indirectly joined in the order shown in Table 85;
(ii) the multi-epitope construct of any of (a), (b), (h)-(n), (z)-(cc), (ee) or (ff), wherein the HPV CTL epitopes are directly or indirectly joined in the order shown in Table 52A;
(ii) the multi-epitope construct of any of (a), (b), (h)-(n), (z)-(cc), (ee) or (ff), wherein the HPV CTL epitopes are directly or indirectly joined in the order shown in Table 52B;
(jj) the multi-epitope construct of any of (a), (c), (h)-(n), (z)-(cc), (ee) or (ff), wherein the HPV CTL epitopes are directly or indirectly joined in the order shown in Table 74;
(kk) the multi-epitope construct of any of (a), (c), (h)-(n), (z)-(cc), (ee) or (ff), wherein the HPV CTL epitopes are directly or indirectly joined in the order shown in Table 75;
(ll) the multi-epitope construct of any of (a), (d), (h)-(n), (z)-(cc), (ee) or (ff), wherein the HPV CTL epitopes are directly or indirectly joined in the order shown in Table 83;
(mm) the multi-epitope construct of any of (r), (t), (x)-(bb), (dd) or (ff), wherein the HPV HTL epitopes are directly or indirectly joined in the order shown in Table 58A;
(nn) the multi-epitope construct of any of (r), (t), (x)-(bb), (dd) or (ff), wherein the HPV HTL epitopes are directly or indirectly joined in the order shown in Table 58B;
(oo) the multi-epitope construct of any of (u), (v), (x)-(bb), (dd) or (ff), wherein the HPV HTL epitopes are directly or indirectly joined in the order of the HTL epitopes shown in Table 70;
(pp) the multi-epitope construct of any of (u), (w), (x)-(bb), (dd) or (ff), wherein the HPV HTL epitopes are directly or indirectly joined in the order shown in Table 80;
(qq) the multi-epitope construct of any of (e), (f), (h)-(n), (r), (s), or (x)-(ff), wherein the HPV HTL epitopes are directly or indirectly joined in the order shown in Table 78;
(rr) the multi-epitope construct of (e), (f), (h)-(n), (u), (v), or (x)-(ff), wherein said HPV CTL epitopes and said HPV HTL epitopes are directly or indirectly joined in the order shown in Table 70;
(ss) the multi-epitope construct of (e), (g), (h)-(n), (u), (v), or (x)-(ff), wherein said HPV CTL epitopes and said HPV HTL epitopes are directly or indirectly joined in the order shown in Table 71;
(tt) the multi-epitope construct of (a), (b), (h)-(n), (r), (t), or (x)-(ff), wherein said HPV CTL epitopes and said HPV HTL epitopes are directly or indirectly joined in the order shown in Table 63A;
(uu) the multi-epitope construct of (a), (b), (h)-(n), (r), (t), or (x)-(ff), wherein said HPV CTL epitopes and said HPV HTL epitopes are directly or indirectly joined in the order shown in Table 63C;
(vv) the multi-epitope construct of (a), (b), (h)-(n), (r), (t), or (x)-(ff), wherein said HPV CTL epitopes and said HPV HTL epitopes are directly or indirectly joined in the order shown in Table 63B;
(xx) the multi-epitope construct of (a), (b), (h)-(n), (r), (t), or (x)-(ff), wherein said HPV CTL epitopes and said HPV HTL epitopes are directly or indirectly joined in the order shown in Table 63D;
(yy) the multi-epitope construct of (a), (c), (h)-(n), (r), (s), or (x)-(ff), wherein said HPV CTL epitopes and said HPV HTL epitopes are directly or indirectly joined in the order shown in Table 84;
(zz) the multi-epitope construct of any of (a) to (ff), wherein said construct encodes a polypeptide comprising or consisting of an amino acid sequence selected from the group consisting of: the amino acid sequence shown in Table 50C, the amino acid sequence shown in Table 54A, the amino acid sequence shown in Table 54B, the amino acid sequence shown in Table 59, the amino acid sequence shown in Table 61, the amino acid sequence shown in Table 65A, the amino acid sequence shown in Table 65B, the amino acid sequence shown in Table 65C, the amino acid sequence shown in Table 65D, the amino acid sequence shown in Table 69, the amino acid sequence shown in Table 72A, the amino acid sequence shown in Table 72E, the amino acid sequence shown in Table 73A, the amino acid sequence shown in Table 76A, the amino acid sequence shown in Table 76C, the amino acid sequence shown in Table 79A, the amino acid sequence shown in Table 79B, the amino acid sequence shown in Table 81, and a combination of two or more of said amino acid sequences; and
(aaa) the multi-epitope construct of any of (a) to (ff), wherein said construct comprises a nucleic acid sequence selected from the group consisting of: the nucleotide sequence in Table 49C, the nucleotide sequence in Table 53A, the nucleotide sequence in Table 53B, the nucleotide sequence in Table 59, the nucleotide sequence in Table 61, the nucleotide sequence in Table 64A, the nucleotide sequence in Table 64B, the nucleotide sequence in Table 64C, the nucleotide sequence in Table 64D, the nucleotide sequence in Table 72B, the nucleotide sequence in Table 72F, the nucleotide sequence in Table 73B, the nucleotide sequence in Table 76B, the nucleotide sequence in Table 76D, the nucleotide sequence in Table 79A, the nucleotide sequence in Table 79B, the nucleotide sequence in Table 81, and a combination of two or more of said nucleotide sequences.

2. The multi-epitope construct of claim 1, further comprising one or more regulatory sequences.

3. The multi-epitope construct of claim 2, wherein said one or more regulatory sequences comprises an IRES element.

4. The multi-epitope construct of claim 2, wherein said one or more regulatory sequences comprises a promoter.

5. The multi-epitope construct of claim 4, wherein said promoter is a CMV promoter.

6. A vector comprising the multi-epitope construct of claim 1.

7. The vector of claim 6, wherein said vector is an expression vector.

8. A polynucleotide comprising a first multi-epitope constrcut, and a second multi-epitope construct, each according to claim 1, a first and a second multi-epitope constructs, said first multi-epitope construct comprising a polynucleotide encoding one or more HPV epitopes, and said second multi-epitope construct comprising a polynucleotide encoding one or more HPV HTL epitopes, wherein said first and second multi-epitope constructs are not directly joined, or are not joined in the same frame.

9. The polynucleotide of claim 8, wherein said first and second multi-epitope constructs are operably linked to at least one regulatory sequence.

10. The polynucleotide of claim 9, wherein said at least one regulatory sequence is selected from the group consisting of: a promoter, an IRES element, and a combination thereof.

11. The polynucleotide of claim 10, wherein said promoter is a CMV promoter.

12. The polynucleotide of claim 8, wherein said first and second multi-epitope constructs have a structure selected from the group consisting of the structure shown in any one of Tables 47C, 52B, 58A, 63A-D, 70, 71, 74, 75, 78, 80, 82, 83, 84, 85 and a combination of said structures.

13. The polynucleotide of claim 8, wherein said second multi-epitope construct encodes a polypeptide comprising or consisting of an amino acid sequence selected from the group consisting the amino acid sequence shown in Table 50C, the amino acid sequence shown in Table 54A, the amino acid sequence shown in Table 54B, the amino acid sequence shown in Table 59, the amino acid sequence shown in Table 61, the amino acid sequence shown in Table 65A, the amino acid sequence shown in Table 65B, the amino acid sequence shown in Table 65C, the amino acid sequence shown in Table 65D, the amino acid sequence shown in Table 69, the amino acid sequence shown in Table 72A, the amino acid sequence shown in Table 72E, the amino acid sequence shown in Table 73A, the amino acid sequence shown in Table 76A, the amino acid sequence shown in Table 76C, the amino acid sequence shown in Table 79A, the amino acid sequence shown in Table 79B, the amino acid sequence shown in Table 81, and a combination of two or more of said amino acid sequences.

14. The polynucleotide of claim 8, wherein the second multi-epitope construct comprises a nucleotide sequence selected from the group consisting of: the nucleotide sequence in Table 49C, the nucleotide sequence in Table 53A, the nucleotide sequence in Table 53B, the nucleotide sequence in Table 59, the nucleotide sequence in Table 61, the nucleotide sequence in Table 64A, the nucleotide sequence in Table 64B, the nucleotide sequence in Table 64C, the nucleotide sequence in Table 64D, the nucleotide sequence in Table 72B, the nucleotide sequence in Table 72F, the nucleotide sequence in Table 73B, the nucleotide sequence in Table 76B, the nucleotide sequence in Table 76D, the nucleotide sequence in Table 79A, the nucleotide sequence in Table 79B, the nucleotide sequence in Table 81, and a combination of two or more of said nucleotide sequences.

15. A vector comprising the polynucleotide of claim 8.

16. The vector of claim 15, wherein said vector is an expression vector.

17. A polypeptide comprising an amino acid sequence encoded by the polynucleotide of claim 1

18. The polypeptide of claim 17, which comprises an amino acid sequence selected from the group consisting of: the amino acid sequence shown in Table 50C, the amino acid sequence shown in Table 54A, the amino acid sequence shown in Table 54B, the amino acid sequence shown in Table 59, the amino acid sequence shown in Table 61, the amino acid sequence shown in Table 65A, the amino acid sequence shown in Table 65B, the amino acid sequence shown in Table 65C, the amino acid sequence shown in Table 65D, the amino acid sequence shown in Table 69, the amino acid sequence shown in Table 72A, the amino acid sequence shown in Table 72E, the amino acid sequence shown in Table 73A, the amino acid sequence shown in Table 76A, the amino acid sequence shown in Table 76C, the amino acid sequence shown in Table 79A, the amino acid sequence shown in Table 79B, the amino acid sequence shown in Table 81, and a combination of two or more of said amino acid sequences.

19. A composition comprising the polynucleotide of claim 1; and a carrier.

20. A cell comprising the polynucleotide of claim 1.

21. A method of inducing an immune response against human papillomavirus virus (HPV) in an individual in need thereof, comprising administering to said individual the composition of claim 19.

22. A method of making the polynucleotide of claim 1 comprising culturing the cell of claim 20, and recovering said polynucleotide.
